(19) United States
(12) Patent Application Publication
(10) Pub. No.: US 2025/0019453 A1
(43) Pub. Date: Jan. 16, 2025

(54) IL2RB/IL2G SYNTHETIC CYTOKINES

(71) Applicant: Synthekine, Inc., Menlo Park, CA (US)

(72) Inventors: Robert Kastelein, Menlo Park, CA (US); Deepti Rokkam, Menlo Park, CA (US); Patrick J. Lupardus, Menlo Park, CA (US); Rene De Waal Malefyt, Menlo Park, CA (US); Sandro Vivona, Menlo Park, CA (US)

(21) Appl. No.: 18/799,473

(22) Filed: Aug. 9, 2024

Related U.S. Application Data
(63) Continuation of application No. 18/017,836, filed on Jan. 24, 2023.
(60) Provisional application No. 63/061,562, filed on Aug. 5, 2020.
(60) Provisional application No. 63/078,745, filed on Sep. 15, 2020.
(60) Provisional application No. 63/135,884, filed on Jan. 11, 2021.
(60) Provisional application No. 63/136,095, filed on Jan. 11, 2021.
(60) Provisional application No. 63/136,098, filed on Jan. 11, 2021.

(51) Int. Cl. C07K 16/28; A61K 45/06

(52) U.S. Cl. CPC C07K 16/2866; A61K 45/06; C07K 2317/22; C07K 2317/24; C07K 2317/565; C07K 2317/569; C07K 2317/92; C07K 2319/00

(57) ABSTRACT

Provided herein are IL2R binding molecules that bind to IL2Rb and IL2Rg and comprise an IL2Rg sdAb and an anti-IL2Rg V_{H}H antibody.

### CROSS-REFERENCES TO RELATED APPLICATIONS

This application is a Continuation of U.S. application Ser. No. 18/017,836, filing date Jan. 24, 2023, which is national stage application under 35 U.S.C. 371 of PCT/US2021/044853, filed Aug. 5, 2021, which claims priority to U.S. Provisional Application No. 63/061,562, filed Aug. 5, 2020, U.S. Provisional Application No. 63/078,745, filed Sep. 15, 2020, U.S. Provisional Application No. 63/135,884, filed Jan. 11, 2021, U.S. Provisional Application No. 63/136,095, filed Jan. 11, 2021, and U.S. Provisional Application No. 63/136,098, filed Jan. 11, 2021, the disclosures of which are hereby incorporated by reference in their entirety for all purposes.

### SEQUENCE LISTING

The instant application contains a Sequence Listing which has been submitted electronically in XML format and is hereby incorporated by reference in its entirety. Said XML copy, created on Sep. 25, 2024, is named 106249-1458969-004230US_SL.xml and is 680,724 bytes in size.

### BACKGROUND OF THE DISCLOSURE

The present disclosure relates to synthetic mimetics of naturally occurring IL2 which are agonists of the IL2 receptor (IL2R).

In one embodiment, the IL2Rb is the human IL2Rb. The human CD122 (hCD122) is expressed as a 551 amino acid pre-protein, the first 26 amino acids comprising a signal sequence which is post-translationally cleaved in the mature 525 amino acid protein. Amino acids 27-240 (amino acids 1-214 of the mature protein) correspond to the extracellular domain, amino acids 241-265 (amino acids 225-239 of the mature protein) correspond to the transmembrane domain and amino acids 266-551 (amino acids 240-525 of the mature protein) correspond to the intracellular domain. UniProt Reference Number 14784. The canonical full length hIL2Rb precursor is a polypeptide having the amino acid sequence:

(SEQ ID NO: 1)
MAAPALSWRLPLLILLLPLATSWASAAVNGTSQFTCFYNSRANISCVWSQD
GALQDTSCQVHAWPDRRRWNQTCELLPVSQASWACNLILGAPDSQKLTTVD
IVTLRVLCREGVRWRVMAIQDFKPFENLRLMAPISLQVVHVETHRCNISWE
ISQASHYFERHLEFEARTLSPGHTWEEAPLLTLKQKQEWICLETLTPDTQY
EFQVRVKPLQGEFTTWSPWSQPLAFRTKPAALGKDTIPWLGHLLVGLSGAF
GFIILVYLLINCRNTGPWLKKVLKCNTPDPSKFFSQLSSEHGGDVQKWLSS
PFPSSSFSPGGLAPEISPLEVLERDKVTQLLLQQDKVPEPASLSSNHSLTS
CFTNQGYFFFHLPDALEIEACQVYFTYDPYSEEDPDEGVAGAPTGSSPQPL
QPLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGVS
FPWSRPPGQGEFRALNARLPLNTDAYLSLQELQGQDPTHLV

To generate sdAbs against hIL2Rb, the extracellular domain of the hIL2Rb protein was used as an immunogen The extracellular domain of the mature (lacking the signal sequence) hIL2Rb possesses the amino acid sequence:

(SEQ ID NO: 2)
AVNGTSQFTCFYNSRANISCVWSQDGALQDTSCQVHAWPDRRRWNQTCEL
LPVSQASWACNLILGAPDSQKLTTVDIVTLRVLCREGVRWRVMAIQDFKP
FENLRLMAPISLQVVHVETHRCNISWEISQASHYFERHLEFEARTLSPGH
TWEEAPLLTLKQKQEWICLETLTPDTQYEFQVRVKPLQGEFTTWSPWSQP
LAFRTKPAALGKDT

For purposes of the present disclosure, the numbering of amino acid residues of the human IL2Rb polypeptides as described herein is made in accordance with the numbering of this canonical sequence (UniProt ID: P14784. Amino acids 1-26 of SEQ ID NO:1 are identified as the signal peptide of the IL2Rb, amino acids 27-240 of SEQ ID NO:1 are identified as the extracellular domain, amino acids 241-265 of SEQ ID NO:1 are identified as the transmembrane domain, and amino acids 266-551 of SEQ ID NO:1 are identified as the intracellular domain.

In one embodiment, the IL2Rb is the murine IL2Rb. The murine CD122 (mCD122) is expressed as a 539 amino acid precursor, the first 26 amino acids comprising a signal sequence which is post-translationally cleaved to provide the mature 525 amino acid protein. Amino acids 27-240 (amino acids 1-214 of the mature protein) correspond to the extracellular domain, amino acids 241-268 (amino acids 225-242 of the mature protein) correspond to the transmembrane domain and amino acids 269-539 (amino acids 243-513 of the mature protein) correspond to the intracellular domain. The canonical full length mIL2Rb precursor protein including the signal sequence is a polypeptide of the amino acid sequence:

(SEQ ID NO: 3)
MATIALPWSLSLYVFLLLLATPWASAAVKNCSHLECFYNSRANVSCMWSH
EEALNVTTCHVHAKSNLRHWNKTCELTLVRQASWACNLILGSFPESQSLT
SVDLLDINVVCWEEKGWRRVKTCDFHPFDNLRLVAPHSLQVLHIDTQRCN
ISWKVSQVSHYIEPYLEFEARRRLLGHSWEDASVLSLKQRQQWLFLEMLI
PSTSYEVQVRVKAQRNNTGTWSPWSQPLTFRTRPADPMKEILPMSWLRYL
LLVLGCFSGFFSCVYILVKCRYLGPWLKTVLKCHIPDPSEFFSQLSSQHG
GDLQKWLSSPVPLSFFSPSGPAPEISPLEVLDGDSKAVQLLLLQKDSAPL
PSPSGHSQASCFTNQGYFFFHLPNALEIESCQVYFTYDPCVEEEVEEDGS
RLPEGSPHPPLLPLAGEQDDYCAFPPRDDLLLFSPSLSTPNTAYGGSRAP
EERSPLSLHEGLPSLASRDLMGLQRPLERMPEGDGEGLSANSSGEQASVP
EGNLHGQDQDRGQGPILTLNTDAYLSLQELQAQDSVHLI

To generate sdAbs against mIL2Rb, the extracellular domain of the mIL2Rb protein was used as an immunogen. The extracellular domain of the mature (lacking the signal sequence) hIL2Rb possesses the amino acid sequence (amino acids 27-240):

(SEQ ID NO: 4)
AVKNCSHLECFYNSRANVSCMWSHEEALNVTTCHVHAKSNLRHWNKTCEL
TLVRQASWACNLILGSFPESQSLTSVDLLDINVVCWEEKGWRRVKTCDFH
PFDNLRLVAPHSLQVLHIDTQRCNISWKVSQVSHYIEPYLEFEARRRLLG
HSWEDASVLSLKQRQQWLFLEMLIPSTSYEVQVRVKAQRNNTGTWSPWSQ
PLTFRTRPADPMKE

For purposes of the present disclosure, the numbering of amino acid residues of the murine IL2Rb polypeptides as described herein is made in accordance with the numbering of this canonical sequence (UniProt ID: P16297; Amino acids 1-26 of SEQ ID NO:3 are identified as the signal peptide of the IL2Rb, amino acids 27-240 of SEQ ID NO:3 are identified as the extracellular domain, amino acids 241-268 of SEQ ID NO:3 are identified as the transmembrane domain, and amino acids 269-539 of SEQ ID NO:3 are identified as the intracellular domain.

### IL2R′

The IL2Rg binding molecules of the present disclosure specifically bind to the extracellular domain of the IL2Rg.

### Human IL2Rg

The IL2Rg binding molecules of the present disclosure specifically bind to the extracellular domain of the IL2Rg (CD132). In one embodiment, the IL2Rg is the human IL2Rg. The canonical full length IL2Rg (including the signal peptide) is a polypeptide possessing the amino acid sequence:

(SEQ ID NO: 5)
MLKPSLPFTSLLFLQLPLLGVGLNTTILTPNGNEDTTADFFLTTMPTDSL
SVSTLPLPEVQCFVFNVEYMNCTWNSSSEPQPTNLTLHYWYKNSDNDKVQ
KCSHYLFSEEITSGCQLQKKEIHLYQTFVVQLQDPREPRRQATQMLKLQN
LVIPWAPENLTLHKLSESQLELNWNNRFLNHCLEHLVQYRTDWDHSWTEQ
SVDYRHKFSLPSVDGQKRYTFRVRSRFNPLCGSAQHWSEWSHPIHWGSNT
SKENPFLFALEAVVISVGSMGLIISLLCVYFWLERTMPRIPTLKNLEDLV
TEYHGNFSAWSGVSKGLAESLQPDYSERLCLVSEIPPKGGALGEGPGASP
CNQHSPYWAPPCYTLKPET.

To generate sdAbs against the human IL2Rg, the extracellular domain of the hIL2Rg protein was used as an immunogen. The extracellular domain of the mature (lacking the signal sequence) hIL2Rg possesses the amino acid sequence:

(SEQ ID NO: 6)
LNTTILTPNGNEDTTADFFLTTMPTDSLSVSTLPLPEVQCFVFNVEYMNC
TWNSSSEPQPTNLTLHYWYKNSDNDKVQKCSHYLFSEEITSGCQLQKKEI
HLYQTFVVQLQDPREPRRQATQMLKLQNLVIPWAPENLTLHKLSESQLEL
NWNNRFLNHCLEHLVQYRTDWDHSWTEQSVDYRHKFSLPSVDGQKRYTFR
VRSRFNPLCGSAQHWSEWSHPIHWGSNTSKENPFLFALEA

For purposes of the present disclosure, the numbering of amino acid residues of the human IL2Rg (hIL2Rg) polypeptides as described herein is made in accordance with the numbering of this canonical sequence (UniProt ID: 31785; SEQ ID NO: 5). Amino acids 1-22 of SEQ ID NO:5 are identified as the signal peptide of hIL2Rg, amino acids 23-262 of SEQ ID NO: 5 are identified as the extracellular domain, amino acids 263-283 SEQ ID NO: 5 are identified as the transmembrane domain, and amino acids 284-269 of SEQ ID NO:5 are identified as the intracellular domain.

### Murine IL2Rg

In one embodiment, the IL2Rg is the murine IL2Rg. The murine CD132 (mCD132) is expressed as a 369 amino acid precursor, the first 22 amino acids comprising a signal sequence which is post-translationally cleaved to provide the mature 353 amino acid protein. Amino acids 23-263 (amino acids 1-214 of the mature protein) correspond to the extracellular domain, amino acids 264-284 (amino acids 242-266 of the mature protein) correspond to the transmembrane domain and amino acids 285-369 (amino acids 263-347 of the mature protein) correspond to the intracellular domain. The canonical full length mIL2Rg precursor protein including the signal sequence is a polypeptide of the amino acid sequence:

(SEQ ID NO: 7)
MLKLLLSPRSFLVLQLLLLRAGWSSKVLMSSANEDIKADLILTSTAPEHL
SAPTLPLPEVQCFVFNIEYMNCTWNSSSEPQATNLTLHYRYKVSDNNTFQ
ECSHYLFSKEITSGCQIQKEDIQLYQTFVVQLQDPQKPQRRAVQKLNLQN
LVIPRAPENLTLSNLSESQLELRWKSRHIKERCLQYLVQYRSNRDRSWTE
LIVNHEPRFSLPSVDELKRYTFRVRSRYNPICGSSQQWSKWSQPVHWGSH
TVEENPSLFALEAVLIPVGTMGLIITLIFVYCWLERMPPIPPIKNLEDLV
TEYQGNFSAWSGVSKGLTESLQPDYSERFCHVSEIPPKGGALGEGPGGSP
CSLHSPYWPPPCYSLKPEA

In one embodiment, the IL2Rg is the murine IL2Rg. The murine CD132 (mCD132) is expressed as a 369 amino acid precursor, the first 22 amino acids comprising a signal sequence which is post-translationally cleaved to provide the mature 353 amino acid protein. Amino acids 23-263 (amino acids 1-214 of the mature protein) correspond to the extracellular domain, amino acids 264-284 (amino acids 242-266 of the mature protein) correspond to the transmembrane domain and amino acids 285-369 (amino acids 263-347 of the mature protein) correspond to the intracellular domain. The canonical full length mIL2Rg precursor protein including the signal sequence is a polypeptide of the amino acid sequence:

To generate sdAbs against mIL2Rg, the extracellular domain of the mIL2Rg protein was used as an immunogen. The extracellular domain of the mature (lacking the signal sequence) hIL2Rg possesses the amino acid sequence (amino acids 23-263):

(SEQ ID NO: 8)
WSSKVLMSSANEDIKADLILTSTAPEHLSAPTLPLPEVQCFVFNIEYMNC
TWNSSSEPQATNLTLHYRYKVSDNNTFQECSHYLFSKEITSGCQIQKEDI
QLYQTFVVQLQDPQKPQRRAVQKLNLQNLVIPRAPENLTLSNLSESQLEL
RWKSRHIKERCLQYLVQYRSNRDRSWTELIVNHEPRFSLPSVDELKRYTF
RVRSRYNPICGSSQQWSKWSQPVHWGSHTVEENPSLFALEA

For purposes of the present disclosure, the numbering of amino acid residues of the murine IL2Rg polypeptides as described herein is made in accordance with the numbering of this canonical sequence (UniProt ID: P34902). Amino acids 1-22 of SEQ ID NO:7 are identified as the signal peptide of the IL2Rg, amino acids 23-263 of SEQ ID NO:7 are identified as the extracellular domain, amino acids 264-284 of SEQ ID NO:7 are identified as the transmembrane domain, and amino acids 285-369 of SEQ ID NO:7 are identified as the intracellular domain.

### IL2

IL2 is a monomeric polypeptide which is an agonist of the IL2R. The amino acid sequence for human IL2 is set forth under UniProt ID: P60568 and is set forth below as SEQ ID NO: 9 below:

(SEQ ID NO: 9)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT

The amino acid sequence of mature murine IL2 is set forth under UniProt ID: P04351 and is set forth below as SEQ ID NO: 10:

(SEQ ID NO: 10)
MYSMQLASCVTLTLVLLVNSAPTSSSTSSSTAEAQQQQQQQQQQQQHLEQ
LLMDLQELLSRMENYRNLKLPRMLTFKFYLPKQATELKDLQCLEDELGPL
RHVLDLTQSKSFQLEDAENFISNIRVTVVKLKGSDNTFECQFDDESATVV
DFLRRWIAFCQSIISTSPQ

IL2 is a pluripotent cytokine which is produced by antigen activated T cells. IL2 exerts a wide spectrum of effects on the immune system and plays important roles in regulating both immune activation, suppression and homeostasis. IL2 promotes the proliferation and expansion of activated T lymphocytes, induces proliferation and activation of naïve T cells, potentiates B cell growth, and promotes the proliferation and expansion of NK cells. Human interleukin 2 (IL2) is a 4 alpha-helix bundle cytokine of 133 amino acids. IL2 is a member of the IL2 family of cytokines which includes IL2, IL-4, IL-7, IL 9, IL-15 and IL21.

### IL2/IL2 Receptor Interaction:

Monomeric IL2 forms a complex with both the trimeric “high affinity” form of the IL2 receptor and the dimeric intermediate affinity receptor (Wang, et al. (2005) Science 310:159-1163) through binding to the extracellular domains of the receptor components expressed on the cell surface. The binding of IL2 to CD25 induces a conformational change in IL2 facilitating increased binding to CD122. IL2 mutants, mimicking the CD25 binding-induced conformational change demonstrate increased binding to CD122 (Levin, et al. (2012) Nature 484(7395): 529-533). The association of CD132 provides formation of the dimeric intermediate-affinity or trimeric high-affinity receptor complexes which are associated with intracellular signaling. In addition to providing intracellular signaling via the JAK/STAT pathway (e.g. phosphorylation of STAT5) and other cellular systems, the interaction of hIL2 with the hIL2 high affinity trimeric receptor on a cell initiates a process by which CD122 is internalized, the membrane bound form of CD25 is released from the activated cell as a soluble protein (referred to as “soluble CD25” or “sCD25”) as well as triggering the release of IL2 endogenously produced by the activated cell which is capable of acting in an autocrine and/or paracrine fashion.

CD25 (also referred to interchangeably herein as IL2Ra and IL2Ra) is a 55 kD polypeptide that is constitutively expressed in Treg cells and inducibly expressed on other T cells in response to activation. hIL2 binds to hCD25 with a K_{d} of approximately 10⁻⁸M. CD25 is also referred to in the literature as the “low affinity” IL2 receptor. The human CD25 (“hCD25”) is expressed as a 272 amino acid pre-protein comprising a 21 amino acid signal sequence which is post-translationally removed to render a 251 amino acid mature protein. Amino acids 22-240 (amino acids 1-219 of the mature protein) correspond to the extracellular domain. Amino acids 241-259 (amino acids 220-238 of the mature protein) correspond to transmembrane domain. Amino acids 260-272 (amino acids 239-251 of the mature protein) correspond to intracellular domain. The intracellular domain of CD25 is comparatively small (13 amino acids) and has not been associated with any independent signaling activity. The IL2/CD25 complex has not been observed to produce a detectable intracellular signaling response. Human CD25 nucleic acid and protein sequences may be found as Genbank accession numbers NM__000417 and NP_0004Q8 respectively.

IL2 is a pluripotent cytokine which is produced by antigen activated T cells. IL2 exerts a wide spectrum of effects on the immune system and plays important roles in regulating both immune activation, suppression and homeostasis. IL2 promotes the proliferation and expansion of activated T lymphocytes, induces proliferation and activation of naïve T cells, potentiates B cell growth, and promotes the proliferation and expansion of NK cells. Human interleukin 2 (IL2) is a 4 alpha-helix bundle cytokine of 133 amino acids. IL2 is a member of the IL2 family of cytokines which includes IL2, IL-4, IL-7, IL 9, IL-15 and IL21.

### IL2 Receptor Expression on Various Cell Types

The IL2 receptors are expressed on the surface of most lymphatic cells, in particular on T cells, NK cells, and B cells, but the expression level is variable and is dependent on a variety of factors include the activation stage of the cell. Inactive T cells and NK cells express almost exclusively express the intermediate-affinity dimeric IL2 receptor, consisting of the two receptor subunits, CD122 and CD132 and demonstrates comparatively low responsiveness to IL2 since they predominantly express the intermediate affinity CD122/CD132 complex which has comparatively low affinity for IL2 relative to the CD25/CD122/CD132 high affinity receptor. In contrast, activated T cells and regulatory T cells express the trimeric high-affinity IL2 receptor consisting of CD25, CD122 and CD132. TCR activated T cells (i.e., so called “antigen experienced” T cells) express the high-affinity trimeric IL2 receptor. T cells, including tumor infiltrating T cells (“TILs”) and tumor recognizing cells, upregulate CD25 and CD122 upon receiving a T cell receptor (TCR) signal (Kalia, et al. (2010) Immunity 32(1): 91-103. The upregulation of CD25 and CD122 receptor in response to receiving a T cell receptor (TCR) signal renders the antigen activated T cell highly sensitive to the IL2 cytokine. Although, Tregs constitutively express CD25, and therefore express the high affinity trimeric IL2 receptor, TCR-activated T cells express higher levels of the trimeric receptor than regulatory T cells. As a consequence, the expansion of antigen activated T cells in antigen-challenged hosts significantly outpaces the expansion of Tregs. (Humblet-Baron, et al. (2016) J Allergy Clin Immunol 138(1): 200-209 e208).

Recombinant hIL2 (sold under the trademark Proleukin) is indicated for the treatment of human adults with metastatic melanoma and metastatic renal cell carcinoma. Therapeutic application of High Dose hIL2 (HD-hIL2) induces tumor rejection in highly immune infiltrated melanomas and renal cell carcinomas (Atkins, et al. (1999) J Clin Oncol 17(7):2105-2116). However, HD-hIL2 therapy is associated with severe dose limiting toxicity, including impaired neutrophil function, fever, hypotension, diarrhea and requires expert management. Dutcher, et al. (2014) J Immunother Cancer 2(1): 26. HD-hIL2 treatment activates most lymphatic cells, including naïve T cells and NK cells, which predominantly express the intermediate affinity CD122/CD132 dimeric receptor and CD25+ regulatory T cells (Tregs), which express the high affinity trimeric receptor (CD25/CD122/CD132). HD-hIL2 monotherapy may also induce generalized capillary leak syndrome which can lead to death. This limits the use of HD-IL2 therapy to mostly younger, very healthy patients with normal cardiac and pulmonary function. HD-IL2 therapy is typically applied in the hospital setting and frequently requires admission to an intensive care unit.

Clinical experience demonstrates that HD-IL2 treatment activates naïve T cells and NK cells, which predominantly express the intermediate affinity receptor as well as CD25+ regulatory T cells (Tregs) which mediate the activity of CD8+ T cells. Due to their constitutive expression of CD25, Tregs are particularly sensitive to IL2. To avoid preferential activation of Tregs, IL2 variants have been developed and introduced into clinical development, which are designed to avoid binding to CD25 and possess enhanced binding to the intermediate affinity CD122/CD132 receptor to activate NK cells and quiescent CD8+ T cells. Such IL2 muteins are often referred to in the literature as “non-α-IL2” or “β/γ-biased IL2” muteins. However, such “non-α-IL2” or “β/γ-biased IL2”, by virtue of their reduced binding to CD25, also avoid binding to the antigen activated T cells which have been identified as the primary mediators of anti-tumor T cell response (Peace, D. J. and Cheever, M. A. (1989) J Exp Med 169(1):161-173).

Additionally, preclinical experiments have implicated NK cells as the dominant mechanism for IL2 mediated acute toxicity. Assier E, et al. (2004) J Immunol 172:7661-7668. As NK cells express the intermediate affinity (CD122/CD132; β/γ-) IL2 receptor, the nature of such β/γ-IL2 muteins is to enhance the proliferation of such NK cells which may lead to enhanced toxicity. Additionally, although Tregs are associated with down-regulation of CD8+ T cells, Tregs have also been shown to limit the IL2 mediated off-tumor toxicity (Li, et al. (2017) Nature Communications 8(1):1762). Although nitric oxide synthase inhibitors have been suggested to ameliorate the symptoms of VLS, the common practice when VLS is observed is the withdrawal of IL2 therapy. To mitigate the VLS associated with HD IL2 treatment, low-dose IL2 regimens have been tested in patients. While low dose IL2 treatment regimens do partially mitigate the VLS toxicity, this lower toxicity was achieved at the expense of optimal therapeutic results in the treatment of neoplasms.

Considering the pluripotent effects of hIL2 and its demonstrated ability to modulate the activities of a wide variety of cell types associated with human disease, the search for IL2 muteins that retain certain desirable features of the native molecule while minimizing undesirable features remain an active area of research with multiple IL2 muteins in clinical development nearly 40 years after its initial discovery.

### SUMMARY OF THE DISCLOSURE

The present disclosure provides compositions useful in the pairing of cellular receptors to generate desirable effects useful in treatment of disease in mammalian subjects.

The present disclosure provides binding molecules that comprise a first domain that binds to IL2Rb of the IL2R receptor and a second domain that binds to IL2Rg of the IL2R receptor, such that upon contacting with a cell expressing IL2Rb and IL2Rg, the IL2R binding molecule causes the functional association of IL2Rb and IL2Rg, thereby resulting in functional dimerization of the receptors and downstream signaling.

Several advantages flow from the binding molecules described herein. As discussed above, the use of IL2 as a therapeutic in mammalian, particularly human, subjects, it may also trigger a number of adverse and undesirable effects by a variety of mechanisms including the presence of IL2Rb and IL2Rg on other cell types. The binding to IL2Rb and IL2Rg on the other cell types may result in undesirable effects and/or undesired signaling on cells expressing IL2Rb and IL2Rg.

The present disclosure is directed to methods and composition that facilitate the modulation of the multiple effects characteristic of IL2 to provide agents having the activation and/or proliferative response of IL2 signaling in a desired cell population or tissue subtype, while exhibiting substantially reduced signaling activity and/or intracellular signaling.

In some embodiments, the IL2R binding molecules described herein are partial agonists of the IL2R. In some embodiments, the binding molecules described herein are designed such that the IL2R binding molecules are full agonists. In some embodiments, the IL2R binding molecules described herein are designed such that the IL2R binding molecules are super agonists.

In some embodiments, the IL2R binding molecules provide substantial IL2 intracellular signaling on the desired cell types, while providing significantly reduced IL2 signaling relative to wild-type IL2 on other undesired cell types. The architecture of the binding molecules of the present disclosure provide multiple means for the modulation of the signaling associated with the dimerization of IL2Rb and IL2g. In some embodiments, the selective may be achieve by selection of binding molecules having differing affinities or causing different Eₘₐₓ for IL2Rb and IL2Rg, as compared to the affinity of IL2 for IL2Rb and IL2Rg. Because different cell types respond to the binding of ligands to its cognate receptor with different sensitivity, modulating the affinity of the dimeric ligand (or its individual binding moieties) for the IL2 receptor relative to wild-type IL2 binding facilitates the stimulation of desired activities while reducing undesired activities on non-target cells.

The present disclosure provides binding molecules that are agonists of the IL2R receptor, the binding molecule comprising:
a first single domain antibody (sdAb) that specifically binds to the extracellular domain of IL2Rb of the IL2R (an “IL2Rb sdAb”), and
a second single domain antibody that specifically binds to extracellular domain IL2Rg of the IL2R (an “IL2Rg sdAb”),
wherein the IL2Rb sdAb and IL2Rg sdAb are stably associated, and wherein contacting a cell expressing IL2Rb and IL2Rg with an effective amount of the binding molecule results in the dimerization of IL2Rb and IL2Rg, and results in intracellular signaling characteristic of the IL2R receptor when activated by its cognate ligand, IL2. In some embodiments, one or both of the sdAbs is a scFv. In some embodiments, one or both of the sdAbs is a VHH.

In some embodiments, one sdAb of the binding molecule is an scFv and the other sdAb is a VHH.

In some embodiments, one sdAb of the binding molecule is an scFv and the other sdAb is a VHH.

In some embodiments, the first and second sdAbs are covalently bound via a chemical linkage.

In some embodiments, the first and second sdAbs are provided as single continuous polypeptide.

In some embodiments, the first and second sdAbs are provided as single continuous polypeptide optionally comprising an intervening polypeptide linker between the amino acid sequences of the first and second sdAbs.

In some embodiments, the binding molecule optionally comprising a linker, is optionally expressed as a fusion protein with an additional amino acid sequence. In some embodiments, the additional amino acid sequence is a purification handle such as a chelating peptide or an additional protein such as a subunit of an Fc molecule.

The disclosure also provides an expression vector comprising a nucleic acid encoding the bispecific binding molecule operably linked to one or more expression control sequences. The disclosure also provides an isolated host cell comprising the expression vector expression vector comprising a nucleic acid encoding the bispecific binding molecule operably linked to one or more expression control sequences functional in the host cell.

In another aspect, the disclosure provides a pharmaceutical composition comprising the IL2R binding molecule described herein and a pharmaceutically acceptable carrier.

In another aspect, the disclosure provides a method of treating an autoimmune or inflammatory disease, disorder, or condition or a viral infection in a subject in need thereof, comprising administering to the subject a therapeutically effective amount of an IL2R binding molecule described herein or a pharmaceutical composition described herein.

In another aspect, the disclosure provides a method of treating neoplastic disease or disorder in a subject in need thereof, comprising administering to the subject a therapeutically effective amount of an IL2R binding molecule described herein or a pharmaceutical composition described herein.

### BRIEF DESCRIPTION OF THE FIGURES

FIG. 1 of the attached drawings provides a schematic representation of one embodiment of the binding molecule of the present disclosure comprising a first single domain antibody (1) and a second single domain antibody (3) and a linker (2) depicted as interacting with a cell membrane (10) associated heterodimeric receptor comprising a first receptor subunit comprising an extracellular domain (4), and transmembrane domain (5) and an intracellular domain (6) interaction of a binding molecule and a second first receptor subunit comprising an extracellular domain (7), and transmembrane domain (8) and an intracellular domain (9) wherein the intracellular domain of the first receptor (6) and the intracellular domain of the second receptor (9) on of a binding molecule are within a proximal distance (11).

FIG. 2 of the attached drawings provides a schematic representation of two illustrative configurations of binding molecules of the present disclosure. Panel A provides a schematic representation of an illustrative single polypeptide chain binding molecule comprising, from amino to carboxy, a first single domain antibody (1) and a second single domain antibody (3) and a linker (2). Panel B provides a schematic representation of a binding molecule comprising a first single domain antibody (1) and a second single domain antibody (3) and a linker (2) and a knob-into-hole Fc domain comprising a first subunit which is a Fc knob (13) and a second subunit which is a Fc hole (14) wherein the single domain antibody is stably associated with the Fc domain via a IgG hinge sequence (12).

FIG. 2 of the attached drawings provides a schematic representation of two illustrative configurations of binding molecules of the present disclosure. Panel A provides a schematic representation of an illustrative binding molecule comprising a first single domain antibody (1) and a second single domain antibody (3) and a linker (2). Panel B provides a schematic representation of a binding molecule comprising two polypeptide chains, the first polypeptide chain comprising (from amino to carboxy) a first single domain antibody (1), a linker sequence, a second single domain antibody (3), an IgG hinge sequence (12) and an Fc knob domain (13) and a second polypeptide comprising an Fc hole (14) wherein the first and second polypeptides are in stable association via the interaction of the knob-into-hole Fc domain.

FIG. 3 of the attached drawings provides a schematic representations of two illustrative configurations of binding molecules of the present disclosure. Panel A provides a schematic representation of an illustrative binding molecule construct comprising two binding molecules each attached to a subunit of a knob-into-hole Fc domain, the construct comprising two polypeptide chains, the first polypeptide chain comprising, from amino to carboxy, a first single domain antibody (1), a linker (2) and a second single domain antibody (3), a IgG hinge sequence (12) and a Fc knob subunit (13) and a second polypeptide chain comprising, from amino to carboxy, a first single domain antibody (1), a linker (2) and a second single domain antibody (3), a IgG hinge sequence (12) and a Fc hole subunit (14) wherein the first and second polypeptides are in stable associate via the interaction of the knob-into-hole Fc domain. Panel B provides schematic representation of a an alternative arrangement of a binding molecule construct comprising two polypeptides a first polypeptide chain comprising, from amino to carboxy, a first single domain antibody (1), a linker (2) and a second single domain antibody (3), an IgG hinge sequence (12) and a Fc knob subunit (13) and a second polypeptide chain comprising, from amino to carboxy, a first second domain antibody (3), a linker (2) and a first single domain antibody (1), a IgG hinge sequence (12) and a Fc hole subunit (14), wherein the first and second polypeptides are in stable association via the interaction of the knob-into-hole Fc domain.

FIG. 4, Panel A provides alternative schematic representations of configurations of the binding molecules of the present disclosure where one single domain antibody is attached to each subunit of a knob-into-hole Fc domain comprising two polypeptides, the first polypeptide comprising from amino to carboxy, a first single domain antibody (1), an IgG hinge sequence (12) and a Fc knob subunit (13), the second polypeptide comprising from amino to carboxy, a second single domain antibody (3), an IgG hinge sequence (12) and a Fc hole subunit (13), wherein the first and second single domain antibodies are in stable associate via the interaction of the knob-into-hole Fc domain.

FIG. 4, Panel B provides a schematic representations of a binding molecule the binding domains are single domain antibodies associated via transition metal coordinate covalent complex. As illustrated, the binding molecules comprises two polypeptide subunits: the first subunit comprising a first single domain antibody (1) is attached via a first linker (15) to a first chelating peptide (17) and second subunit comprising a second single domain antibody (3) is attached via a second linker (16) to a second chelating peptide (18), wherein the first chelating peptide (17) and second chelating peptide (18) form a coordinate covalent complex with a single transition metal ion (“M”). The transition metal ion may be in a kinetically labile or kinetically inert oxidation state.

FIG. 5 of the attached drawings provides data with respect to IL2R binding molecules of the present disclosure on the induction of IFN gamma in NK cells measured by luminescent. This data illustrates that the IL2 binding molecules by varying the sdAb components may provide substantial variations in activity significantly greater than wt IL2 in some instances.

FIG. 6 of the attached drawings provides data from the evaluation of T cell outgrown on PBMCs isolate from two separate donors. As can be seen the data, the IL2R binding molecules enables selective T cell proliferation activity with respect to NK cells, even though the NK cells express the b/g receptors. This demonstrates that variation in receptor binding affinity can be used to modulate the activity of the IL2R binding molecules in selective cell types.

### DETAILED DESCRIPTION OF THE INVENTION

To facilitate the understanding of present disclosure, certain terms and phrases are defined below as well as throughout the specification. The definitions provided herein are non-limiting and should be read in view of the knowledge of one of skill in the art would know.

Before the present methods and compositions are described, it is to be understood that this invention is not limited to a particular method or composition described, as such may, of course, vary. It is also to be understood that the terminology used herein is for the purpose of describing embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); AA or aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=once weekly; QM=once monthly; HPLC=high performance liquid chromatography; BW=body weight; U=unit; ns=not statistically significant; PBS=phosphate-buffered saline; PCR=polymerase chain reaction; HSA=human serum albumin; MSA=mouse serum albumin; DMEM=Dulbeco's Modification of Eagle's Medium; EDTA=ethylenediaminetetraacetic acid.

It will be appreciated that throughout this disclosure reference is made to amino acids according to the single letter or three letter codes. For the reader's convenience, the single and three letter amino acid codes are provided in Table 1 below:

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

### Definitions

Unless otherwise indicated, the following terms are intended to have the meaning set forth below. Other terms are defined elsewhere throughout the specification.

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand.

ACT Cell Product: As used herein, the terms “cell product”, “adoptive cell transfer product” or “ACT cell product” are used interchangeably herein to refer to a population of cells comprising immune cells that have been manipulated ex vivo to be enriched for a desired subpopulation of immune cells for administration to a subject in need of treatment. One example of ACT cell product is a TIL cell product wherein the immune cells that have been manipulated ex vivo are lymphocytes isolated from a tissue sample of a subject suffering from a neoplastic disease. The tissue sample used as a source of the immune cells may be a neoplastic lesion or tumor mass for preparation of a TIL cell product. Alternatively, TILs may be isolated from circulating blood.

Activity: As used herein, the term “activity” is used with respect to a molecule to describe a property of the molecule with respect to a test system (e.g. an assay) or biological or chemical property (e.g. the degree of binding of the molecule to another molecule) or of a physical property of a material or cell (e.g. modification of cell membrane potential). Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT5 phosphorylation]/[mg protein], [T-cell proliferation]/[mg protein], plaque forming units (pfu), etc. As used herein, the term “proliferative activity” refers to an activity that promotes cell proliferation and replication.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g. an ortholog, an IL2 ortholog, an engineered cell expressing an orthogonal receptor, an engineered cell expressing an orthogonal IL2 receptor, a CAR-T cell expressing an orthogonal IL2 receptor, a chemotherapeutic agent, an antibody, or a pharmaceutical formulation comprising one or more of the foregoing). Administration of an agent may be achieved through any of a variety of art recognized methods including but not limited to the topical administration, intravascular injection (including intravenous or intraarterial infusion), intradermal injection, subcutaneous injection, intramuscular injection, intraperitoneal injection, inhalation and the like. The term “administration” includes contact of an agent to the cell, tissue or organ as well as the contact of an agent to a fluid, where the fluid is in contact with the cell, tissue or organ.

Affinity: As used herein the term “affinity” refers to the degree of specific binding of a first molecule (e.g., a ligand) to a second molecule (e.g., a receptor) and is measured by the binding kinetics expressed as K_{d}, a ratio of the dissociation constant between the molecule and its target (K_{off}) and the association constant between the molecule and its target (Kₒₙ).

Agonist: As used herein, the term “agonist” refers to a first agent that specifically binds a second agent (“target”) and interacts with the target to cause or promote an increase in the activation of the target. In some instances, agonists are activators of receptor proteins that modulate cell activation, enhance activation, sensitize cells to activation by a second agent, or up-regulate the expression of one or more genes, proteins, ligands, receptors, biological pathways, that may result in cell proliferation or pathways that result in cell cycle arrest or cell death such as by apoptosis. In some embodiments, an agonist is an agent that binds to a receptor and alters the receptor state, resulting in a biological response. The response mimics the effect of the endogenous activator of the receptor. The term “agonist” includes partial agonists, full agonists and superagonists. An agonist may be described as a “full agonist” when such agonist which leads to a substantially full biological response (i.e., the response associated with the naturally occurring ligand/receptor binding interaction) induced by the receptor under study, or a partial agonist. In contrast to agonists, antagonists may specifically bind to a receptor but do not result in the signal cascade typically initiated by the receptor and may modify the actions of an agonist at that receptor. Inverse agonists are agents that produce a pharmacological response that is opposite in direction to that of an agonist. A “superagonist” is a type of agonist that is capable of producing a maximal response greater than the endogenous agonist for the target receptor, and thus has an activity of more than 100% of the native ligand. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay.

Antagonist: As used herein, the term “antagonist” or “inhibitor” refers to a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist.

Inhibitors are molecules that decrease, block, prevent, delay activation, inactivate, desensitize, or down-regulate, e.g., a gene, protein, ligand, receptor, biological pathway, or cell.

Antibody: As used herein, the term “antibody” refers collectively to: (a) glycosylated and non-glycosylated immunoglobulins (including but not limited to mammalian immunoglobulin classes IgG1, IgG2, IgG3 and IgG4) that specifically binds to a target molecule and (b) immunoglobulin derivatives including but not limited to IgG(1-4)deltaC_{H}2, F(ab′)₂, Fab, ScFv, V_{H}, V_{L}, tetrabodies, triabodies, diabodies, dsFv, F(ab′)₃, scFv-Fc and (scFv)₂ that competes with the immunoglobulin from which it was derived for binding to the target molecule. The term antibody is not restricted to immunoglobulins derived from any particular mammalian species and includes murine, human, equine, and camelids antibodies (e.g., human antibodies). The term “antibody” encompasses antibodies isolatable from natural sources or from animals following immunization with an antigen and as well as engineered antibodies including monoclonal antibodies, bispecific antibodies, trispecific, chimeric antibodies, humanized antibodies, human antibodies, CDR-grafted, veneered, or deimmunized (e.g., to remove T-cell epitopes) antibodies. The term “human antibody” includes antibodies obtained from human beings as well as antibodies obtained from transgenic mammals comprising human immunoglobulin genes such that, upon stimulation with an antigen the transgenic animal produces antibodies comprising amino acid sequences characteristic of antibodies produced by human beings. The term “antibody” should not be construed as limited to any particular means of synthesis and includes naturally occurring antibodies isolatable from natural sources and as well as engineered antibody molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries.

Binding molecule: As used herein, the term “binding molecule” refers to a molecule that can bind to the extracellular domain of two cell surface receptors. In some embodiments, a binding molecule specifically binds to two different receptors (or domains or subunits thereof) such that the receptors (or domains or subunits) are maintained in proximity to each other such that the receptors (or domains or subunits), including domains thereof (e.g., intracellular domains) interact with each other and result in downstream signaling.

CDR: As used herein, the term “CDR” or “complementarity determining region” is intended to mean the non-contiguous antigen combining sites found within the variable region of both heavy and light chain immunoglobulin polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. In the context of the present disclosure, the numbering of the CDR positions is provided according to Kabat numbering conventions.

Comparable: As used herein, the term “comparable” is used to describe the degree of difference in two measurements of an evaluable quantitative or qualitative parameter. For example, where a first measurement of an evaluable quantitative parameter and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

Effective Concentration (EC): As used herein, the terms “effective concentration” or its abbreviation “EC” are used interchangeably to refer to the concentration of an agent (e.g., an hIL2 mutein) in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC₄₀, EC₅₀, etc.) the subscript refers to the percentage of the Emax of the biological observed at that concentration.

For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results in the maximal (100%) response of a measurable parameter in response to such an agent. Similarly, the term EC₅₀ (which is commonly used in the field of pharmacodynamics) refers to the concentration of an agent sufficient to result in the half-maximal (50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect. The EC of a particular effective concentration of a test agent may be abbreviated with respect to the particular parameter and test system.

Extracellular Domain: As used herein the term “extracellular domain” or its abbreviation “ECD” refers to the portion of a cell surface protein (e.g. a cell surface receptor) which is outside of the plasma membrane of a cell. The term “ECD” may include the extra-cytoplasmic portion of a transmembrane protein or the extra-cytoplasmic portion of a cell surface (or membrane associated protein).

Identity: As used herein, the term “percent (%) sequence identity” or “substantially identical” used in the context of nucleic acids or polypeptides, refers to a sequence that has at least 50% sequence identity with a reference sequence. Alternatively, percent sequence identity can be any integer from 50% to 100%. In some embodiments, a sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined with BLAST using standard parameters, as described below. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned. Algorithms that are suitable for determining percent sequence identity and sequence similarity are the BLAST and BLAST 2.0 algorithms, which are described in Altschul et al. (1990) J Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) website. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)). The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10-5, and most preferably less than about 10⁻²⁰.

Intracellular Signaling: As used herein, the terms “intracellular signaling” and “downstream signaling” are used interchangeably to refer to the cellular signaling process that is caused by the interaction of the intracellular domains (ICDs) of two or more cell surface receptors that are in proximity of each other. In receptor complexes via the JAK/STAT pathway, the association of the ICDS of the receptor subunits brings the JAK domains of the ICDs into proximity which initiates a phosphorylation cascade in which STAT molecules are phosphorylated and translocate to the nucleus associating with particular nucleic acid sequences resulting in the activation and expression of particular genes in the cell. The binding molecules of the present disclosure provide intracelluar signaling characteristic of the IL2R receptor when activated by its natural cognate IL2. To measure downstream signaling activity, a number of methods are available. For example, in some embodiments, one can measure JAK/STAT signaling by the presence of phosphorylated receptors and/or phosphorylated STATs. In other embodiments, the expression of one or more downstream genes, whose expression levels can be affected by the level of downstream signaling caused by the binding molecule, can also be measured.

Ligand: As used herein, the term “ligand” refers to a molecule that exhibits specific binding to a receptor and results in a change in the biological activity of the receptor so as to effect a change in the activity of the receptor to which it binds. In one embodiment, the term “ligand” refers to a molecule, or complex thereof, that can act as an agonist or antagonist of a receptor. As used herein, the term “ligand” encompasses natural and synthetic ligands. “Ligand” also encompasses small molecules, e.g., peptide mimetics of cytokines and peptide mimetics of antibodies. The complex of a ligand and receptor is termed a “ligand-receptor complex.”

As used herein, the term “linker” refers to a linkage between two elements, e.g., protein domains. A linker can be a covalent bond or a peptide linker. The term “bond” refers to a chemical bond, e.g., an amide bond or a disulfide bond, or any kind of bond created from a chemical reaction, e.g., chemical conjugation. The term “peptide linker” refers to an amino acid or polypeptide that may be employed to link two protein domains to provide space and/or flexibility between the two protein domains.

Modulate: As used herein, the terms “modulate”, “modulation” and the like refer to the ability of a test agent to affect a response, either positive or negative or directly or indirectly, in a system, including a biological system or biochemical pathway.

Multimerization: As used herein, the term “multimerization” refers to two or more cell surface receptors, or domains or subunits thereof, being brought in close proximity to each other such that the receptors, or domains or subunits thereof, can interact with each other and cause intracellular signaling.

N-Terminus: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. The terms “immediately N-terminal” or “immediately C-terminal” are used to refer to a position of a first amino acid residue relative to a second amino acid residue where the first and second amino acid residues are covalently bound to provide a contiguous amino acid sequence.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the

Operably Linked: The term “operably linked” is used herein to refer to the relationship between nucleic acid sequences encoding differing functions when combined into a single nucleic acid sequence that, when introduced into a cell, provides a nucleic acid which is capable of effecting the transcription and/or translation of a particular nucleic acid sequence in a cell. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, certain genetic elements such as enhancers need not be contiguous with respect to the sequence to which they provide their effect.

Partial Agonist: As used herein, the term “partial agonist” refers to a molecule that specifically binds that bind to and activate a given receptor but possesses only partial activation of the receptor relative to a full agonist. Partial agonists may display both agonistic and antagonistic effects. For example, when both a full agonist and partial agonist are present, the partial agonist acts as a competitive antagonist by competing with the full agonist for the receptor binding resulting in a net decrease in receptor activation relative to the contact of the receptor with the full agonist in the absence of the partial agonist. Clinically, partial agonists can be used to activate receptors to give a desired submaximal response when inadequate amounts of the endogenous ligand are present, or they can reduce the overstimulation of receptors when excess amounts of the endogenous ligand are present. The maximum response (Emax) produced by a partial agonist is called its intrinsic activity and may be expressed on a percentage scale where a full agonist produces a 100% response. In some embodiments, the IL2R binding molecule has a reduced Eₘₐₓ compared to the Eₘₐₓ caused by IL2. Eₘₐₓ reflects the maximum response level in a cell type that can be obtained by a ligand (e.g., a binding molecule described herein or the native cytokine (e.g., IL2)). In some embodiments, the IL2R binding molecule described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ caused by IL2. In other embodiments, the Eₘₐₓ of the IL2R binding molecule described herein is greater (e.g., at least 1%, 5%, 10%, 15%, 20%, 25%, 30%, 35%, 40%, 45%, or 50% greater) than the Eₘₐₓ of the natural ligand, IL2. In some embodiments, by varying the linker length of the IL2R binding molecule, the Eₘₐₓ of the IL2R binding molecule can be changed. The IL2R binding molecule can cause Eₘₐₓ in the most desired cell types, and a reduced Eₘₐₓ in other cell types.

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; fusion proteins of immunologically active proteins (e.g. antigenic diphtheria or tetanus toxin fragments) and the like.

As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present state to a more deleterious state.

Proximity: As used herein, the term “proximity” refers to the spatial proximity or physical distance between two cell surface receptors, or domains or subunits thereof, after a binding molecule described herein binds to the two cell surface receptors, or domains or subunits thereof. In some embodiments, after the binding molecule binds to the cell surface receptors, or domains or subunits thereof, the spatial proximity between the cell surface receptors, or domains or subunits thereof, can be, e.g., less than about 500 angstroms, such as e.g., a distance of about 5 angstroms to about 500 angstroms. In some embodiments, the spatial proximity amounts to less than about 5 angstroms, less than about 20 angstroms, less than about 50 angstroms, less than about 75 angstroms, less than about 100 angstroms, less than about 150 angstroms, less than about 250 angstroms, less than about 300 angstroms, less than about 350 angstroms, less than about 400 angstroms, less than about 450 angstroms, or less than about 500 angstroms. In some embodiments, the spatial proximity amounts to less than about 100 angstroms. In some embodiments, the spatial proximity amounts to less than about 50 angstroms. In some embodiments, the spatial proximity amounts to less than about 20 angstroms. In some embodiments, the spatial proximity amounts to less than about 10 angstroms. In some embodiments, the spatial proximity ranges from about 10 to 100 angstroms, from about 50 to 150 angstroms, from about 100 to 200 angstroms, from about 150 to 250 angstroms, from about 200 to 300 angstroms, from about 250 to 350 angstroms, from about 300 to 400 angstroms, from about 350 to 450 angstroms, or about 400 to 500 angstroms. In some embodiments, the spatial proximity amounts to less than about 250 angstroms, alternatively less than about 200 angstroms, alternatively less than about 150 angstroms, alternatively less than about 120 angstroms, alternatively less than about 100 angstroms, alternatively less than about 80 angstroms, alternatively less than about 70 angstroms, or alternatively less than about 50 angstroms.

Receptor: As used herein, the term “receptor” refers to a polypeptide having a domain that specifically binds a ligand that binding of the ligand results in a change to at least one biological property of the polypeptide. In some embodiments, the receptor is a “soluble” receptor that is not associated with a cell surface. In some embodiments, the receptor is a cell surface receptor that comprises an extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of the ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface molecule not associated with any intracellular signaling alone but upon ligand binding facilitates the formation of a multimeric complex that results in intracellular signaling.

Recombinant: As used herein, the term “recombinant” is used as an adjective to refer to the method by a polypeptide, nucleic acid, or cell that was modified using recombinant DNA technology. A recombinant protein is a protein produced using recombinant DNA technology and may be designated as such using the abbreviation of a lower case “r” (e.g., rhIL2) to denote the method by which the protein was produced. Similarly, a cell is referred to as a “recombinant cell” if the cell has been modified by the incorporation (e.g., transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in an evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors. In contrast, the terms “inhibition”, “down-regulation” and the like refer to the opposite effects.

Single Domain Antibody (sdAb): The term “single-domain antibody” or “sdAbs,” refers to an antibody having a single (only one) monomeric variable antibody domain. A sdAb is able to bind selectively to a specific antigen. A VHH antibody, further defined below, is an example of a sdAb.

Specifically Binds: As used herein, the term “specifically bind” refers to the degree of selectivity or affinity for which one molecule binds to another. In the context of binding pairs (e.g., a binding molecule described herein/receptor, a ligand/receptor, antibody/antigen, antibody/ligand, antibody/receptor binding pairs), a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample.

Stably Associated: As used herein, the term “stably associated” or “in stable association with” are used to refer to the various means by which one molecule (e.g., a polypeptide) may be associated with another molecule over an extended period of time. The stable association of one molecule to another may be effected by a variety of means, including covalent bonding and non-covalent interactions. In some embodiments, stable association of two molecules may be effected by covalent bonds such as peptide bonds. In other embodiments, stable association of two molecules may be effected by non-covalent interactions. Examples of non-covalent interactions which may provide a stable association between two molecules include electrostatic interactions (e.g., hydrogen bonding, ionic bonding, halogen binding, dipole-dipole interactions, Van der Waals forces and t-effects including cation-π interactions, anion-π interactions and π-π interactions) and hydrophobilic/hydrophilic interactions. In some embodiments, the stable association of sdAbs of the binding molecules of the present disclosure may be effected by non-covalent interactions. In one embodiment, the non-covalent stable association of the sdAbs of the binding molecules may be achieved by conjugation of the sdAbs to “knob-into-hole” modified Fc monomers. An Fc “knob” monomer stably associates non-covalently with an Fc “hole” monomer. Conjugation of a first sdAb which specifically binds to the extracellular domain of a first subunit of a heterodimeric receptor to an “Fc knob” monomer and conjugation of a second sdAb which specifically binds to the extracellular domain of a second subunit of a heterodimeric receptor to an “Fc hole” monomer provides stable association of the first and second sdAbs.

Subject: The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Substantially: As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher of a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Suffering From: As used herein, the term “suffering from” refers to a determination made by a physician with respect to a subject based on the available information accepted in the field for the identification of a disease, disorder or condition including but not limited to X-ray, CT-scans, conventional laboratory diagnostic tests (e.g., blood count), genomic data, protein expression data, immunohistochemistry, that the subject requires or will benefit from treatment. The term suffering from is typically used in conjunction with a particular disease state such as “suffering from a neoplastic disease” refers to a subject which has been diagnosed with the presence of a neoplasm.

Therapeutically Effective Amount: As used herein, the term The phrase “therapeutically effective amount” is used in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, modification of biomarker levels, increase in duration of survival, extended duration of progression free survival, extension of the time to progression, increased time to treatment failure, extended duration of event free survival, extension of time to next treatment, improvement objective response rate, improvement in the duration of response, and the like that that are relied upon by clinicians in the field for the assessment of an improvement in the condition of the subject in response to administration of an agent.

Treat: The terms “treat”, “treating”, treatment” and the like refer to a course of action (such as administering a binding molecule described herein, or a pharmaceutical composition comprising the same) initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

VHH: As used herein, the term “VHH” is a type of sdAb that has a single monomeric heavy chain variable antibody domain. Such antibodies can be found in or produced from Camelid mammals (e.g., camels, llamas) which are naturally devoid of light chains. V_{H}Hs can be obtained from immunization of camelids (including camels, llamas, and alpacas (see, e.g., Hamers-Casterman, et al. (1993) Nature 363:446-448) or by screening libraries (e.g., phage libraries) constructed in VHH frameworks. Antibodies having a given specificity may also be derived from non-mammalian sources such as V_{H}Hs obtained from immunization of cartilaginous fishes including, but not limited to, sharks. In a particular embodiment, a VHH in a bispecific V_{H}H² binding molecule described herein binds to a receptor (e.g., the first receptor or the second receptor of the natural or non-natural receptor pairs) if the equilibrium dissociation constant between the VHH and the receptor is greater than about 10⁶ M, alternatively greater than about 108 M, alternatively greater than about 10¹⁰ M, alternatively greater than about 10¹¹ M, alternatively greater than about 10¹⁰ M, greater than about 10¹² M as determined by, e.g., Scatchard analysis (Munsen, et al. 1980 Analyt. Biochem. 107:220-239). Standardized protocols for the generation of single domain antibodies from camelids are well known in the scientific literature. See, e.g., Vincke, et al (2012) Chapter 8 in Methods in Molecular Biology, Walker, J. editor (Humana Press, Totowa NJ). Specific binding may be assessed using techniques known in the art including but not limited to competition ELISA, BIACORE® assays and/or KINEXA® assays. In some embodiments, a V_{H}H described herein can be humanized to contain human framework regions. Examples of human germlines that could be used to create humanized V_{H}Hs include, but are not limited to, VH3-23 (e.g., UniProt ID: P01764), VH3-74 (e.g., UniProt ID: A0A0B4J1X5), VH3-66 (e.g., UniProt ID: A0A0C4DH42), VH3-30 (e.g., UniProt ID: P01768), VH3-11 (e.g., UniProt ID: P01762), and VH3-9 (e.g., UniProt ID: P01782).

V_{H}H²: As used herein, the term “V_{H}H²” and “bispecific V_{H}H²” and “V_{H}H dimer” refers to are used interchangeably to refer to a subtype of the binding molecules of the present disclosure wherein the first and second sdAbs are both V_{H}Hs and first V_{H}H binding to a first receptor, or domain or subunit thereof, and a second V_{H}H binding to a second receptor, or domain or subunit thereof.

Wild Type: As used herein, the term “wild type” or “WT” or “native” is used to refer to an amino acid sequence or a nucleotide sequence that is found in nature and that has not been altered by the hand of man.
I. IL2R BINDING MOLECULES
II. HETERODIMERIZATION OF THE INTERMEDIATE AFFINITY INTERLEUKIN-2 RECEPTORS (IL-2R), IL-2Rβ AND IL-2Rγ, INITIATES A SIGNALING CASCADE IN T AND NK CELLS THAT ULTIMATELY RESULTS IN PROLIFERATION AND PRODUCTION OF INTERFERON-GAMMA (IFN-γ). BINDING OF IL2 IN A DIMERIC COMPLEX WITH INTERMEDIATE AFFINITY OR IN THE HIGH AFFINITY TRIMERIC COMPLEX, INCLUDING IL-2Rα, ON ACTIVATED T CELLS AND TREGS LEADS TO TRANS-PHOSPHORYLATION OF SIGNALING MOTIFS ON THE IL-2Rβ AND IL-2Rγ INTRACELLULAR DOMAINS BY THE ASSOCIATED JAK KINASES. THE FUNCTION OF THE IL2 IS TO BRING THE RECEPTOR CHAINS IN CLOSE PROXIMITY TO PRODUCE AN OPTIMAL LEVEL OF PHOSPHORYLATION AND ACTIVATION OF ASSOCIATED STAT TRANSCRIPTION FACTORS.
III. HERE WE ESTABLISHED A WAY TO BRING IL-2Rβ AND IL-2Rγ CHAINS TOGETHER IN A CYTOKINE-INDEPENDENT MANNER THAT RESULTS IN FUNCTIONAL ACTIVATION. HUMAN IL-2Rβ AND IL-2Rγ SPECIFIC HEAVY CHAIN SINGLE DOMAIN NANOBODIES (V_{H}H) WERE GENERATED BY CAMEL IMMUNIZATION AND SCREENING OF V_{H}H LIBRARIES PREPARED FROM PERIPHERAL BLOOD CELLS FOR BINDING. TEN IL-2Rβ V_{H}H SEGMENTS AND SIX IL-2Rγ V_{H}H SEGMENTS, ALL WITH LOW NANOMOLAR AFFINITY, WERE IDENTIFIED AND COUPLED AS IL-2Rβ/IL-2Rγ V_{H}H DIMERS IN ALL POSSIBLE COMBINATIONS IN BOTH AMINO-CARBOXY AND CARBOXY-AMINO ORIENTATIONS YIELDING 120 DIFFERENT PROTEINS.
IV. FORTY-TWO OF THE IL-2Rβ/IL-2Rγ SYNTHEKINES FROM THE PANEL WERE FUNCTIONAL AND INDUCED PSTAT-5 PHOSPHORYLATION IN THE IL-2 DEPENDENT NK CELL LINE NKL.
V. THE BIOLOGICAL ACTIVITY OF THESE IL-2Rβ/IL-2Rγ SYNTHEKINES WAS FURTHER CONFIRMED ON PRIMARY CELLS. IL-2Rβ/IL-2Rγ SYNTHEKINES INDUCED PSTAT5 PHOSPHORYLATION ON NK CELLS ISOLATED FROM HUMAN PERIPHERAL BLOOD AND CULTURE WITH IL-2Rβ/IL-2Rγ SYNTHEKINES RESULTED IN PROLIFERATION AND PRODUCTION OF HIGH BUT VARIED LEVELS OF IFN-γ.
VI. THE ACTIVITY OF IL-2Rβ/IL-2Rγ SYNTHEKINES ON PRIMARY T CELLS WAS ALSO EXAMINED. SIMILAR TO NK CELLS, IL-2Rβ/IL-2Rγ SYNTHEKINES INDUCED PSTAT5 PHOSPHORYLATION, PROLIFERATION AND IFN-γ PRODUCTION BY CD4 POSITIVE AND CD8 POSITIVE T CELL BLASTS GENERATED AFTER CD3/CD28 ACTIVATION OF HUMAN PBMC. IN CONCLUSION, WE HAVE GENERATED A SERIES OF FUNCTIONAL IL-2Rβ/IL-2Rγ SYNTHEKINES EACH WITH UNIQUE SIGNALING STRENGTHS, WHICH WILL NOW BE FURTHER ANALYZED FOR POTENTIAL THERAPEUTIC APPLICATION IN TUMOR IMMUNOLOGY AND AUTO-IMMUNITY.
VII.

The present disclosure provides binding molecules that are agonists of the IL2R receptor, the binding molecule comprising:
a) a first single domain antibody (sdAb) that specifically binds to the extracellular domain of IL2Rb of the IL2R (an “IL2Rb sdAb”), and
b) a second single domain antibody that specifically binds to extracellular domain IL2Rg of the IL2R (an “IL2Rg sdAb”), wherein the IL2Rb sdAb and IL2Rg sdAb are stably associated and wherein contacting a cell expressing IL2Rb and IL2Rg with an effective amount of the binding molecule results in the dimerization of IL2Rb and IL2Rg, and results in intracellular signaling characteristic of the IL2R receptor when activated by its natural cognate IL2. In some embodiments, one or both of the sdAbs is a an scFv. In some embodiments, one or both of the sdAbs is a V_{H}H.

As used herein, the term “IL2R receptor” or “IL2R” refers to the heterodimeric intermediate affinity receptor formed by subunits IL2Rb and IL2Rg, when associated with the cognate IL2.

The IL2 receptor (IL2R) includes CD25 subunit (CD25; also called IL2Rg subunit or IL2Ra subunit), CD122 subunit (CD122; also called IL2Rβ subunit or IL2Rb subunit), and CD132 subunit (CD132; also called IL2Rγ subunit or IL2Rg subunit). Provided herein is an IL2R binding protein that specifically binds to CD122 and CD132. In some embodiments, the IL2R binding protein binds to a mammalian cell expressing both CD122 and CD132. In some embodiments, the IL2R binding protein can be a bispecific VHH2 as described below. In other embodiments, the IL2R binding protein can include a first domain that is a VHH and a second domain which can be a fragment of IL2 or, for example, a scFv.

In some embodiments, the IL2R binding protein has a reduced Eₘₐₓ compared to the Eₘₐₓ caused by IL2. Eₘₐₓ reflects the maximum response level in a cell type that can be obtained by a ligand (e.g., a binding protein described herein or the native cytokine (e.g., IL2)). In some embodiments, the IL2R binding protein described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ caused by IL2. In some embodiments, by varying the linker length of the IL2R binding protein, the Eₘₐₓ of the IL2R binding protein can be changed. The IL2R binding protein can cause Eₘₐₓ in the most desired cell types (e.g., CD8⁺ T cells), and a reduced Eₘₐₓ in other cell types (e.g., macrophages). In some embodiments, the Eₘₐₓ in macrophages caused by an IL2R binding protein described herein is between 1% and 100% (e.g., between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ in T cells (e.g., CD8⁺ T cells) caused by the IL2R binding protein. In other embodiments, the Eₘₐₓ of the IL2R binding protein described herein is greater (e.g., at least 1%, 5%, 10%, 15%, 20%, 25%, 30%, 35%, 40%, 45%, or 50% greater) than the Eₘₐₓ of the natural ligand, IL2.

The IL2R binding protein can be a bispecific VHH2 that has a first VHH binding to CD122 (an antiCD122 VHH antibody) and a second VHH binding to CD132 (an antiCD132 VHH antibody) and causes the dimerization of the two receptor subunits and downstream signaling when bound to a cell expressing CD122 and CD132, e.g., a T cell (e.g., a CD8+ T cell or a CD4+ T cell), a macrophage, and/or a Treg cell.

The IL2 receptor (IL2R) includes IL2Rb subunit (IL2Rb) and IL2Rg subunit (IL2Rg). Provided herein is an IL2R binding molecule that specifically binds to IL2Rb and IL2Rg. In some embodiments, the IL2R binding molecule binds to a mammalian cell expressing both IL2Rb and IL2Rg. In some embodiments, the IL2R binding molecule can be a bispecific VHH2 as described below.

### Single Domain Antibody

The IL2R binding molecules of the present invention comprise two or more single domain antibodies. The term “single domain antibody” (sdAb) as used herein refers to an antibody fragment consisting of a monomeric variable antibody domain that is able to bind specifically to an antigen and compete for binding with the parent antibody from which it is derived. The term “single domain antibody” includes scFv and VHH molecules. In some embodiments, one or both of the sdAbs of the cytokine receptor binding molecule is an scFv. In some embodiments, one or both of the sdAbs is a VHH. In some embodiments, one or both of the sdAbs is a scFv.

The term single domain antibody includes engineered sdAbs including but not limited to chimeric sdAbs, CDR grafted sdAbs and humanized sdAbs. In some embodiments, the one or more of the sdAbs for incorporation into the IL2R binding molecules of the present disclosure are CDR grafted. CDRs obtained from antibodies, heavy chain antibodies, and sdAbs derived therefrom may be grafted onto alternative frameworks as described in Saerens, et al. (2005) J. Mol Biol 352:597-607 to generate CDR-grafted sdAbs. Any framework region can be used with the CDRs as described herein.

In some embodiments, one or more of the sdAbs for incorporation into the IL2R binding molecules is a chimeric sdAb, in which the CDRs are derived from one species (e.g., camel) and the framework and/or constant regions are derived from another species (e.g., human or mouse). In specific embodiments, the framework regions are human or humanized sequences. Thus, IL2R binding molecules comprising one or more humanized sdAbs are considered within the scope of the present disclosure.

In some embodiments, one or more of the sdAb of the cytokine receptor binding molecules of the present disclosure is a VHH. As used herein, the term “VHH” refers to a single domain antibody derived from camelid antibody typically obtained from immunization of camelids (including camels, llamas and alpacas (see, e.g., Hamers-Casterman, et al. (1993) Nature 363:446-448). VHHs are also referred to as heavy chain antibodies or Nanobodies® as Single domain antibodies may also be derived from non-mammalian sources such as VHHs obtained from IgNAR antibodies immunization of cartilaginous fishes including, but not limited to, sharks. A VHH is a type of single-domain antibody (sdAb) containing a single monomeric variable antibody domain. Like a full-length antibody, it is able to bind selectively to a specific antigen.

The complementary determining regions (CDRs) of VHHs are within a single-domain polypeptide. VHHs can be engineered from heavy-chain antibodies found in camelids. An exemplary VHH has a molecular weight of approximately 12-15 kDa which is much smaller than traditional mammalian antibodies (150-160 kDa) composed of two heavy chains and two light chains. VHHs can be found in or produced from Camelidae mammals (e.g., camels, llamas, dromedary, alpaca, and guanaco) which are naturally devoid of light chains. Descriptions of sdAbs and VHHS can be found in, e.g., De Greve et al., Curr Opin Biotechnol. 61:96-101, 2019; Ciccarese, et al., Front Genet. 10:997, 2019; Chanier and Chames, Antibodies (Basel) 8(1), 2019; and De Vlieger et al., Antibodies (Basel) 8(1), 2018. The CDRs derived from camelid VHHs may be used to prepare CDR-grafted VHHs which may be incorporated in the IL2R binding molecules.

In some embodiments, the VHH for incorporation into the IL2R binding molecule of the present disclosure is a humanized VHH containing human framework regions. The techniques for humanization of camelid single domain antibodies are well known in the art. See, e.g., Vincke, et al. (2009) General Strategy to Humanize a Camelid Single-domain Antibody and Identification of a Universal Humanized Nanobody Scaffold J. Biol. Chem. 284(5)3273-3284. Human framework regions useful in the preparation of humanized V_{H}Hs include, but are not limited to, VH3-23 (e.g., UniProt ID: P01764), VH3-74 (e.g., UniProt ID: A0A0B4J1X5), VH3-66 (e.g., UniProt ID: A0A0C4DH42), VH3-30 (e.g., UniProt ID: P01768), VH3-11 (e.g., UniProt ID: P01762), and VH3-9 (e.g., UniProt ID: P01782).

### Stably Associated:

The IL2R binding molecules of the present disclosure comprise a single domain antibody that selectively binds to the extracellular domain of IL2Rg (an “IL2Rg sdAb”) in stable association with a single domain antibody that selectively binds to the extracellular domain of IL2Rb (an “IL2Rb sdAb”). As used herein, the terms “stably associated” or “in stable association with” are used to refer to the various means by which one molecule (e.g., a polypeptide) may be thermodynamically and/or kinetically associated with another molecule. The stable association of one molecule to another may be achieved by a variety of means, including covalent bonding and non-covalent interactions.

In some embodiments, stable association of the IL2Rg sdAb and IL2Rb sdAb may be achieved by a covalent bond such as a peptide bond. In some embodiments, the covalent linkage between the first and second binding domains is a covalent bond between the C-terminus of the first binding domain and the N-terminus of the second binding domain.

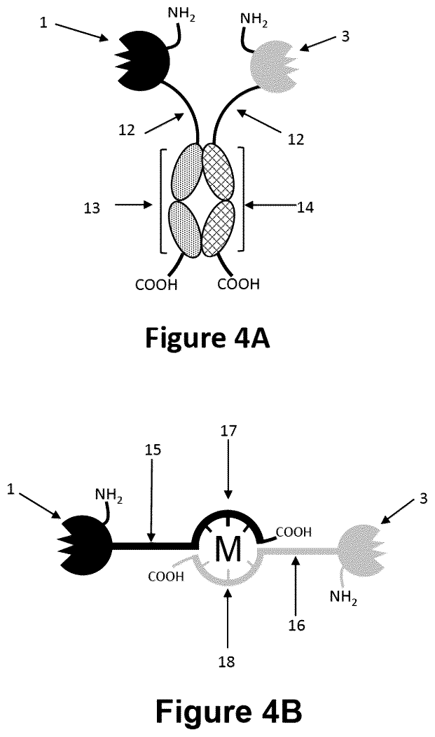

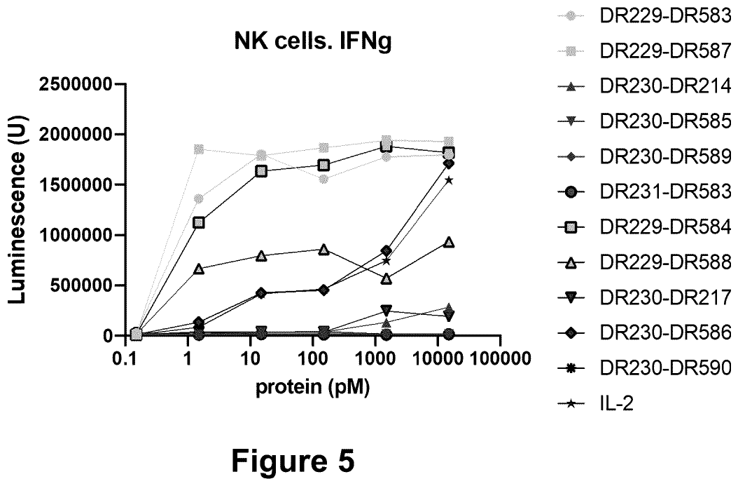

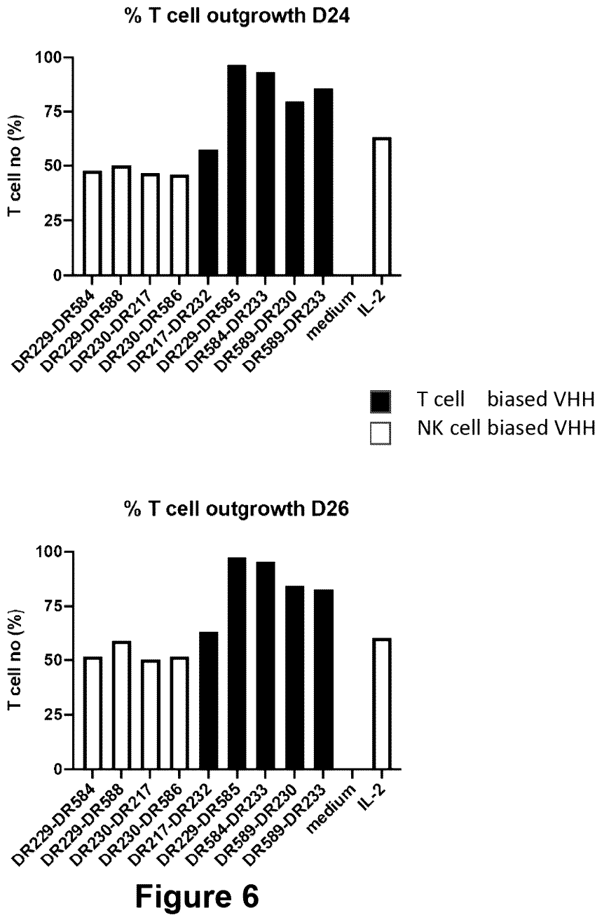

In some embodiments, the covalent linkage of the IL2Rg sdAb and IL2Rb sdAb of the IL2R binding protein is effected by a coordinate covalent linkage. The present disclosure provides examples of single domain antibodies comprising a chelating peptide. The chelating peptide results in a coordinate covalent linkage to a transition metal ion. In some embodiments, a transition metal ion is capable of forming a coordinate covalent linkage with two or more chelating peptides. Consequently, the first and second binding domains may each comprise a chelating peptide and a stable association of the binding domains by each subunit forming a coordinate covalent complex with a transition metal ion. In some embodiments, the transition metal ion is selected from vanadium, manganese, iron, iridium, osmium, rhenium platinum, palladium, cobalt, chromium or ruthenium. A schematic illustration of this configuration is provided in FIG. 4, Panel B of the attached drawings. It should be noted that in each of the configurations illustrated in FIG. 4, Panels A and B, the N-terminal domain of the single domain antibody is presented to the environment facilitating enhanced exposure of the CDRs of the sdAb to the target cytokine receptor ECD. The formation of the coordinate covalent linkage is favored when the transition metal ion is in a kinetically labile oxidation state, for example Co(II), Cr(II), or Ru(III). Following complexation, the oxidation state of the transition metal may be changed (oxidized or reduced) to a kinetically inert oxidation state, for example Co(III), Cr(III), or Ru(II), to provide a kinetically inert coordinate covalent complex. The formation of kinetically inert and kinetically labile coordinate covalent complexes between proteins comprising chelating peptides via a transition metal are described in more detail in Anderson, et al. U.S. Pat. No. 5,439,928 issued Aug. 8, 1995.

In some embodiments, the covalent linkage of the IL2Rg sdAb and IL2Rb sdAb of the IL2R binding molecule may further comprise a linker. Linkers are molecules selected from the group including, but not limited to, peptide linkers and chemical linkers. In some embodiments, the linker a joins the C-terminus of the IL2Rg sdAb to the N-terminus of the IL2Rb sdAb. In some embodiments, the linker joins the C-terminus of the IL2Rb sdAb to the N-terminus of the IL2Rg sdAb.

### Peptide Linkers

In some embodiments, the stable association of the first and second domains may be achieved by covalent linkage of the C-terminus of the first binding domain and the N-terminus of the second binding domain via a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids). Examples of flexible peptide linkers include glycine polymers (G)n, glycine-alanine polymers, alanine-serine polymers, glycine-serine polymers (for example, (GmSo)n (SEQ ID NO: 464), (GSGGS)n (SEQ ID NO: 465), (GmSoGm)n (SEQ ID NO: 466), (GmSoGmSoGm)n (SEQ ID NO: 467), (GSGGSm)n (SEQ ID NO: 468), (GSGSmG)n (SEQ ID NO: 469) and (GGGSm)n (SEQ ID NO: 470), and combinations thereof, where m, n, and o are each independently selected from an integer of at least 1 to 20 (e.g., 1-18, 216, 3-14, 4-12, 5-10, 1, 2, 3, 4, 5, 6, 7, 8, 9, or 10), and other flexible linkers. Glycine and glycine-serine polymers are relatively unstructured, and therefore may serve as a neutral tether between components. Exemplary flexible linkers include the linkers of but are not limited to GGGS (SEQ ID NO:11), GGGGS (SEQ ID NO: 12), GGSG (SEQ ID NO: 13), GGSGG (SEQ ID NO: 14), GSGSG (SEQ ID NO: 15), GSGGG (SEQ ID NO: 16), GGGSG (SEQ ID NO: 17) and GSSSG (SEQ ID NO: 18). In yet other embodiments, a peptide linker can contain 4 to 20 amino acids including mixtures of the above motifs of GGSG (SEQ ID NO: 13), e.g., GGSGGGSG (SEQ ID NO:19), GGSGGGSGGGSG (SEQ ID NO:20), GGSGGGSGGGSGGGSG (SEQ ID NO:21), or GGSGGGSGGGSGGGSGGGSG (SEQ ID NO:22). In other embodiments, a peptide linker can contain motifs of GGSG (SEQ ID NO:13), e.g., GGSGGGSG (SEQ ID NO: 19), GGSGGGSGGGSG (SEQ ID NO:20), GGSGGGSGGGSGGGSG (SEQ ID NO:21), or GGSGGGSGGGSGGGSGGGSG (SEQ ID NO:22).

In some embodiments, the covalent linkage of the first and second domains may be achieved by a chemical linker. Examples of chemical linkers include aryl acetylene, ethylene glycol oligomers containing 2-10 monomer units, diamines, diacids, amino acids, or combinations thereof.

In some embodiments, stable association the IL2Rg sdAb and IL2Rb sdAb of the IL2R binding protein is be effected by non-covalent interaction. Examples of non-covalent interactions that provide a stable association between two molecules include electrostatic interactions (e.g., hydrogen bonding, ionic bonding, halogen binding, dipole-dipole interactions, Van der Waals forces and p-effects including cation-p interactions, anion-p interactions and p-p interactions) and hydrophobilic/hydrophilic interactions. In some embodiments, the stable association of sdAbs of the binding molecules of the present disclosure may be effected by non-covalent interactions.

In one embodiment, the non-covalent stable association of the IL2Rg sdAb and IL2Rb sdAb of the IL2R binding molecule may be achieved by conjugation a sdAb each monomer of a “knob-into-hole” engineered Fc dimer. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g., tyrosine or tryptophan) creating a projection from the surface (“knob”) and ii) in the CH3 domain of a second heavy chain, an amino acid residue is replaced with an amino acid residue having a smaller side chain (e.g., alanine or threonine), thereby generating a cavity (“hole”) within at interface in the second CH3 domain within which the protruding side chain of the first CH3 domain (“knob”) is received by the cavity in the second CH3 domain. The knob-into-hole modification is more fully described in Ridgway, et al. (1996) Protein Engineering 9(7):617-621 and U.S. Pat. No. 5,731,168, issued Mar. 24, 1998, U.S. Pat. No. 7,642,228, issued Jan. 5, 2010, U.S. Pat. No. 7,695,936, issued Apr. 13, 2010, and U.S. Pat. No. 8,216,805, issued Jul. 10, 2012. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 and Y349 which results in a stabilizing disulfide bridge between the two antibody heavy chains in the Fc region (Carter, et al. (2001) Immunol Methods 248, 7-15). The knob-into-hole format is used to facilitate the expression of a first polypeptide (e.g., an IL2Rb binding sdAb) on a first Fc monomer with a “knob” modification and a second polypeptide on the second Fc monomer possessing a “hole” modification to facilitate the expression of heterodimeric polypeptide conjugates. The knob-into-hole format is used to facilitate the expression of a first polypeptide on a first Fc monomer with a “knob” modification and a second polypeptide on the second Fc monomer possessing a “hole” modification to facilitate the expression of heterodimeric polypeptide conjugates. One embodiment of an IL2R binding molecule wherein the IL2Rg sdAb and IL2Rb sdAb are in stable, non-covalent association is wherein each sdAb of the IL2R binding molecule is covalently bonded, optionally including a linker, to each subunit of the knob-into-hole Fc dimer as illustrated in FIG. 4, Panel A of the attached drawings.

### Generation and Evaluation of IL2Rg Single Domain Antibodies

To generate sdAbs against the hIL2Rα, the extracellular domain of the hIL2Ra protein may be used an immunogen. The extracellular domain of the mature (lacking the signal sequence) hIL2Ra possesses the amino acid sequence has the amino acid sequence

(SEQ ID NO: 453)
HGTELPSPPSVWFEAEFFHHILHWTPIPNQSESTCYEVALLRYGIESWNS
ISNCSQTLSYDLTAVTLDLYHSNGYRARVRAVDGSRHSNWTVTNTRFSVD
EVTLTVGSVNLEIHNGFILGKIQLPRPKMAPANDTYESIFSHFREYEIAI
RKVPGNFTFTHKKVKHENFSLLTSGEVGEFCVQVKPSVASRSNKGMWSKE
ECISLTRQYFTVTN

In some embodiments, when employed as an immunogen or an immunogenic composition, the hIL2Rg ECD may be provided as a domain of a fusion protein with an immunomodulatory protein.

To generate sdAbs against the mIL10Ra, the extracellular domain of the mIL10Ra protein may be used an immunogen. The extracellular domain of the extracellular domain of mIL10Ra possesses the amino acid sequence has the amino acid sequence

(SEQ ID NO: 455)
LEFIAYGTELPSPSYVWFEARFFQHILHWKPIPNQSESTYYEVALKQYGN
STWNDIHICRKAQALSCDLTTFTLDLYHRSYGYRARVRAVDNSQYSNWTT
TETRFTVDEVILTVDSVTLKAMDGIIYGTIHPPRPTITPAGDEYEQVFKD
LRVYKISIRKFSELKNATKRVKQETFTLTVPIGVRKFCVKVLPRLESRIN
KAEWSEEQCLLITTEQYFTVTNLSI

In some embodiments, when employed as an immunogen or a immunogenic composition, the mIL2Rg ECD may be provided as a domain of a fusion protein with an immunomodulatory protein.

A series of hIL2Rb sdAbs were generated in substantial accordance with the teaching of Examples 1-4 herein. Briefly, a camel was sequentially immunized with the ECD of the human IL2Rb over a period several weeks of by the subcutaneous an adjuvanted composition containing a recombinantly produced fusion proteins comprising the extracellular domain of hIL2Rb, the human IgG1 hinge domain and the human IgG1 heavy chain Fc. Following immunization, RNAs extracted from a blood sample of appropriate size VHH-hinge-CH2-CH3 species were transcribed to generate DNA sequences, digested to identify the approximately 400 bp fragment comprising the nucleic acid sequence encoding the VHH domain was isolated. The isolated sequence was digested with restriction endonucleases to facilitate insertion into a phagemid vector for in frame with a sequence encoding a his-tag and transformed into E. coli to generate a phage library. Multiple rounds of bio-panning of the phage library were conducted to identify VHHs that bound to the ECD of IL2Rb (human or mouse as appropriate). Individual phage clones were isolated for periplasmic extract ELISA (PE-ELISA) in a 96-well plate format and selective binding confirmed by colorimetric determination. The IL2Rb binding molecules that demonstrated specific binding to the IL2Rb antigen were isolated and sequenced and sequences analyzed to identify VHH sequences, CDRs and identify unique VHH clonotypes. As used herein, the term “clonotypes” refers a collection of binding molecules that originate from the same B-cell progenitor cell, in particular collection of antigen binding molecules that belong to the same germline family, have the same CDR3 lengths, and have 70% or greater homology in CDR3 sequence.

The amino acid sequences of VHH molecules demonstrating specific binding to the hIL2Rg ECD antigen (hIL2Rg VHHs) are provided in Table 8 and the CDRs isolated from such VHHs are provided in Table 4. Nucleic acid sequences encoding the VHHs of Table 8 are provided in Table 12.

To confirm and evaluate binding affinities of the binding of the IL2Rg sdAbs, a representative example from each clonotype generated was selected for evaluation of binding via SPR. Evaluation of binding affinity of the hIL2Rg VHHs for ECD corresponding to SEQ ID NOS 159, 161, 162, 163, 165, 167 and 170 was conducted using surface plasmon resonance (SPR) in substantial accordance with the teaching of Example 5. Buffer-subtracted sensograms were processed with Biacore T200 Evaluation Software and globally fit with a 1:1 Langmuir binding model (bulk shift set to zero) to extract kinetics and affinity constants (kₐ, k_{d}, K_{D}). R_{MAX}<100 RU indicates surface density compatible with kinetics analysis. Calculated Rₘₐₓ values were generated using the equation: Rmax=Load (RU)×valency of ligand×(Molecular weight of analyte/Molecular weight of ligand). Surface activity was defined as the ratio of experimental/calculated Rmax. The results of these binding affinity experiments are provided in Table 17 below. The data provided in Table 17 demonstrates that the IL2Rg single domain antibodies generated possessed specific binding to the ECD of hIL2Rg.

Tables 2 and 3 provide CDRs useful in the preparation of IL2Rb sdAbs for incorporation into the binding molecules of the present disclosure. In some embodiments, the IL2Rb sdAb is a single domain antibody comprising one or more anti-human IL2Rb CDRs in a row of Table 2, wherein each CDR independently comprises 0, 1, 2, or 3 amino acid changes relative to the sequence of Table 2. In some embodiments, the IL2Rb sdAb is a single domain antibody comprising one or more anti-murine IL2Rb CDRs in a row of Table 3, wherein each CDR independently comprises 0, 1, 2, or 3 amino acid changes relative to the sequence of Table 3.

In some embodiments, the IL2Rb sdAb comprises a sequence having at least 90% (e.g., 91%, 92%, 93%, 94%, 95%, 96%, 97%, 98%, 99%, or 100%) sequence identity to a sequence of any one the of IL2Rb sdAbs provided in a row of Table 6. In certain embodiments, the binding molecule comprises a sequence that is substantially identical to a sequence of any one listed in a row of Table 6.

In some embodiments, the IL2Rb sdAb comprises a sequence having at least 90% (e.g., 91%, 92%, 93%, 94%, 95%, 96%, 97%, 98%, 99%, or 100%) sequence identity to a sequence of any one the of anti-murine IL2Rb sdAbs provided in a row of Table 7. In certain embodiments, the binding molecule comprises a sequence that is substantially identical to a sequence of any one listed in a row of Table 7.

In another aspect, the disclosure provides an isolated nucleic acid encoding an IL2Rb sdAb described herein. Table 11 provides DNA sequences encoding the mIL2Rb sdAbs of Table 7. In certain embodiments, the present disclosure provides an isolated nucleic acid comprising a sequence having at least 90% (e.g., 91%, 92%, 93%, 94%, 95%, 96%, 97%, 98%, 99%, or 100%) sequence identity to a DNA sequence listed in a row of Table 11.

### Exemplary Anti IL2Rg Single Domain Antibodies

Table 4 and Table 5 provide CDRs useful in the preparation of IL2Rg sdAbs. In some embodiments, the anti-IL2Rg sdAb is a single domain antibody comprising one or more anti-human IL2Rg CDRs in a row of Table 4, wherein each CDR independently comprises 0, 1, 2, or 3 amino acid changes relative to the sequence of Table 4. In some embodiments, the IL2Rb sdAb is a single domain antibody comprising one or more anti-murine IL2Rg CDRs in a row of Table 5, wherein each CDR independently comprises 0, 1, 2, or 3 amino acid changes relative to the sequence of Table 5.

In some embodiments, the anti-IL2Rg sdAb comprises a sequence having at least 90% (e.g., 91%, 92%, 93%, 94%, 95%, 96%, 97%, 98%, 99%, or 100%) sequence identity to a sequence of any one the of anti-IL2Rg sdAbs provided in a row of Table 8 or Table 9. In certain embodiments, the binding molecule comprises a sequence that is substantially identical to a sequence of any one of listed in a row of Table 8 or Table 9.

In another aspect, the disclosure provides an isolated nucleic acid encoding IL2Rg sdAb described herein. Table 12 and Table 13 provide DNA sequences encoding the anti-IL2Rg sdAbs of Table 8 or Table 9, respectively. In certain embodiments, the present disclosure provides an isolated nucleic acid comprising a sequence having at least 90% (e.g., 91%, 92%, 93%, 94%, 95%, 96%, 97%, 98%, 99%, or 100%) sequence identity to a DNA sequence listed in a row Table 12 or Table 13.

### Anti IL2R V_{H}H Dimer Bispecific Binding Molecules

### A. “Forward Orientation”

In some embodiments, the IL2R binding molecule of the present disclosure comprises a polypeptide of the structure:

H₂N-[IL2Rb sdAb]-[L]ₓ-[IL2Rg sdAb]-[TAG]_{y}-COOH
wherein and L is a polypeptide linker of 1-50 amino acids and x=0 or 1, and TAG is a chelating peptide or a subunit of an Fc domain and y=0 or 1.

In some embodiments, a IL2R binding molecule of the foregoing structure comprises a polypeptide from amino to carboxy terminus:
(a) an IL2Rb sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR1 in a row of Table 2 or Table 3.
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR2 in a row of Table 2 or Table 3; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR3 in a row of Table 2 or Table 3;
(b) polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an IL2Rg sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR1 in a row of Table 4 or Table 5;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR2 in a row of Table 4 or Table 5; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR3 in a row of Table 4 or Table 5.

In some embodiments, the IL2R binding molecule comprises an IL2Rb sdAb comprising a CDR1, a CDR2, and a CDR3 listed in a row of Table 2 or Table 3, and an IL2Rg sdAb comprising a CDR1, a CDR2, and a CDR3 as listed in a row of Table 4 or Table 5.

In some embodiments, the IL2Rb sdAb of the IL2R binding molecule comprises a sequence having at least 90% (e.g., 91%, 92%, 93%, 94%, 95%, 96%, 97%, 98%, 99%, or 100%) sequence identity to a sequence of any one the of IL2Rb sdAbs provided in Table 6 or Table 7. In some embodiments, the IL2Rg sdAb the IL2R binding molecule comprises a sequence having at least 90% (e.g., 91%, 92%, 93%, 94%, 95%, 96%, 97%, 98%, 99%, or 100%) sequence identity to a sequence of any one the of IL2Rg sdAbs provided in Table 8 or Table 9.

### B. “Reverse Orientation”

In some embodiments, the IL2R binding molecule comprises a polypeptide of the structure:

H₂N-[IL2Rg sdAb]-[L]ₓ-[IL2Rb sdAb]-[TAG]_{y}-COOH
wherein and L is a polypeptide linker of 1-50 amino acids and x=0 or 1, and TAG is a chelating peptide or a subunit of an Fc domain and y=0 or 1.

In some embodiments, a IL2R binding molecule of the foregoing structure comprises a polypeptide from amino to carboxy terminus:
(a) an IL2Rg sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR1 in a row of Table 4 or Table 5;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR2 in a row of Table 4 or Table 5; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR3 in a row of Table 4 or Table 5;
(b) polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an IL2Rb sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR1 in a row of Table 2 or Table 3.
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR2 in a row of Table 2 or Table 3; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of the sequence of any CDR3 in a row of Table 2 or Table 3.

In some embodiments, the binding molecule comprises an IL2Rg sdAb comprising a CDR1, a CDR2, and a CDR3 as listed in a row of Table 4 or Table 5, and the IL2Rb sdAb and a CDR1, a CDR2, and a CDR3 as listed in a row of Table 2 or Table 3.

In some embodiments, the IL2Rg sdAb comprises a sequence having at least 90% (e.g., 91%, 92%, 93%, 94%, 95%, 96%, 97%, 98%, 99%, or 100%) sequence identity to a sequence listed in a row of Table 8 or Table 9. In some embodiments, the IL2Rb sdAb comprises a sequence having at least 90% (e.g., 91%, 92%, 93%, 94%, 95%, 96%, 97%, 98%, 99%, or 100%) sequence identity to a sequence listed in a row of Table 6 or Table 7.

### Linkers

A linker can be used to join the IL2Rb sdAb and the IL2Rb sdAb antibody. A linker is a linkage between two linker is a linkage between the two sdAbs in the binding molecule, e.g., protein domains. For example, a linker can simply be a covalent bond or a peptide linker. In some embodiments, the sdAbs in a binding molecule are joined directly (i.e., via a covalent bond). In a bispecific VHH2 binding molecule described herein, a linker is a linkage between the two VHHs in the binding molecule. A In some embodiments, the linker is a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids).

Examples of flexible linkers include glycine polymers (G)n, glycine-alanine polymers, alanine-serine polymers, glycine-serine polymers (for example, (GmSo)n (SEQ ID NO: 464), (GSGGS)n (SEQ ID NO: 465), (GmSoGm)n (SEQ ID NO: 466), (GmSoGmSoGm)n (SEQ ID NO: 467), (GSGGSm)n (SEQ ID NO: 468), (GSGSmG)n (SEQ ID NO: 469) and (GGGSm)n (SEQ ID NO: 470), and combinations thereof, where m, n, and o are each independently selected from an integer of at least 1 to 20, e.g., 1-18, 216, 3-14, 4-12, 5-10, 1, 2, 3, 4, 5, 6, 7, 8, 9, or 10), and other flexible linkers. Glycine and glycine-serine polymers are relatively unstructured, and therefore may serve as a neutral tether between components Exemplary flexible linkers include, but are not limited to GGGS (SEQ ID NO:11), GGGGS (SEQ ID NO: 12), GGSG (SEQ ID NO: 13), GGSGG (SEQ ID NO: 14), GSGSG (SEQ ID NO: 15), GSGGG (SEQ ID NO: 16), GGGSG (SEQ ID NO: 17) and GSSSG (SEQ ID NO: 18). In yet other embodiments, a peptide linker can contain 4 to 20 amino acids including mixtures of the above motifs of GGSG (SEQ ID NO:456), e.g., GGSGGGSG (SEQ ID NO:457), GGSGGGSGGGSG (SEQ ID NO:458), GGSGGGSGGGSGGGSG (SEQ ID NO:459), or GGSGGGSGGGSGGGSGGGSG (SEQ ID NO:460). In other embodiments, a peptide linker can contain motifs of GGSG (SEQ ID NO:456), e.g., GGSGGGSG (SEQ ID NO:457), GGSGGGSGGGSG (SEQ ID NO:458), GGSGGGSGGGSGGGSG (SEQ ID NO:459), or GGSGGGSGGGSGGGSGGGSG (SEQ ID NO:460).

A linker can also be a chemical linker, such as a synthetic polymer, e.g., a polyethylene glycol (PEG) polymer.

The length of the linker between two sdAb in a binding molecule can be used to modulate the proximity of the two sdAb of the binding molecule. By varying the length of the linker, the overall size and length of the binding molecule can be tailored to bind to specific cell receptors or domains or subunits thereof. For example, if the binding molecule is designed to bind to two receptors or domains or subunits thereof that are located close to each other on the same cell, then a short linker can be used. In another example, if the binding molecule is designed to bind to two receptors or domains or subunits there of that are located on two different cells, then a long linker can be used.

In some embodiments, a linker joins the C-terminus of the IL2Rb sdAb in the binding molecule to the N-terminus of the IL2Rg sdAb in the binding molecule. In other embodiments, a linker joins the C-terminus of the IL2Rg sdAb in the binding molecule to the N-terminus of the IL2Rb sdAb in the binding molecule.

Modulation of sdAb Binding Affinity:

In some embodiments, the activity and/or specificity of the IL2R binding molecule of the present disclosure may be modulated by the respective binding affinities of the sdAbs for their respective receptor subunits.

It will be appreciated by one of skill in the art that the binding of the first sdAb of the IL2R binding molecule to the first receptor subunit ECD on the cell surface will enhance the probability of a binding interaction between the second sdAb of the IL2R binding molecule with the ECD of the second receptor subunit. This cooperative binding effect may result in a IL2R binding molecule which has a very high affinity for the receptor and a very slow “off rate” from the receptor. Typical VHH single domain antibodies have an affinity for their targets of from about 10−5M to about 10−10M. In those instances such slow off-rate kinetics are desirable in the IL2R binding molecule, the selection of sdAbs having high affinities (about 10−7M to about 10−10M) for incorporation into the IL2R binding molecule are favored.

Naturally occurring cytokine ligands for typically do not exhibit a similar affinity for each subunit of a heterodimeric receptor. Consequently, in designing a IL2R binding molecule which is a mimetic of the cognate cytokine IL2 as contemplated by some embodiments of the present disclosure, selection of sdAbs for the first and second IL2R receptor subunit have an affinity similar to (e.g., having an affinity about 10 fold, alternatively about 20 fold, or alternatively about 50 fold higher or lower than) the cognate IL2 for the respective receptor subunit may be used.

In some embodiments, the IL2R binding molecules of the present disclosure are partial agonists of the IL2R receptor. As such, the activity of the binding molecule may be modulated by selecting sdAb which have greater or lesser affinity for either one or both of the IL2R receptor subunits. As some heterodimeric cytokine receptors are comprised of a “proprietary subunit” (i.e., a subunit which is not naturally a subunit of another multimeric receptor) and a second “common” subunit (such as CD132) which is a shared component of multiple cytokine receptors), selectivity for the formation of such receptor may be enhanced by employing first sdAb which has a higher affinity for the proprietary receptor subunit and second sdAB which exhibits a lower affinity for the common receptor subunit. Additionally, the common receptor subunit may be expressed on a wider variety of cell types than the proprietary receptor subunit. In some embodiments wherein the receptor is a heterodimeric receptor comprising a proprietary subunit and a common subunit, the first sdAb of the IL2R binding molecule exhibits a significantly greater (more than 10 times greater, alternatively more than 100 times greater, alternatively more than 1000 times greater) affinity for the proprietary receptor than the second sdAb of the IL2R binding molecule for the common receptor subunit. In one embodiment, the present disclosure provides a IL2R binding molecule wherein the affinity of the IL2Rb sdAb of has an affinity of more than 10 times greater, alternatively more than 100 times greater, alternatively more than 1000 times greater) affinity IL2Rg sdAb common receptor subunit.

### Illustrative IL2R Binding Molecules

A series of illustrative IL2R binding molecules of the present disclosure were prepared in accordance with the teaching of the Examples. Briefly, camel with a fragments of the extracellular domains of IL2Rb and IL2Rg of the IL2R receptor and single domain antibody sequences isolated in accordance with the teaching of the Examples. Nucleic acid sequences were isolated from the antibody producing cells of the camels and these were used for the construction of nucleic acid sequences optimized for the expression control system were generated. In particular, modification of nucleic acid sequences to facilitate insertion into the expression vector were performed, for example avoid undesired restriction sites and codon optimized for the host cell line in accordance with procedures well known in the art.

### Binding Experiments

All experiments were conducted in 10 mM Hepes, 150 mM NaCl, 0.05% (v/v) Polysorbate 20 (PS20) and 3 mM EDTA (HBS-EP+ buffer) on a Biacore T200 instrument equipped with a Protein A chip (Cytiva). Mono-Fc V_{H}H ligands were flowed at 5 μl/min for variable time ranging from 18 to 300 seconds, reaching the capture loads listed in the tables below.

Following ligand capture, injections of a 2-fold dilution series of his-tagged cytokine receptors typically comprising at least five concentrations between 1 μM and 1 nM were performed in either high performance or single cycle kinetics mode. Surface regeneration was achieved by flowing 10 mM glycine-HCl, pH 1.5 (60 seconds, 50 μL/min). Buffer-subtracted sensograms were processed with Biacore T200 Evaluation Software and globally fit with a 1:1 Langmuir binding model (bulk shift set to zero) to extract kinetics and affinity constants (kₐ, k_{d}, K_{D}). R_{MAX}<100 RU indicates surface density compatible with kinetics analysis.

Calculated Rmax were generated using the equation Rmax=Load (RU)×valency of ligand×(Molecular weight of analyte/Molecular weight of ligand. Surface activity was defined as the ratio experimental/calculated Rmax. See tables 16 and 17 below for sample information and experimental results.

### III. Modifications to Extend Duration of Action In Vivo

The IL2R binding molecule described herein can be modified to provide for an extended lifetime in vivo and/or extended duration of action in a subject. In some embodiments, the binding molecule can be conjugated to carrier molecules to provide desired pharmacological properties such as an extended half-life. In some embodiments, the binding molecule can be covalently linked to the Fc domain of IgG, albumin, or other molecules to extend its half-life, e.g., by pegylation, glycosylation, and the like as known in the art. In some embodiments, the IL2R binding molecule modified to provide an extended duration of action in a mammalian subject has a half-life in a mammalian of greater than 4 hours, alternatively greater than 5 hours, alternatively greater than 6 hours, alternatively greater than 7 hours, alternatively greater than 8 hours, alternatively greater than 9 hours, alternatively greater than 10 hours, alternatively greater than 12 hours, alternatively greater than 18 hours, alternatively greater than 24 hours, alternatively greater than 2 days, alternatively greater than 3 days, alternatively greater than 4 days, alternatively greater than 5 days, alternatively greater than 6 days, alternatively greater than 7 days, alternatively greater than 10 days, alternatively greater than 14 days, alternatively greater than 21 days, or alternatively greater than 30 days.

Modifications of the IL2R binding molecule to provide an extended duration of action in a mammalian subject include (but are not limited to);
conjugation of the IL2R binding molecule to one or more carrier molecules,
conjugation IL2R binding molecule to protein carriers molecules, optionally in the form of a fusion protein with additional polypeptide sequences (e.g, IL2R binding molecule-Fc fusions) and
conjugation to polymers, (e.g. water soluble polymers to provide a PEGylated IL2R binding molecule).

It should be noted that the more than one type of modification that provides for an extended duration of action in a mammalian subject may be employed with respect to a given IL2R binding molecule. For example, IL2R binding molecule of the present disclosure may comprise both amino acid substitutions that provide for an extended duration of action as well as conjugation to a carrier molecule such as a polyethylene glycol (PEG) molecule.

### Protein Carrier Molecules.

Examples of protein carrier molecules which may be covalently attached to the IL2R binding molecule to provide an extended duration of action in vivo include, but are not limited to albumins, antibodies and antibody fragments such and Fc domains of IgG molecules

### Fc Fusions:

In some embodiments, the IL2R binding molecule is conjugated to a functional domain of an Fc-fusion chimeric polypeptide molecule. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by digestion of IgG with papain. IgG Fc has a molecular weight of approximately 50 kDa. The binding molecule described herein can be conjugated to the entire Fc region, or a smaller portion that retains the ability to extend the circulating half-life of a chimeric polypeptide of which it is a part. In addition, full-length or fragmented Fc regions can be variants of the wild-type molecule. In a typical presentation, each monomer of the dimeric Fc can carry a heterologous polypeptide, the heterologous polypeptides being the same or different.

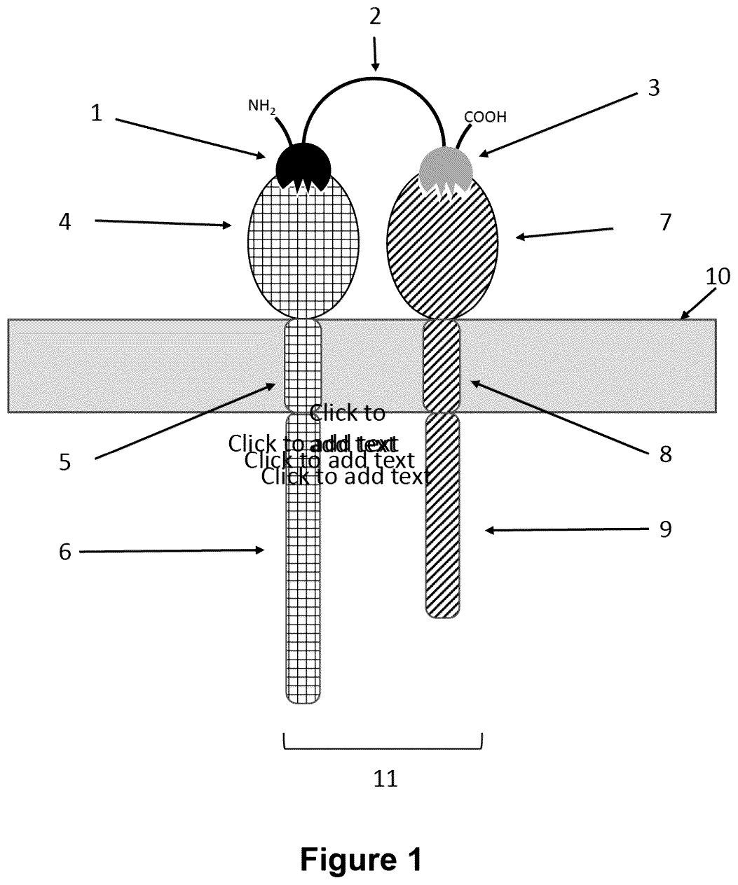

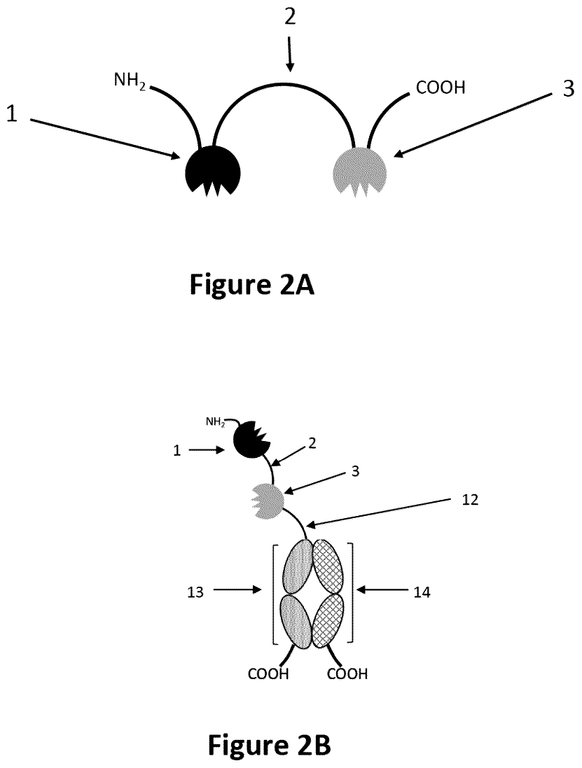

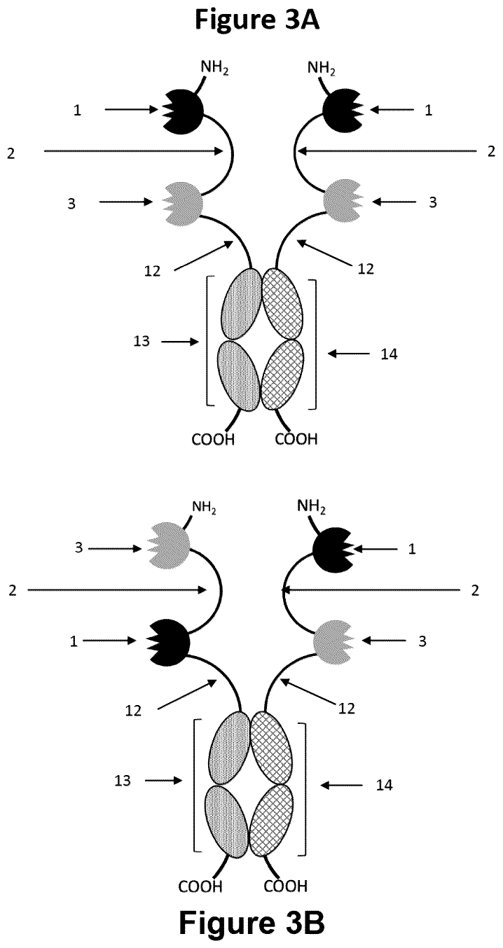

Illustrative examples of Fc formats useful for IL2R binding molecules of the present disclosure are provided schematically in FIGS. 1-FIG. 4B of the attached drawings.

### Linkage of Binding Molecule to Fc

As indicated, the linkage of the IL2R binding molecule to the Fc subunit may incorporate a linker molecule as described below between the sdAb and Fc subunit. In some embodiments, the IL2R binding molecule is expressed as a fusion protein with the Fc domain incorporating an amino acid sequence of a hinge region of an IgG antibody. The Fc domains engineered in accordance with the foregoing may be derived from IgG1, IgG2, IgG3 and IgG4 mammalian IgG species. In some embodiments, the Fc domains may be derived from human IgG1, IgG2, IgG3 and IgG4 IgG species. In some embodiments, the hinge region is the hinge region of an IgG1. In one particular embodiment, the IL2R binding is linked to an Fc domain using a human IgG1 hinge domain.

### Knob-Into-Hole Fc Format

In some embodiments, when the IL2R binding molecule described herein is to be administered in the format of an Fc fusion, particularly in those situations when the polypeptide chains conjugated to each subunit of the Fc dimer are different, the Fc fusion may be engineered to possess a “knob-into-hole modification.” The knob-into-hole modification is more fully described in Ridgway, et al. (1996) Protein Engineering 9(7):617-621 and U.S. Pat. No. 5,731,168, issued Mar. 24, 1998. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g., tyrosine or tryptophan) creating a projection from the surface (“knob”), and ii) in the CH3 domain of a second heavy chain, an amino acid residue is replaced with an amino acid residue having a smaller side chain (e.g., alanine or threonine), thereby generating a cavity (“hole”) at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 and Y349 which results in a stabilizing disulfide bridge between the two antibody heavy chains in the Fc region (Carter, et al. (2001) Immunol Methods 248, 7-15).

The knob-into-hole format is used to facilitate the expression of a first polypeptide on a first Fc monomer with a “knob” modification and a second polypeptide on the second Fc monomer possessing a “hole” modification to facilitate the expression of heterodimeric polypeptide conjugates. In some embodiments, the IL2R binding molecule covalently linked to a single subunit of the Fc as illustrated in FIG. 4A, a IL2R binding molecule is provided on each of the subunits of the Fc as illustrated in FIG. 4B.

### Albumin Carrier Molecules

In some embodiments, the IL2R binding molecule conjugated to an is albumin molecule (e.g., human serum albumin) which is known in the art to facilitate extended exposure in vivo. In one embodiment of the invention, the IL2R binding molecule is conjugated to albumin via chemical linkage or expressed as a fusion protein with an albumin molecule referred to herein as an IL2R binding molecule albumin fusion.” The term “albumin” as used in the context of hIL2 mutein albumin fusions include albumins such as human serum albumin (HSA), cyno serum albumin, and bovine serum albumin (BSA). In some embodiments, the HSA the HSA comprises a C34S or K573P amino acid substitution relative to the wild-type HSA sequence According to the present disclosure, albumin can be conjugated to an IL2R binding molecule at the carboxyl terminus, the amino terminus, both the carboxyl and amino termini, and internally (see, e.g., U.S. Pat. Nos. 5,876,969 and 7,056,701). In the HAS IL2R binding molecule contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising an IL2R binding molecule fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. As an alternative to chemical linkage between the IL2R binding molecule and the albumin molecule the IL2R binding molecule—albumin complex may be provided as a fusion protein comprising an albumin polypeptide sequence and an IL2R binding molecule recombinantly expressed in a host cell as a single polypeptide chain, optionally comprising a linker molecule between the albumin and IL2R binding molecule. Such fusion proteins may be readily prepared through recombinant technology to those of ordinary skill in the art. Nucleic acid sequences encoding such fusion proteins may be ordered from any of a variety of commercial sources. The nucleic acid sequence encoding the fusion protein is incorporated into an expression vector operably linked to one or more expression control elements, the vector introduced into a suitable host cell and the fusion protein isolated from the host cell culture by techniques well known in the art.

### Polymeric Carriers

In some embodiments, extended in vivo duration of action of the IL2R binding molecule may be achieved by conjugation to one or more polymeric carrier molecules such as XTEN polymers or water soluble polymers.

### XTEN Conjugates

The IL2R binding molecule may further comprise an XTEN polymer. The XTEN polymer may be is conjugated (either chemically or as a fusion protein) the hIL2 mutein provides extended duration of akin to PEGylation and may be produced as a recombinant fusion protein in E. coli. XTEN polymers suitable for use in conjunction with the IL2R binding molecule of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE DOI:10.1371/journal.pone.0157193-6-13, 2016. The XTEN polymer may incorporate a protease sensitive cleavage site between the XTEN polypeptide and the hIL2 mutein such as an MMP-2 cleavage site.

### Water Soluble Polymers

In some embodiments, the IL2R binding molecule can be conjugated to one or more water-soluble polymers. Examples of water soluble polymers useful in the practice of the present disclosure include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol), polysaccharides), poly-alpha-hydroxy acid), polyvinyl alcohol (PVA), polyphosphazene, polyoxazolines (POZ), poly(N-acryloylmorpholine), or a combination thereof.

In some embodiments, IL2R binding molecule can be conjugated to one or more polyethylene glycol molecules or “PEGylated.” Although the method or site of PEG attachment to the binding molecule may vary, in certain embodiments the PEGylation does not alter, or only minimally alters, the activity of the binding molecule.

PEGs suitable for conjugation to a polypeptide sequence are generally soluble in water at room temperature, and have the general formula

R(O-CH2-CH2)nO-R,

where R is hydrogen or a protective group such as an alkyl or an alkanol group, and where n is an integer from 1 to 1000. When R is a protective group, it generally has from 1 to 8 carbons. The PEG can be linear or branched. Branched PEG derivatives, “star-PEGs” and multi-armed PEGs are contemplated by the present disclosure.

In some embodiments, selective PEGylation of the IL2R binding molecule, for example, by the incorporation of non-natural amino acids having side chains to facilitate selective PEG conjugation, may be employed. Specific PEGylation sites can be chosen such that PEGylation of the binding molecule does not affect its binding to the target receptors.

In some instances, the sequences of IL2R binding molecules provided herein possess an N-terminal glutamine (“1Q”) residue. N-terminal glutamine residues have been observed to spontaneously cyclyize to form pyroglutamate (pE) at or near physiological conditions. (See e.g., Liu, et al (2011) J. Biol. Chem. 286(13): 11211-11217). In some embodiments, the formation of pyroglutamate complicates N-terminal PEG conjugation particularly when aldehyde chemistry is used for N-terminal PEGylation. Consequently, when PEGylating the IL2R binding molecules of the present disclosure, particularly when aldehyde chemistry is to be employed, the IL2R binding molecules possessing an amino acid at position 1 (e.g., 1Q) are substituted at position 1 with an alternative amino acid or are deleted at position 1 (e.g., des-1Q). In some embodiments, the IL2R binding molecules of the present disclosure comprise an amino acid substitution selected from the group Q1E and Q1D.

In certain embodiments, the increase in half-life is greater than any decrease in biological activity. PEGs suitable for conjugation to a polypeptide sequence are generally soluble in water at room temperature, and have the general formula R(O-CH2-CH2)nO-R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the binding molecule can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa, or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O-CH2-CH2)nO-R, where R is hydrogen or a protective group such as an alkyl or an alkanol group, and where n is an integer from 1 to 1000. When R is a protective group, it generally has from 1 to 8 carbonst

Two widely used first generation activated monomethoxy PEGs (mPEGs) are succinimdyl carbonate PEG (SC-PEG; see, e.g., Zalipsky, et al. (1992) Biotechnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the alpha-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein.

The PEG can be bound to a binding molecule of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of the binding molecules is facilitated by the incorporation of non-natural amino acids bearing unique side chains to facilitate site specific PEGylation. The incorporation of non-natural amino acids into polypeptides to provide functional moieties to achieve site specific PEGylation of such polypeptides is known in the art. See e.g., Ptacin et al., PCT International Application No. PCT/US2018/045257 filed Aug. 3, 2018 and published Feb. 7, 2019 as International Publication Number WO 2019/028419A1.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present disclosure include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g., Sunbright® ME-200AL, NOF), a 20 kDa linear PEG-NHS ester (e.g., Sunbright® ME-200C5, Sunbright® ME-200AS, Sunbright® ME-200GS, Sunbright® ME-200HS, NOF), a 20 kDa 2-arm branched PEG-aldehyde the 20 kDA PEG-aldehyde comprising two 10 kDA linear PEG molecules (e.g., Sunbright® GL2-200AL3, NOF), a 20 kDa 2-arm branched PEG-NHS ester the 20 kDA PEG-NHS ester comprising two 10 kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright® GL2-400AL3), a 40 kDa 2-arm branched PEG-NHS ester the 40 kDA PEG-NHS ester comprising two 20 kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

In some embodiments, a linker can used to join the IL2R binding molecule and the PEG molecule. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers are described in Section IV. Further, a multimer (e.g., 1, 2, 3, 4, 5, 6, 7, 8, 9, 10, 10-20, 20-30, or 30-50) of these linker sequences may be linked together to provide flexible linkers that may be used to conjugate two molecules. Alternative to a polypeptide linker, the linker can be a chemical linker, e.g., a PEG-aldehyde linker. In some embodiments, the binding molecule is acetylated at the N-terminus by enzymatic reaction with N-terminal acetyltransferase and, for example, acetyl CoA. Alternatively, or in addition to N-terminal acetylation, the binding molecule can be acetylated at one or more lysine residues, e.g., by enzymatic reaction with a lysine acetyltransferase. See, for example Choudhary et al. (2009) Science 325 (5942):834-840.

### Fatty Acid Carriers

In some embodiments an IL2R binding molecule having an extended duration of action in a mammalian subject and useful in the practice of the present disclosure is achieved by covalent attachment of the IL2R binding molecule to a fatty acid molecule as described in Resh (2016) Progress in Lipid Research 63: 120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine —SH groups such as DHHC proteins catalyze S-palmitoylation. Palmitoleylation of serine and threonine residues is typically achieved enzymatically using PORCN enzymes. In some embodiments, the IL2R binding molecule is acetylated at the N-terminus by enzymatic reaction with N-terminal acetyltransferase and, for example, acetyl CoA. Alternatively, or in addition to N-terminal acetylation, the IL2R binding molecule is acetylated at one or more lysine residues, e.g., by enzymatic reaction with a lysine acetyltransferase. See, for example Choudhary et al. (2009) Science 325 (5942):834L2 ortho840.

### Modifications to Provide Additional Functions

In some embodiments, embodiment, the IL2R binding molecule may comprise a functional domain of a chimeric polypeptide. IL2R binding molecule fusion proteins of the present disclosure may be readily produced by recombinant DNA methodology by techniques known in the art by constructing a recombinant vector comprising a nucleic acid sequence comprising a nucleic acid sequence encoding the IL2R binding molecule in frame with a nucleic acid sequence encoding the fusion partner either at the N-terminus or C-terminus of the IL2R binding molecule, the sequence optionally further comprising a nucleic acid sequence in frame encoding a linker or spacer polypeptide.

### FLAG Tags

In other embodiments, the IL2R binding molecule can be modified to include an additional polypeptide sequence that functions as an antigenic tag, such as a FLAG sequence.

FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the binding molecule further comprises a C-terminal c-myc epitope tag.

### Chelating Peptides

In one embodiment, the present disclosure provides a IL2R binding molecule comprising one or more transition metal chelating polypeptide sequences known as chelating peptides. A chelating peptide is a polypeptide of the formula:

(His)ₐ-(AA)_{b}-(His)_{c}

wherein “His” is the amino acid histidine; “AA” is an amino acid other than proline; is a histidine residuea=an integer from 0 to 10; b=an integer from 0 to 4; c=an integer from 0-10; and random, block and alternating copolymers thereof. In some embodiments, the chelating peptide has and amino acid sequence selected from the group consisting of SEQ ID NOS: 507-521. The incorporation of such a transition metal chelating domain facilitates purification immobilized metal affinity chromatography (IMAC) as described in Smith, et al. U.S. Pat. No. 4,569,794 issued Feb. 11, 1986. Examples of transition metal chelating polypeptides useful in the practice of the present IL2R binding molecule are described in Smith, et al. supra and Dobeli, et al. U.S. Pat. No. 5,320,663 issued May 10, 1995, the entire teachings of which are hereby incorporated by reference. Particular transition metal chelating polypeptides useful in the practice of the present IL2R binding molecule are polypeptides comprising 3-6 contiguous histidine residues (SEQ ID NO: 471) such as a six-histidine (His)₆ (SEQ ID NO:472) peptide and are frequently referred to in the art as “His-tags.” In addition to providing a purification “handle” for the recombinant proteins or to facilitate immobilization on SPR sensor chips, such the conjugation of the hIL2R binding molecule to a chelating peptide facilitates the targeted delivery to IL2R expressing cells of transition metal ions as kinetically inert or kinetically labile complexes in substantial accordance with the teaching of Anderson, et al., (U.S. Pat. No. 5,439,829 issued Aug. 8, 1995 and Hale, J. E (1996) Analytical Biochemistry 231(1):46-49.

Particular transition metal chelating polypeptides useful in the practice of the present disclosure are peptides comprising 3-6 contiguous histidine residues (SEQ ID NO: 471) such as a six-histidine peptide (His)6 (SEQ ID NO:472) are frequently referred to in the art as “His-tags.” In some embodiments, a purification handle is a polypeptide having the sequence Ala-Ser-His-His-His-His-His-His (“ASH6”) (SEQ ID NO: 23) or Gly-Ser-His-His-His-His-His-His-His-His (“GSH8”) (SEQ ID NO: 24).

### Targeting Moieties:

In some embodiments, IL2R binding molecule is conjugated to molecule which provides (“targeting domain”) to facilitate selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between IL2R binding molecule sequence and the sequence of the targeting domain of the fusion protein.

In other embodiments, a chimeric polypeptide including a IL2R binding molecule and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In some embodiments, the targeting moiety is an antibody that specifically binds to at least one cell surface molecule associated with a tumor cell (i.e. at least one tumor antigen) wherein the cell surface molecule associated with a tumor cell is selected from the group consisting of GD2, BCMA, CD19, CD33, CD38, CD70, GD2, IL3RB2, CD19, mesothelin, Her2, EpCam, Mucd, ROR1, CD133, CEA, EGRFRVIII, PSCA, GPC3, Pan-ErbB and FAP.

### Elimination of N-Linked Glycosylation Sites

In some embodiments, it is possible that an amino acid sequence (particularly a CDR sequence) of the IL2Rg or IL2Rb sdAb may contain a glycosylation motif, particularly an N-linked glycosylation motif of the sequence Asn-X-Ser (N-X-S) or Asn-X-Thr (N-X-T), wherein X is any amino acid except for proline. In such instances, it is desirable to eliminate such N-linked glycosylation motifs by modifying the sequence of the N-linked glycosylation motif to prevent glycosylation. In some embodiments, the elimination of the Asn-X-Ser (N-X-S)N-linked glycosylation motif may be achieved by the incorporation of conservative amino acid substitution of the Asn (N) residue and/or Ser (S) residue of the Asn-X-Ser (N-X-S)N-linked glycosylation motif. In some embodiments, the elimination of the Asn-X-Thr (N-X-T) N-linked glycosylation motif may be achieved by the incorporation of conservative amino acid substitution of the Asn (N) residue and/or Thr (T) residue of the Asn-X-Thr (N-X-T)N-linked glycosylation motif. In some embodiments, elimination of the glycosylation site is not required when the IL2R binding molecule comprising the IL2Rg or IL2Rb sdAb is expressed in procaryotic host cells. Since procaryotic cells do not provide a mechanism for glycosylation of recombinant proteins, when employing a procaryotic expression system to produce a recombinant IL2R binding molecule comprising the IL2Rg or IL2Rb sdAb the modification of the sequence to eliminate the N-linked glycosylation sites may be obviated.

### Recombinant Production

Alternatively, the IL2R binding molecules of the present disclosure are produced by recombinant DNA technology. In the typical practice of recombinant production of polypeptides, a nucleic acid sequence encoding the desired polypeptide is incorporated into an expression vector suitable for the host cell in which expression will be accomplish, the nucleic acid sequence being operably linked to one or more expression control sequences encoding by the vector and functional in the target host cell. The recombinant protein may be recovered through disruption of the host cell or from the cell medium if a secretion leader sequence (signal peptide) is incorporated into the polypeptide.

### Construction of Nucleic Acid Sequences Encoding the IL2R Binding Molecule

In some embodiments, the IL2R binding molecule is produced by recombinant methods using a nucleic acid sequence encoding the IL2R binding molecule (or fusion protein comprising the IL2R binding molecule). The nucleic acid sequence encoding the desired hIL2R binding molecule can be synthesized by chemical means using an oligonucleotide synthesizer.

The nucleic acid molecules are not limited to sequences that encode polypeptides; some or all of the non-coding sequences that lie upstream or downstream from a coding sequence (e.g., the coding sequence of IL-2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules encoding the IL2R binding molecule (and fusions thereof) may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences encoding the IL2R binding molecule may be obtained from various commercial sources that provide custom made nucleic acid sequences. Amino acid sequence variants of the IL2R binding molecules of the present disclosure are prepared by introducing appropriate nucleotide changes into the coding sequence based on the genetic code which is well known in the art. Such variants represent insertions, substitutions, and/or specified deletions of, residues as noted. Any combination of insertion, substitution, and/or specified deletion is made to arrive at the final construct, provided that the final construct possesses the desired biological activity as defined herein.

Methods for constructing a DNA sequence encoding a IL2R binding molecule and expressing those sequences in a suitably transformed host include, but are not limited to, using a PCR-assisted mutagenesis technique. Mutations that consist of deletions or additions of amino acid residues to a IL2R binding molecule can also be made with standard recombinant techniques. In the event of a deletion or addition, the nucleic acid molecule encoding a IL2R binding molecule is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

A IL2R binding molecule of the present disclosure may be produced recombinantly not only directly, but also as a fusion polypeptide with a heterologous polypeptide, e.g. a signal sequence or other polypeptide having a specific cleavage site at the N-terminus or C-terminus of the mature IL2R binding molecule. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. The inclusion of a signal sequence depends on whether it is desired to secrete the IL2R binding molecule from the recombinant cells in which it is made. If the chosen cells are prokaryotic, it generally is preferred that the DNA sequence not encode a signal sequence. When the recombinant host cell is a yeast cell such as Saccharomyces cerevisiae, the alpha mating factor secretion signal sequence may be employed to achieve extracellular secretion of the IL2R binding molecule into the culture medium as described in Singh, U.S. Pat. No. 7,198,919 B1 issued Apr. 3, 2007.

In the event the IL2R binding molecule to be expressed is to be expressed as a chimera (e.g., a fusion protein comprising a IL2R binding molecule and a heterologous polypeptide sequence), the chimeric protein can be encoded by a hybrid nucleic acid molecule comprising a first sequence that encodes all or part of the IL2R binding molecule and a second sequence that encodes all or part of the heterologous polypeptide. For example, subject IL2R binding molecules described herein may be fused to a hexa-/octa-histidine tag (SEQ ID NO: 472 and 473, respectively) to facilitate purification of bacterially expressed protein, or to a hemagglutinin tag to facilitate purification of protein expressed in eukaryotic cells. By first and second, it should not be understood as limiting to the orientation of the elements of the fusion protein and a heterologous polypeptide can be linked at either the N-terminus and/or C-terminus of the IL2R binding molecule. For example, the N-terminus may be linked to a targeting domain and the C-terminus linked to a hexa-histidine tag (SEQ ID NO: 472) purification handle.

The complete amino acid sequence of the polypeptide (or fusion/chimera) to be expressed can be used to construct a back-translated gene. A DNA oligomer containing a nucleotide sequence coding a IL2R binding molecule can be synthesized. For example, several small oligonucleotides coding for portions of the desired polypeptide can be synthesized and then ligated. The individual oligonucleotides typically contain 5′ or 3′ overhangs for complementary assembly.

### Codon Optimization:

In some embodiments, the nucleic acid sequence encoding the IL2R binding molecule may be “codon optimized” to facilitate expression in a particular host cell type. Techniques for codon optimization in a wide variety of expression systems, including mammalian, yeast and bacterial host cells, are well known in the and there are online tools to provide for a codon optimized sequences for expression in a variety of host cell types. See e.g. Hawash, et al., (2017) 9:46-53 and Mauro and Chappell in Recombinant Protein Expression in Mammalian Cells: Methods and Protocols, edited by David Hacker (Human Press New York). Additionally, there are a variety of web based on-line software packages that are freely available to assist in the preparation of codon optimized nucleic acid sequences.

### Expression Vectors:

Once assembled (by synthesis, site-directed mutagenesis or another method), the nucleic acid sequence encoding an a IL2R binding molecule will be inserted into an expression vector. A variety of expression vectors for uses in various host cells are available and are typically selected based on the host cell for expression. An expression vector typically includes, but is not limited to, one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors.

To facilitate efficient expression of the recombinant polypeptide, the nucleic acid sequence encoding the polypeptide sequence to be expressed is operably linked to transcriptional and translational regulatory control sequences that are functional in the chosen expression host.

### Selectable Marker:

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

### Regulatory Control Sequences:

Expression vectors for a IL2R binding molecules of the present disclosure contain a regulatory sequence that is recognized by the host organism and is operably linked to nucleic acid sequence encoding the IL2R binding molecule. The terms “regulatory control sequence,” “regulatory sequence” or “expression control sequence” are used interchangeably herein to refer to promoters, enhancers, and other expression control elements (e.g., polyadenylation signals). See, for example, Goeddel (1990) in Gene Expression Technology: Methods in Enzymology 185 (Academic Press, San Diego CA USA Regulatory sequences include those that direct constitute expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. In selecting an expression control sequence, a variety of factors understood by one of skill in the art are to be considered. These include, for example, the relative strength of the sequence, its controllability, and its compatibility with the actual DNA sequence encoding the subject a IL2R binding molecule, particularly as regards potential secondary structures.

### Promoters:

In some embodiments, the regulatory sequence is a promoter, which is selected based on, for example, the cell type in which expression is sought. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

Transcription from vectors in mammalian host cells may be controlled, for example, by promoters obtained from the genomes of viruses such as polyoma virus, fowlpox virus, adenovirus (such as human adenovirus serotype 5), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

### Enhancers:

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Additional examples of marker or reporter genes include beta-lactamase, chloramphenicol acetyltransferase (CAT), adenosine deaminase (ADA), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). Those of skill in the art can readily determine whether a given regulatory element or selectable marker is suitable for use in a particular experimental context.

Proper assembly of the expression vector can be confirmed by nucleotide sequencing, restriction mapping, and expression of a biologically active polypeptide in a suitable host.

### Host Cells:

The present disclosure further provides prokaryotic or eukaryotic cells that contain and express a nucleic acid molecule that encodes a IL2R binding molecule. A cell of the present disclosure is a transfected cell, i.e., a cell into which a nucleic acid molecule, for example a nucleic acid molecule encoding a mutant IL-2 polypeptide, has been introduced by means of recombinant DNA techniques. The progeny of such a cell are also considered within the scope of the present disclosure.

Host cells are typically selected in accordance with their compatibility with the chosen expression vector, the toxicity of the product coded for by the DNA sequences of this invention, their secretion characteristics, their ability to fold the polypeptides correctly, their fermentation or culture requirements, and the ease of purification of the products coded for by the DNA sequences. Suitable host cells for cloning or expressing the DNA in the vectors herein are the prokaryote, yeast, or higher eukaryote cells.

In some embodiments the recombinant IL2R binding molecule can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerevisiae include pYepSecl (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif), and pPicZ (Invitrogen Corporation, San Diego, Calif)); or mammalian cells (mammalian expression vectors include pCDM8 (Seed (1987) Nature 329:840) and pMT2PC (Kaufman et al. (1987) EMBO J. 6:187:195)).

Examples of useful mammalian host cell lines are mouse L cells (L-M[TK−], ATCC #CRL-2648), monkey kidney CV1 line transformed by SV40 (COS-7, ATCC CRL 1651); human embryonic kidney line (HEK293 or HEK293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In mammalian cells, the expression vector's control functions are often provided by viral regulatory elements. For example, commonly used promoters are derived from polyoma, Adenovirus 2, cytomegalovirus, and Simian Virus 40.

The IL2R binding molecule may be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

In some embodiments, a IL2R binding molecule obtained will be glycosylated or unglycosylated depending on the host organism used to produce the mutein. If bacteria are chosen as the host then the a IL2R binding molecule produced will be unglycosylated. Eukaryotic cells, on the other hand, will typically result in glycosylation of the IL2R binding molecule.

For other additional expression systems for both prokaryotic and eukaryotic cells, see Chapters 16 and 17 of Sambrook et al. (1989) Molecular Cloning: A Laboratory Manual (2nd ed., Cold Spring Harbor Laboratory Press, Plainview, N.Y.). See, Goeddel (1990) in Gene Expression Technology: Methods in Enzymology 185 (Academic Press, San Diego, Calif.).

### Transfection:

The expression constructs of the can be introduced into host cells to thereby produce a IL2R binding molecule disclosed herein. The expression vector comprising a nucleic acid sequence encoding IL2R binding molecule is introduced into the prokaryotic or eukaryotic host cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals. To facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

### Cell Culture:

Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

### Recovery of Recombinant Proteins:

Recombinantly produced IL2R binding molecule polypeptides can be recovered from the culture medium as a secreted polypeptide if a secretion leader sequence is employed. Alternatively, the IL2R binding molecule polypeptides can also be recovered from host cell lysates. A protease inhibitor, such as phenyl methyl sulfonyl fluoride (PMSF) may be employed during the recovery phase from cell lysates to inhibit proteolytic degradation during purification, and antibiotics may be included to prevent the growth of adventitious contaminants.

Various purification steps are known in the art and find use, e.g. affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural specific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column.

A recombinantly IL2R binding molecule by the transformed host can be purified according to any suitable method. Recombinant IL2R binding molecules can be isolated from inclusion bodies generated in E. coli, or from conditioned medium from either mammalian or yeast cultures producing a given mutein using cation exchange, gel filtration, and or reverse phase liquid chromatography. The substantially purified forms of the recombinant a IL2R binding molecule can be purified from the expression system using routine biochemical procedures, and can be used, e.g., as therapeutic agents, as described herein.

In some embodiments, where the IL2R binding molecule is expressed with a purification tag as discussed above, this purification handle may be used for isolation of the IL2R binding molecule from the cell lysate or cell medium. Where the purification tag is a chelating peptide, methods for the isolation of such molecules using immobilized metal affinity chromatography are well known in the art. See, e.g., Smith, et al. U.S. Pat. No. 4,569,794.

The biological activity of the IL2R binding molecules recovered can be assayed for activating by any suitable method known in the art and may be evaluated as substantially purified forms or as part of the cell lysate or cell medium when secretion leader sequences are employed for expression.

### Methods of Use

The present disclosure provides methods of use of IL2R binding molecules of the present disclosure in the treatment of a subject suffering from a neoplastic disease by the administration to the subject of therapeutically effective amount of an IL2R binding molecule, a nucleic acid encoding an IL2R binding molecule, a recombinant viral or non-viral vector encoding an IL2R binding molecules, or a recombinantly modified cell that expresses an IL2R binding molecules

The determination of whether a subject is “suffering from a neoplastic disease” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment.

The adaptive immune system recognizes the display of certain cell surface proteins in response to tumor mutations facilitating the recognition and elimination of neoplastic cells. Tumors that possess a higher tumor mutation burden (TMB) are more likely to exhibit such “tumor antigens.” Indeed, clinical experience shows that tumors comprised of neoplastic cells exhibiting a high tumor mutation burden are more likely to respond to immune therapies, including immune checkpoint blockade (Rizvi, et al. (2015) Science 348(6230): 124-128; Marabelle, et al. (2020) Lancet Oncol 21(10):1353-1365). Tumor mutation burden is useful as a biomarker to identify tumors with an increased sensitivity to immune therapies such as those provided in the present disclosure.

In some embodiments, the neoplastic disease is characterized by the presence in the subject of a benign neoplasm. Examples of benign neoplasms amenable to treatment using the compositions and methods of the present disclosure include but are not limited to adenomas, fibromas, hemangiomas, and lipomas. Examples of pre-malignant neoplasms amenable to treatment using the compositions and methods of the present disclosure include but are not limited to hyperplasia, atypia, metaplasia, and dysplasia. Examples of malignant neoplasms amenable to treatment using the compositions and methods of the present disclosure include but are not limited to carcinomas (cancers arising from epithelial tissues such as the skin or tissues that line internal organs), leukemias, lymphomas, and sarcomas typically derived from bone fat, muscle, blood vessels or connective tissues). Also included in the term neoplasms are viral induced neoplasms such as warts and EBV induced disease (i.e., infectious mononucleosis), scar formation, hyperproliferative vascular disease including intimal smooth muscle cell hyperplasia, restenosis, and vascular occlusion and the like.

The term “neoplastic disease” includes cancers characterized by solid tumors and non-solid tumors including but not limited to breast cancers; sarcomas (including but not limited to osteosarcomas and angiosarcomas and fibrosarcomas), leukemias, lymphomas, genitourinary cancers (including but not limited to ovarian, urethral, bladder, and prostate cancers); gastrointestinal cancers (including but not limited to colon esophageal and stomach cancers); lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas, astrocytomas, myelodysplastic disorders; cervical carcinoma-in-situ; intestinal polyposes; oral leukoplakias; histiocytoses, hyperprofroliferative scars including keloid scars, hemangiomas; hyperproliferative arterial stenosis, psoriasis, inflammatory arthritis; hyperkeratoses and papulosquamous eruptions including arthritis.

The term neoplastic disease includes carcinomas. The term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The term neoplastic disease includes adenocarcinomas. An “adenocarcinoma” refers to a carcinoma derived from glandular tissue or in which the tumor cells form recognizable glandular structures.

As used herein, the term “hematopoietic neoplastic disorders” refers to neoplastic diseases involving hyperplastic/neoplastic cells of hematopoietic origin, e.g., arising from myeloid, lymphoid or erythroid lineages, or precursor cells thereof.

Myeloid neoplasms include, but are not limited to, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage. Exemplary myeloid disorders amenable to treatment in accordance with the present disclosure include, but are not limited to, acute promyeloid leukemia (APML), acute myelogenous leukemia (AML) and chronic myelogenous leukemia (CML).

Lymphoid neoplasms include, but are not limited to, precursor lymphoid neoplasms, mature B-cell neoplasms, mature T-cell neoplasms, Hodgkin's Lymphoma, and immunodeficiency-associated lymphoproliferative disorders. Exemplary lymphic disorders amenable to treatment in accordance with the present disclosure include, but are not limited to, acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM).

In some instances, the hematopoietic neoplastic disorder arises from poorly differentiated acute leukemias (e.g., erythroblastic leukemia and acute megakaryoblastic leukemia). As used herein, the term “hematopoietic neoplastic disorders” refers malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), Hodgkin's disease and Reed-Steinberg disease.

In some embodiments, the compositions and methods of the present disclosure are useful in the treatment of neoplastic disease associated with the formation of solid tumors exhibiting an intermediate or high tumor mutational burden (TMB). In some embodiments, the compositions and compositions and methods of the present disclosure are useful in the treatment of immune sensitive solid tumors exhibiting an intermediate or high tumor mutational burden (TMB). Examples of neoplastic diseases associated with the formation of solid tumors having an intermediate or high tumor mutational burden amenable to treatment with the compositions and methods of the present disclosure include, but are not limited to, non-small cell lung cancer and renal cell cancer. In one embodiment, the compositions and methods are useful in the treatment of non-small cell lung cancer (NSCLC) exhibiting an intermediate or high TMB. NSCLC cells typically harbor a significant number of mutations and are therefore more sensitive to immune therapies. The current standard of care for NSCLC is stratified by the cancer initiating mechanisms and generally follows the recommendations of NCCN or ASCO. A large proportion of NSCLC has increased TMB and is therefore initially more sensitive to immune therapies. However, most tumors eventually relapse on immune checkpoint inhibition. Patients with relapsed tumors typically show reduced T cell infiltration in the tumor, systemic T cell exhaustion and a suppressed immune response compared to the lesions prior to immune checkpoint inhibition. Therefore, improved immune therapies are required, re-activating and expanding the exhausted, rare tumor infiltrating T cells.

Combination of IL2R Binding Molecules with Supplemental Therapeutic Agents:

The present disclosure provides for the use of the IL2R binding molecules of the present disclosure in combination with one or more additional active agents (“supplemental agents”). Such further combinations are referred to interchangeably as “supplemental combinations” or “supplemental combination therapy” and those therapeutic agents that are used in combination with IL2R binding molecules of the present disclosure are referred to as “supplemental agents.” As used herein, the term “supplemental agents” includes agents that can be administered or introduced separately, for example, formulated separately for separate administration (e.g., as may be provided in a kit) and/or therapies that can be administered or introduced in combination with the hIL2R binding molecules.

As used herein, the term “in combination with” when used in reference to the administration of multiple agents to a subject refers to the administration of a first agent at least one additional (i.e. second, third, fourth, fifth, etc.) agent to a subject. For purposes of the present invention, one agent (e.g. hIL2R binding molecule) is considered to be administered in combination with a second agent (e.g. a modulator of an immune checkpoint pathway) if the biological effect resulting from the administration of the first agent persists in the subject at the time of administration of the second agent such that the therapeutic effects of the first agent and second agent overlap. For example, the PD1 immune checkpoint inhibitors (e.g. nivolumab or pembrolizumab) are typically administered by IV infusion every two weeks or every three weeks while the hIL2R binding molecules of the present disclosure are typically administered more frequently, e.g. daily, BID, or weekly. However, the administration of the first agent (e.g. pembrolizumab) provides a therapeutic effect over an extended time and the administration of the second agent (e.g. an hIL2R binding molecule) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the hIL2R binding molecule and the supplemental agent(s) are administered or applied sequentially, e.g., where one agent is administered prior to one or more other agents. In other embodiments, the hIL2R binding molecule and the supplemental agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

### Supplemental Agents Useful in the Treatment of Neoplastic Disease:

In some embodiments, the supplemental agent is a chemotherapeutic agent. In some embodiments the supplemental agent is a “cocktail” of multiple chemotherapeutic agents. IN some embodiments the chemotherapeutic agent or cocktail is administered in combination with one or more physical methods (e.g. radiation therapy). The term “chemotherapeutic agents” includes but is not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins such as bleomycin A2, cactinomycin, calicheamicin, carabicin, caminomycin, carzinophilin, chromomycins, dactinomycin, daunorubicin and derivatives such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C; mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, dideazatetrahydrofolic acid, and folinic acid; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel and doxetaxel; chlorambucil; gemcitabine; 6-thioguanine; mercaptopurine; methotrexate; platinum and platinum coordination complexes such as cisplatin, oxaplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylomithine (DMFO); retinoic acid; esperamicins; capecitabine; taxanes such as paclitaxel, docetaxel, cabazitaxel; carminomycin, adriamycins such as 4′-epiadriamycin, 4-adriamycin-14-benzoate, adriamycin-14-octanoate, adriamycin-14-naphthaleneacetate; cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplemental agent isone or more chemical or biological agents identified in the art as useful in the treatment of neoplastic disease, including, but not limited to, a cytokines or cytokine antagonists such as IL-12, INFα, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the hIL2R binding molecule is administered in combination with BRAF/MEK inhibitors, kinase inhibitors such as sunitinib, PARP inhibitors such as olaparib, EGFR inhibitors such as osimertinib (Ahn, et al. (2016) J Thorac Oncol 11:S115), IDO inhibitors such as epacadostat, and oncolytic viruses such as talimogene laherparepvec (T-VEC).

In some embodiments, a “supplemental agent” is a therapeutic antibody (including bi-specific and tri-specific antibodies which bind to one or more tumor associated antigens including but not limited to bispecific T cell engagers (BITEs), dual affinity retargeting (DART) constructs, and trispecific killer engager (TriKE) constructs).

In some embodiments, the therapeutic antibody is an antibody that binds to at least one tumor antigen selected from the group consisting of HER2 (e.g. trastuzumab, pertuzumab, ado-trastuzumab emtansine), nectin-4 (e.g. enfortumab), CD79 (e.g. polatuzumab vedotin), CTLA4 (e.g. ipilumumab), CD22 (e.g. moxetumomab pasudotox), CCR4 (e.g. magamuizumab), IL23p19 (e.g. tildrakizumab), PDL1 (e.g. durvalumab, avelumab, atezolizumab), IL17a (e.g. ixekizumab), CD38 (e.g. daratumumab), SLAMF7 (e.g. elotuzumab), CD20 (e.g. rituximab, tositumomab, ibritumomab and ofatumumab), CD30 (e.g. brentuximab vedotin), CD33 (e.g. gemtuzumab ozogamicin), CD52 (e.g. alemtuzumab), EpCam, CEA, fpA33, TAG-72, CAIX, PSMA, PSA, folate binding protein, GD2 (e.g. dinuntuximab), GD3, IL6 (e.g. silutxumab) GM2, Le^{y}, VEGF (e.g. bevacizumab), VEGFR, VEGFR2 (e.g. ramucirumab), PDGFRα (e.g. olartumumab), EGFR (e.g. cetuximab, panitumumab and necitumumab), ERBB2 (e.g. trastuzumab), ERBB3, MET, IGF1R, EPHA3, TRAIL R1, TRAIL R2, RANKL RAP, tenascin, integrin αVβ3, and integrin α4β1.

Examples of antibody therapeutics which are FDA approved and may be used as supplemental agents for use in the treatment of neoplastic disease include those provided in the table below.

| Name | Tradename(s) | Target; format | Indication |
|---|---|---|---|
| [fam]-trastuzumab | Enhertu | HER2; Humanized IgG1 | HER2+ breast |
| deruxtecan | | ADC | cancer |
| Enfortumab vedotin | Padcev | Nectin-4; Human IgG1 ADC | Urothelial cancer |
| Polatuzumab vedotin | Polivy | CD79b; Humanized IgG1 | Diffuse large B-cell |
| | | ADC | lymphoma |
| Cemiplimab | Libtayo | PD-1; Human mAb | Cutaneous |
| | | | squamous cell |
| | | | carcinoma |
| Moxetumomab | Lumoxiti | CD22; Murine IgG1 dsFv | Hairy cell leukemia |
| pasudotox | | immunotoxin | |
| Mogamuizumab | Poteligeo | CCR4; Humanized IgG1 | Cutaneous T cell |
| | | | lymphoma |
| Tildrakizumab | Ilumya | IL23p19; Humanized IgG1 | Plaque psoriasis |
| Ibalizumab | Trogarzo | CD4; Humanized IgG4 | HIV infection |
| Durvalumab | IMFINZI | PD-L1; Human IgG1 | Bladder cancer |
| Inotuzumab | BESPONSA | CD22; Humanized IgG4, | Hematological |
| ozogamicin | | ADC | malignancy |
| Avelumab | Bavencio | PD-L1; Human IgG1 | Merkel cell |
| | | | carcinoma |
| Atezolizumab | Tecentriq | PD-L1; Humanized IgG1 | Bladder cancer |
| Olaratumab | Lartruvo | PDGRFα; Human IgG1 | Soft tissue sarcoma |
| Ixekizumab | Taltz | IL-17a; Humanized IgG4 | Psoriasis |
| Daratumumab | Darzalex | CD38; Human IgG1 | Multiple myeloma |
| Elotuzumab | Empliciti | SLAMF7; Humanized IgG1 | Multiple myeloma |
| Necitumumab | Portrazza | EGFR; Human IgG1 | Non-small cell lung |
| | | | cancer |
| Dinutuximab | Unituxin | GD2; Chimeric IgG1 | Neuroblastoma |
| Nivolumab | Opdivo | PD1; Human IgG4 | Melanoma, non- |
| | | | small cell lung |
| | | | cancer |
| Blinatumomab | Blincyto | CD19, CD3; Murine | Acute |
| | | bispecific tandem scFv | lymphoblastic |
| | | | leukemia |
| Pembrolizumab | Keytruda | PD1; Humanized IgG4 | Melanoma |
| Ramucirumab | Cyramza | VEGFR2; Human IgG1 | Gastric cancer |
| Siltuximab | Sylvant | IL-6; Chimeric IgG1 | Castleman disease |
| Obinutuzumab | Gazyva | CD20; Humanized IgG1; | Chronic |
| | | Glycoengineered | lymphocytic |
| | | | leukemia |
| Ado-trastuzumab | Kadcyla | HER2; Humanized IgG1, | Breast cancer |
| emtansine | | ADC | |
| Pertuzumab | Perjeta | HER2; Humanized IgG1 | Breast Cancer |
| Brentuximab vedotin | Adcetris | CD30; Chimeric IgG1, ADC | Hodgkin |
| | | | lymphoma, systemic |
| | | | anaplastic large cell |
| | | | lymphoma |
| Ipilimumab | Yervoy | CTLA-4; Human IgG1 | Metastatic |
| | | | melanoma |
| Ofatumumab | Arzerra | CD20; Human IgG1 | Chronic |
| | | | lymphocytic |
| | | | leukemia |
| Certolizumab pegol | Cimzia | TNF; Humanized Fab, | Crohn disease |
| | | pegylated | |
| Catumaxomab | Removab | EPCAM/CD3; Rat/mouse | Malignant ascites |
| | | bispecific mAb | |
| Panitumumab | Vectibix | EGFR; Human IgG2 | Colorectal cancer |
| Bevacizumab | Avastin | VEGF; Humanized IgG1 | Colorectal cancer |
| Cetuximab | Erbitux | EGFR; Chimeric IgG1 | Colorectal cancer |
| Tositumomab-I131 | Bexxar | CD20; Murine IgG2a | Non-Hodgkin |
| | | | lymphoma |
| Ibritumomab tiuxetan | Zevalin | CD20; Murine IgG1 | Non-Hodgkin |
| | | | lymphoma |
| Gemtuzumab | Mylotarg | CD33; Humanized IgG4, | Acute myeloid |
| ozogamicin | | ADC | leukemia |
| Trastuzumab | Herceptin | HER2; Humanized IgG1 | Breast cancer |
| Infliximab | Remicade | TNF; Chimeric IgG1 | Crohn disease |
| Rituximab | MabThera, Rituxan | CD20; Chimeric IgG1 | Non-Hodgkin |
| | | | lymphoma |
| Edrecolomab | Panorex | EpCAM; Murine IgG2a | Colorectal cancer |

In some embodiments, where the antibody is a bispecific antibody targeting a first and second tumor antigen such as HER2 and HER3 (abbreviated HER2×HER3), FAP×DR-5 bispecific antibodies, CEA×CD3 bispecific antibodies, CD20×CD3 bispecific antibodies, EGFR-EDV-miR16 trispecific antibodies, gp100×CD3 bispecific antibodies, Ny-eso×CD3 bispecific antibodies, EGFR×cMet bispecific antibodies, BCMA×CD3 bispecific antibodies, EGFR-EDV bispecific antibodies, CLEC12A×CD3 bispecific antibodies, HER2×HER3 bispecific antibodies, Lgr5×EGFR bispecific antibodies, PD1×CTLA-4 bispecific antibodies, CD123×CD3 bispecific antibodies, gpA33×CD3 bispecific antibodies, B7-H3×CD3 bispecific antibodies, LAG-3×PD1 bispecific antibodies, DLL4×VEGF bispecific antibodies, Cadherin-P×CD3 bispecific antibodies, BCMA×CD3 bispecific antibodies, DLL4×VEGF bispecific antibodies, CD20×CD3 bispecific antibodies, Ang-2×VEGF-A bispecific antibodies,

CD20×CD3 bispecific antibodies, CD123×CD3 bispecific antibodies, SSTR2×CD3 bispecific antibodies, PD1×CTLA-4 bispecific antibodies, HER2×HER2 bispecific antibodies, GPC3×CD3 bispecific antibodies, PSMA×CD3 bispecific antibodies, LAG-3×PD-L1 bispecific antibodies, CD38×CD3 bispecific antibodies, HER2×CD3 bispecific antibodies, GD2×CD3 bispecific antibodies, and CD33×CD3 bispecific antibodies. Such therapeutic antibodies may be further conjugated to one or more chemotherapeutic agents (e.g antibody drug conjugates or ADCs) directly or through a linker, especially acid, base or enzymatically labile linkers.

In some embodiments, a supplemental agent is one or more non-pharmacological modalities (e.g., localized radiation therapy or total body radiation therapy or surgery). By way of example, the present disclosure contemplates treatment regimens wherein a radiation phase is preceded or followed by treatment with a treatment regimen comprising an IL2R binding molecule and one or more supplemental agents. In some embodiments, the present disclosure further contemplates the use of an IL2R binding molecule in combination with surgery (e.g. tumor resection). In some embodiments, the present disclosure further contemplates the use of an IL2R binding molecule in combination with bone marrow transplantation, peripheral blood stem cell transplantation or other types of transplantation therapy.

In some embodiments, a “supplemental agent” is an immune checkpoint modulator for the treatment and/or prevention neoplastic disease in a subject as well as diseases, disorders or conditions associated with neoplastic disease. The term “immune checkpoint pathway” refers to biological response that is triggered by the binding of a first molecule (e.g. a protein such as PD1) that is expressed on an antigen presenting cell (APC) to a second molecule (e.g. a protein such as PDL1) that is expressed on an immune cell (e.g. a T-cell) which modulates the immune response, either through stimulation (e.g. upregulation of T-cell activity) or inhibition (e.g. downregulation of T-cell activity) of the immune response. The molecules that are involved in the formation of the binding pair that modulate the immune response are commonly referred to as “immune checkpoints.” The biological responses modulated by such immune checkpoint pathways are mediated by intracellular signaling pathways that lead to downstream immune effector pathways, such as cell activation, cytokine production, cell migration, cytotoxic factor secretion, and antibody production. Immune checkpoint pathways are commonly triggered by the binding of a first cell surface expressed molecule to a second cell surface molecule associated with the immune checkpoint pathway (e.g. binding of PD1 to PDL1, CTLA4 to CD28, etc.). The activation of immune checkpoint pathways can lead to stimulation or inhibition of the immune response.

In one embodiment, the immune checkpoint pathway modulator is an antagonist of a negative immune checkpoint pathway that inhibits the binding of PD1 to PDL1 and/or PDL2 (“PD1 pathway inhibitor”). PD1 pathway inhibitors result in the stimulation of a range of favorable immune response such as reversal of T-cell exhaustion, restoration cytokine production, and expansion of antigen-dependent T-cells. PD1 pathway inhibitors have been recognized as effective variety of cancers receiving approval from the USFDA for the treatment of variety of cancers including melanoma, lung cancer, kidney cancer, Hodgkins lymphoma, head and neck cancer, bladder cancer and urothelial cancer.

The term PD1 pathway inhibitors includes monoclonal antibodies that interfere with the binding of PD1 to PDL1 and/or PDL2. Antibody PD1 pathway inhibitors are well known in the art. Examples of commercially available PD1 pathway inhibitors that monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda® MK-3475, lambrolizumab, commercially available from Merck and Company, Kenilworth NJ), and atezolizumab (Tecentriq®, Genentech/Roche, South San Francisco CA). Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011.

In some embodiments, the methods of the disclosure may include the combination of the administration of an IL2R binding molecules with supplemental agents in the form of cell therapies for the treatment of neoplastic, autoimmune or inflammatory diseases. Examples of cell therapies that are amenable to use in combination with the methods of the present disclosure include but are not limited to engineered T cell products comprising one or more activated CAR-T cells, engineered TCR cells, tumor infiltrating lymphocytes (TILs), engineered Treg cells.

### Cell Therapy Agents and Methods as Supplementary Agents:

In some embodiments, the methods of the disclosure may include the combination of the administration of an IL2R binding molecules with supplementary agents in the form of cell therapies for the treatment of neoplastic, autoimmune or inflammatory diseases. Examples of cell therapies that are amenable to use in combination with the methods of the present disclosure include but are not limited to engineered T cell products comprising one or more activated CAR-T cells, engineered TCR cells, tumor infiltrating lymphocytes (TILs), engineered Treg cells. As engineered T-cell products are commonly activated ex vivo prior to their administration to the subject and therefore provide upregulated levels of CD25, cell products comprising such activated engineered T cells types are amenable to further support via the administration of an CD25 biased IL2R binding molecule as described herein.

In some embodiments of the methods of the present disclosure, the supplementary agent is a “chimeric antigen receptor T-cell” and “CAR-T cell” are used interchangeably to refer to a T-cell that has been recombinantly modified to express a chimeric antigen receptor. As used herein, the terms As used herein, the terms “chimeric antigen receptor” and “CAR” are used interchangeably to refer to a chimeric polypeptide comprising multiple functional domains arranged from amino to carboxy terminus in the sequence: (a) an antigen binding domain (ABD), (b) a transmembrane domain (TD); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface of a cell transformed with an expression vector comprising a nucleic acid sequence encoding the CAR. CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3(4):388-398; Jensen and Riddell (2015) Current Opinions in Immunology 33:9-15; Gross, et al. (1989) PNAS (USA) 86(24):10024-10028; Curran, et al. (2012) J Gene Med 14(6):405-15. Examples of commercially available CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis).

In some embodiments, the CAR-T cells comprise an antigen binding domain (ABD) refers to a polypeptide that specifically binds to an antigen expressed on the surface of a target cell. In some embodiments, the CAR-T cells useful as supplementary agents comprise and ABD is a polypeptide that specifically binds to a cell surface molecule associated with a tumor cell is selected from the group consisting of GD2, BCMA, CD19, CD33, CD38, CD70, GD2, IL3Rβ2, CD19, mesothelin, Her2, EpCam, Mucd, ROR1, CD133, CEA, EGRFRVIII, PSCA, GPC3, Pan-ErbB and FAP. In some embodiments, the ABD is an antibody (as defined hereinabove to include molecules such as one or more VHHs, scFvs, etc.) that specifically binds to at least one cell surface molecule associated with a tumor cell (i.e. at least one tumor antigen) wherein the cell surface molecule associated with a tumor cell is selected from the group consisting of GD2, BCMA, CD19, CD33, CD38, CD70, GD2, IL3Rβ2, CD19, mesothelin, Her2, EpCam, Mucd, ROR1, CD133, CEA, EGRFRVIII, PSCA, GPC3, Pan-ErbB and FAP.

In some embodiments, the engineered T cell is allogeneic with respect to the individual that is treated. Graham et al. (2018) Cell 7(10) E155. In some embodiments an allogeneic engineered T cell is fully HLA matched. However not all patients have a fully matched donor and a cellular product suitable for all patients independent of HLA type provides an alternative. If the T cells used in the practice of the present invention are allogeneic T cells, such cells may be modified to reduce graft versus host disease. For example, the engineered cells of the present invention may be TCRαβ receptor knock-outs achieved by gene editing techniques. TCRαβ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-CreI homing endonuclease, etc. See, for example, Eyquem et al. (2017) Nature 543:113-117, in which the TRAC coding sequence is replaced by a CAR coding sequence; and Georgiadis et al. (2018) Mol. Ther. 26:1215-1227, which linked CAR expression with TRAC disruption by clustered regularly interspaced short palindromic repeats (CRISPR)/Cas9 without directly incorporating the CAR into the TRAC loci. An alternative strategy to prevent GVHD modifies T cells to express an inhibitor of TCRαβ signaling, for example using a truncated form of CD3 (as a TCR inhibitory molecule.

### Chemokine and Cytokine Agents as Supplementary Agents:

In some embodiments the IL2R binding molecule is administered in combination with additional cytokines including but not limited to IL-7, IL-12, IL-15 and IL-18 including analogs and variants of each thereof.

### Activation-Induced Cell Death Inhibitors

In some embodiments the IL2R binding molecule is administered in combination with one or more supplementary agents that inhibit Activation-Induced Cell Death (AICD). AICD is a form of programmed cell death resulting from the interaction of Fas receptors (e.g., Fas, CD95) with Fas ligands (e.g., FasL, CD95 ligand), helps to maintain peripheral immune tolerance. The AICD effector cell expresses FasL, and apoptosis is induced in the cell expressing the Fas receptor. Activation-induced cell death is a negative regulator of activated T lymphocytes resulting from repeated stimulation of their T-cell receptors. Examples of agents that inhibit AICD that may be used in combination with the IL2R binding molecules described herein include but are not limited to cyclosporin A (Shih, et al., (1989) Nature 339:625-626, IL-16 and analogs (including rhIL-16, Idziorek, et al., (1998) Clinical and Experimental Immunology 112:84-91), TGFb1 (Genesteir, et al., (1999) J Exp Med189(2): 231-239), and vitamin E (Li-Weber, et al., (2002) J Clin Investigation 110(5):681-690).

### Physical Methods:

In some embodiments, the supplementary agent is a anti-neoplastic physical methods including but not limited to radiotherapy, cryotherapy, hyperthermic therapy, surgery, laser ablation, and proton therapy.

### Dosage:

Dosage, toxicity and therapeutic efficacy of such subject IL2R binding molecules or nucleic acids compounds can be determined by standard pharmaceutical procedures in cell cultures or experimental animals. The data obtained from the cell culture assays and animal studies can be used in formulating a range of dosage for use in humans. The dosage of such compounds lies preferably within a range of circulating concentrations that include the ED50 with minimal acceptable toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 as determined in cell culture. Such information can be used to more accurately determine useful doses in humans. Levels in plasma may be measured, for example, by high performance liquid chromatography.

As defined herein, a therapeutically effective amount of a subject IL2R binding molecule (i.e., an effective dosage) depends on the polypeptide selected. For instance, single dose amounts in the range of approximately 0.001 to 0.1 mg/kg of patient body weight can be administered; in some embodiments, about 0.005, 0.01, 0.05 mg/kg may be administered. In some embodiments, 600,000 IU/kg is administered (IU can be determined by a lymphocyte proliferation bioassay and is expressed in International Units (IU) as established by the World Health Organization 1st International Standard for Interleukin-2 (human)).

In some embodiments, the pharmaceutically acceptable forms of the IL2R binding molecules of the present disclosure are administered to a subject in accordance with a “low-dose” treatment protocol as described in Klatzman, et al. U.S. Pat. Nos. 9,669,071 and 10,293,028B2 the entire teachings of which are herein incorporated by reference. Additional low dose protocols are described in Smith, K. A. (1993) Blood 81(6):1414-1423, He, et al., (2016) Nature Medicine 22(9): 991-993

### Prophylactic Applications

In some embodiments where the IL2R binding molecule is used in prophylaxis of disease, the supplementary agent may be a vaccine. The IL2R binding molecule of the present invention may be administered to a subject in combination with vaccines as an adjuvant to enhance the immune response to the vaccine in accordance with the teaching of Doyle, et al U.S. Pat. No. 5,800,819 issued Sep. 1, 1998. Examples of vaccines that may be combined with the IL2R binding molecule of the present invention include are HSV vaccines, Bordetella pertussis, Escherichia coli vaccines, pneumococcal vaccines including multivalent pneumococcal vaccines such as Prevnar® 13, diptheria, tetanus and pertussis vaccines (including combination vaccines such as Pediatrix®) and Pentacel®), varicella vaccines, Haemophilus influenzae type B vaccines, human papilloma virus vaccines such as Garasil®, polio vaccines, Leptospirosis vaccines, combination respiratory vaccine, Moraxella vaccines, and attenuated live or killed virus vaccine products such as bovine respiratory disease vaccine (RSV), multivalent human influenza vaccines such as Fluzone® and Quadravlent Fluzone®), feline leukemia vaccine, transmissible gastroenteritis vaccine, COVID-19 vaccine, and rabies vaccine.

### Pharmaceutical Formulations

In some embodiments, the subject IL2R binding molecule (and/or nucleic acids encoding the IL2R binding molecule or recombinant cells incorporating a nucleic acid sequence and modified to express the IL2R binding molecule) can be incorporated into compositions, including pharmaceutical compositions. Such compositions typically include the polypeptide or nucleic acid molecule and a pharmaceutically acceptable carrier. A pharmaceutical composition is formulated to be compatible with its intended route of administration and is compatible with the therapeutic use for which the IL2R binding molecule is to be administered to the subject in need of treatment or prophylaxis.

### Carriers:

Carriers include a sterile diluent such as water for injection, saline solution, fixed oils, polyethylene glycols, glycerine, propylene glycol or other synthetic solvents. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. For intravenous administration, suitable carriers include physiological saline, bacteriostatic water, Cremophor EL™ (BASF, Parsippany, N.J.) or phosphate buffered saline (PBS).

### Buffers:

The term buffers includes buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5).

### Dispersions:

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Preservatives:

The pharmaceutical formulations for parenteral administration to a subject should be sterile and should be fluid to facilitate easy syringability. It should be stable under the conditions of manufacture and storage and are preserved against the contamination. Prevention of the action of microorganisms can be achieved by various antibacterial and antifungal agents, for example, agents such as benzyl alcohol or methyl parabens; antioxidants such as ascorbic acid or sodium bisulfite; chelating agents such as ethylenediaminetetraacetic acid, parabens, chlorobutanol, phenol, ascorbic acid, thimerosal, and the like. Sterile solutions can be prepared by incorporating the active compound in the required amount in an appropriate solvent with one or a combination of ingredients enumerated above, as required, followed by filtered sterilization.

### Tonicity Agents:

In many cases, it will be preferable to include isotonic agents, for example, sugars, polyalcohols such as mannitol, sorbitol, sodium chloride in the composition.

### Routes of Administration:

In some embodiments of the therapeutic methods of the present disclosure involve the administration of a pharmaceutical formulation comprising a IL2R binding molecule (and/or nucleic acids encoding the IL2R binding molecule or recombinantly modified host cells expressing the IL2R binding molecule) to a subject in need of treatment. The pharmaceutical formulation comprising a IL2R binding molecules of the present disclosure may be administered to a subject in need of treatment or prophylaxis by a variety of routes of administration, including parenteral administration, oral, topical, or inhalation routes.

### Parenteral Administration:

In some embodiments, the methods of the present disclosure involve the parenteral administration of a pharmaceutical formulation comprising a IL2R binding molecule (and/or nucleic acids encoding the IL2R binding molecule or recombinantly modified host cells expressing the IL2R binding molecule) to a subject in need of treatment. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion. The parenteral preparation can be enclosed in ampoules, disposable syringes or multiple dose vials made of glass or plastic. In one embodiment, the formulation is provided in a prefilled syringe for parenteral administration.

### Oral Administration:

In some embodiments, the methods of the present disclosure involve the oral administration of a pharmaceutical formulation comprising a IL2R binding molecule (and/or nucleic acids encoding the IL2R binding molecule or recombinantly modified host cells expressing the IL2R binding molecule) to a subject in need of treatment. Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

### Inhalation Formulations:

In some embodiments, the methods of the present disclosure involve the inhaled administration of a pharmaceutical formulation comprising a IL2R binding molecule (and/or nucleic acids encoding the IL2R binding molecule or recombinantly modified host cells expressing the IL2R binding molecule) to a subject in need of treatment. In the event of administration by inhalation, subject IL2R binding molecules, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

### Mucosal and Transdermal Formulations:

In some embodiments, the methods of the present disclosure involve the mucosal or transdermal administration of a pharmaceutical formulation comprising a IL2R binding molecule (and/or nucleic acids encoding the IL2R binding molecule or recombinantly modified host cells expressing the IL2R binding molecule) to a subject in need of treatment. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories (e.g., with conventional suppository bases such as cocoa butter and other glycerides) or retention enemas for rectal delivery. For transdermal administration, the active compounds are formulated into ointments, salves, gels, or creams as generally known in the art and may incorporate permeation enhancers such as ethanol or lanolin.

### Extended Release and Depot Formulations:

In some embodiments of the method of the present disclosure, the IL2R binding molecule is administered to a subject in need of treatment in a formulation to provide extended release of the IL2R binding molecule agent. Examples of extended release formulations of the injectable compositions can be brought about by including in the composition an agent which delays absorption, for example, aluminum monostearate and gelatin. In one embodiment, the subject IL2R binding molecules or nucleic acids are prepared with carriers that will protect the IL2R binding molecules against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Administration of Nucleic Acids Encoding the IL2R Binding Molecule:

In some embodiments of the method of the present disclosure, delivery of the IL2R binding molecule to a subject in need of treatment is achieved by the administration of a nucleic acid encoding the IL2R binding molecule. Methods for the administration nucleic acid encoding the IL2R binding molecule to a subject is achieved by transfection or infection using methods known in the art, including but not limited to the methods described in McCaffrey et al. (Nature (2002) 418:6893), Xia et al. (Nature Biotechnol. (2002) 20:1006-1010), or Putnam (Am. J. Health Syst. Pharm. (1996) 53: 151-160 erratum at Am. J. Health Syst. Pharm. (1996) 53:325). In some embodiments, the IL2R binding molecule is administered to a subject by the administration of a pharmaceutically acceptable formulation of recombinant expression vector comprising a nucleic acid sequence encoding the IL2R binding molecule operably linked to one or more expression control sequences operable in a mammalian subject. In some embodiments, the expression control sequence may be selected that is operable in a limited range of cell types (or single cell type) to facilitate the selective expression of the IL2R binding molecule in a particular target cell type. In one embodiment, the recombinant expression vector is a viral vector. In some embodiments, the recombinant vector is a recombinant viral vector. In some embodiments the recombinant viral vector is a recombinant adenoassociated virus (rAAV) or recombinant adenovirus (rAd), in particular a replication deficient adenovirus derived from human adenovirus serotypes 3 and/or 5. In some embodiments, the replication deficient adenovirus has one or more modifications to the E1 region which interfere with the ability of the virus to initiate the cell cycle and/or apoptotic pathways in a human cell. The replication deficient adenoviral vector may optionally comprise deletions in the E3 domain. In some embodiments the adenovirus is a replication competent adenovirus. In some embodiments the adenovirus is a replication competent recombinant virus engineered to selectively replicate in the target cell type.

In some embodiments, particularly for administration of IL2R binding molecules to the subject, particular for treatment of diseases of the intestinal tract or bacterial infections in a subject, the nucleic acid encoding the IL2R binding molecule may be delivered to the subject by the administration of a recombinantly modified bacteriophage vector encoding the IL2R binding molecule. As used herein, the terms ‘procaryotic virus,” “bacteriophage” and “phage” are used interchangeably hereinto describe any of a variety of bacterial viruses that infect and replicate within a bacterium. Bacteriophage selectively infect procaryotic cells, restricting the expression of the IL2R binding molecule to procaryotic cells in the subject while avoiding expression in mammalian cells. A wide variety of bacteriophages capable of selection a broad range of bacterial cells have been identified and characterized extensively in the scientific literature. In some embodiments, the phage is modified to remove adjacent motifs (PAM). Elimination of the of Cas9 sequences from the phage genome reduces ability of the Cas9 endonuclease of the target procaryotic cell to neutralize the invading phage encoding the IL2R binding molecule.

### Administration of Recombinantly Modified Cells Expressing the IL2R Binding Molecule:

In some embodiments of the method of the present disclosure, delivery of the IL2R binding molecule to a subject in need of treatment is achieved by the administration of recombinant host cells modified to express the IL2R binding molecule may be administered in the therapeutic and prophylactic applications described herein. In some embodiments, the recombinant host cells are mammalian cells, e.g., human cells.

In some embodiments, the nucleic acid sequence encoding the IL2R binding molecule (or vectors comprising same) may be maintained extrachromosomally in the recombinantly modified host cell for administration. In other embodiments, the nucleic acid sequence encoding the IL2R binding molecule may be incorporated into the genome of the host cell to be administered using at least one endonuclease to facilitate incorporate insertion of a nucleic acid sequence into the genomic sequence of the cell. As used herein, the term “endonuclease” is used to refer to a wild-type or variant enzyme capable of catalyzing the cleavage of bonds between nucleic acids within a DNA or RNA molecule, preferably a DNA molecule. Endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogenous DNA at this locus by gene repair mechanism. Examples of rare-cutting endonucleases include homing endonucleases (Grizot, et al (2009) Nucleic Acids Research 37(16):5405-5419), chimeric Zinc-Finger nucleases (ZFN) resulting from the fusion of engineered zinc-finger domains (Porteus M and Carroll D., Gene targeting using zinc finger nucleases (2005) Nature Biotechnology 23(3):967-973, a TALEN-nuclease, a Cas9 endonuclease from CRISPR system as or a modified restriction endonuclease to extended sequence specificity (Eisenschmidt, et al. 2005; 33(22): 7039-7047).

In some embodiments, particularly for administration of IL2R binding molecules to the intestinal tract, the IL2R binding molecule may be delivered to the subject by a recombinantly modified procaryotic cell (e.g., Lactobacillus lacti). The use of engineered procaryotic cells for the delivery of recombinant proteins to the intestinal tract are known in the art. See, e.g. Lin, et al. (2017) Microb Cell Fact 16:148. In some embodiments, the engineered bacterial cell expressing the IL2R binding molecule may be administered orally, typically in aqueous suspension, or rectally (e.g. enema).

### Prophylactic Applications

In some embodiments where the IL2R binding molecule is used in prophylaxis of disease, the supplementary agent may be a vaccine. The IL2R binding molecule of the present invention may be administered to a subject in combination with vaccines as an adjuvant to enhance the immune response to the vaccine in accordance with the teaching of Doyle, et al U.S. Pat. No. 5,800,819 issued Sep. 1, 1998. Examples of vaccines that may be combined with the IL2R binding molecule of the present invention include are HSV vaccines, Bordetella pertussis, Escherichia coli vaccines, pneumococcal vaccines including multivalent pneumococcal vaccines such as Prevnar® 13, diptheria, tetanus and pertussis vaccines (including combination vaccines such as Pediatrix®) and Pentacel®), varicella vaccines, Haemophilus influenzae type B vaccines, human papilloma virus vaccines such as Garasil®, polio vaccines, Leptospirosis vaccines, combination respiratory vaccine, Moraxella vaccines, and attenuated live or killed virus vaccine products such as bovine respiratory disease vaccine (RSV), multivalent human influenza vaccines such as Fluzone® and Quadravlent Fluzone®), feline leukemia vaccine, transmissible gastroenteritis vaccine, COVID-19 vaccine, and rabies vaccine.

Kits: The present disclosure also contemplates kits comprising pharmaceutical compositions IL2R binding molecules and a pharmaceutical composition thereof. The kits are generally in the form of a physical structure housing various components, as described below, and can be utilized, for example, in practicing the methods described above. A kit may comprise a IL2R binding molecule in the form of a pharmaceutical composition suitable for administration to a subject that is ready for use or in a form or requiring preparation for example, thawing, reconstitution or dilution prior to administration. When the IL2R binding molecule is in a form that needs to be reconstituted by a user, the kit may also comprise a sterile container providing a reconstitution medium comprising buffers, pharmaceutically acceptable excipients, and the like. A kit of the present disclosure can be designed for conditions necessary to properly maintain the components housed therein (e.g., refrigeration or freezing). A kit may further contain a label or packaging insert including identifying information for the components therein and instructions for their use. Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial). Labels or inserts may be provided in a physical form or a computer readable medium. In some embodiments, the actual instructions are not present in the kit, but rather the kit provides a means for obtaining the instructions from a remote source, e.g., via an internet site, including by secure access by providing a password (or scannable code such as a barcode or QR code on the container of the IL2R binding molecule or kit comprising) in compliance with governmental regulations (e.g., HIPAA) are provided.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure.

### EXAMPLES

### Example 1—V_{H}H Generation

Camels were acclimated at research facility for at least 7 days before immunization. Antigen was diluted with 1×PBS (antigen total about 1 mg). The quality of the antigen was assessed by SDS-PAGE to ensure purity (e.g., >80%). For the first time, 10 mL CFA (then followed 6 times using IFA) was added into mortar, then 10 mL antigen in 1×PBS was slowly added into the mortar with the pestle grinding. The antigen and CFA/IFA were ground until the component showed milky white color and appeared hard to disperse. Camels were injected with antigen emulsified in CFA subcutaneously at at least six sites on the body, injecting about 2 mL at each site (total of 10 mL per camel). A stronger immune response was generated by injecting more sites and in larger volumes. The immunization was conducted every week (7 days), for 7 times. The needle was inserted into the subcutaneous space for 10 to 15 seconds after each injection to avoid leakage of the emulsion. Alternatively, a light pull on the syringe plunger also prevented leakage. The blood sample was collected three days later after 7th immunization.

After immunization, the library was constructed. Briefly, RNA was extracted from blood and transcribed to cDNA. The VHH regions were obtained via two-step PCR, which fragment about 400 bp. The PCR outcomes and the vector of pMECS phagemid were digested with Pst I and Not I, subsequently, ligated to pMECS/Nb recombinant. After ligation, the products were transformed into Escherichia coli (E. coli) TG1 cells by electroporation. Then, the transformants were enriched in growth medium and planted on plates. Finally, the library size was estimated by counting the number of colonies.

Library biopanning was conducted to screen candidates against the antigens after library construction. Phage display technology was applied in this procedure. Positive colonies were identified by PE-ELISA.

### Example 2—Recombinant Production and Purification

Codon optimized DNA inserts were cloned into modified pcDNA3.4 (Genscript) for small scale expression in HEK293 cells in 24 well plates. The binding molecules were purified in substantial accordance with the following procedure. Using a Hamilton Star automated system, 96×4 mL of supernatants in 4×24-well blocks were re-arrayed into 4×96-well, 1 mL blocks. PhyNexus micropipette tips (Biotage, San Jose CA) holding 80 μL of Ni-Excel IMAC resin (Cytiva) are equilibrated wash buffer: PBS pH 7.4, 30 mM imidazole. PhyNexus tips were dipped and cycled through 14 cycles of 1 mL pipetting across all 4×96-well blocks. PhyNexus tips were washed in 2×1 mL blocks holding wash buffer. PhyNexus tips were eluted in 3×0.36 mL blocks holding elution buffer: PBS pH 7.4, 400 mM imidazole. PhyNexus tips were regenerated in 3×1 mL blocks of 0.5 M sodium hydroxide.

The purified protein eluates were quantified using a Biacore® T200 as in substantial accordance with the following procedure. 10 μL of the first 96×0.36 mL eluates were transferred to a Biacore® 96-well microplate and diluted to 60 uL in HBS-EP+ buffer (10 mM Hepes pH 7.4, 150 mM NaCl, 1 mM EDTA, 0.05% Tween 20). Each of the 96 samples was injected on a CM5 series S chip previously functionalized with anti-histidine capture antibody (Cytiva): injection is performed for 18 seconds at 5 μL/min. Capture levels were recorded 60 seconds after buffer wash. A standard curve of known VHH concentrations (270, 90, 30, 10, 3.3, 1.1 μg/mL) was acquired in each of the 4 Biacore chip flow cells to eliminate cell-to-cell surface variability. The 96 captures were interpolated against the standard curve using a non-linear model including specific and unspecific, one-site binding. Concentrations in the first elution block varied from 12 to 452 μg/mL corresponding to a 4-149 μg. SDS-PAGE analysis of 5 randomly picked samples was performed to ensure molecular weight of eluates corresponded to expected values (˜30 kDa).

The concentration of the proteins was normalized using the Hamilton Star automated system in substantial accordance with the following procedure. Concentration values are imported in an Excel spreadsheet where pipetting volumes were calculated to perform dilution to 50 μg/mL in 0.22 mL. The spreadsheet was imported in a Hamilton Star method dedicated to performing dilution pipetting using the first elution block and elution buffer as diluent. The final, normalized plate was sterile filtered using 0.22 μm filter plates (Corning).

### Example 3—ELISA Studies for IL2Rb VHH

The single domain antibodies of the present disclosure were obtained from camels by immunization with an extracellular domain of a IL2Rb receptor. IL2Rb VHH molecules of the present disclosure of the present disclosure were generated in substantial accordance with the teaching of the Examples. Briefly, a camel was sequentially immunized with the ECD of the human IL2Rb and mouse IL2Rb over a period several weeks of by the subcutaneous an adjuvanted composition containing a recombinantly produced fusion proteins comprising the extracellular domain of the IL2Rb, the human IgG1 hinge domain and the human IgG1 heavy chain Fc. Following immunization, RNAs extracted from a blood sample of appropriate size VHH-hinge-CH2-CH3 species were transcribed to generate DNA sequences, digested to identify the approximately 400 bp fragment comprising the nucleic acid sequence encoding the VHH domain was isolated. The isolated sequence was digested with restriction endonucleases to facilitate insertion into a phagemid vector for in frame with a sequence encoding a his-tag and transformed into E. coli to generate a phage library. Multiple rounds of biopanning of the phage library were conducted to identify VHHs that bound to the ECD of IL2Rb (human or mouse as appropriate). Individual phage clones were isolated for periplasmic extract ELISA (PE-ELISA) in a 96-well plate format and selective binding confirmed by colorimetric determination. The IL2Rb binding molecules that demonstrated specific binding to the IL2Rb antigen were isolated and sequenced and sequences analyzed to identify VHH sequences, CDRs and identify unique VHH clonotypes. As used herein, the term “clonotypes” refers a collection of binding molecules that originate from the same B-cell progenitor cell, in particular collection of antigen binding molecules that belong to the same germline family, have the same CDR3 lengths, and have 70% or greater homology in CDR3 sequence. The VHH molecules demonstrating specific binding to the hIL2Rb ECD antigen (anti-human IL2Rb VHHs) and the CDRs isolated from such VHHs are provided in Table 6. The VHH molecules demonstrating specific binding to the mIL2Rb ECD antigen (anti-mouse IL2Rb VHHs) and the CDRs isolated from such VHHs are provided in Table 3. Nucleic acid sequences encoding the VHHs of Table 7 are provided in and 11.

To more fully characterize the binding properties and evaluate binding affinity of the VHH molecules generated in accordance with the foregoing, representative examples of each of the human VHH clonotypes were subjected to analysis by surface plasmon resonance in substantial accordance with the teaching of Example 5 herein. The results of these SPR studies are summarized in Table 17 below.

### Example 4. Evaluation of Binding Affinity Via Surface Plasmon Resonance for IL2Rb VHH

A representative example from each hIL2Rb VHH clonotype generated as descried above was selected for evaluation of binding via SPR as follows. Evaluation of binding affinity of the hIL2Rb binding molecules shown in Table 16 was conducted using surface plasmon resonance (SPR) in substantial accordance with the following procedure. All experiments were conducted in 10 mM Hepes, 150 mM NaCl, 0.05% (v/v) Polysorbate 20 (PS20) and 3 mM EDTA (HBS-EP+ buffer) on a Biacore T200 instrument equipped with a Protein A derivatized sensor chip (Cytiva). Mono-Fc VHH ligands were flowed at 5 μl/min for variable time ranging from 18 to 300 seconds, reaching the capture loads listed in the tables below. Following ligand capture, injections of a 2-fold dilution series of the extracellular domain of the IL2Rb-receptor modified to incorporate a C-terminal poly-His sequence, typically comprising at least five concentrations between 1 μM and 1 nM, were performed in either high performance or single cycle kinetics mode. Surface regeneration was achieved by flowing 10 mM glycine-HCl, pH 1.5 (60 seconds, 50 L/min). Buffer-subtracted sensograms were processed with Biacore T200 Evaluation Software and globally fit with a 1:1 Langmuir binding model (bulk shift set to zero) to extract kinetics and affinity constants (kₐ, k_{d}, K_{D}). R_{MAX}<100 RU indicates surface density compatible with kinetics analysis. Calculated Rₘₐₓ values were generated using the equation: Rmax=Load (RU)×valency of ligand×(Molecular weight of analyte/Molecular weight of ligand). Surface activity was defined as the ratio of experimental/calculated Rmax. The results of these binding affinity experiments are provided in Table 16.

TABLE 16
anti-hIL2Rb Mono-Fc VHHs binding to hIL2Rb-his

| | | | | | | | Calc. | |
|---|---|---|---|---|---|---|---|---|
| | SEQ | k_{ON} | k_{OFF} | Affinity | Rmax | Load | Rmax | Surface |
| Ligand | ID NO | (1/Ms) | (1/s) | (nM) | (RU) | (RU) | (RU) | Activity |
| hIL2Rb_VHH1 | 25 | 1.98E+07 | 1.99E−02 | 1 | 17.6 | 62.4 | 48 | 37% |
| hIL2Rb_VHH2 | 26 | 1.39E+05 | 2.24E−03 | 16 | 5.4 | 26.8 | 20 | 26% |
| hIL2Rb_VHH3 | 27 | 1.57E+05 | 6.99E−03 | 47 | 14.7 | 36.8 | 28 | 52% |
| hIL2Rb_VHH4 | 28 | 6.00E+05 | 2.05E−03 | 3.4 | 24.4 | 33.2 | 25 | 96% |
| hIL2Rb_VHH5 | 29 | 3.82E+06 | 1.54E−03 | 0.4 | 32.3 | 100 | 77 | 42% |
| hIL2Rb_VHH6 | 30 | 1.90E+07 | 2.29E−02 | 1.2 | 38.9 | 96.9 | 74 | 53% |
| hIL2Rb_VHH7 | 31 | 2.86E+06 | 3.17E−03 | 1.1 | 32.1 | 98.3 | 75 | 43% |
| hIL2Rb_VHH8 | 32 | IND | ND | ND | ~5 | 279 | 213 | <5% |

### Example 5—ELISA Studies for IL2R2 VHH

The single domain antibodies of the present disclosure were obtained from camels by immunization with an extracellular domain of a IL2Rg receptor (CD132). IL2Rg VHH molecules of the present disclosure of the present disclosure were generated in substantial accordance with the teaching of the Examples. Briefly, a camel was sequentially immunized with the ECD of the human IL2Rg and mouse IL2Rg over a period several weeks of by the subcutaneous an adjuvanted composition containing a recombinantly produced fusion proteins comprising the extracellular domain of the IL2Rg, the human IgG1 hinge domain and the human IgG1 heavy chain Fc. Following immunization, RNAs extracted from a blood sample of appropriate size VHH-hinge-CH2-CH3 species were transcribed to generate DNA sequences, digested to identify the approximately 400 bp fragment comprising the nucleic acid sequence encoding the VHH domain and isolated. The isolated sequence was digested with restriction endonucleases to facilitate insertion into a phagemid vector for in frame with a sequence encoding a his-tag and transformed into E. coli to generate a phage library. Multiple rounds of biopanning of the phage library were conducted to identify VHHs that bound to the ECD of IL2Rg (human or mouse as appropriate). Individual phage clones were isolated for periplasmic extract ELISA (PE-ELISA) in a 96-well plate format and selective binding confirmed by colorimetric determination. The IL2Rg binding molecules that demonstrated specific binding to the IL2Rg antigen were isolated and sequenced and sequences analyzed to identify VHH sequences, CDRs and identify unique VHH clonotypes. As used herein, the term “clonotypes” refers a collection of binding molecules that originate from the same B-cell progenitor cell, in particular collection of antigen binding molecules that belong to the same germline family, have the same CDR3 lengths, and have 70% or greater homology in CDR3 sequence. The VHH molecules demonstrating specific binding to the hIL2Rg ECD antigen (anti-human IL2Rg VHHs) and the CDRs isolated from such VHHs are provided in Table 8. The VHH molecules demonstrating specific binding to the mIL2Rg ECD antigen (anti-mouse IL2Rg VHHs) and the CDRs isolated from such VHHs are provided in Table 5. Nucleic acid sequences encoding the VHHs of Table 8 and 9 are provided in Tables 12 and 13, respectively.

### Example 6. Evaluation of Binding Affinity Via Surface Plasmon Resonance for IL2R VHH

To more fully characterize the binding properties and evaluate binding affinity of the VHH molecules generated in accordance with the foregoing, representative examples of each of the human VHH clonotypes were subjected to analysis by surface plasmon resonance in substantial accordance with the teaching of the examples herein. The results of these SPR studies are summarized in Table 17 below.

TABLE 17
anti-hIL2Rg Mono-Fc VHHs binding to hIL2Rg-his
(Antigen: Sino Biological, Catalog# 10555)

| | | | | | | | Calc. | |
|---|---|---|---|---|---|---|---|---|
| | SEQ | k_{ON} | k_{OFF} | Affinity | Rmax | Load | Rmax | Surface |
| Ligand | ID NO | (1/Ms) | (1/s) | (nM) | (RU) | (RU) | (RU) | Activity |
| hIL-2Rg_VHH8 | 306 | 3.66E+05 | 8.12E−04 | 2.2 | 41.6 | 49.3 | 38 | 110% |
| hIL-2Rg_VHH15 | 313 | 9.68E+04 | 2.51E−03 | 26 | 47.1 | 84 | 64 | 73% |
| hIL-2Rg_VHH19 | 317 | 2.85E+06 | 6.93E−03 | 2.4 | 7 | 28 | 21 | 33% |
| hIL-2Rg_VHH20 | 318 | 1.92E+05 | 2.70E−03 | 14.1 | 227 | 103 | 475.4 | 48% |
| hIL-2Rg_VHH21 | 319 | 6.56E+04 | 1.13E−03 | 17.2 | 14.6 | 57.5 | 245.8 | 6% |
| hIL-2Rg_VHH22 | 320 | 5.54E+05 | 3.54E−03 | 6.4 | 39.6 | 47.8 | 37 | 108% |

As illustrated by the data presented in Table 17, the hIL2Rg binding molecules generated in accordance with the teaching of present disclosure exhibit specific binding and provided a range of affinities to the extracellular domain of hIL2Rg.

### Example 7 Evaluation of Binding Affinity Via Surface Plasmon Resonance for IL2IL2Rg VHH

### Murine Dimer Constructs

Additional experiments were conducted with murine dimer constructs. All experiments were conducted in 10 mM Hepes, 150 mM NaCl, 0.05% (v/v) Polysorbate 20 (PS20) and 3 mM EDTA (HBS-EP+ buffer) on a Biacore T200 instrument equipped with Protein A or CAP biotin chips (Cytiva). For experiments on Protein A chips, Fc-fused ligands were flowed at 5 μl/min for variable time ranging from 18 to 300 seconds, reaching the capture loads listed in the tables below.

Following ligand capture, injections of a 2-fold dilution series of analyte typically comprising at least five concentrations between 1 μM and 1 nM were performed in either high performance or single cycle kinetics mode. Surface regeneration was achieved by flowing 10 mM glycine-HCl, pH 1.5 (60 seconds, 50 μL/min). Buffer-subtracted sensograms were processed with Biacore T200 Evaluation Software and globally fit with a 1:1 Langmuir binding model (bulk shift set to zero) to extract kinetics and affinity constants (ka, kd, KD). RMAX<100 RU indicates surface density compatible with kinetics analysis.

Experiments on CAP chips were performed as described above with an additional capture step of Biotin CAPture reagent (10 seconds, 40 uL/min) performed prior to capture of biotinylated ligands.

Calculated Rmax were generated using the equation Rmax=Load (RU)×valency of ligand×(Molecular weight of analyte/Molecular weight of ligand. Surface activity was defined as the ratio experimental/calculated Rmax. See tables below for sample information and experimental results.

Results for Anti-mIL2Rb/mIL2Rg dual VHHs binding to mIL2Rb-Fc were as follows:

TABLE 18

| | | | | | | | Calc. | |
|---|---|---|---|---|---|---|---|---|
| | | | | Affinity | Rmax | Load | Rmax | Surface |
| Analyte | Ligand | k_{ON} (1/Ms) | k_{OFF} (1/s) | (nM) | (RU) | (RU) | (RU) | Activity |
| DR870-his | mIL2Rb-Fc | 4.0E+04 | 1.6E−03 | 39 | 13.9 | 181 | 169 | 8% |
| DR871-his | (SinoBiological | <5.2E+05 | 9.9E−04 | >1.9 | 18.4 | 175 | 164 | 11% |
| DR873-his | cat#50792) | 5.2E+04 | 1.6E−03 | 31 | 16.6 | 172 | 160 | 10% |

Results for Anti-mIL2Rb/mIL2Rg dual VHHs binding to mIL2Rg-Fc (Sino Biological, catalog #50087) were as follows:

TABLE 19

| | | | | | | | Calc. | |
|---|---|---|---|---|---|---|---|---|
| | | | | Affinity | Rmax | Load | Rmax | Surface |
| Analyte | Ligand | k_{ON} (1/Ms) | k_{OFF} (1/s) | (nM) | (RU) | (RU) | (RU) | Activity |
| DR870-his | mIL2Rg-Fc | 3.2E+06 | 1.6E−03 | 0.49 | 68.5 | 198 | 148 | 46% |
| DR871-his | (SinoBiological | 2.8E+06 | <1E−05 | <0.01 | 87.8 | 200 | 149 | 59% |
| DR873-his | cat#50087) | 6.1E+06 | 4.4E−04 | 0.072 | 80.9 | 195 | 146 | 56% |

TABLE 20
Anti hIL10Ra/hIL10Rb dual VHH binding to human and cynomolgus IL10Ra and IL10Rb

| | | | | | | | Calc. | |
|---|---|---|---|---|---|---|---|---|
| | | | | Affinity | Rmax | Load | Rmax | Surface |
| Analyte | Ligand | k_{ON} (1/Ms) | k_{OFF} (1/s) | (nM) | (RU) | (RU) | (RU) | Activity |
| hIL10Ra_VHH17 | hIL10Ra-Fc (R&D | 2.1E+06 | 4.7E−04 | 0.23 | 51.2 | 127 | 71 | 72% |
| Q1E N29Q- | Systems cat | | | | | | | |
| hIL10Rb_VHH16- | #9044) | | | | | | | |
| 0PEG | CIL10Ra-Fc | 2.5E+06 | 6.6E−04 | 0.27 | 63.1 | 146 | 82 | 77% |
| lot#P210301BJ2 | (lot#P210115SV6) | | | | | | | |
| | hIL10Rb-Fc | 7.7E+05 | 1.6E−03 | 2.05 | 52.1 | 116 | 65 | 80% |
| | (SinoBiological | | | | | | | |
| | cat#10945) | | | | | | | |
| | CIL10Rb-Fc (lot# | 7.5E+05 | 1.5E−03 | 2.01 | 46.9 | 89.6 | 50 | 93% |
| | P210115SV7) | | | | | | | |

TABLE 21
Anti-hIL10Ra/hIL10Rb dual VHH binding to hIL10Rb-his
(Sino Biological cat#10945-H08H)

| | | | | | | | Calc. | |
|---|---|---|---|---|---|---|---|---|
| | | k_{ON} | k_{OFF} | Affinity | Rmax | Load | Rmax | |
| Analyte | Ligand | (1/Ms) | (1/s) | (nM) | (RU) | (RU) | (RU) | Activity |
| hIL10Rb- | hIL10Ra_VHH17- | 1.6E+05 | 4.5E−04 | 2.9 | 30 | 62 | 86.8 | 34% |
| his | hIL10Rb_VHH16 | | | | | | | |
| (Sino | biotin | | | | | | | |
| Biological | hIL10Rb_VHH12- | 4.4E+05 | 6.8E−04 | 1.5 | 32 | 53 | 74.2 | 42% |
| cat#10945) | hIL10Ra_VHH8 | | | | | | | |
| | biotin | | | | | | | |
| | hIL10Rb_VHH1- | 6.6E+05 | 1.8E−03 | 2.7 | 31 | 90 | 126 | 25% |
| | hIL10Ra_VHH9 | | | | | | | |
| | biotin | | | | | | | |
| | hIL10Ra_VHH12- | 1.6E+06 | 3.0E−03 | 1.9 | 100 | 84 | 117.6 | 85% |
| | hIL10Rb_VHH27 | | | | | | | |
| | biotin | | | | | | | |

### Example 8. Evaluation of Activity of IL-2 VHH Dimers on NKL Cells

The IL2 VHH dimers were evaluated for activity in NKL cells (Robertson, et al (1996) Experimental Hematology 24(3):406-15). NKL cells are an IL-2 dependent human cell line that expresses IL-2R3 and IL-2Rγ chains and can respond to IL-2 by phosphorylation of STAT5 and proliferation.

NKL were contacted with purified VHH dimers to examine induction of STAT5 phosphorylation as follows: Cells were seeded in growth medium consisting of RPMI 1640 (ThermoFisher), 10 percent fetal bovine serum (ThermoFisher), 1 percent penicillin/streptomycin (ThermoFisher), 1 percent glutamax (ThermoFisher) at 0.5 million cells per ml. After two days of culture, cells were seeded into 96-well plates (Falcon) at 100 thousand cells per well in 90 μl DPBS prewarmed at 37 degrees centigrade. Ten μl of each of the 120 purified VHH dimers in DPBS at 300 nM was added to the cells and plates were transferred to a humidified incubator (ThermoFisher) and incubated at 37 degrees centigrade, 5 percent carbon dioxide for 20 minutes.

Plates were removed from the incubator and 100 μl 2× Complete Lysis buffer (Tris Lysis Buffer, Protease Inhibitor Solution, Phosphatase Inhibitor I, Phosphatase Inhibitor II) was added according to manufacturer's instructions (MSD Phospho-STAT Panel K15202D). Plates were incubated on ice for 15 minutes and centrifuged for 5 minutes at 600×g and Lysates were transferred to a new 96 well plate.

The level of phospho-STAT5 induction in the lysate was measured using the MSD multi-spot assay system with the Phospho-STAT panel kit (K15202D) according to manufacturer's instructions. MSD 96 well assay plates were washed 3 times with 1×Tris wash buffer and 150 μl Blocker A solution was added to each well. Plates were incubated on an orbital shaker (VWR Scientific) for 60 minutes at room temperature and washed 3 times with 1×Tris wash buffer. Cell lysates (25 μl) were added to the plate. Plates were incubated on an orbital shaker (VWR Scientific) for 60 minutes at room temperature and washed 3 times with 1×Tris wash buffer. Detection antibody solution (25 μl) was added to the plate. Plates were incubated on an orbital shaker (VWR Scientific) for 60 minutes at room temperature and washed 3 times with 1×Tris wash buffer. 150 μl 1× Read Buffer T was added to each well and emitted light intensity was read in luminescence units on a MSD Quickplex SQ120 instrument.

For measurement of proliferation, NKL were contacted with purified VHH dimers as follows: Cells were seeded in growth medium consisting of RPMI 1640 (ThermoFisher), 10 percent fetal bovine serum (ThermoFisher), 1 percent penicillin/streptomycin (ThermoFisher), 1 percent glutamax (ThermoFisher) at 0.5 million cells per ml. After two days of culture, cells were seeded into 96-well plates (Falcon) at 25 thousand cells per well in 90 μl growth medium. Ten μl of each of the 120 purified VHH dimers in DPBS at 300 nM was added to the cells and plates were transferred to a humidified incubator (ThermoFisher) and incubated at 37 degrees centigrade, 5 percent carbon dioxide for 72 hrs.

Plates were removed from the incubator and kept at room temperature for 30 minutes. Cells were lysed by adding 100 μl per well of Celltiterglo (Promega). Cell lysates were mixed on an orbital shaker (VWR Scientific) for two minutes at 200 rpm then held at room temperature for 10 minutes. Luminescence for NKL lysates were read as counts per second in an Envision 2103 Multilabel Plate Reader (Perkin Elmer).

To compare the effect of each IL-2 VHH dimer upon pSTAT5 induction and NKL cell proliferation, luminescence values from pSTAT and celltiterglo measurements were compared to those obtained for control cells treated with growth medium alone and control cells treated with human IL-2 at 100 μM. IL-2 VHH dimers were identified that induced higher luminescence signals for pSTAT5 induction and cell proliferation than media control but lower than IL-2 at the concentrations used. The data from these experiments is presented in Table 22.

TABLE 22
Activity of VHH dimers on NKL cell pSTAT5 induction and proliferation.

| | N-terminal VHH- | | |
|---|---|---|---|
| VHH SEQ ID | C-terminal VHH | pSTAT5 (LU) | Proliferation (LU) |
| DR632 | DR229-DR214 | 595 | 116506 |
| DR633 | DR229-DR217 | 870 | 138124 |
| DR634 | DR229-DR583 | 1189 | 136398 |
| DR635 | DR229-DR584 | 6264 | 178418 |
| DR636 | DR229-DR585 | 912 | 115410 |
| DR637 | DR229-DR586 | 850 | 122216 |
| DR638 | DR229-DR587 | 4495 | 148994 |
| DR639 | DR229-DR588 | 3077 | 177470 |
| DR640 | DR229-DR589 | 1145 | 137282 |
| DR641 | DR229-DR590 | 697 | 132522 |
| DR642 | DR230-DR214 | 3212 | 147492 |
| DR643 | DR230-DR217 | 5300 | 177402 |
| DR644 | DR230-DR583 | 1034 | 132806 |
| DR645 | DR230-DR584 | 2968 | 152106 |
| DR646 | DR230-DR585 | 1095 | 124118 |
| DR647 | DR230-DR586 | 5234 | 176110 |
| DR648 | DR230-DR587 | 3966 | 154232 |
| DR649 | DR230-DR588 | 579 | 135958 |
| DR650 | DR230-DR589 | 861 | 138590 |
| DR651 | DR230-DR590 | 742 | 140664 |
| DR652 | DR231-DR214 | 2626 | 168884 |
| DR653 | DR231-DR217 | 475 | 127516 |
| DR654 | DR231-DR583 | 923 | 144488 |
| DR655 | DR231-DR584 | 968 | 126880 |
| DR656 | DR231-DR585 | 1366 | 121026 |
| DR657 | DR231-DR586 | 1041 | 146186 |
| DR658 | DR231-DR587 | 683 | 105546 |
| DR659 | DR231-DR588 | 1037 | 152634 |
| DR660 | DR231-DR589 | 781 | 126740 |
| DR661 | DR231-DR590 | 888 | 136314 |
| DR662 | DR232-DR214 | 861 | 112768 |
| DR663 | DR232-DR217 | 1055 | 139844 |
| DR664 | DR232-DR583 | 967 | 119004 |
| DR665 | DR232-DR584 | 1105 | 143704 |
| DR666 | DR232-DR585 | 1188 | 130088 |
| DR667 | DR232-DR586 | 1110 | 136678 |
| DR668 | DR232-DR587 | 944 | 103200 |
| DR669 | DR232-DR588 | 931 | 147228 |
| DR670 | DR232-DR589 | 1245 | 122194 |
| DR671 | DR232-DR590 | 1599 | 163078 |
| DR672 | DR233-DR214 | 1587 | 115320 |
| DR673 | DR233-DR217 | 1653 | 150560 |
| DR674 | DR233-DR583 | 3347 | 137342 |
| DR675 | DR233-DR584 | 3561 | 167770 |
| DR676 | DR233-DR585 | 1409 | 169444 |
| DR677 | DR233-DR586 | 845 | 135714 |
| DR678 | DR233-DR587 | 6138 | 184958 |
| DR679 | DR233-DR588 | 1810 | 156864 |
| DR680 | DR233-DR589 | 2073 | 122214 |
| DR681 | DR233-DR590 | 1374 | 153052 |
| DR682 | DR234-DR214 | 1055 | 110310 |
| DR683 | DR234-DR217 | 1006 | 140478 |
| DR684 | DR234-DR583 | 725 | 107838 |
| DR685 | DR234-DR584 | 1070 | 139478 |
| DR686 | DR234-DR585 | 868 | 120032 |
| DR687 | DR234-DR586 | 1173 | 128462 |
| DR688 | DR234-DR587 | 997 | 136754 |
| DR689 | DR234-DR588 | 1227 | 149798 |
| DR690 | DR234-DR589 | 1280 | 126096 |
| DR691 | DR234-DR590 | 917 | 139710 |
| DR692 | DR214-DR229 | 898 | 108572 |
| DR693 | DR214-DR230 | 917 | 130734 |
| DR694 | DR214-DR231 | 984 | 113610 |
| DR695 | DR214-DR232 | 1262 | 137054 |
| DR696 | DR214-DR233 | 1603 | 96674 |
| DR697 | DR214-DR234 | 758 | 132076 |
| DR698 | DR217-DR229 | 1051 | 124264 |
| DR699 | DR217-DR230 | 837 | 141240 |
| DR700 | DR217-DR231 | 881 | 174410 |
| DR701 | DR217-DR232 | 2092 | 163990 |
| DR702 | DR217-DR233 | 1078 | 136290 |
| DR703 | DR217-DR234 | 734 | 140046 |
| DR704 | DR583-DR229 | 722 | 118478 |
| DR705 | DR583-DR230 | 1150 | 136916 |
| DR706 | DR583-DR231 | 752 | 113692 |
| DR707 | DR583-DR232 | 1119 | 130934 |
| DR708 | DR583-DR233 | 772 | 100954 |
| DR709 | DR583-DR234 | 1070 | 138480 |
| DR710 | DR584-DR229 | 812 | 113468 |
| DR711 | DR584-DR230 | 1091 | 134004 |
| DR712 | DR584-DR231 | 992 | 133920 |
| DR713 | DR584-DR232 | 1044 | 149190 |
| DR714 | DR584-DR233 | 923 | 116526 |
| DR715 | DR584-DR234 | 1003 | 136656 |
| DR716 | DR585-DR229 | 2639 | 156810 |
| DR717 | DR585-DR230 | 2103 | 158976 |
| DR718 | DR585-DR231 | 6348 | 167952 |
| DR719 | DR585-DR232 | 1957 | 158644 |
| DR720 | DR585-DR233 | 1999 | 145766 |
| DR721 | DR585-DR234 | 2778 | 168984 |
| DR722 | DR586-DR229 | 4231 | 151054 |
| DR723 | DR586-DR230 | 881 | 125438 |
| DR724 | DR586-DR231 | 5638 | 180326 |
| DR725 | DR586-DR232 | 910 | 126750 |
| DR726 | DR586-DR233 | 1132 | 125440 |
| DR727 | DR586-DR234 | 701 | 127484 |
| DR728 | DR587-DR229 | 859 | 104160 |
| DR729 | DR587-DR230 | 948 | 124594 |
| DR730 | DR587-DR231 | 662 | 102252 |
| DR731 | DR587-DR232 | 1055 | 127720 |
| DR732 | DR587-DR233 | 862 | 108350 |
| DR733 | DR587-DR234 | 771 | 133262 |
| DR734 | DR588-DR229 | 800 | 119826 |
| DR735 | DR588-DR230 | 1090 | 141022 |
| DR736 | DR588-DR231 | 1406 | 145904 |
| DR737 | DR588-DR232 | 932 | 136648 |
| DR738 | DR588-DR233 | 847 | 104204 |
| DR739 | DR588-DR234 | 1020 | 137990 |
| DR740 | DR589-DR229 | 947 | 131632 |
| DR741 | DR589-DR230 | 808 | 144776 |
| DR742 | DR589-DR231 | 1013 | 135948 |
| DR743 | DR589-DR232 | 938 | 138268 |
| DR744 | DR589-DR233 | 807 | 136268 |
| DR745 | DR589-DR234 | 638 | 132428 |
| DR746 | DR590-DR229 | 1304 | 132472 |
| DR747 | DR590-DR230 | 1622 | 157594 |
| DR748 | DR590-DR231 | 890 | 132586 |
| DR749 | DR590-DR232 | 432 | 117108 |
| DR750 | DR590-DR233 | 1041 | 142346 |
| DR751 | DR590-DR234 | 605 | 114290 |
| Media | n/a | 1194 | 147254 |
| hIL-2 | n/a | 7571 | 205850 |

### Example 9. Evaluation of Activity of IL-2 VHH Dimers on Primary NK Cells

The IL2 VHH dimers were evaluated for activity in Primary NK cells isolated from PBMC. Primary NK cells express IL-2Rβ and IL-2Rγ chains and can respond to IL-2 by phosphorylation of STAT5, proliferation and the production of IFN-γ.

PBMC were isolated from human Buffy Coats or Leucocyte Reduction System Chambers (LRSC) using the Custom Sedimentation Kit (Miltenyi, #130-126-357) and Custom Buffy Coat/LRSC PBMC Isolation kits (Miltenyi, 130-126-448) using protocol Cust5 on an autoMACS Pro Separator (Miltenyi) according to manufacturer's instructions. Purified PBMC were counted on a Vi-cell XR (Beckman Coulter) or Vi-cell Blue (Beckman Coulter) cell viability analyzer.

NK cells were isolated from human PBMC using CD56 microbeads (Miltenyi, 130-050-401) on an autoMACS Pro Separator (Miltenyi) with protocol possel according to manufacturer's instructions. Purified NK cells were counted on a Vi-cell XR (Beckman Coulter) or Vi-cell Blue (Beckman Coulter) cell viability analyzer.

NK cells were contacted with purified VHH dimers to examine induction of STAT5 phosphorylation as follows: Cells were seeded into 96-well plates (Falcon) at 100 thousand cells per well in 95 μl DPBS prewarmed at 37 degrees centigrade. Five μl of each of the 120 purified VHH dimers in DPBS at 300 nM was added to the cells and plates were transferred to a humidified incubator (ThermoFisher) and incubated at 37 degrees centigrade, 5 percent carbon dioxide for 20 minutes.

Plates were removed from the incubator and 100 μl 2× Complete Lysis buffer (Tris Lysis Buffer, Protease Inhibitor Solution, Phosphatase Inhibitor I, Phosphatase Inhibitor II) was added according to manufacturer's instructions (MSD Phospho-STAT Panel K15202D). Plates were incubated on ice for 15 minutes and centrifuged for 5 minutes at 600×g and Lysates were transferred to a new 96 well plate.

The level of phospho-STAT5 induction in the lysate was measured using the MSD multi-spot assay system with the Phospho-STAT panel kit (K15202D) according to manufacturer's instructions. MSD 96 well assay plates were washed 3 times with 1×Tris wash buffer and 150 μl Blocker A solution was added to each well. Plates were incubated on an orbital shaker (VWR Scientific) for 60 minutes at room temperature and washed 3 times with 1×Tris wash buffer. Cell lysates (25 μl) were added to the plate. Plates were incubated on an orbital shaker (VWR Scientific) for 60 minutes at room temperature and washed 3 times with 1×Tris wash buffer. Detection antibody solution (25 μl) was added to the plate. Plates were incubated on an orbital shaker (VWR Scientific) for 60 minutes at room temperature and washed 3 times with 1×Tris wash buffer. 150 μl 1× Read Buffer T was added to each well and emitted light intensity was read in luminescence units on a MSD Quickplex SQ120 instrument.

For measurement of proliferation, NK were contacted with purified VHH dimers as follows: Cells were seeded into 96-well plates (Falcon) at 100 thousand cells per well in 190 μl in growth medium consisting of Yssel's medium (Iscove's modified Dulbecco's Medium (ThermoFisher), 0.25% w/v percent human albumin (Sigma), 1 percent penicillin/streptomycin (ThermoFisher), 1 percent ITS-X Insulin, Transferrin, Selenium (Gibco), 30 mg/L Tansferrin (Roche), 2 mg/L Palmitic Acid (Sigma), 1 percent LA-OA-Albumin Linoleic Acid, Oleic Acid (Sigma), 1 percent human serum (Gemini) (Yssel et al (1984) J Immunol Methods 72: 219-227). Ten μl of each of the 120 purified VHH dimers in DPBS at 300 nM was added to the cells and plates were transferred to a humidified incubator (ThermoFisher) and incubated at 37 degrees centigrade, 5 percent carbon dioxide for 72 hrs.

Plates were removed from the incubator and kept at room temperature for 30 minutes.

One hundred microliter of the cell culture supernatants was transferred to a new 96 well plate for measurement of IFN-γ levels.

Cells were lysed by adding 100 μl per well of Celltiterglo (Promega). Cell lysates were mixed on an orbital shaker (VWR Scientific) for two minutes at 200 rpm then held at room temperature for 10 minutes. Luminescence for NK cell lysates were read as counts per second in an Envision 2103 Multilabel Plate Reader (Perkin Elmer).

The level of IFN-γ in the supernatants was measured using the MSD multi-spot assay system with the V-PLEX human IFN-γ kit (K151QOD-4) according to manufacturer's instructions. MSD 96 well assay plates were washed 3 times with 1×Tris wash buffer and 50 μl of culture supernatants diluted 100 fold in diluent 2 were added to each well. Plates were incubated on an orbital shaker (VWR Scientific) for 120 minutes at room temperature and washed 3 times with 1×Tris wash buffer. Detection antibody solution (25 μl) was added to the plate. Plates were incubated on an orbital shaker (VWR Scientific) for 120 minutes at room temperature and washed 3 times with 1×Tris wash buffer. 150 μl 2× Read Buffer T was added to each well and emitted light intensity was read in luminescence units on a MSD Quickplex SQ120 instrument.

To compare the effect of each IL-2 VHH dimer upon pSTAT5 induction, NK cell proliferation and IFN-γ production, luminescence values from pSTAT, celltiterglo and IFN-γ measurements were compared to those obtained for control cells treated with growth medium alone and control cells treated with human IL-2 at 100 μM. IL-2 VHH dimers were identified that induced higher luminescence signals for pSTAT5 induction, cell proliferation and IFN-γ production than media control but lower than IL-2 at the concentrations used. The data from these experiments is presented in Table 23.

TABLE 23
Activity of VHH dimers on NK cell pSTAT5 induction,
proliferation and IFN-γ production.

| VHH SEQ | VHH DR1 DR2 | pSTAT5 | Proliferation | IFN-γ Production |
|---|---|---|---|---|
| ID | ID | (LU) | (LU) | (LU) |
| DR632 | DR229-DR214 | 574 | 59374 | 144618 |
| DR633 | DR229-DR217 | 339 | 84476 | 80516 |
| DR634 | DR229-DR583 | 2165 | 129450 | 1883810 |
| DR635 | DR229-DR584 | 15080 | 158792 | 1911538 |
| DR636 | DR229-DR585 | 3220 | 66196 | 36726 |
| DR637 | DR229-DR586 | 280 | 85292 | 142020 |
| DR638 | DR229-DR587 | 24366 | 133116 | 1907171 |
| DR639 | DR229-DR588 | 3561 | 140702 | 1128817 |
| DR640 | DR229-DR589 | 1756 | 140980 | 1137944 |
| DR641 | DR229-DR590 | 195 | 50668 | 36793 |
| DR642 | DR230-DR214 | 13823 | 132720 | 1849412 |
| DR643 | DR230-DR217 | 24065 | 156030 | 1850211 |
| DR644 | DR230-DR583 | 556 | 135388 | 795501 |
| DR645 | DR230-DR584 | 7741 | 150146 | 1960761 |
| DR646 | DR230-DR585 | 1789 | 72526 | 129630 |
| DR647 | DR230-DR586 | 21564 | 155762 | 1921088 |
| DR648 | DR230-DR587 | 13437 | 144822 | 1845712 |
| DR649 | DR230-DR588 | 236 | 64048 | 61590 |
| DR650 | DR230-DR589 | 349 | 47462 | 23788 |
| DR651 | DR230-DR590 | 338 | 49600 | 13376 |
| DR652 | DR231-DR214 | 12830 | 171010 | 1901886 |
| DR653 | DR231-DR217 | 223 | 57866 | 73604 |
| DR654 | DR231-DR583 | 220 | 49196 | 38744 |
| DR655 | DR231-DR584 | 2826 | 82024 | 641257 |
| DR656 | DR231-DR585 | 6038 | 127580 | 1901425 |
| DR657 | DR231-DR586 | 213 | 57212 | 133136 |
| DR658 | DR231-DR587 | 202 | 46432 | 83387 |
| DR659 | DR231-DR588 | 214 | 78720 | 218983 |
| DR660 | DR231-DR589 | 204 | 50996 | 31698 |
| DR661 | DR231-DR590 | 192 | 48910 | 12924 |
| DR662 | DR232-DR214 | 224 | 53038 | 127725 |
| DR663 | DR232-DR217 | 546 | 118242 | 787105 |
| DR664 | DR232-DR583 | 1126 | 91608 | 431059 |
| DR665 | DR232-DR584 | 221 | 45064 | 17316 |
| DR666 | DR232-DR585 | 255 | 49560 | 55300 |
| DR667 | DR232-DR586 | 604 | 117570 | 658694 |
| DR668 | DR232-DR587 | 277 | 60456 | 57260 |
| DR669 | DR232-DR588 | 456 | 59204 | 23328 |
| DR670 | DR232-DR589 | 400 | 81900 | 215999 |
| DR671 | DR232-DR590 | 1875 | 138056 | 1279164 |
| DR672 | DR233-DR214 | 1358 | 120064 | 1690293 |
| DR673 | DR233-DR217 | 3071 | 147252 | 1195243 |
| DR674 | DR233-DR583 | 5420 | 123220 | 1520704 |
| DR675 | DR233-DR584 | 8803 | 146160 | 1922798 |
| DR676 | DR233-DR585 | 580 | 55708 | 44387 |
| DR677 | DR233-DR586 | 836 | 107368 | 451720 |
| DR678 | DR233-DR587 | 16689 | 160706 | 1877120 |
| DR679 | DR233-DR588 | 5952 | 151432 | 1840663 |
| DR680 | DR233-DR589 | 5639 | 122932 | 1574319 |
| DR681 | DR233-DR590 | 1228 | 83802 | 79218 |
| DR682 | DR234-DR214 | 1175 | 94996 | 266392 |
| DR683 | DR234-DR217 | 480 | 82018 | 66068 |
| DR684 | DR234-DR583 | 214 | 46514 | 9713 |
| DR685 | DR234-DR584 | 881 | 97470 | 451812 |
| DR686 | DR234-DR585 | 246 | 56082 | 79541 |
| DR687 | DR234-DR586 | 289 | 63300 | 122522 |
| DR688 | DR234-DR587 | 2121 | 99810 | 1299512 |
| DR689 | DR234-DR588 | 3040 | 115836 | 1546871 |
| DR690 | DR234-DR589 | 1389 | 108534 | 851698 |
| DR691 | DR234-DR590 | 262 | 52358 | 33530 |
| DR692 | DR214-DR229 | 586 | 80168 | 341600 |
| DR693 | DR214-DR230 | 336 | 44468 | 32214 |
| DR694 | DR214-DR231 | 214 | 45548 | 13781 |
| DR695 | DR214-DR232 | 467 | 57448 | 50327 |
| DR696 | DR214-DR233 | 356 | 62564 | 20592 |
| DR697 | DR214-DR234 | 515 | 73146 | 128229 |
| DR698 | DR217-DR229 | 242 | 65260 | 373385 |
| DR699 | DR217-DR230 | 307 | 68008 | 108166 |
| DR700 | DR217-DR231 | 802 | 76818 | 68805 |
| DR701 | DR217-DR232 | 10020 | 152808 | 1716480 |
| DR702 | DR217-DR233 | 905 | 77978 | 114876 |
| DR703 | DR217-DR234 | 419 | 86778 | 390416 |
| DR704 | DR583-DR229 | 242 | 35080 | 18481 |
| DR705 | DR583-DR230 | 203 | 46406 | 26339 |
| DR706 | DR583-DR231 | 211 | 34682 | 13676 |
| DR707 | DR583-DR232 | 193 | 38418 | 20801 |
| DR708 | DR583-DR233 | 212 | 40002 | 28936 |
| DR709 | DR583-DR234 | 203 | 47088 | 25068 |
| DR710 | DR584-DR229 | 195 | 38422 | 71711 |
| DR711 | DR584-DR230 | 182 | 36094 | 16215 |
| DR712 | DR584-DR231 | 206 | 42562 | 17139 |
| DR713 | DR584-DR232 | 201 | 44564 | 10805 |
| DR714 | DR584-DR233 | 203 | 31896 | 17361 |
| DR715 | DR584-DR234 | 192 | 37140 | 30424 |
| DR716 | DR585-DR229 | 3207 | 135888 | 1415371 |
| DR717 | DR585-DR230 | 2034 | 125778 | 1860610 |
| DR718 | DR585-DR231 | 12281 | 115728 | 1665366 |
| DR719 | DR585-DR232 | 5818 | 131414 | 1334193 |
| DR720 | DR585-DR233 | 2254 | 136050 | 995884 |
| DR721 | DR585-DR234 | 10913 | 137718 | 1937087 |
| DR722 | DR586-DR229 | 7654 | 148710 | 1704185 |
| DR723 | DR586-DR230 | 293 | 74564 | 119540 |
| DR724 | DR586-DR231 | 10577 | 165338 | 1928068 |
| DR725 | DR586-DR232 | 442 | 55684 | 38001 |
| DR726 | DR586-DR233 | 184 | 36126 | 54648 |
| DR727 | DR586-DR234 | 195 | 38650 | 15553 |
| DR728 | DR587-DR229 | 205 | 40442 | 60701 |
| DR729 | DR587-DR230 | 221 | 42958 | 54437 |
| DR730 | DR587-DR231 | 210 | 43068 | 30615 |
| DR731 | DR587-DR232 | 217 | 47702 | 49737 |
| DR732 | DR587-DR233 | 233 | 42812 | 596023 |
| DR733 | DR587-DR234 | 221 | 48304 | 562814 |
| DR734 | DR588-DR229 | 594 | 113408 | 641028 |
| DR735 | DR588-DR230 | 528 | 119102 | 757479 |
| DR736 | DR588-DR231 | 1909 | 138570 | 1455626 |
| DR737 | DR588-DR232 | 360 | 84578 | 308300 |
| DR738 | DR588-DR233 | 266 | 48734 | 29645 |
| DR739 | DR588-DR234 | 580 | 108850 | 279562 |
| DR740 | DR589-DR229 | 312 | 115378 | 517465 |
| DR741 | DR589-DR230 | 647 | 113032 | 181177 |
| DR742 | DR589-DR231 | 330 | 58200 | 102522 |
| DR743 | DR589-DR232 | 318 | 47630 | 12119 |
| DR744 | DR589-DR233 | 801 | 96038 | 709867 |
| DR745 | DR589-DR234 | 206 | 47518 | 552283 |
| DR746 | DR590-DR229 | 375 | 110814 | 638377 |
| DR747 | DR590-DR230 | 809 | 124594 | 713175 |
| DR748 | DR590-DR231 | 474 | 129464 | 407832 |
| DR749 | DR590-DR232 | 231 | 59380 | 100488 |
| DR750 | DR590-DR233 | 275 | 62470 | 32777 |
| DR751 | DR590-DR234 | 189 | 40034 | 30496 |
| Media | n/a | 705 | 37693 | 16672 |
| hIL-2 | n/a | 50261 | 132827 | 1793477 |

LU: Luminescence Units.
n/a : not applicable

### Example 10. Evaluation of Activity of IL-2 VHH Dimers on Primary CD8 Positive T Cells Blasts

The IL2 VHH dimers were evaluated for activity in Primary CD8 T cells isolated from activated PBMC. Primary CD8 positive T cells blasts express IL-2Rβ3 and IL-2Rγ chains and can respond to IL-2 by phosphorylation of STATS, proliferation and the production of IFN-γ.

PBMC were isolated from human Buffy Coats or Leucocyte Reduction System Chambers (LRSC) using the Custom Sedimentation Kit (Miltenyi, #130-126-357) and Custom Buffy Coat/LRSC PBMC Isolation kits (Miltenyi, 130-126-448) using protocol Cust5 on an autoMACS Pro Separator (Miltenyi) according to manufacturer's instructions. Purified PBMC were counted on a Vi-cell XR (Beckman Coulter) or Vi-cell Blue (Beckman Coulter) cell viability analyzer.

PBMC were cultured on growth medium consisting of Yssel's medium (Iscove's modified Dulbecco's Medium (ThermoFisher), 0.25% w/v percent human albumin (Sigma), 1 percent penicillin/streptomycin (ThermoFisher), 1 percent ITS-X Insulin, Transferrin, Selenium (Gibco), 30 mg/L Tansferrin (Roche), 2 mg/L Palmitic Acid (Sigma), 1 percent LA-OA-Albumin Linoleic Acid, Oleic Acid (Sigma), 1 percent human serum (Gemini) (Yssel et al (1984) J Immunol Methods 72: 219-227) at 1 million cells per mL with 1 μg/mL anti-CD3 mAb OKT3 (BioXcell) and 1 μg/mL anti-CD28 mAb CD28.2 (BioXcell) in 100 mL in a T150 cell culture flask (Falcon) at 37 degrees centigrade, 5 percent carbon dioxide for 5 days.

Primary CD8 positive T cell blasts were isolated from activated human PBMC using CD8 microbeads (Miltenyi, 130-045-201) on an autoMACS Pro Separator (Miltenyi) with protocol possel according to manufacturer's instructions. Purified primary CD8 T cell blasts were counted on a Vi-cell XR (Beckman Coulter) or Vi-cell Blue (Beckman Coulter) cell viability analyzer.

Purified primary CD8 T cell blasts were contacted with purified VHH dimers to examine induction of STAT5 phosphorylation as follows: Cells were seeded into 96-well plates (Falcon) at 100 thousand cells per well in 95 μl DPBS prewarmed at 37 degrees centigrade. Five μl of each of the 120 purified VHH dimers in DPBS at 300 nM was added to the cells and plates were transferred to a humidified incubator (ThermoFisher) and incubated at 37 degrees centigrade, 5 percent carbon dioxide for 20 minutes.

Plates were removed from the incubator and 100 μl 2× Complete Lysis buffer (Tris Lysis Buffer, Protease Inhibitor Solution, Phosphatase Inhibitor I, Phosphatase Inhibitor II) was added according to manufacturer's instructions (MSD Phospho-STAT Panel K15202D). Plates were incubated on ice for 15 minutes and centrifuged for 5 minutes at 600×g and Lysates were transferred to a new 96 well plate.

The level of phospho-STAT5 induction in the lysate was measured using the MSD multi-spot assay system with the Phospho-STAT panel kit (K15202D) according to manufacturer's instructions. MSD 96 well assay plates were washed 3 times with 1×Tris wash buffer and 150 μl Blocker A solution was added to each well. Plates were incubated on an orbital shaker (VWR Scientific) for 60 minutes at room temperature and washed 3 times with 1×Tris wash buffer. Cell lysates (25 μl) were added to the plate. Plates were incubated on an orbital shaker (VWR Scientific) for 60 minutes at room temperature and washed 3 times with 1×Tris wash buffer. Detection antibody solution (25 μl) was added to the plate. Plates were incubated on an orbital shaker (VWR Scientific) for 60 minutes at room temperature and washed 3 times with 1×Tris wash buffer. 150 μl 1× Read Buffer T was added to each well and emitted light intensity was read in luminescence units on a MSD Quickplex SQ120 instrument.

For measurement of proliferation, purified primary CD8 T cell blasts were contacted with purified VHH dimers as follows: Cells were seeded into 96-well plates (Falcon) at 100 thousand cells per well in 190 μl in growth medium consisting of Yssel's medium. Ten 1 of each of the 120 purified VHH dimers in DPBS at 300 nM was added to the cells and plates were transferred to a humidified incubator (ThermoFisher) and incubated at 37 degrees centigrade, 5 percent carbon dioxide for 72 hrs.

Plates were removed from the incubator and kept at room temperature for 30 minutes.

One hundred microliter of the cell culture supernatants was transferred to a new 96 well plate for measurement of IFN-γ levels.

Cells were lysed by adding 100 μl per well of Celltiterglo (Promega). Cell lysates were mixed on an orbital shaker (VWR Scientific) for two minutes at 200 rpm then held at room temperature for 10 minutes. Luminescence for primary CD8 T cell blast lysates were read as counts per second in an Envision 2103 Multilabel Plate Reader (Perkin Elmer).

The level of IFN-γ in the supernatants was measured using the MSD multi-spot assay system with the V-PLEX human IFN-γ kit (K151QOD-4) according to manufacturer's instructions. MSD 96 well assay plates were washed 3 times with 1×Tris wash buffer and 50 μl of culture supernatants diluted 10 fold in diluent 2 were added to each well. Plates were incubated on an orbital shaker (VWR Scientific) for 120 minutes at room temperature and washed 3 times with 1×Tris wash buffer. Detection antibody solution (25 μl) was added to the plate. Plates were incubated on an orbital shaker (VWR Scientific) for 120 minutes at room temperature and washed 3 times with 1×Tris wash buffer. 150 μl 2× Read Buffer T was added to each well and emitted light intensity was read in luminescence units on a MSD Quickplex SQ120 instrument.

To compare the effect of each IL-2 VHH dimer upon pSTAT5 induction, primary CD8 T cell blast proliferation and IFN-γ production, luminescence values from pSTAT, celltiterglo and IFN-γ measurements were compared to those obtained for control cells treated with growth medium alone and control cells treated with human IL-2 at 100 μM. IL-2 VHH dimers were identified that induced higher luminescence signals for pSTAT5 induction, cell proliferation and IFN-γ production than media control but lower than IL-2 at the concentrations used. The data from these experiments is presented in Table 24.

TABLE 24
Activity of VHH dimers on primary CD8 T cell blasts pSTAT5 induction,
proliferation and IFN-γ production.

| VHH SEQ | VHH DR1 DR2 | pSTAT5 | Proliferation | IFN-γ Production |
|---|---|---|---|---|
| ID | ID | (LU) | (LU) | (LU) |
| DR632 | DR229-DR214 | 1975 | 74694 | 357 |
| DR633 | DR229-DR217 | 2802 | 97528 | 400 |
| DR634 | DR229-DR583 | 7119 | 169082 | 3050 |
| DR635 | DR229-DR584 | 69738 | 199810 | 6050 |
| DR636 | DR229-DR585 | 4305 | 96650 | 364 |
| DR637 | DR229-DR586 | 3152 | 96020 | 407 |
| DR638 | DR229-DR587 | 67754 | 196596 | 4429 |
| DR639 | DR229-DR588 | 29763 | 186708 | 2653 |
| DR640 | DR229-DR589 | 15645 | 166848 | 1413 |
| DR641 | DR229-DR590 | 2566 | 59040 | 176 |
| DR642 | DR230-DR214 | 37103 | 169256 | 2125 |
| DR643 | DR230-DR217 | 83861 | 199372 | 5021 |
| DR644 | DR230-DR583 | 5757 | 150086 | 1077 |
| DR645 | DR230-DR584 | 46494 | 234368 | 5761 |
| DR646 | DR230-DR585 | 11691 | 100966 | 305 |
| DR647 | DR230-DR586 | 88561 | 207848 | 4615 |
| DR648 | DR230-DR587 | 55433 | 193526 | 3168 |
| DR649 | DR230-DR588 | 2353 | 89156 | 388 |
| DR650 | DR230-DR589 | 4356 | 59650 | 146 |
| DR651 | DR230-DR590 | 4380 | 60920 | 157 |
| DR652 | DR231-DR214 | 62461 | 214638 | 5290 |
| DR653 | DR231-DR217 | 2241 | 68142 | 356 |
| DR654 | DR231-DR583 | 3512 | 64188 | 131 |
| DR655 | DR231-DR584 | 10601 | 96790 | 480 |
| DR656 | DR231-DR585 | 31725 | 149116 | 2033 |
| DR657 | DR231-DR586 | 5973 | 68418 | 193 |
| DR658 | DR231-DR587 | 2572 | 50934 | 232 |
| DR659 | DR231-DR588 | 2262 | 78852 | 281 |
| DR660 | DR231- DR589 | 4159 | 70632 | 202 |
| DR661 | DR231-DR590 | 4637 | 61506 | 186 |
| DR662 | DR232-DR214 | 3144 | 58280 | 243 |
| DR663 | DR232-DR217 | 3041 | 127604 | 929 |
| DR664 | DR232-DR583 | 10939 | 99388 | 378 |
| DR665 | DR232-DR584 | 4927 | 60210 | 137 |
| DR666 | DR232-DR585 | 2936 | 57460 | 161 |
| DR667 | DR232-DR586 | 3221 | 123146 | 979 |
| DR668 | DR232-DR587 | 4857 | 77298 | 196 |
| DR669 | DR232-DR588 | 7147 | 78226 | 194 |
| DR670 | DR232-DR589 | 3756 | 90720 | 349 |
| DR671 | DR232-DR590 | 6860 | 142454 | 1306 |
| DR672 | DR233-DR214 | 19482 | 170126 | 1188 |
| DR673 | DR233-DR217 | 15296 | 188860 | 2153 |
| DR674 | DR233-DR583 | 29097 | 175798 | 2296 |
| DR675 | DR233-DR584 | 16694 | 182374 | 4191 |
| DR676 | DR233-DR585 | 6983 | 102448 | 241 |
| DR677 | DR233-DR586 | 5021 | 112872 | 686 |
| DR678 | DR233-DR587 | 71073 | 196456 | 5977 |
| DR679 | DR233-DR588 | 9245 | 172512 | 4357 |
| DR680 | DR233-DR589 | 35467 | 140116 | 3008 |
| DR681 | DR233-DR590 | 14232 | 110932 | 411 |
| DR682 | DR234-DR214 | 6798 | 113696 | 840 |
| DR683 | DR234-DR217 | 6461 | 87860 | 332 |
| DR684 | DR234-DR583 | 3136 | 48908 | 166 |
| DR685 | DR234-DR584 | 11098 | 112190 | 672 |
| DR686 | DR234-DR585 | 5015 | 68192 | 277 |
| DR687 | DR234-DR586 | 5052 | 76202 | 230 |
| DR688 | DR234-DR587 | 20746 | 113120 | 1705 |
| DR689 | DR234-DR588 | 29909 | 153806 | 933 |
| DR690 | DR234-DR589 | 8260 | 106106 | 565 |
| DR691 | DR234-DR590 | 3327 | 63728 | 159 |
| DR692 | DR214-DR229 | 7796 | 81058 | 283 |
| DR693 | DR214-DR230 | 5864 | 74190 | 202 |
| DR694 | DR214-DR231 | 4098 | 53506 | 133 |
| DR695 | DR214-DR232 | 4808 | 73792 | 184 |
| DR696 | DR214-DR233 | 4670 | 75228 | 310 |
| DR697 | DR214-DR234 | 4911 | 76072 | 217 |
| DR698 | DR217-DR229 | 4409 | 59082 | 172 |
| DR699 | DR217-DR230 | 3101 | 67000 | 232 |
| DR700 | DR217-DR231 | 6717 | 72854 | 210 |
| DR701 | DR217-DR232 | 40130 | 151010 | 2879 |
| DR702 | DR217-DR233 | 6388 | 83172 | 208 |
| DR703 | DR217-DR234 | 2112 | 73744 | 291 |
| DR704 | DR583-DR229 | 3828 | 47968 | 192 |
| DR705 | DR583-DR230 | 3977 | 53554 | 199 |
| DR706 | DR583-DR231 | 2996 | 43828 | 265 |
| DR707 | DR583-DR232 | 4123 | 51940 | 255 |
| DR708 | DR583-DR233 | 4414 | 50450 | 166 |
| DR709 | DR583-DR234 | 3552 | 52870 | 168 |
| DR710 | DR584-DR229 | 4380 | 41228 | 231 |
| DR711 | DR584-DR230 | 3083 | 45988 | 231 |
| DR712 | DR584-DR231 | 4739 | 47854 | 160 |
| DR713 | DR584-DR232 | 4168 | 50962 | 164 |
| DR714 | DR584-DR233 | 3885 | 44376 | 154 |
| DR715 | DR584-DR234 | 3414 | 49586 | 228 |
| DR716 | DR585-DR229 | 47906 | 161498 | 2271 |
| DR717 | DR585-DR230 | 19829 | 163070 | 2292 |
| DR718 | DR585-DR231 | 99121 | 176680 | 7211 |
| DR719 | DR585-DR232 | 41612 | 145996 | 1674 |
| DR720 | DR585-DR233 | 29633 | 144204 | 1251 |
| DR721 | DR585-DR234 | 48028 | 167206 | 4122 |
| DR722 | DR586-DR229 | 68270 | 171340 | 4087 |
| DR723 | DR586-DR230 | 2669 | 75522 | 468 |
| DR724 | DR586-DR231 | 78186 | 211052 | 4012 |
| DR725 | DR586-DR232 | 2018 | 61316 | 210 |
| DR726 | DR586-DR233 | 3643 | 49710 | 227 |
| DR727 | DR586-DR234 | 555 | 40706 | 211 |
| DR728 | DR587-DR229 | 3292 | 34304 | 251 |
| DR729 | DR587-DR230 | 3154 | 43390 | 226 |
| DR730 | DR587-DR231 | 2908 | 44146 | 203 |
| DR731 | DR587-DR232 | 1983 | 46354 | 206 |
| DR732 | DR587-DR233 | 2854 | 51346 | 390 |
| DR733 | DR587-DR234 | 2417 | 53770 | 294 |
| DR734 | DR588-DR229 | 4609 | 91694 | 676 |
| DR735 | DR588-DR230 | 2445 | 81412 | 856 |
| DR736 | DR588-DR231 | 9338 | 109598 | 1810 |
| DR737 | DR588-DR232 | 2946 | 70134 | 545 |
| DR738 | DR588-DR233 | 2876 | 55678 | 218 |
| DR739 | DR588-DR234 | 2011 | 94516 | 710 |
| DR740 | DR589-DR229 | 3880 | 81458 | 613 |
| DR741 | DR589-DR230 | 3493 | 88854 | 593 |
| DR742 | DR589-DR231 | 2713 | 56044 | 297 |
| DR743 | DR589-DR232 | 1579 | 48310 | 166 |
| DR744 | DR589-DR233 | 3969 | 75144 | 622 |
| DR745 | DR589-DR234 | 1741 | 46104 | 283 |
| DR746 | DR590-DR229 | 2869 | 91978 | 577 |
| DR747 | DR590-DR230 | 2890 | 128022 | 1905 |
| DR748 | DR590-DR231 | 4161 | 94978 | 962 |
| DR749 | DR590-DR232 | 1252 | 47614 | 366 |
| DR750 | DR590-DR233 | 2182 | 65174 | 275 |
| DR751 | DR590-DR234 | 955 | 41294 | 220 |
| Media | n/a | 5263 | 46428 | 232 |
| hIL-2 | n/a | 130278 | 143593 | 8956 |

LU: Luminescence Units.
n/a : not applicable

### Example 11. Evaluation of Activity of IL-2 VHH Dimers on Human CD4 Positive T Cell Clone 3F8

The IL2 VHH dimers were evaluated for activity in CD4 positive human T cell clone 3F8 cells. The CD4 positive T cell clone 3F8 was generated by activation of PBMC of a healthy donor with the EBV transformed B cell line JY in two successive rounds of Mixed Leukocyte Reactions followed by single cell cloning by limited dilution as described (Yssel and Spits (2002) Current Protocols in Immunology 7.19.1-7.19.12). The CD4 positive T cell clone 3F8 expresses IL-2Rβ and IL-2Rγ chains and proliferates in response to IL-2.

For measurement of proliferation, 3F8 cells were contacted with purified VHH dimers as follows: Cells were grown in growth medium consisting of Yssel's medium (Iscove's modified Dulbecco's Medium (ThermoFisher), 0.25% w/v percent human albumin (Sigma), 1 percent penicillin/streptomycin (ThermoFisher), 1 percent ITS-X Insulin, Transferrin, Selenium (Gibco), 30 mg/L Tansferrin (Roche), 2 mg/L Palmitic Acid (Sigma), 1 percent LA-OA-Albumin Linoleic Acid, Oleic Acid (Sigma), 1 percent human serum (Gemini) (Yssel et al (1984) J Immunol Methods 72: 219-227) at 0.2 million cells per ml with 50 Gy irradiated JY cells at 0.1 million cells per well and 40 Gy irradiated allogeneic PBMC at 1 million cells per mL. After ten days of culture and expansion with human IL-2 at 100 μM, cells were washed and seeded into black, clear bottom 96 well plates (Costar) at 50 thousand cells per well in 90 μl growth medium. Ten μl of each of the 120 purified VHH dimers in DPBS at 300 nM was added to the cells and plates were transferred to a humidified incubator (ThermoFisher) and incubated at 37 degrees centigrade, 5 percent carbon dioxide for 72 hrs.

Plates were removed from the incubator and cells were lysed by adding 100 μl per well of Celltiterglo (Promega). Cell lysates were mixed on an orbital shaker (VWR Scientific) for two minutes at 200 rpm then held at room temperature for 10 minutes. Luminescence for 3F8 cell lysates were read as counts per second in an Envision 2103 Multilabel Plate Reader (Perkin Elmer).

To compare the effect of each IL-2 VHH dimer upon 3F8 cell proliferation, luminescence values from celltiterglo measurements were compared to those obtained for control cells treated with growth medium alone and control cells treated with human IL-2 at 100 μM. IL-2 VHH dimers were identified that induced higher luminescence signals for 3F8 cell proliferation than media control but lower than IL-2 at the concentrations used. The data from these experiments is presented in Table 25.

TABLE 25
Activity of VHH dimers on human CD4 positive T cell clone 3F8

| VHH SEQ ID | VHH DR1 DR2 ID | Proliferation (LU) |
|---|---|---|
| DR632 | DR229-DR214 | 33888 |
| DR633 | DR229-DR217 | 41194 |
| DR634 | DR229-DR583 | 66462 |
| DR635 | DR229-DR584 | 76938 |
| DR636 | DR229-DR585 | 44314 |
| DR637 | DR229-DR586 | 46870 |
| DR638 | DR229-DR587 | 87848 |
| DR639 | DR229-DR588 | 71832 |
| DR640 | DR229-DR589 | 64522 |
| DR641 | DR229-DR590 | 43324 |
| DR642 | DR230-DR214 | 84204 |
| DR643 | DR230-DR217 | 79866 |
| DR644 | DR230-DR583 | 57066 |
| DR645 | DR230-DR584 | 72772 |
| DR646 | DR230-DR585 | 47376 |
| DR647 | DR230-DR586 | 73828 |
| DR648 | DR230-DR587 | 72194 |
| DR649 | DR230-DR588 | 48562 |
| DR650 | DR230-DR589 | 45092 |
| DR651 | DR230-DR590 | 41826 |
| DR652 | DR231-DR214 | 71060 |
| DR653 | DR231-DR217 | 31888 |
| DR654 | DR231-DR583 | 37502 |
| DR655 | DR231-DR584 | 41534 |
| DR656 | DR231-DR585 | 78076 |
| DR657 | DR231-DR586 | 53584 |
| DR658 | DR231-DR587 | 40568 |
| DR659 | DR231-DR588 | 52996 |
| DR660 | DR231-DR589 | 41636 |
| DR661 | DR231-DR590 | 41330 |
| DR662 | DR232-DR214 | 49936 |
| DR663 | DR232-DR217 | 71038 |
| DR664 | DR232-DR583 | 69286 |
| DR665 | DR232-DR584 | 41762 |
| DR666 | DR232-DR585 | 50210 |
| DR667 | DR232-DR586 | 74854 |
| DR668 | DR232-DR587 | 51260 |
| DR669 | DR232-DR588 | 52574 |
| DR670 | DR232-DR589 | 62090 |
| DR671 | DR232-DR590 | 71850 |
| DR672 | DR233-DR214 | 63810 |
| DR673 | DR233-DR217 | 66648 |
| DR674 | DR233-DR583 | 78250 |
| DR675 | DR233-DR584 | 79716 |
| DR676 | DR233-DR585 | 47408 |
| DR677 | DR233-DR586 | 60714 |
| DR678 | DR233-DR587 | 84816 |
| DR679 | DR233-DR588 | 70776 |
| DR680 | DR233-DR589 | 72106 |
| DR681 | DR233-DR590 | 71270 |
| DR682 | DR234-DR214 | 59464 |
| DR683 | DR234-DR217 | 62038 |
| DR684 | DR234-DR583 | 40188 |
| DR685 | DR234-DR584 | 62736 |
| DR686 | DR234-DR585 | 48414 |
| DR687 | DR234-DR586 | 59830 |
| DR688 | DR234-DR587 | 71834 |
| DR689 | DR234-DR588 | 75204 |
| DR690 | DR234-DR589 | 75612 |
| DR691 | DR234-DR590 | 48538 |
| DR692 | DR214-DR229 | 52982 |
| DR693 | DR214-DR230 | 57408 |
| DR694 | DR214-DR231 | 41318 |
| DR695 | DR214-DR232 | 63280 |
| DR696 | DR214-DR233 | 59748 |
| DR697 | DR214-DR234 | 50344 |
| DR698 | DR217-DR229 | 46812 |
| DR699 | DR217-DR230 | 55404 |
| DR700 | DR217-DR231 | 47242 |
| DR701 | DR217-DR232 | 62282 |
| DR702 | DR217-DR233 | 62942 |
| DR703 | DR217-DR234 | 58558 |
| DR704 | DR583-DR229 | 36402 |
| DR705 | DR583-DR230 | 41940 |
| DR706 | DR583-DR231 | 36666 |
| DR707 | DR583-DR232 | 38206 |
| DR708 | DR583-DR233 | 32604 |
| DR709 | DR583-DR234 | 37410 |
| DR710 | DR584-DR229 | 35512 |
| DR711 | DR584-DR230 | 37716 |
| DR712 | DR584-DR231 | 37102 |
| DR713 | DR584-DR232 | 40972 |
| DR714 | DR584-DR233 | 27114 |
| DR715 | DR584-DR234 | 39740 |
| DR716 | DR585-DR229 | 81372 |
| DR717 | DR585-DR230 | 72588 |
| DR718 | DR585-DR231 | 80658 |
| DR719 | DR585-DR232 | 70762 |
| DR720 | DR585-DR233 | 88944 |
| DR721 | DR585-DR234 | 72802 |
| DR722 | DR586-DR229 | 74538 |
| DR723 | DR586-DR230 | 50868 |
| DR724 | DR586-DR231 | 70232 |
| DR725 | DR586-DR232 | 54800 |
| DR726 | DR586-DR233 | 35400 |
| DR727 | DR586-DR234 | 40006 |
| DR728 | DR587-DR229 | 35520 |
| DR729 | DR587-DR230 | 52430 |
| DR730 | DR587-DR231 | 34202 |
| DR731 | DR587-DR232 | 42902 |
| DR732 | DR587-DR233 | 44550 |
| DR733 | DR587-DR234 | 49704 |
| DR734 | DR588-DR229 | 38080 |
| DR735 | DR588-DR230 | 55892 |
| DR736 | DR588-DR231 | 87242 |
| DR737 | DR588-DR232 | 65336 |
| DR738 | DR588-DR233 | 40950 |
| DR739 | DR588-DR234 | 94516 |
| DR740 | DR589-DR229 | 81458 |
| DR741 | DR589-DR230 | 88854 |
| DR742 | DR589-DR231 | 56044 |
| DR743 | DR589-DR232 | 48310 |
| DR744 | DR589-DR233 | 75144 |
| DR745 | DR589-DR234 | 46104 |
| DR746 | DR590-DR229 | 91978 |
| DR747 | DR590-DR230 | 128022 |
| DR748 | DR590-DR231 | 94978 |
| DR749 | DR590-DR232 | 47614 |
| DR750 | DR590-DR233 | 65174 |
| DR751 | DR590-DR234 | 41294 |
| Media | n/a | 46428 |
| hIL-2 | n/a | 143593 |

### Example 12. Evaluation of Activity of IL-2 VHH Dimers on Non-Activated PBMC

The IL2 VHH dimers were evaluated for activity in non-activated PBMC. Several cell types including NK cells, CD4 T cells, CD8 T cell, regulatory T cells and NKT cells express IL-2Rβ and IL-2Rγ chains and can respond to IL-2 by proliferation and the production of IFN-γ.

PBMC were isolated from human Buffy Coats or Leucocyte Reduction System Chambers (LRSC) using the Custom Sedimentation Kit (Miltenyi, #130-126-357) and Custom Buffy Coat/LRSC PBMC Isolation kits (Miltenyi, 130-126-448) using protocol Cust5 on an autoMACS Pro Separator (Miltenyi) according to manufacturer's instructions. Purified PBMC were counted on a Vi-cell XR (Beckman Coulter) or Vi-cell Blue (Beckman Coulter) cell viability analyzer.

PBMC from 2 different donors were contacted with purified VHH dimers to examine proliferation and the production of IFN-γ as follows: Cells were seeded into 24-well plates (Corning) at 1 million cells per well in 1 mL growth medium consisting of Yssel's medium (Iscove's modified Dulbecco's Medium (ThermoFisher), 0.25% w/v percent human albumin (Sigma), 1 percent penicillin/streptomycin (ThermoFisher), 1 percent ITS-X Insulin, Transferrin, Selenium (Gibco), 30 mg/L Tansferrin (Roche), 2 mg/L Palmitic Acid (Sigma), 1 percent LA-OA-Albumin Linoleic Acid, Oleic Acid (Sigma), 1 percent human serum (Gemini) (Yssel et al (1984) J Immunol Methods 72: 219-227). Twenty five μl of each of the 120 purified VHH dimers in DPBS at 300 nM was added to the cells and plates were transferred to a humidified incubator (ThermoFisher) and incubated at 37 degrees centigrade, 5 percent carbon dioxide for 168 hrs.

Plates were removed from the incubator and 100 μl of the cell culture supernatants was transferred to a new 96 well plate for measurement of IFN-γ levels.

To examine proliferation in these cultures, cells were harvested from wells that still contained viable cells upon visual inspection and were phenotyped for CD3, CD4, CD8, CD25 and CD56 expression. Cells were washed in PBS and incubated in PBS with 1/1000 dilution of fix viability dye ef506 for 15 min on ice and quenched in FACS buffer consisting of PBS, 2 mM EDTA, 0.5% BSA. Cells were washed and stained with CD56-BV421, CD25-PE, CD3-BB515, CD4-BV786 and CD8-APC-Cy7 antibody-conjugates (All Biolegend) according to manufacturer's recommendation for 30 min on ice. Cells were washed, fixed with 0.1% paraformaldehyde and analyzed on an Aurora Flow Cytometer (Cytek) with SpectroFlo software.

The level of IFN-γ in the supernatants was measured using the MSD multi-spot assay system with the V-PLEX human IFN-γ kit (K151QOD-4) according to manufacturer's instructions. MSD 96 well assay plates were washed 3 times with 1× Tris wash buffer and 50 μl of culture supernatants diluted 100 fold in diluent 2 were added to each well. Plates were incubated on an orbital shaker (VWR Scientific) for 120 minutes at room temperature and washed 3 times with 1×Tris wash buffer. Detection antibody solution (25 μl) was added to the plate. Plates were incubated on an orbital shaker (VWR Scientific) for 120 minutes at room temperature and washed 3 times with 1×Tris wash buffer. 150 μl 2× Read Buffer T was added to each well and emitted light intensity was read in luminescence units on a MSD Quickplex SQ120 instrument.

To compare the effect of each IL-2 VHH dimer upon IFN-γ production, luminescence values from IFN-γ measurements were compared to those obtained for control cells treated with growth medium alone and control cells treated with human IL-2 at 100 μM. IL-2 VHH dimers were identified that induced higher luminescence signals for IFN-γ production than media control. IL-2 VHH dimers that induced survival and proliferation of PBMC subpopulations were identified and some showed a bias in activity towards T cells. The data from these experiments is presented in Tables 26 and Table 27.

TABLE 26
Activity of VHH dimers on PBMC IFN-γ production and
phenotype Donor 1.

| | | IFN-γ | T cells | NK cells |
|---|---|---|---|---|
| VHH SEQ | VHH DR1 | Production | (% CD3 | (% CD56 |
| ID | DR2 ID | (LU) | positive) | positive) |
| DR632 | DR229-DR214 | 135 | | |
| DR633 | DR229-DR217 | 104 | | |
| DR634 | DR229-DR583 | 16798 | 48.49 | 35.09 |
| DR635 | DR229-DR584 | 186103 | 47.77 | 50.35 |
| DR636 | DR229-DR585 | 2396 | 96.58 | 2.03 |
| DR637 | DR229-DR586 | 212 | | |
| DR638 | DR229-DR587 | 399282 | 41.98 | 55.82 |
| DR639 | DR229-DR588 | 5160 | 50.32 | 47.22 |
| DR640 | DR229-DR589 | 786 | | |
| DR641 | DR229-DR590 | 94 | | |
| DR642 | DR230-DR214 | 9540 | 42.72 | 55.39 |
| DR643 | DR230-DR217 | 43321 | 46.6 | 51.51 |
| DR644 | DR230-DR583 | 14668 | 62.91 | 33.69 |
| DR645 | DR230-DR584 | 1462066 | 36.3 | 60.92 |
| DR646 | DR230-DR585 | 258 | | |
| DR647 | DR230-DR586 | 50480 | 46 | 51.99 |
| DR648 | DR230-DR587 | 70954 | 42.48 | 55.24 |
| DR649 | DR230-DR588 | 301 | | |
| DR650 | DR230-DR589 | 85 | | |
| DR651 | DR230-DR590 | 76 | | |
| DR652 | DR231-DR214 | 167926 | 44.52 | 53.45 |
| DR653 | DR231-DR217 | 633 | | |
| DR654 | DR231-DR583 | 275 | | |
| DR655 | DR231-DR584 | 18389 | 74.95 | 21.18 |
| DR656 | DR231-DR585 | 552204 | 38.8 | 59.01 |
| DR657 | DR231-DR586 | 87 | | |
| DR658 | DR231-DR587 | 97 | | |
| DR659 | DR231-DR588 | 83 | | |
| DR660 | DR231-DR589 | 87 | | |
| DR661 | DR231-DR590 | 82 | | |
| DR662 | DR232-DR214 | 94 | | |
| DR663 | DR232-DR217 | 283 | | |
| DR664 | DR232-DR583 | 113 | | |
| DR665 | DR232-DR584 | 79 | | |
| DR666 | DR232-DR585 | 161 | | |
| DR667 | DR232-DR586 | 239 | | |
| DR668 | DR232-DR587 | 81 | | |
| DR669 | DR232-DR588 | 247 | | |
| DR670 | DR232-DR589 | 90 | | |
| DR671 | DR232-DR590 | 526 | | |
| DR672 | DR233-DR214 | 1087 | | |
| DR673 | DR233-DR217 | 8738 | 41.99 | 55.81 |
| DR674 | DR233-DR583 | 2842 | 38.7 | 59.4 |
| DR675 | DR233-DR584 | 32239 | 44.85 | 53.27 |
| DR676 | DR233-DR585 | 83 | | |
| DR677 | DR233-DR586 | 298 | | |
| DR678 | DR233-DR587 | 32319 | 46.56 | 51.48 |
| DR679 | DR233-DR588 | 121672 | 36.13 | 61.88 |
| DR680 | DR233-DR589 | 11020 | 46.18 | 52.08 |
| DR681 | DR233-DR590 | 159 | | |
| DR682 | DR234-DR214 | 3571 | 59.92 | 36.44 |
| DR683 | DR234-DR217 | 94 | | |
| DR684 | DR234-DR583 | 82 | | |
| DR685 | DR234-DR584 | 516 | | |
| DR686 | DR234-DR585 | 168 | | |
| DR687 | DR234-DR586 | 85 | | |
| DR688 | DR234-DR587 | 2812 | 39.99 | 57.85 |
| DR689 | DR234-DR588 | 890 | | |
| DR690 | DR234-DR589 | 246 | | |
| DR691 | DR234-DR590 | 85 | | |
| DR692 | DR214-DR229 | 88 | | |
| DR693 | DR214-DR230 | 74 | | |
| DR694 | DR214-DR231 | 76 | | |
| DR695 | DR214-DR232 | 828 | | |
| DR696 | DR214-DR233 | 111 | | |
| DR697 | DR214-DR234 | 132 | | |
| DR698 | DR217-DR229 | 78 | | |
| DR699 | DR217-DR230 | 104 | | |
| DR700 | DR217-DR231 | 96 | | |
| DR701 | DR217-DR232 | 2570 | 57.26 | 40.3 |
| DR702 | DR217-DR233 | 90 | | |
| DR703 | DR217-DR234 | 162 | | |
| DR704 | DR583-DR229 | 85 | | |
| DR705 | DR583-DR230 | 83 | | |
| DR706 | DR583-DR231 | 81 | | |
| DR707 | DR583-DR232 | 80 | | |
| DR708 | DR583-DR233 | 77 | | |
| DR709 | DR583-DR234 | 82 | | |
| DR710 | DR584-DR229 | 67 | | |
| DR711 | DR584-DR230 | 181 | | |
| DR712 | DR584-DR231 | 94 | | |
| DR713 | DR584-DR232 | 117 | | |
| DR714 | DR584-DR233 | 1932 | 92.95 | 3.96 |
| DR715 | DR584-DR234 | 76 | | |
| DR716 | DR585-DR229 | 5178 | 36.89 | 61.04 |
| DR717 | DR585-DR230 | 14665 | 48.32 | 49.33 |
| DR718 | DR585-DR231 | 79834 | 40.28 | 58.21 |
| DR719 | DR585-DR232 | 20846 | 38.28 | 59.66 |
| DR720 | DR585-DR233 | 734 | | |
| DR721 | DR585-DR234 | 68144 | 40.48 | 57.36 |
| DR722 | DR586-DR229 | 5762 | 45.21 | 53 |
| DR723 | DR586-DR230 | 120 | | |
| DR724 | DR586-DR231 | 12094 | 46.7 | 51.26 |
| DR725 | DR586-DR232 | 170 | | |
| DR726 | DR586-DR233 | 79 | | |
| DR727 | DR586-DR234 | 79 | | |
| DR728 | DR587-DR229 | 92 | | |
| DR729 | DR587-DR230 | 78 | | |
| DR730 | DR587-DR231 | 93 | | |
| DR731 | DR587-DR232 | 90 | | |
| DR732 | DR587-DR233 | 503 | | |
| DR733 | DR587-DR234 | 81 | | |
| DR734 | DR588-DR229 | 878 | | |
| DR735 | DR588-DR230 | 630 | | |
| DR736 | DR588-DR231 | 136028 | 41.38 | 56.82 |
| DR737 | DR588-DR232 | 567 | | |
| DR738 | DR588-DR233 | 224 | | |
| DR739 | DR588-DR234 | 1454 | | |
| DR740 | DR589-DR229 | 1272 | 78.22 | 17.89 |
| DR741 | DR589-DR230 | 2216 | 79.76 | 17 |
| DR742 | DR589-DR231 | 296 | | |
| DR743 | DR589-DR232 | 98 | | |
| DR744 | DR589-DR233 | 1099 | 85.55 | 10.34 |
| DR745 | DR589-DR234 | 91 | | |
| DR746 | DR590-DR229 | 868 | | |
| DR747 | DR590-DR230 | 3385 | 41.18 | 56.19 |
| DR748 | DR590-DR231 | 834 | | |
| DR749 | DR590-DR232 | 90 | | |
| DR750 | DR590-DR233 | 115 | | |
| DR751 | DR590-DR234 | 98 | | |
| Media | n/a | 235 | | |
| hIL-2 | n/a | 338863 | 63.24 | 34.52 |

LU: Luminescence Units.
n/a: not applicable

TABLE 27
Activity of VHH dimers on PBMC IFN-γ production and
phenotype Donor 2.

| VHH | | IFN-γ | T cells | NK cells |
|---|---|---|---|---|
| SEQ | VHH DR1 | Production | (% CD3 | (% CD56 |
| ID | DR2 ID | (LU) | positive) | positive) |
| DR632 | DR229-DR214 | 117 | | |
| DR633 | DR229-DR217 | 163 | | |
| DR634 | DR229-DR583 | 5188 | 62.22 | 35.73 |
| DR635 | DR229-DR584 | 11871 | 51.63 | 46.16 |
| DR636 | DR229-DR585 | 127 | 97.46 | 0.3 |
| DR637 | DR229-DR586 | 126 | | |
| DR638 | DR229-DR587 | 40748 | 50.69 | 47.15 |
| DR639 | DR229-DR588 | 2237 | 58.85 | 39.54 |
| DR640 | DR229-DR589 | 3128 | | |
| DR641 | DR229-DR590 | 74 | | |
| DR642 | DR230-DR214 | 1043 | 58.95 | 39.26 |
| DR643 | DR230-DR217 | 12027 | 50.19 | 47.74 |
| DR644 | DR230-DR583 | 1395 | 77.48 | 20 |
| DR645 | DR230-DR584 | 147962 | 56.88 | 41.48 |
| DR646 | DR230-DR585 | 105 | | |
| DR647 | DR230-DR586 | 24426 | 51.6 | 46.43 |
| DR648 | DR230-DR587 | 16661 | 53.06 | 45.12 |
| DR649 | DR230-DR588 | 107 | | |
| DR650 | DR230-DR589 | 86 | | |
| DR651 | DR230-DR590 | 101 | | |
| DR652 | DR231-DR214 | 21818 | 49.74 | 47.99 |
| DR653 | DR231-DR217 | 265 | | |
| DR654 | DR231-DR583 | 81 | | |
| DR655 | DR231-DR584 | 8336 | 81.11 | 15.93 |
| DR656 | DR231-DR585 | 165479 | 55.4 | 42.95 |
| DR657 | DR231-DR586 | 81 | | |
| DR658 | DR231-DR587 | 101 | | |
| DR659 | DR231-DR588 | 85 | | |
| DR660 | DR231-DR589 | 85 | | |
| DR661 | DR231-DR590 | 77 | | |
| DR662 | DR232-DR214 | 77 | | |
| DR663 | DR232-DR217 | 232 | | |
| DR664 | DR232-DR583 | 101 | | |
| DR665 | DR232-DR584 | 82 | | |
| DR666 | DR232-DR585 | 87 | | |
| DR667 | DR232-DR586 | 180 | | |
| DR668 | DR232-DR587 | 89 | | |
| DR669 | DR232-DR588 | 82 | | |
| DR670 | DR232-DR589 | 84 | | |
| DR671 | DR232-DR590 | 250 | | |
| DR672 | DR233-DR214 | 323 | | |
| DR673 | DR233-DR217 | 1977 | 65.3 | 32.57 |
| DR674 | DR233-DR583 | 2565 | 61.54 | 36.29 |
| DR675 | DR233-DR584 | 41300 | 42.86 | 55.03 |
| DR676 | DR233-DR585 | 82 | | |
| DR677 | DR233-DR586 | 242 | | |
| DR678 | DR233-DR587 | 18981 | 53.53 | 44.54 |
| DR679 | DR233-DR588 | 5117 | 57.75 | 40.55 |
| DR680 | DR233-DR589 | 2370 | 63.81 | 34.23 |
| DR681 | DR233-DR590 | 154 | | |
| DR682 | DR234-DR214 | 1462 | 68.15 | 29.23 |
| DR683 | DR234-DR217 | 145 | | |
| DR684 | DR234-DR583 | 109 | | |
| DR685 | DR234-DR584 | 840 | | |
| DR686 | DR234-DR585 | 89 | | |
| DR687 | DR234-DR586 | 79 | | |
| DR688 | DR234-DR587 | 653 | 58.97 | 38.6 |
| DR689 | DR234-DR588 | 332 | | |
| DR690 | DR234-DR589 | 132 | | |
| DR691 | DR234-DR590 | 73 | | |
| DR692 | DR214-DR229 | 101 | | |
| DR693 | DR214-DR230 | 88 | | |
| DR694 | DR214-DR231 | 76 | | |
| DR695 | DR214-DR232 | 78 | | |
| DR696 | DR214-DR233 | 92 | | |
| DR697 | DR214-DR234 | 117 | | |
| DR698 | DR217-DR229 | 82 | | |
| DR699 | DR217-DR230 | 173 | | |
| DR700 | DR217-DR231 | 100 | | |
| DR701 | DR217-DR232 | 1227 | 63.05 | 35.35 |
| DR702 | DR217-DR233 | 96 | | |
| DR703 | DR217-DR234 | 1143 | | |
| DR704 | DR583-DR229 | 185 | | |
| DR705 | DR583-DR230 | 81 | | |
| DR706 | DR583-DR231 | 84 | | |
| DR707 | DR583-DR232 | 96 | | |
| DR708 | DR583-DR233 | 83 | | |
| DR709 | DR583-DR234 | 84 | | |
| DR710 | DR584-DR229 | 72 | | |
| DR711 | DR584-DR230 | 75 | | |
| DR712 | DR584-DR231 | 75 | | |
| DR713 | DR584-DR232 | 91 | | |
| DR714 | DR584-DR233 | 63 | 95.35 | 2.33 |
| DR715 | DR584-DR234 | 228 | | |
| DR716 | DR585-DR229 | 1273 | 55.37 | 42.69 |
| DR717 | DR585-DR230 | 26060 | 45.59 | 51.88 |
| DR718 | DR585-DR231 | 38220 | 46.94 | 50.75 |
| DR719 | DR585-DR232 | 1850 | 88.89 | 6.13 |
| DR720 | DR585-DR233 | 766 | | |
| DR721 | DR585-DR234 | 24693 | 48.32 | 49.32 |
| DR722 | DR586-DR229 | 5851 | 52.03 | 46.36 |
| DR723 | DR586-DR230 | 173 | | |
| DR724 | DR586-DR231 | 3693 | 57.36 | 40.62 |
| DR725 | DR586-DR232 | 93 | | |
| DR726 | DR586-DR233 | 85 | | |
| DR727 | DR586-DR234 | 91 | | |
| DR728 | DR587-DR229 | 340 | | |
| DR729 | DR587-DR230 | 95 | | |
| DR730 | DR587-DR231 | 237 | | |
| DR731 | DR587-DR232 | 99 | | |
| DR732 | DR587-DR233 | 113 | | |
| DR733 | DR587-DR234 | 142 | | |
| DR734 | DR588-DR229 | 4151 | | |
| DR735 | DR588-DR230 | 625 | | |
| DR736 | DR588-DR231 | 2595 | 55.41 | 42.05 |
| DR737 | DR588-DR232 | 923 | | |
| DR738 | DR588-DR233 | 104 | | |
| DR739 | DR588-DR234 | 830 | | |
| DR740 | DR589-DR229 | 873 | 86.55 | 6.04 |
| DR741 | DR589-DR230 | 629 | 84.17 | 9.79 |
| DR742 | DR589-DR231 | 198 | | |
| DR743 | DR589-DR232 | 82 | | |
| DR744 | DR589-DR233 | 625 | 82.76 | 8.27 |
| DR745 | DR589-DR234 | 81 | | |
| DR746 | DR590-DR229 | 212 | | |
| DR747 | DR590-DR230 | 2272 | 68.05 | 29.44 |
| DR748 | DR590-DR231 | 1050 | | |
| DR749 | DR590-DR232 | 386 | | |
| DR750 | DR590-DR233 | 138 | | |
| DR751 | DR590-DR234 | 88 | | |
| Media | n/a | 226 | | |
| hIL-2 | n/a | 174981 | 64.09 | 33.48 |

LU: Luminescence Units.
n/a: not applicable

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

### Tables

TABLE 2
anti-human IL2Rb sdAb CDRs

| | | SEQ | | SEQ | | SEQ |
|---|---|---|---|---|---|---|
| Name of | | ID | | ID | | ID |
| VHH | CDR 1 | NO: | CDR 2 | NO: | CDR 3 | NO: |
| hIL2Rb_VHH-1 | YTYDTSDMS | 25 | DIDSGDWAAYADAVK | 33 | SYWKWGKLNNF | 41 |
| | | | G | | | |
| hIL2Rb_VHH-2 | FTFSNYWIF | 26 | TSNTGGDTTKYADSV | 34 | GRCARSG | 42 |
| | | | KG | | | |
| hIL2Rb_VHH-3 | FRFSNYGMS | 27 | YINGDGSRTHYADSV | 35 | GLSRDGWSLSAAS | 43 |
| | | | KG | | | |
| hIL2Rb_VHH-4 | YTTYSFNYM | 28 | VIYTGGGSTLYADSV | 36 | DDQRFASPLYAYFG | 44 |
| | G | | KG | | Y | |
| hIL2Rb_VHH-5 | DTKSIRCMG | 29 | AIDREGFATYADSVY | 37 | QNMCRVVRGAMTGV | 45 |
| | | | D | | DY | |
| hIL2Rb_VHH-6 | YTASRYCMA | 30 | AIHPGGGTTYYADSV | 38 | GSLWVPFGDRCAAN | 46 |
| | | | KG | | Y | |
| hIL2Rb_VHH-7 | YEYCRIHMT | 31 | SIGSDGRKTYANSVT | 39 | EYLYGLGCPDGSAY | 47 |
| | | | G | | | |
| hIL2Rb_VHH-8 | YTYSSYYCM | 32 | AIDSDGSTSYADSVK | 40 | SYEVVDCYPSGYGQ | 48 |
| | G | | G | | DY | |

TABLE 3
murine IL2Rb sdAb CDRs

| | | SEQ | | SEQ | | SEQ |
|---|---|---|---|---|---|---|
| | | ID | | ID | | ID |
| Name | CDR | NO: | CDR | NO: | CDR | NO: |
| DR857 | FTFSLYDMS | 49 | GINSGGYSTYYAASA | 102 | RGLTSPYVIPNI | 125 |
| | | | KG | | | |
| DR1448 | KSFSDYPLG | 50 | HISWSGKLTYYRSTV | 103 | MKLFNYGGRYCVLKPLTM | 126 |
| | | | KG | | YQQ | |
| DR1449 | RSFSGYAIG | 51 | VVSWRGSSTYYADS | 104 | VPSGRSWYGRNRY | 127 |
| | | | VKG | | | |
| DR1450 | RSINYYRMG | 52 | AIKWGGDGVYADSV | 105 | MPLSSWSRGGYLEV | 128 |
| | | | KG | | | |
| DR1451 | RFSWGNYAMY | 53 | AIGRNSMATYYRDS | 106 | KFMVADGWSRQYDY | 129 |
| | | | AKG | | | |
| DR1452 | RTFRRFMG | 54 | AINWPGGGTYYGDS | 107 | TRKYNLYKFAD | 130 |
| | | | VKG | | | |
| DR1453 | RIFNTYSMG | 55 | AIRWSGGTTYYTDSV | 108 | RVRLSNTALLQRY | 131 |
| | | | KG | | | |
| DR1454 | RTFGDYPIG | 56 | SISWGGSRQYYTDSV | 109 | RVRLSNTALLQRY | 132 |
| | | | KG | | | |
| DR1455 | RTFNSYAMG | 57 | VITWNSGRTYYADS | 110 | APWAHNRE | 133 |
| | | | VKG | | | |
| DR1456 | LTFRTYYMS | 58 | VISWIGSTTLYADSV | 111 | NFLREGKREPRY | 134 |
| | | | KG | | | |
| DR1457 | RIFNTYSMG | 59 | AIRWSGGTTYYTDSV | 112 | RVFARRY | 135 |
| | | | KG | | | |
| DR1458 | RTLSTYAMG | 60 | AIRWASGRTYYGDS | 113 | RSRPYLNYGDFGY | 136 |
| | | | VKG | | | |
| DR1459 | RTISTYAMV | 61 | VISRSGDRTYYADSV | 114 | GGYTGIETITA | 137 |
| | | | KG | | | |
| DR1460 | SIFNNNAVY | 62 | LITIGGRTGYADSVK | 115 | GLKFGFNFYSKTAYDY | 138 |
| | | | G | | | |
| DR1461 | RIFNTYSMG | 63 | AIRWSGGTTYYTDSV | 116 | VPSGRSWYGRNRY | 139 |
| | | | KG | | | |
| DR1462 | RTFGYVAMG | 64 | SINWSGGSTAYADSV | 117 | STRFYIATMEQGSYDY | 140 |
| | | | KG | | | |
| DR1463 | RSFRSYAIG | 65 | AISYDGRRTYYGRSL | 118 | HRSGTMFARYGMDY | 141 |
| | | | KD | | | |
| DR1464 | RTFSSYAMG | 66 | AISRSGGYTSYADSV | 119 | LIAPFYYGMDY | 142 |
| | | | KG | | | |
| DR1465 | PTFTSYTMG | 67 | VISKGGRTYYADSVK | 120 | QRVGATSKYEYDY | 143 |
| | | | G | | | |
| DR1466 | FTFSTDWMY | 68 | LINTDGTSTSYTKSV | 121 | GRTYWFYAMDY | 144 |
| | | | KG | | | |
| DR1467 | RISNYAMG | 69 | VITRSGGSTYYADSV | 122 | RRSQKLVTFGAEYPW | 145 |
| | | | KG | | | |
| DR1468 | RTGTHYAMG | 70 | LILWNGEFTTYKDSV | 123 | RVFARRY | 146 |
| | | | KG | | | |
| DR1469 | FTFSNYWM | 71 | HINTNGGNTYYRHSV | 124 | ANSDVGLGYYGMDY | 147 |
| | Y | | KG | | | |
| DR1470 | SIFNNNAVY | 72 | LITIGGRTGYADSVK | 461 | RPGYWSSSYDY | 148 |
| | | | G | | | |
| DR1471 | RAPASYAMA | 73 | AINWSGRRTYYADS | 462 | YLSGTYY | 149 |
| | | | VKG | | | |
| DR1472 | LASSSFFMT | 74 | TISWTGRTSYYAASV | 463 | YPRTLVRNREPIH | 150 |
| | | | KG | | | |

TABLE 4
human IL2Rg sdAb CDRs

| | | SEQ | | SEQ | | SEQ |
|---|---|---|---|---|---|---|
| | | ID | | ID | | ID |
| Name | CDR | NO: | CDR | NO: | CDR | NO: |
| hIL2Rg_ | FTFDDSD | 151 | TISSDGSTYYADSV | 174 | DFMIAIQAPGAGC | 197 |
| VHH-1 | MG | | KG | | | |
| hIL2Rg_ | FSFSSYPM | 152 | TIASDGGSTAYAAS | 175 | GYGDGTPA | 198 |
| VHH-2 | T | | VEG | | | |
| hIL2Rg_ | FTFDDRE | 153 | TISSDGSTYYADSV | 176 | DFMIAIQAPGAGC | 199 |
| VHH-3 | MN | | KG | | | |
| hIL2Rg_ | FTFDDSD | 154 | TISSDGNTYYTDSV | 177 | EPRGYYSNYGGRRECNY | 200 |
| VHH-4 | MG | | KG | | | |
| hIL2Rg_ | FSFSSYPM | 155 | TIASDGGSTAYAAS | 178 | GYGDGTPA | 201 |
| VHH-5 | T | | IVEG | | | |
| hIL2Rg_ | FTFSNAH | 156 | SIYSGGSTWYADSV | 179 | NRLHYYSDDDSL | 202 |
| VHH-6 | MS | | KG | | | |
| hIL2Rg_ | FTFDDRE | 157 | TISSDGSTYYADSV | 180 | DFMIAIQAPGAGC | 203 |
| VHH-7 | MN | | KG | | | |
| hIL2Rg_ | YTFSSYC | 158 | ALGGGSTYYADSV | 181 | AWVACLEFGGSWYDLAR | 204 |
| VHH-8 | MG | | KG | | YKH | |
| hIL2Rg_ | FTFDDSD | 159 | TISSDGSTYYADSV | 182 | EPRGYYSNYGGRRECNY | 205 |
| VHH-9 | MC | | KG | | | |
| hIL2Rg_ | SIYSSAYI | 160 | GIYTRDGSTAYADS | 183 | GRRTKSYVYIFRPEEYNY | 206 |
| VHH-10 | G | | VKG | | | |
| hIL2Rg_ | FTFSSAH | 161 | SIYSGGGTFYADSV | 184 | NRLHYYSDDDSL | 207 |
| VHH-11 | MS | | KG | | | |
| hIL2Rg_ | FTFSNAH | 162 | SIYSGGSTWYADSV | 185 | NRLHYYSDDDSL | 208 |
| VHH-12 | MS | | KG | | | |
| hIL2Rg_ | FIFDDSDM | 163 | TISSDGSTYYADSV | 186 | EPRGYYSNYGGRRECNY | 209 |
| VHH-13 | G | | KG | | | |
| hIL2Rg_ | FTADDSD | 164 | TISSDGSTYYADSV | 187 | EPRGYYSNYGGRRECNY | 210 |
| VHH-14 | MG | | KG | | | |
| hIL2Rg_ | FTFSSAH | 165 | SIYSGGGTFYADSV | 188 | NRLHYYSDDDSL | 211 |
| VHH-15 | MS | | KG | | | |
| hIL2Rg_ | FTFSNAH | 166 | SIYSGGSTWYADSV | 189 | NRLHYYSDDDSL | 212 |
| VHH-16 | MS | | KG | | | |
| hIL2Rg_ | FTFSNAH | 167 | SIYSGGSTWYADSV | 190 | NRLHYYSDDDSL | 213 |
| VHH-17 | MS | | KG | | | |
| hIL2Rg_ | FTFSSYPM | 168 | VEG | 191 | GYGDGTPA | 214 |
| VHH-18 | T | | TIASDGGSTAYAAS | | | |
| hIL2Rg_ | FTFDDRE | 169 | TISSDGSTYYADSV | 192 | DFMIAIQAPGAGC | 215 |
| VHH-19 | MN | | KG | | | |
| hIL2Rg_ | FTFDDSD | 170 | TISSDGSTYYADSV | 193 | EPRGYYSNYGGRRECNY | 216 |
| VHH-20 | MG | | KG | | | |
| hIL2Rg_ | YTSCMG | 171 | TIYTRGRSIYYADSV | 194 | GGYSWSAGCEFNY | 217 |
| VHH-21 | | | KG | | | |
| hIL2Rg_ | FSFSSYPM | 172 | TIASDGGSTAYAAS | 195 | GYGDGTPA | 218 |
| VHH-22 | | | VEG | | | |
| hIL2Rg_ | FSFSSYPM | 173 | TIASDGGSTAYAAS | 196 | GYGDGTPA | 219 |
| VHH-23 | T | | VEG | | | |

TABLE 5
mouse IL2Rg sdAb CDRs

| | | SEQ | | SEQ | | SEQ |
|---|---|---|---|---|---|---|
| | | ID | | ID | | ID |
| Name | CDR 1 | NO: | CDR 2 | NO: | CDR 3 | NO: |
| DR604 | YGYNYIG | 220 | VIYTGGGDTYYA | 235 | SVYACLRGGHDEYAH | 250 |
| | | | DSVKG | | | |
| mIL2Rg_ | STYANYL | 221 | AIYSGGGSTYYA | 236 | ASAVKGDKGDIVVVVTGT | 251 |
| VHH2 | MG | | DSVKG | | QRMEYDY | |
| mIL2Rg_ | FTFDESV | 222 | IISSDDNTYYDDS | 237 | RRRRPVYDSDYELRPRPLC | 252 |
| VHH3 | MS | | VKG | | GDFGV | |
| mIL2Rg_ | LPFDEDD | 223 | SISSDGTAYYADS | 238 | GVHRQFGGSSSCGDAFYG | 253 |
| VHH4 | MG | | VKG | | MDY | |
| mIL2Rg_ | DVYGRN | 224 | VGYSVVTTTYYA | 239 | DGNLWRGLRPSEYTY | 254 |
| VHH5 | SMA | | DSVKG | | | |
| mIL2Rg_ | FPYSRYC | 225 | AIEPDGSTSYADS | 240 | DERCFYLKDYDLRRPAQY | 255 |
| VHH6 | MG | | VKG | | RY | |
| mIL2Rg_ | FTFDESD | 226 | VITSDDNPYYDD | 241 | RSRQPVYSRDYELRPRPLC | 256 |
| VHH7 | MG | | SVKG | | GDFGV | |
| mIL2Rg_ | FTFDDFD | 227 | TISDDGSTYYADS | 242 | EGALGSKTNCGWVGNFG | 257 |
| VHH8 | MG | | VKG | | Y | |
| mIL2Rg_ | FTFDDFD | 228 | TISDDGSTYYADS | 243 | EGALGSKTNCGWVGNFG | 258 |
| VHH9 | MG | | VKG | | Y | |
| mIL2Rg_ | FTFDDFD | 229 | TISDDGSTYYADS | 244 | EGALGSKTNCGWVGNFG | 259 |
| VHH10 | MG | | VKG | | Y | |
| mIL2Rg_ | FTFSDRD | 230 | TISDDGSTYYADS | 245 | EGALGSKTNCGWVGNFG | 260 |
| VHH11 | MG | | VKG | | | |
| mIL2Rg_ | YGYNYIG | 231 | VIYIGGGDTYYA | 246 | RYCVGSVYACLRGGHDEY | 261 |
| VHH12 | | | DSVKG | | AH | |
| mIL2Rg_ | YGYNYIG | 232 | VIYTGGGDTYYA | 247 | RYCVGSVYACLRGGHDEY | 262 |
| VHH13 | | | DSVKG | | AH | |
| mIL2Rg_ | FTFDDFD | 233 | TISDDGSTYYANS | 248 | EGALGSKTNCGWVGNFG | 263 |
| VHH14 | MG | | VKG | | Y | |
| mIL2Rg_ | FTFDDFD | 234 | TISDDGSTYYADS | 249 | EGALGSKMNCGWVGNFG | 264 |
| VHH15 | MG | | VKG | | Y | |

TABLE 6
human anti-IL2Rb VHH Amino Acid Sequences

| | | SEQ |
|---|---|---|
| | | ID |
| Name | VHH Sequence | NO: |
| hIL2Rb_ | QVQLQESGGGSVQAGGSLRLSCVGSGYTYDTSDMSWYRQAPGKER | 265 |
| VHH-1 | EFVSDIDSGDWAAYADAVKGRFTISRDNAKKTVYLQMNSLEPEDTA | |
| | MYYCKASYWKWGKLNNFWGPGTQVTVSS | |
| hIL2Rb_ | QVQLQESGGGLVQPGGSLRLSCVASGFTFSNYWIFWVRQAAGKGLE | 266 |
| VHH-2 | WLSTSNTGGDTTKYADSVKGRFTISRDSAKNTEYLQMNSLKPEDTA | |
| | VYYCETGRCARSGGYQGTQVTVSS | |
| hIL2Rb_ | QVQLQESGGGLVQPGGSLKLSCAASGFRFSNYGMSWVRQAPGEGLE | 267 |
| VHH-3 | WVSYINGDGSRTHYADSVKGRFTISRDNAKNTLYLQLNSLKTEDTA | |
| | MYYCEKGLSRDGWSLSAASRGQGTQVTVSS | |
| hIL2Rb_ | QVQLQESGGGSVQTGGSLRLSCAVSGYTTYSFNYMGWFRQAPGKE | 268 |
| VHH-4 | REGVAVIYTGGGSTLYADSVKGRFTISQDNAKNTVYLQMNSLKPED | |
| | TAMYYCAADDQRFASPLYAYFGYWGQGTQVTVSS | |
| hIL2Rb_ | QVQLQESGGGSVQVGGSLRLSCATSGDTKSIRCMGWFRQTPGKERE | 269 |
| VHH-5 | GIAAIDREGFATYADSVYDRFTIAQDNAQNTLYLEMNALKPEDTAM | |
| | YYCAAQNMCRVVRGAMTGVDYWGKGTQVTVSS | |
| hIL2Rb_ | QVQLQESGGGSVQAGGSLRLSCAASEYTASRYCMAWFRQAPGKER | 270 |
| VHH-6 | EGVAAIHPGGGTTYYADSVKGRFSISQDSADNTLYLQMNSLKPEDTA | |
| | MYYCAAGSLWVPFGDRCAANYWGQGTQVTVSS | |
| hIL2Rb_ | QVQLQESGGGSVQAGGSLRLSCAASGYEYCRIHMTWYRQGPGKER | 271 |
| VHH-7 | EFVSSIGSDGRKTYANSVTGRFTISRDNANHTVYLQMNSLSPEDTAM | |
| | YYCKTEYLYGLGCPDGSAYWGQGTQVTVSS | |
| hIL2Rb_ | QVQLQESGGGSVQVGGSLKLSCAASGYTYSSYYCMGWFRQAPGKE | 272 |
| VHH-8 | REGVAAIDSDGSTSYADSVKGRFTISQDDAKNTLYLQMNSLKPEDTA | |
| | MYYCAASYEVVDCYPSGYGQDYWGKGTQVTVSS | |

TABLE 7
murine anti-IL2Rb VHH Amino Acid Sequences

| | | VHH |
|---|---|---|
| | VHH AA Sequence | SEQ ID |
| Name | (CDRs Underlined) | NO: |
| DR857 | QVQLQESGGGLVQPGGSLRLSCAASGFTFSLYDMSWVRQAPG | 273 |
| | KGLEWVSGINSGGYSTYYAASAKGRFTISRDNAKNTLYLQLSS | |
| | VKTEDTAMYYCAQRGLTSPYVIPNIRLQGTQVTVSS | |
| DR1448 | EVQLVESGGRLVQAGDSLRLSCVASGKSFSDYPLGWFRQAPGK | 274 |
| | AREYVAHISWSGKLTYYRSTVKGRFTISRDNAENKLYLQMNAL | |
| | KPEDTAVYYCAAMKLFNYGGRYCVLKPLTMYQQWSQGTQVT | |
| | VSS | |
| DR1449 | EVQLVESGGGLVQAGGSLRLSCAASGRSFSGYAIGWFRQAPGK | 275 |
| | EREFVAVVSWRGSSTYYADSVKGRFTISRDNAKGTVYLQMNSL | |
| | KPEDTAAYYCAAVPSGRSWYGRNRYWGQGTQVTVSS | |
| DR1450 | EVQLVESGGGLVQAGGSLRLSCVISGRSINYYRMGWFRQAPGN | 276 |
| | RRQFVAAIKWGGDGVYADSVKGRFTISRDNTKNTVYLQMDSL | |
| | KPEDTGTYYCAKMPLSSWSRGGYLEVWGQGTLVTVSS | |
| DR1451 | EVQLVESGGGLVQAGDSLRLSCAASERFSWGNYAMYWFRQAP | 277 |
| | GKEREFVAAIGRNSMATYYRDSAKGRFVISRDNAKNTLYLEMN | |
| | ALKPEDTARYYCAAKFMVADGWSRQYDYWGQGTLVTVSS | |
| DR1452 | EVQLVESGGGLVQAGGALRLSCAASGRTFRRFMGWFRQAPGK | 278 |
| | EREFVAAINWPGGGTYYGDSVKGRFTISRDNAKNTVYLQMNSL | |
| | KPEDTANYYCAATRKYNLYKFADWGQGTQVTVSS | |
| DR1453 | EVQLVESGGRLVQAGDSLRLSCVASGRIFNTYSMGWFRQVPGK | 279 |
| | ERDFVAAIRWSGGTTYYTDSVKGRFTISRDNAKNTVYLQMNSL | |
| | KPEDTAVYYCWVRVRLSNTALLQRYWGQGTLVTVSS | |
| DR1454 | EVQLVESGGGLVQAGGSLRLFCASSERTFGDYPIGWFRQAPGK | 280 |
| | EREFVASISWGGSRQYYTDSVKGRFTITRDNDKNTVYLQMNSL | |
| | KPEDTAVYYCWVRVRLSNTALLQRYWGQGTLVTVSS | |
| DR1455 | EVQLVESGGGLVQTGGSLRLSCAASGRTFNSYAMGWFRQSPG | 281 |
| | KEREFVAVITWNSGRTYYADSVKGRFTISRDNAKNTVYLQMNS | |
| | LKPEDTAVYYCNSAPWAHNREWGQGTLVTVSS | |
| DR1456 | EVQLVESGGGLVQAGGSLRLSCAASGLTFRTYYMSWFRQAPG | 282 |
| | KEREFVGVISWIGSTTLYADSVKGRFSISRDNAKNTVYLQMNN | |
| | LKPEDTAVYYCAANFLREGKREPRYWGQGTQVTVSS | |
| DR1457 | EVQLVESGGRLVQAGDSLRLSCVASGRIFNTYSMGWFRQVPGK | 283 |
| | ERDFVAAIRWSGGTTYYTDSVKGRFTISRDNAKNTVYLQMNSL | |
| | KPEDTAVYYCYLRVFARRYWGQGTQVTVSS | |
| DR1458 | EVQLVESGGGLVQAGGSLRLSCAASGRTLSTYAMGWFRQAPG | 284 |
| | KEREFVAAIRWASGRTYYGDSVKGRFTISRDSAKNTVYLQMNS | |
| | LKPEDTAVYYCAARSRPYLNYGDFGYWGQGTQVTVSS | |
| DR1459 | EVQLVESGGGLVQAGGSLRLSCAASGRTISTYAMVWFRQASG | 285 |
| | KEREFVGVISRSGDRTYYADSVKGRFTISRDNLGNIVRLQLNSL | |
| | KPEDTAVYYCARGGYTGIETITARGRGTLVTVSS | |
| DR1460 | EVQLVESGGGLVQTGDSLRLSCAAPESIFNNNAVYWYRQFPGK | 286 |
| | EREYVGLITIGGRTGYADSVKGRFTISRDNANNVAFLQMDSLKP | |
| | EDTAVYYCATGLKFGFNFYSKTAYDYWGQGTQVTVSS | |
| DR1461 | EVQLVESGGRLVQAGDSLRLSCVASGRIFNTYSMGWFRQVPGK | 287 |
| | ERDFVAAIRWSGGTTYYTDSVKGRFTISRDNAKNTVYLQMKDL | |
| | KPQDTAVYYCAAVPSGRSWYGRNRYWGQGTLVTVSS | |
| DR1462 | EVQLVESGGGLVQAGGSLRLSCVSSGRTFGYVAMGWFRQAPG | 288 |
| | KEREFVASINWSGGSTAYADSVKGRFTISRDNAKNTVYLQMNS | |
| | LKPEDTAVYYCAGSTRFYIATMEQGSYDYWGQGTQVTVSS | |
| DR1463 | EVQLVESGGSVVQPGDSLRLACTASGRSFRSYAIGWFRQASGK | 289 |
| | ERVFVAAISYDGRRTYYGRSLKDRFTISRDNAKNTVYLQMNSL | |
| | KPEDTAVYYCATHRSGTMFARYGMDYWGKGTLVTVSS | |
| DR1464 | EVQLVESGGGLVQAGGSLRLSCAASGRTFSSYAMGWFRQAPG | 290 |
| | KEREFVTAISRSGGYTSYADSVKGRFTISRDNAKNTVYLQMNSL | |
| | KPEDTAVYYCAKLIAPFYYGMDYWTKGTQVTVSS | |
| DR1465 | EVQLVESGGGLMQAGGALRLSCTASGPTFTSYTMGWFRQSPG | 291 |
| | KRREFVAVISKGGRTYYADSVKGRFTISRDNAKNTFYLQMSSL | |
| | KPEDTAVYYCAGQRVGATSKYEYDYWGQGTQVTVSS | |
| DR1466 | EVQLVESGGGLVRAGGSLRLSCAASGFTFSTDWMYWVRRAPG | 292 |
| | KGLEWVSLINTDGTSTSYTKSVKGRFTVSRDNAKNTLYLQMNS | |
| | LKPEDTALYYCARGRTYWFYAMDYWGKGTQVTVSS | |
| DR1467 | EVQLVESGGGLVQAGDSLRLSCAASGRISNYAMGWFRQAPGK | 293 |
| | EREFVAVITRSGGSTYYADSVKGRFTISRDNGKNTIDLQMNRLK | |
| | PEDTAVYYCAVRRSQKLVTFGAEYPWWGQGTLVTVSS | |
| DR1468 | EVQLVESGGGLVQAGGSLRLSCTTSGRTGTHYAMGWFRQAPG | 294 |
| | KEREFVSLILWNGEFTTYKDSVKGRFTISREKGENTVYLQMNSL | |
| | KPEDTAVYYCYLRVFARRYWGQGTQVTVSS | |
| DR1469 | EVQLVESGGGLVQPGGSLRLSCEVSGFTFSNYWMYWIRQAPGK | 295 |
| | GLEWVSHINTNGGNTYYRHSVKGRFTISRDNAKNTLYLQMNG | |
| | LKSEDTAVYYCAKANSDVGLGYYGMDYWGKGTQVTVSS | |
| DR1470 | EVQLVESGGGSVQPGGSLRLSCAAPESIFNNNAVYWYRQFPGK | 296 |
| | EREYVGLITIGGRTGYADSVKGRFTISRDNANNVAFLQMDNLK | |
| | PEDTAVYYCAARPGYWSSSYDYWGQGTQVTVSS | |
| DR1471 | EVQLVESGGGLVQAGGSLRLSCVFSGRAPASYAMAWFRQAVG | 297 |
| | NEREFVAAINWSGRRTYYADSVKGRFTISKDNAQNTAYLQMT | |
| | NLEPEDTATYYCNAYLSGTYYWGQGTQVTVSS | |
| DR1472 | EVQLVESGGGLVRAGDSLRLSCAVSGLASSSFFMTWFRQGQGK | 298 |
| | EREFVATISWTGRTSYYAASVKGRFTVSRDNAKNTVYLQMNSL | |
| | NSEDTAVYFCAAYPRTLVRNREPIHWGQGTQVTVSS | |

TABLE 8
human anti-IL2Rg VHH Amino Acid Sequences

| | VHH Sequence | SEQ ID |
|---|---|---|
| Name | (CDRs are underlined) | NO: |
| hIL2Rg_ | QVQLQESGGGSVQAGGSLRLSCAASGFTFDDSDMGWYRQ | 299 |
| VHH-1 | APGNECDLVSTISSDGSTYYADSVKGRFTISQDNAKNTVYL | |
| | QMDSVKPEDTAVYYCAADFMIAIQAPGAGCWGQGTQVTV | |
| | SS | |
| hIL2Rg_ | QVQLQESGGGSVPAGGSLKLSCAASGFSFSSYPMTWARQA | 300 |
| VHH-2 | PGKGLEWVSTIASDGGSTAYAASVEGRFTISRDNAKSTLYL | |
| | QLNSLKTEDTAMYYCTKGYGDGTPAPGQGTQVTVSS | |
| hIL2Rg_ | QVQLQESGGGSVQTGGSLRLSCTASGFTFDDREMNWYRQ | 301 |
| VHH-3 | APGNECELVSTISSDGSTYYADSVKGRFTISQDNAKNTVYL | |
| | QMDSVKPEDTAVYYCAADFMIAIQAPGAGCWGQGTQVTV | |
| | SS | |
| hIL2Rg_ | QVQLQESGGGSVQAGGSLRLSCTASGFTFDDSDMGWYRQ | 302 |
| VHH-4 | APGNECELVSTISSDGNTYYTDSVKGRFTISQDNAKNTVYL | |
| | QMNSLGPEDTAVYYCAAEPRGYYSNYGGRRECNYWGQG | |
| | TQVTVSS | |
| hIL2Rg_ | QVQLQESGGGSVQAGGSLRLSCAASGFSFSSYPMTWARQA | 303 |
| VHH-5 | PGKGLEWVSTIASDGGSTAYAASVEGRFTISRDNAKSTLYL | |
| | QLNSLKTEDTAMYYCTKGYGDGTPAPGQGTQVTVSS | |
| hIL2Rg_ | QVQLQESGGGAVQAGGSLRLSCAASGFTFSNAHMSWVRQ | 304 |
| VHH-6 | APGKGREWISSIYSGGSTWYADSVKGRFTISRDNSKNTLYL | |
| | QLNSLKTEDTAMYYCAENRLHYYSDDDSLRGQGTQVTVS | |
| | S | |
| hIL2Rg_ | QVQLQESGGGLVQPGGSLRLSCAASGFTFDDREMNWYRQ | 305 |
| VHH-7 | APGNECELVSTISSDGSTYYADSVKGRFTISQDNAKNTVYL | |
| | QMDSVKPEDTAVYYCAADFMIAIQAPGAGCWGQGTQVTV | |
| | SS | |
| hIL2Rg_ | QVQLQESGGGSVQAGGSLRLSCVASGYTFSSYCMGWFRQ | 306 |
| VHH-8 | APGKEREGVAALGGGSTYYADSVKGRFTISQDNAKNTLYL | |
| | QMNSLKPEDTAMYYCAAAWVACLEFGGSWYDLARYKH | |
| | WGQGTQVTVSS | |
| hIL2Rg_ | QVQLQESGGGSVQAGGSLRLSCTASGFTFDDSDMGWYRQ | 307 |
| VHH-9 | APGGECELVTISSDGSTYYADSVKGRFTISQDNAKNTVYLQ | |
| | MNSLKPEDTAVYYCAAEPRGYYSNYGGRRECNYWGQGT | |
| | QVTVSS | |
| hIL2Rg_ | QVQLQESGGGSVQAGGSLRLSCAASGSIYSSAYIGWFRQAP | 308 |
| VHH-10 | GKKREGVAGIYTRDGSTAYADSVKGRFTISQDSAKKTVYL | |
| | QMNSLKPEDTAMYYCAAGRRTKSYVYIFRPEEYNYWGQG | |
| | TQVTVSS | |
| hIL2Rg_ | QVQLQESGGGSVQAGGSLRLSCAASGFTFSSAHMSWVRQ | 309 |
| VHH-11 | APGKGREWIASIYSGGGTFYADSVKGRFTISRDNAKNTLYL | |
| | QLNSLKTEDTAMYYCATNRLHYYSDDDSLRGQGTQVTVS | |
| | S | |
| hIL2Rg_ | QVQLQESGGGSVQAGGSLRLSCAASGFTFSNAHMSWVRQ | 310 |
| VHH-12 | APGKGREWISSIYSGGSTWYADSVKGRFTISRDNSKNTLYL | |
| | QLNSLKTEDTAMYYCAENRLHYYSDDDSLRGQGTQVTVS | |
| | S | |
| hIL2Rg_ | QVQLQESGGGSVQAGGSLRLSCTASRFIFDDSDMGWYRQA | 311 |
| VHH-13 | PGNECELVSTISSDGSTYYADSVKGRFTISRDNAKNTVYLQ | |
| | MNSLKPEDTAVYYCAAEPRGYYSNYGGRRECNYWGQGT | |
| | QVTVSS | |
| hIL2Rg_ | QVQLQESGGGSVQAGGSLKLSCTVSGFTADDSDMGWYRQ | 312 |
| VHH-14 | GPGNECELVTISSDGSTYYADSVKGRFTISQDNAKNTVYLQ | |
| | MNSLKPEDTAVYYCAAEPRGYYSNYGGRRECNYWGQGT | |
| | QVTVSS | |
| hIL2Rg_ | QVQLQESGGGLVQPGGSLRLSCAASGFTFSSAHMSWVRQA | 313 |
| VHH-15 | PGKGREWIASIYSGGGTFYADSVKGRFTISRDNAKNTLYLQ | |
| | LNSLKAEDTAMYYCATNRLHYYSDDDSLRGQGTQVTVSS | |
| hIL2Rg_ | QVQLQESGGGLVQPGGSLRLSCVASGFTFSNAHMSWVRQ | 314 |
| VHH-16 | APGKGREWISSIYSGGSTWYADSVKGRFTISRDNSKNTLYL | |
| | QLNSLKTEDTAMYYCAENRLHYYSDDDSLRGQGTQVTVS | |
| | S | |
| hIL2Rg_ | QVQLQESGGGLVQPGGSLRLSCAASGFTFSNAHMSWVRQ | 315 |
| VHH-17 | APGKGREWISSIYSGGSTWYADSVKGRFTISRDNSKNTLYL | |
| | QLNSLKTEDTAMYYCAENRLHYYSDDDSLRGQGTQVTVS | |
| | S | |
| hIL2Rg_ | QVQLQESGGGLVQPGGSLRLSCAASGFTFSSYPMTWARQA | 316 |
| VHH-18 | PGKGLEWVSTIASDGGSTAYAASVEGRFTISRDNAKSTLYL | |
| | QLNSLKTEDTAMYYCTKGYGDGTPAPGQGTQVTVSS | |
| hIL2Rg_ | QVQLQESGGGSVQAGGSLRLSCTASGFTFDDREMNWYRQ | 317 |
| VHH-19 | APGNECELVSTISSDGSTYYADSVKGRFTISQDNAKNTVYL | |
| | QMDSVKPEDTAVYYCAADFMIAIQAPGAGCWGQGTQVTV | |
| | SS | |
| hIL2Rg_ | QVQLQESGGGSVQAGGSLRLSCTASGFTFDDSDMGWYRQ | 318 |
| VHH-20 | APGNECELVSTISSDGSTYYADSVKGRFTISQDNAKNTVYL | |
| | QMNSLKPEDTAVYYCAAEPRGYYSNYGGRRECNYWGQG | |
| | TQVTVSS | |
| hIL2Rg_ | QVQLQESGGGSVQAGGSLRLSCVASGYTSCMGWFRQAPG | 319 |
| VHH-21 | KEREAVATIYTRGRSIYYADSVKGRFTISQDNAKNTLYLQ | |
| | MNSLKPEDIAMYSCAAGGYSWSAGCEFNYWGQGTQVTVS | |
| | S | |
| hIL2Rg_ | QVQLQESGGGLVQPGGSLRLSCTASGFSFSSYPMTWARQA | 320 |
| VHH-22 | PGKGLEWVSTIASDGGSTAYAASVEGRFTISRDNAKSTLYL | |
| | QLNSLKTEDTAMYYCTKGYGDGTPAPGQGTQVTVSS | |
| hIL2Rg_ | QVQLQESGGGLVQPGGSLRLSCAASGFSFSSYPMTWARQA | 321 |
| VHH-23 | PGKGLEWVSTIASDGGSTAYAASVEGRFTISRDNAKSTLYL | |
| | QLNSLKTEDTAMYYCTKGYGDGTPAPGQGTQVTVSS | |

TABLE 9
murine anti-IL2Rg VHH Amino Acid Sequences

| | VHH AA Sequence | SEQ ID |
|---|---|---|
| Name | (CDRs Underlined) | NO: |
| mIL2Rg_ | QVQLQESGGGSVLAGGSLRLSCVASGYGYNYIGWFRQTPGKEREG | 322 |
| VHH1 | VAVIYTGGGDTYYADSVKGRFTASRDNAKSTLYLQMNSLEPEDTA | |
| | MYYGVARYCVGSVYACLRGGHDEYAHWGQGTQVTVSS | |
| mIL2Rg_ | QVQLQESGGGSVQPGGSLRLSCAASGSTYANYLMGWFRQAPGKE | 323 |
| VHH2 | REGVAAIYSGGGSTYYADSVKGRFTISQDNAKNTLYLQMNSLKPE | |
| | DTAMYYCAAASAVKGDKGDIVVVVTGTQRMEYDYWGHGTQVT | |
| | VSS | |
| mIL2Rg_ | QVQLQESGGGSVQAGASLRLSCSVSGFTFDESVMSWLRQGPGNEC | 324 |
| VHH3 | DAVAIISSDDNTYYDDSVKGRFTISEDNAKNMVYLQMNSLKPEDT | |
| | AVYYCAARRRRPVYDSDYELRPRPLCGDFGVWGQGTQVTVSS | |
| mIL2Rg_ | QVQLQESGGGSVQAGGSLRLSCIGSGLPFDEDDMGWYRQAPGNE | 325 |
| VHH4 | VICELVSSISSDGTAYYADSVKGRFTISRDNAKNTVLLQMNSLKPEDT | |
| | AVYYCAAGVHRQFGGSSSCGDAFYGMDYWGKGTQVTVSS | |
| mIL2Rg_ | QVQLQESGGGSVQAGGSLRLSCVASGDVYGRNSMAWFRQAPGKE | 326 |
| VHH5 | REGVAVGYSVVTTTYYADSVKGRFTISEDNDKNTVYLEMNSLKPE | |
| | DTAMYYCAADGNLWRGLRPSEYTYWGQGTQVTVSS | |
| mIL2Rg_ | QVQLQESGGGSVQAGGSLRLSCATSGFPYSRYCMGWFRQAPGKE | 327 |
| VHH6 | REGVAAIEPDGSTSYADSVKGRFTISQDNAVNTLYLQMNNLKPED | |
| | TAMYYCAADERCFYLKDYDLRRPAQYRYWGQGTQVTVSS | |
| mIL2Rg_ | QVQLQESGGGLVQPGGSLRLSCTVSGFTFDESDMGWLRQNPGNEC | 328 |
| VHH7 | GVVSVITSDDNPYYDDSVKGRFTISEDNAKNMVYLQMNSLKPEDT | |
| | GVYYCATRSRQPVYSRDYELRPRPLCGDFGVWGQGTQVTVSS | |
| mIL2Rg_ | QVQLQESGGGSVQAGGSLRLSCTASGFTFDDFDMGWYRQAPGNE | 329 |
| VHH8 | CELVSTISDDGSTYYADSVKGRSSISRDNAKNTVYLQMNRLKPEDT | |
| | GVYYCAAEGALGSKTNCGWVGNFGYWGQGTQVTVSS | |
| mIL2Rg_ | QVQLQESGGGSVQAGGSLRLSCAASGFTFDDFDMGWYRQAPGNE | 330 |
| VHH9 | VICELVSTISDDGSTYYADSVKGRSSISRDNAKNTVYLQMNSLKPEDT | |
| | AVYYCAAEGALGSKTNCGWVGNFGYWGQGTQVTVSS | |
| mIL2Rg_ | QVQLQESGGGLVQPGGSLRLSCAASGFTFDDFDMGWYRQAPGNE | 331 |
| VHH10 | VICELVSTISDDGSTYYADSVKGRSSISRDNAKSTVYLQMNRLKPEDT | |
| | GVYYCAAEGALGSKTNCGWVGNFGYWGQGTQVTVSS | |
| mIL2Rg_ | QVQLQESGGGLVQPGGSLKLSCAASGFTFSDRDMGWYRQAPGNE | 332 |
| VHH11 | CERVSTISDDGSTYYADSVKGRSSISRDNAKNTVYLQMNSLKPEDT | |
| | AVYYCAAEGALGSKTNCGWVGNFGYWGQGTQVTVSS | |
| mIL2Rg_ | QVQLQESGGGSVLAGGSLRLSCVASGYGYNYIGWFRQTPGKEREG | 333 |
| VHH12 | VIVAVIYIGGGDTYYADSVKGRFTASRDNAKSTLYLQMNSLEPEDTA | |
| | MYYCVARYCVGSVYACLRGGHDEYAHWGQGTQVTVSS | |
| mIL2Rg_ | QVQLQESGGGSVLAGGSLRLSCVASGYGYNYIGWFRQTPGKEREG | 334 |
| VHH13 | VAVIYTGGGDTYYADSVKGRFTASRDNAKSTLYLQMNSLEPEDTA | |
| | MYYCVARYCVGSVYACLRGGHDEYAHWGQGTQVTVSS | |
| mIL2Rg_ | QVQLQESGGGSVQAGGSLRLSCAASGFTFDDFDMGWYRQAPGNE | 335 |
| VHH14 | CELVSTISDDGSTYYANSVKGRSSISRDNAKNMVYLQMNSLKPED | |
| | TAVYYCAAEGALGSKTNCGWVGNFGYWGQGTQVTVSS | |
| mIL2Rg_ | QVQLQESGGGSVQAGGSLRLSCTASGFTFDDFDMGWYRQAPGNE | 336 |
| VHH15 | CELVSTISDDGSTYYADSVKGRSSISRDNAKNTVYLQMNRLKPEDT | |
| | GVYYCAAEGALGSKMNCGWVGNFGYWGQGTQVTVSS | |

TABLE 10
IL10Rb sdAb VHH DNA SEQUENCE

| Table 10. hIL10Ra VHH DNA Sequences | | SEQ ID |
|---|---|---|
| Name | Sequence | NO: |
| hIL10Ra_ | CAGGTTCAGCTTCAGGAGTCCGGTGGAGGCTCCATCCAGGCCGGG | 337 |
| VHH1 | GGCTCTCTCCGCCTGTCTTGCGCCGCTTCCAGATACCTCTACAGTA | |
| | TCGACTACATGGCTTGGTTTCGTCAGAGCCCAGGAAAAGAGCGGG | |
| | AACCCGTGGCAGTAATCTACACTGCCTCAGGTGCCACATTTTACC | |
| | CCGACTCTGTCAAGGGCAGGTTCACCATCTCTCAGGATAATGCCA | |
| | AGATGACAGTGTACTTGCAGATGAACTCCCTGAAATCTGAGGATA | |
| | CCGCTATGTATTACTGTGCCGCAGTGCGCAAGACCGATTCTTACC | |
| | TGTTCGACGCTCAGAGTTTTACCTACTGGGGCCAGGGCACTCAGG | |
| | TCACCGTCAGCAGC | |
| hIL10Ra_ | CAGGTGCAGTTGCAGGAGTCCGGCGGGGGTTCCGTGCAAGCAGG | 338 |
| VHH2 | CGGATCTCTGCGCCTGTCCTGCGTGGCCTCTCGTTATTTGTATAGC | |
| | ACCAACTACATGGCTTGGTTCCGTCAGTCCCCAGGCAAAGAGCGC | |
| | GAAGCCGTAGCCGTAATCTATACGGCCTCTGGGGCAACACTCTAT | |
| | ACCGACTCAGTGAAGGGACGCTTCACGATTTCCCAAGACAATGCA | |
| | AAGATGACCGTGTACTTGCAGATGAACCGCCTGAAGAGCGAGGA | |
| | CACGGCTATGTATTACTGCGCAGCCGTGCGCAAGACCGACTCCTA | |
| | CTTGTTTGACGCTCAGTCCTTCACTTATTGGGGCCAGGGTACACA | |
| | GGTCACCGTGAGCAGT | |
| hIL10Ra_ | CAAGTACAGCTCCAGGAGAGCGGCGGTGGATCTATCCAAGCAGG | 339 |
| VHH3 | GGGTAGCCTTAGGTTGTCCTGTGTGGCGTCCAGATACCTGTATAG | |
| | CACGAACTACATGGCATGGTTCAGACAGTCCCCAGGCAAGGAAC | |
| | GCGAGGCAGTCGCCGTTATTTACACTGCATCTGGGGCCACCCTCT | |
| | ATACGGACAGCGTGAAGGGAAGGTTTACAATCTCCCAGGACAAC | |
| | GCGAAGATGACCGTGTACCTTCAGATGAACCGCCTGAAGTCCGAG | |
| | GACACCGCCATGTATTACTGTGCAGCGGTGCGCAAGACCGACAGC | |
| | TATCTGTTCGACGCGCAGTCATTCACTTATTGGGGCCAAGGAACC | |
| | CAAGTGACCGTCAGCTCA | |
| hIL10Ra_ | CAGGTGCAGCTCCAAGAGTCCGGGGGAGGCTCTATCCAGGCGGG | 340 |
| VHH4 | AGGCAGTCTGCGCTTGTCCTGCGCCGCAAGTCGTTATCTGTACTCC | |
| | ATTGATTACATGGCATGGTTCCGCCAGTCCCCAGGTAAGGAACGT | |
| | GAACCTGCCGCTGTGATCTACACCGCTTCTGGAGCAACCTTTTATC | |
| | CTGATAGCGTTAAGGGTCGCTTCACCATCTCTCAGGATAACGCCA | |
| | AAATGACAGTGTACCTCCAGATGAACAGCCTGAAGTCTGAGGAC | |
| | ACTGCCATGTACTATTGTGCGGCTGTGCGCAAGACCGACTCCTAT | |
| | CTGTTTGATGCACAGAGCTTTACCTATTGGGGTCAGGGCACCCAG | |
| | GTGACTGTGTCTAGC | |
| hIL10Ra_ | CAGGTCCAGTTGCAGGAGTCCGGTGGAGGTTCCATCCAGGCGGGT | 341 |
| VHH5 | GGGTCCCTTCGTCTCTCCTGCGTGGCCTCTAAGTACCTGTATTCAA | |
| | CCAACTACATGGCATGGTTCAGACAGTCTCCCGGCAAAGAGCGTG | |
| | AGGCAGTGGCCGCGATCTATACAGCTTCTGGGGCCACCCTGTACT | |
| | CTGATTCCAATAAGGGAAGGTTCACTATCTCACAGGATAACGCCA | |
| | AAATGACCGTCTACCTTCAGATGAACAGCCTCAAGTCTGAAGACA | |
| | CGGCAATGTATTACTGTGCAGCCGTGCGCAAAACTGGGAGCTACC | |
| | TGTTTGACGCTCAGTCTTTCACTTATTGGGGCCAGGGTACGCAGG | |
| | TGACAGTCTCTTCT | |
| hIL10Ra_ | CAGGTGCAACTCCAGGAGAGCGGAGGCGGTTCTGTTCAGGCAGG | 342 |
| VHH6 | AGGTTCCCTGAGACTGTCCTGTGCCGCGTCTCGCTTTACGTATTCA | |
| | TCCTACTGCATGGGATGGTTCAGACAAGCGCCGGGGAAAGAAAG | |
| | GGAAGGCGTGGCCTCCATTGACTCCGACGGCTCAACTTCATACAC | |
| | TGATAGCGTGAAAGGCCGGTTCACCATCTCTAAGGACAACGCGA | |
| | AGAACACCCTGTATCTCCAGATGAACAGCCTCAAGCCTGAGGATA | |
| | CTGCCATGTACTATTGCGCACTCGACCTGATGTCTACTGTGGTCCC | |
| | AGGCTTCTGCGGGTTCCTGCTCTCTGCTGGCATGGACTACTGGGG | |
| | GAAGGGCACTCAGGTAACGGTTAGCTCC | |
| hIL10Ra_ | CAGGTGCAGCTTCAGGAATCTGGCGGGGGCTCCGTGCAGGCCGG | 343 |
| VHH7 | GGGCTCCCTCAGACTTTCCTGTGCCGTCTCCGGTTACACATTTAAC | |
| | AGTAACTGTATGGGCTGGTTCCGCCAGGCACCAGGCAAGGAGAG | |
| | GGAAGGTGTGGCCACAATCTATACTGGTGTTGGGAGTACGTACTA | |
| | TGCTGATTCCGTGAAAGGTCGCTTCACAATTTCCCAGGACAACGC | |
| | GAAGAACACTGTGTACTTGCAGATGAATAGCCTGAAGCCTGAAG | |
| | ATACCGCAATGTATTACTGCGCTGCCGAGCCACTCTCCCGCGTAT | |
| | ATGGTGGAAGTTGCCCCACCCCCACTTTCGGTTACTGGGGCCAGG | |
| | GCACTCAAGTGACCGTGTCCTCT | |
| hIL10Ra_ | CAGGTTCAGCTTCAGGAGTCTGGGGGCGGTTCAGTGCAGGCTGGC | 344 |
| VHH8 | GGTTCTCTCCGCCTGTCCTGCGCTGCCAGCGGCTATACTTACAGCA | |
| | TGTACTGCATGGGCTGGTTCCGGCAAGCCCCCGGCAAAGAGCGTG | |
| | AGGGCGTCGCTCAAATCAACAGCGACGGGTCAACCAGCTACGCC | |
| | GATTCTGTCAAGGGCAGATTTACTATCAGCAAGGACAACGCCAAA | |
| | AACACACTGTACCTCCAGATGAACTCTTTGAAGCCTGAGGACACC | |
| | GCGATGTATTACTGCGCCGCTGACAGCCGCGTGTACGGTGGCAGC | |
| | TGGTATGAGAGGCTGTGCGGCCCGTACACCTACGAGTACAACTAT | |
| | TGGGGACAGGGCACGCAGGTGACAGTTAGCTCC | |

TABLE 11
murine IL2Rb sdAb VHH DNA SEQUENCE

| | | SEQ ID |
|---|---|---|
| Name | DNA Sequence | NO |
| DR857 | CAGGTGCAGTTGCAGGAGAGCGGGGGCGGTCTGGTCCAGCCGGGC | 345 |
| DNA | GGGTCACTGCGCCTGTCTTGTGCCGCTTCAGGATTTACCTTTAGTTT | |
| | GTACGACATGAGTTGGGTTAGGCAAGCGCCTGGCAAGGGTCTGGA | |
| | GTGGGTGTCTGGCATCAACTCAGGAGGCTATAGCACCTATTACGCG | |
| | GCCTCCGCCAAGGGCCGCTTCACCATCTCTAGGGATAACGCAAAGA | |
| | ACACTCTTTACCTCCAGCTCAGCTCTGTTAAGACTGAGGATACTGCC | |
| | ATGTATTACTGTGCCCAGCGCGGCCTCACCAGCCCGTATGTGATTCC | |
| | GAACATTCGCTTGCAGGGCACACAGGTGACTGTGTCCAGC | |
| DR1448 | GAGGTCCAACTGGTGGAGAGCGGCGGAAGGCTGGTGCAGGCTGGC | 346 |
| DNA | GACTCCCTGCGCTTGAGCTGTGTGGCAAGCGGAAAGTCCTTTTCCG | |
| | ATTACCCTCTCGGTTGGTTCCGTCAGGCTCCTGGAAAAGCTAGGGA | |
| | GTATGTGGCCCACATCTCTTGGAGCGGCAAACTGACTTACTATCGC | |
| | TCAACAGTGAAGGGCCGGTTTACTATCAGCCGCGATAACGCTGAAA | |
| | ATAAACTGTACCTCCAGATGAACGCCCTGAAGCCCGAGGATACTGC | |
| | CGTGTATTACTGTGCTGCCATGAAGTTGTTCAACTATGGCGGGCGTT | |
| | ACTGTGTTCTCAAGCCCCTGACAATGTACCAACAGTGGAGCCAGGG | |
| | TACTCAGGTCACAGTTAGCTCC | |
| DR1449 | GAGGTCCAGCTCGTTGAGAGCGGCGGGGGCCTGGTGCAGGCCGGT | 347 |
| DNA | GGCAGCCTCCGTCTCTCCTGTGCCGCTTCTGGCCGCAGTTTCTCCGG | |
| | GTATGCTATCGGGTGGTTCAGACAGGCACCAGGCAAGGAGCGCGA | |
| | GTTTGTTGCTGTCGTGAGCTGGCGGGGTTCTAGCACCTACTATGCCG | |
| | ACTCAGTCAAGGGCCGCTTCACAATTAGCAGGGACAACGCCAAGG | |
| | GCACTGTATACCTCCAGATGAACTCCCTGAAGCCAGAGGATACCGC | |
| | CGCGTATTACTGCGCTGCCGTGCCATCTGGCCGCTCCTGGTACGGTA | |
| | GGAACCGTTACTGGGGTCAGGGAACTCAGGTCACCGTGTCCTCA | |
| DR1450 | GAAGTGCAGCTCGTTGAAAGCGGCGGGGGCCTCGTGCAAGCTGGA | 348 |
| DNA | GGCTCACTTCGCCTTTCTTGTGTCATCAGTGGCCGCTCTATCAATTA | |
| | TTACCGGATGGGCTGGTTCCGCCAGGCCCCTGGCAACCGCAGGCAA | |
| | TTCGTGGCGGCTATCAAGTGGGGTGGCGACGGTGTGTACGCCGACT | |
| | CCGTGAAGGGGCGCTTTACCATTAGTCGGGACAACACCAAGAACAC | |
| | CGTATACTTGCAGATGGACAGTCTGAAGCCCGAAGACACCGGAAC | |
| | ATATTACTGCGCCAAAATGCCTCTTTCTAGCTGGTCCAGAGGTGGCT | |
| | ACCTTGAGGTGTGGGGTCAAGGCACGCTGGTGACCGTGTCTTCT | |
| DR1451 | GAAGTGCAACTCGTGGAAAGTGGAGGCGGTCTCGTCCAGGCGGGG | 349 |
| DNA | GACAGCCTGCGTCTGTCTTGCGCCGCATCCGAGCGTTTTTCTTGGGG | |
| | CAACTATGCTATGTATTGGTTCAGGCAGGCCCCTGGCAAGGAACGC | |
| | GAGTTCGTGGCTGCCATTGGCCGCAACAGCATGGCCACGTATTACA | |
| | GAGATAGCGCCAAGGGCCGCTTCGTCATCAGCCGTGACAACGCTAA | |
| | GAACACCCTGTACCTGGAAATGAACGCCTTGAAGCCTGAAGATACT | |
| | GCTAGGTACTATTGCGCCGCGAAGTTCATGGTGGCCGACGGCTGGA | |
| | GCAGACAGTATGACTACTGGGGCCAGGGCACTCTGGTAACGGTCTC | |
| | CTCC | |
| DR1452 | GAAGTTCAGCTTGTGGAAAGCGGCGGTGGGCTTGTCCAGGCTGGTG | 350 |
| DNA | GAGCGCTGCGCCTCTCCTGCGCAGCGAGTGGCAGGACCTTCCGCCG | |
| | TTTCATGGGTTGGTTTCGCCAGGCCCCAGGGAAGGAGCGCGAGTTT | |
| | GTTGCTGCCATCAACTGGCCTGGAGGTGGCACCTACTATGGCGATA | |
| | GCGTGAAGGGCCGTTTCACAATCTCCAGGGACAACGCCAAGAATAC | |
| | CGTCTACCTGCAAATGAACTCCCTGAAGCCGGAGGACACCGCGAAC | |
| | TATTACTGCGCCGCAACCCGCAAGTACAACCTGTATAAATTCGCGG | |
| | ACTGGGGCCAGGGCACCCAGGTGACAGTGTCATCT | |
| DR1453 | GAGGTCCAGCTCGTCGAGTCCGGCGGGCGGCTGGTGCAGGCTGGCG | 351 |
| DNA | ACAGCCTTCGCCTGTCCTGTGTGGCATCCGGCAGAATCTTTAACACC | |
| | TACTCAATGGGTTGGTTTAGGCAGGTTCCCGGAAAGGAGAGGGATT | |
| | TCGTGGCTGCCATCAGATGGTCCGGTGGCACCACATATTACACTGA | |
| | TTCTGTCAAGGGGCGCTTCACCATTAGTCGCGATAACGCAAAAAAC | |
| | ACCGTGTACCTGCAAATGAATAGCCTGAAGCCTGAGGACACCGCCG | |
| | TATATTACTGTTGGGTGCGCGTTCGCCTGAGCAACACAGCCCTGCTT | |
| | CAGCGCTACTGGGGTCAGGGAACCTTGGTTACCGTGTCAAGC | |
| DR1454 | GAAGTCCAGCTCGTGGAGTCCGGGGGAGGTCTGGTTCAAGCTGGGG | 352 |
| DNA | GTTCTTTGCGCCTCTTTTGCGCGTCCAGCGAGCGTACTTTCGGAGAT | |
| | TACCCAATCGGATGGTTCCGTCAAGCCCCAGGCAAGGAGCGCGAGT | |
| | TTGTCGCGTCCATCAGCTGGGGTGGCTCACGTCAGTACTATACTGA | |
| | CTCCGTTAAGGGCCGCTTCACGATTACAAGAGATAATGATAAGAAC | |
| | ACCGTGTATCTCCAGATGAACTCCCTCAAGCCCGAGGACACTGCTG | |
| | TTTACTATTGCTGGGTGCGGGTGCGTCTGTCAAACACGGCACTGCTT | |
| | CAGCGCTATTGGGGACAGGGCACCCTGGTCACCGTCTCCTCA | |
| DR1455 | GAAGTCCAGCTGGTCGAGTCAGGCGGGGGACTGGTGCAGACTGGG | 353 |
| DNA | GGTAGTCTGCGCCTGAGCTGCGCAGCTTCAGGAAGAACCTTCAACT | |
| | CCTACGCTATGGGCTGGTTCAGACAGAGCCCAGGCAAAGAGCGGG | |
| | AGTTCGTGGCGGTGATTACGTGGAACTCTGGCCGCACGTACTATGC | |
| | TGACAGTGTCAAAGGCAGATTTACCATCAGTAGGGATAACGCCAAG | |
| | AACACAGTGTATCTCCAGATGAACTCTCTGAAGCCCGAGGATACTG | |
| | CTGTGTATTACTGTAACAGCGCCCCCTGGGCTCACAATCGTGAGTG | |
| | GGGGCAGGGGACCCTCGTTACCGTCAGCAGC | |
| DR1456 | GAGGTGCAGCTGGTGGAATCTGGTGGAGGGCTGGTGCAGGCTGGC | 354 |
| DNA | GGTTCCCTCCGTCTGTCTTGTGCGGCCTCAGGGCTGACCTTCAGGAC | |
| | CTACTATATGTCATGGTTCCGCCAAGCGCCCGGCAAGGAACGCGAG | |
| | TTCGTCGGAGTGATCTCTTGGATCGGCTCCACTACCCTCTACGCCGA | |
| | TTCTGTGAAAGGTAGGTTTTCCATCTCACGCGATAATGCTAAGAAC | |
| | ACCGTCTACCTCCAGATGAATAACTTGAAACCCGAGGACACCGCCG | |
| | TCTACTATTGCGCGGCCAACTTCCTCAGAGAGGGAAAGCGCGAACC | |
| | TCGGTATTGGGGACAAGGGACCCAGGTGACCGTTTCCTCC | |
| DR1457 | GAGGTGCAGTTGGTTGAGTCTGGCGGAAGGCTCGTTCAAGCTGGTG | 355 |
| DNA | ACAGCCTGCGGCTGTCTTGCGTCGCTTCTGGACGCATCTTCAACACA | |
| | TATTCAATGGGCTGGTTCAGACAGGTGCCTGGCAAGGAGCGCGACT | |
| | TCGTGGCAGCTATCCGTTGGAGCGGGGGCACTACGTATTACACCGA | |
| | TTCTGTGAAGGGGCGCTTCACAATCTCCAGGGATAATGCAAAGAAC | |
| | ACCGTGTACCTTCAGATGAACAGCTTGAAGCCTGAAGATACCGCAG | |
| | TGTACTATTGTTATCTGAGGGTGTTCGCTCGGCGCTATTGGGGCCAG | |
| | GGCACACAGGTGACAGTGTCCTCC | |
| DR1458 | GAAGTGCAGCTGGTCGAGAGCGGGGGTGGACTTGTGCAGGCTGGT | 356 |
| DNA | GGCTCCCTTAGGCTGAGCTGCGCCGCTTCCGGCAGAACTCTCTCTAC | |
| | CTATGCTATGGGTTGGTTCCGTCAGGCCCCCGGCAAGGAGCGCGAG | |
| | TTCGTCGCGGCCATCCGCTGGGCTTCTGGCCGTACTTATTACGGTGA | |
| | CAGCGTGAAGGGTCGGTTCACCATCTCTCGTGACAGTGCGAAAAAT | |
| | ACCGTGTACCTCCAGATGAACTCCCTGAAGCCGGAGGACACGGCGG | |
| | TTTATTACTGCGCGGCCAGGAGCAGGCCTTACCTGAACTACGGAGA | |
| | CTTTGGGTACTGGGGCCAGGGGACCCAGGTCACCGTGTCATCC | |
| DR1459 | GAAGTCCAGCTCGTGGAGTCTGGGGGTGGACTCGTACAAGCCGGG | 357 |
| DNA | GGATCACTTCGCTTGTCCTGCGCGGCTTCTGGCAGGACCATCTCAAC | |
| | TTACGCAATGGTTTGGTTCAGGCAAGCCTCTGGTAAGGAGCGTGAG | |
| | TTTGTGGGCGTTATCTCCCGCAGTGGAGACCGCACTTACTATGCTGA | |
| | TTCTGTGAAGGGCAGATTCACTATCAGTCGCGATAATCTGGGCAAC | |
| | ATTGTGCGTTTGCAGCTCAATTCACTTAAACCTGAAGACACAGCCG | |
| | TTTATTACTGCGCACGCGGCGGATATACCGGGATTGAGACAATTAC | |
| | GGCTCGGGGTCGCGGCACATTGGTCACCGTGTCCAGC | |
| DR1460 | GAGGTTCAGCTCGTTGAGAGTGGTGGAGGCCTCGTGCAGACCGGGG | 358 |
| DNA | ATTCCCTTCGCCTTTCCTGTGCAGCTCCAGAGTCCATCTTCAACAAT | |
| | AACGCCGTTTACTGGTACAGGCAGTTCCCCGGCAAGGAGAGGGAGT | |
| | ATGTTGGTCTCATCACCATCGGTGGCAGGACCGGGTACGCGGACTC | |
| | TGTGAAAGGCCGCTTTACCATCTCCAGAGACAACGCCAATAACGTG | |
| | GCCTTTTTGCAGATGGATTCCCTCAAGCCCGAGGATACTGCTGTCTA | |
| | CTATTGTGCCACGGGCTTGAAGTTCGGCTTCAACTTCTACAGTAAG | |
| | ACTGCCTACGACTACTGGGGACAAGGGACCCAGGTGACCGTCAGCT | |
| | CT | |
| DR1461 | GAGGTGCAGCTCGTGGAGTCTGGAGGTCGCCTGGTGCAGGCTGGCG | 359 |
| DNA | ATTCCCTGCGCCTGTCCTGTGTGGCCTCTGGTCGCATTTTCAACACT | |
| | TATTCTATGGGTTGGTTCAGACAGGTTCCTGGAAAGGAAAGAGACT | |
| | TCGTGGCAGCCATTCGGTGGAGTGGTGGCACCACTTATTACACAGA | |
| | CTCCGTGAAGGGTCGCTTTACTATCTCTCGGGATAACGCCAAAAAC | |
| | ACTGTCTACCTCCAGATGAAAGACCTGAAGCCCCAGGACACCGCCG | |
| | TCTATTACTGTGCTGCCGTCCCCTCTGGCCGCAGCTGGTACGGTCGC | |
| | AACCGTTACTGGGGCCAGGGCACTCTGGTGACCGTCAGCTCT | |
| DR1462 | GAGGTGCAGTTGGTGGAGAGCGGCGGTGGCCTGGTCCAGGCGGGC | 360 |
| DNA | GGGTCCCTCCGCCTGAGTTGTGTGTCTTCAGGCCGGACCTTTGGATA | |
| | TGTCGCTATGGGTTGGTTCCGTCAAGCCCCAGGTAAGGAACGCGAG | |
| | TTCGTGGCGAGCATTAACTGGAGCGGCGGGTCCACGGCCTATGCGG | |
| | ACTCCGTAAAGGGCCGGTTCACTATCAGCCGCGACAACGCTAAGAA | |
| | TACCGTGTACTTGCAGATGAACAGCCTGAAGCCTGAGGATACAGCC | |
| | GTGTATTACTGCGCTGGATCAACCCGCTTCTATATCGCGACGATGG | |
| | AACAGGGCTCCTACGATTACTGGGGCCAAGGTACTCAGGTGACCGT | |
| | AAGCAGC | |
| DR1463 | GAGGTGCAACTGGTGGAATCAGGAGGCTCCGTGGTCCAGCCAGGG | 361 |
| DNA | GACAGCCTTCGTCTTGCCTGCACCGCCTCTGGTCGCAGTTTCAGGTC | |
| | TTACGCGATTGGCTGGTTTAGGCAGGCATCCGGCAAGGAAAGGGTG | |
| | TTTGTGGCTGCCATCTCTTATGACGGTAGGCGCACCTACTATGGGCG | |
| | TTCATTGAAGGACCGTTTCACTATCTCTCGGGACAACGCTAAGAAC | |
| | ACAGTGTACTTGCAGATGAACTCCCTCAAGCCCGAGGACACTGCCG | |
| | TGTACTATTGCGCTACCCATCGCTCCGGTACAATGTTCGCTCGGTAT | |
| | GGTATGGATTACTGGGGTAAGGGTACTTTGGTTACCGTGTCCAGC | |
| DR1464 | GAGGTGCAGCTGGTGGAGAGCGGCGGTGGCCTGGTGCAAGCAGGC | 362 |
| DNA | GGATCTCTGCGTCTGTCTTGTGCTGCGTCAGGCCGCACCTTCTCCTC | |
| | TTATGCTATGGGGTGGTTTAGACAAGCTCCTGGAAAGGAGAGGGAG | |
| | TTTGTGACTGCCATCTCCAGATCCGGTGGATACACTAGCTACGCCG | |
| | ATAGTGTTAAGGGCCGGTTCACTATCTCTCGCGACAATGCCAAGAA | |
| | CACCGTGTATCTTCAGATGAACTCCCTGAAACCCGAGGACACCGCC | |
| | GTCTACTATTGTGCGAAACTGATCGCTCCATTCTATTACGGCATGGA | |
| | TTACTGGACCAAGGGGACCCAGGTGACAGTGTCTAGC | |
| DR1465 | GAAGTGCAGCTGGTGGAAAGCGGCGGAGGTCTGATGCAGGCAGGT | 363 |
| DNA | GGAGCCCTTAGGCTCTCTTGTACCGCCTCTGGGCCTACTTTTACCTC | |
| | TTATACGATGGGCTGGTTCCGCCAATCTCCTGGCAAGCGTCGCGAG | |
| | TTTGTGGCCGTCATCTCCAAAGGCGGGCGGACCTATTACGCCGACT | |
| | CCGTGAAGGGACGCTTCACTATTTCCCGCGACAACGCTAAGAATAC | |
| | CTTCTATCTCCAGATGTCCTCTCTGAAGCCTGAGGACACAGCAGTGT | |
| | ATTACTGCGCCGGGCAGCGTGTGGGCGCGACTAGCAAGTATGAGTA | |
| | TGATTACTGGGGGCAGGGCACCCAAGTGACCGTGTCATCC | |
| DR1466 | GAAGTGCAACTGGTGGAGAGCGGAGGGGGTCTGGTACGCGCAGGT | 364 |
| DNA | GGCTCCCTGAGGCTCTCCTGCGCTGCGTCCGGCTTCACTTTTAGTAC | |
| | CGACTGGATGTACTGGGTAAGACGCGCTCCAGGAAAGGGGCTGGA | |
| | GTGGGTGTCCCTTATCAACACTGACGGGACTTCTACCTCCTATACTA | |
| | AGTCTGTGAAGGGGCGCTTCACAGTCTCCCGCGATAATGCCAAGAA | |
| | CACCTTGTACCTTCAGATGAACTCCCTCAAGCCGGAGGACACAGCT | |
| | CTGTATTACTGTGCACGCGGAAGAACCTACTGGTTTTACGCGATGG | |
| | ATTACTGGGGCAAGGGCACCCAGGTGACCGTCTCATCT | |
| DR1467 | GAGGTCCAGTTGGTGGAATCTGGAGGCGGACTGGTGCAGGCTGGA | 365 |
| DNA | GACAGTCTGAGATTGTCTTGTGCCGCTTCTGGCCGGATCAGCAACT | |
| | ACGCAATGGGCTGGTTCCGGCAGGCACCCGGTAAAGAAAGGGAGT | |
| | TCGTCGCTGTCATCACCAGGAGCGGCGGAAGCACATACTATGCTGA | |
| | TAGTGTTAAGGGCCGCTTCACCATTTCCAGAGATAACGGCAAAAAC | |
| | ACGATTGATCTTCAGATGAACAGACTGAAGCCTGAAGACACAGCA | |
| | GTGTACTATTGTGCCGTGAGGCGCAGTCAAAAACTGGTAACCTTTG | |
| | GCGCTGAGTATCCTTGGTGGGGCCAGGGAACATTGGTGACTGTCAG | |
| | CTCC | |
| DR1468 | GAGGTGCAGTTGGTGGAGAGCGGCGGAGGCTTGGTTCAAGCTGGG | 366 |
| DNA | GGCTCACTCAGGCTGTCTTGCACTACCTCTGGGCGTACAGGCACCC | |
| | ATTATGCGATGGGTTGGTTTAGGCAAGCGCCCGGCAAGGAACGCGA | |
| | GTTCGTTAGTCTCATCCTGTGGAACGGCGAGTTTACGACCTATAAA | |
| | GATTCTGTTAAGGGCCGCTTCACCATCTCCCGTGAGAAAGGCGAAA | |
| | ACACGGTCTACTTGCAAATGAACTCTCTGAAACCCGAGGATACTGC | |
| | GGTGTATTACTGCTACCTGAGGGTGTTTGCTAGGCGCTACTGGGGC | |
| | CAGGGAACCCAGGTGACCGTGTCCAGT | |
| DR1469 | GAAGTGCAGCTGGTGGAAAGTGGAGGCGGACTGGTGCAGCCAGGG | 367 |
| DNA | GGCAGCCTCCGCCTTTCTTGTGAGGTGTCCGGCTTTACCTTCAGCAA | |
| | CTACTGGATGTACTGGATTCGCCAAGCCCCTGGGAAGGGACTGGAG | |
| | TGGGTGTCCCACATTAACACCAACGGTGGCAACACTTATTACCGCC | |
| | ATAGTGTTAAAGGTAGATTCACTATCAGCAGGGATAACGCTAAGAA | |
| | TACCCTGTACTTGCAGATGAACGGCCTGAAGTCCGAGGACACCGCT | |
| | GTGTATTACTGTGCCAAGGCTAACTCCGATGTCGGGTTGGGTTATTA | |
| | CGGCATGGATTACTGGGGTAAGGGAACTCAGGTCACAGTGAGTTCT | |
| DR1470 | GAGGTGCAGCTGGTGGAAAGTGGCGGGGGCTCTGTTCAGCCCGGC | 368 |
| DNA | GGATCTCTGCGCCTGAGCTGTGCTGCACCAGAGTCCATCTTCAACA | |
| | ATAACGCTGTTTACTGGTATCGGCAATTTCCGGGCAAAGAAAGGGA | |
| | GTACGTGGGCCTCATCACGATTGGTGGGCGCACTGGATACGCCGAC | |
| | TCTGTCAAGGGCCGCTTTACTATCAGTCGTGATAACGCCAACAATG | |
| | TTGCTTTTCTCCAGATGGATAACCTGAAGCCGGAAGATACTGCGGT | |
| | ATATTACTGTGCCGCTAGGCCTGGATATTGGTCCAGTTCCTACGATT | |
| | ATTGGGGTCAGGGAACCCAAGTAACAGTGTCCTCT | |
| DR1471 | GAAGTGCAGCTGGTGGAAAGCGGCGGTGGCCTCGTGCAGGCGGGC | 369 |
| DNA | GGGTCCCTGAGACTGTCATGCGTCTTCTCTGGCCGCGCCCCGGCTA | |
| | GTTATGCAATGGCTTGGTTTCGCCAGGCCGTGGGCAACGAGAGGGA | |
| | GTTTGTCGCTGCGATCAACTGGTCCGGCAGGCGCACTTACTATGCC | |
| | GACTCAGTGAAGGGCCGCTTCACTATTTCCAAGGACAATGCACAGA | |
| | ACACCGCCTATCTCCAGATGACCAACTTGGAACCAGAGGATACTGC | |
| | CACGTATTACTGTAATGCTTACTTGAGCGGAACATATTACTGGGGC | |
| | CAGGGCACCCAGGTGACCGTCTCTAGC | |
| DR1472 | GAGGTCCAGCTGGTCGAGTCTGGCGGTGGCTTGGTCCGCGCTGGGG | 370 |
| DNA | ACTCACTGCGCCTGAGTTGTGCTGTGTCCGGCCTGGCCAGCTCCTCT | |
| | TTCTTTATGACTTGGTTCCGCCAAGGGCAGGGCAAGGAGCGGGAAT | |
| | TTGTGGCCACTATCAGTTGGACTGGCCGTACATCCTATTACGCTGCC | |
| | AGCGTGAAAGGCCGCTTTACCGTTAGTCGGGACAATGCCAAGAATA | |
| | CCGTGTACCTTCAGATGAACTCTCTGAACTCTGAGGATACAGCAGT | |
| | CTACTTCTGTGCAGCCTACCCGCGTACACTGGTGCGTAATCGCGAG | |
| | CCGATCCATTGGGGTCAGGGAACCCAGGTGACTGTGTCCTCC | |

TABLE 12
anti-IL2Rg VHH DNA sequences

| | | SEQ ID |
|---|---|---|
| Name | Sequence | NO: |
| hIL2Rg_ | CAGGTCCAGCTCCAGGAGAGCGGGGGCGGTTCTGTGCAAGCC | 371 |
| VHH-1 | GGAGGCTCATTGAGACTCTCATGCGCTGCAAGTGGTTTTACCT | |
| | TCGATGACAGCGATATGGGATGGTATCGTCAGGCTCCGGGCA | |
| | ATGAGTGTGATCTGGTCTCCACTATCTCCTCTGATGGTTCCAC | |
| | ATACTATGCTGACTCTGTCAAGGGGCGCTTTACCATCTCCCAA | |
| | GATAATGCCAAGAACACCGTGTACCTTCAGATGGATTCAGTT | |
| | AAGCCCGAGGACACAGCCGTCTATTACTGCGCTGCGGATTTT | |
| | ATGATTGCCATCCAAGCTCCCGGAGCGGGATGCTGGGGCCAG | |
| | GGAACCCAGGTCACTGTGAGCAGT | |
| hIL2Rg_ | CAGGTGCAGTTGCAGGAGTCCGGCGGGGGTTCTGTGCCAGCG | 372 |
| VHH-2 | GGTGGGAGCCTCAAGCTCTCCTGTGCCGCTTCCGGCTTCTCAT | |
| | TCTCCTCTTACCCTATGACCTGGGCACGCCAAGCGCCCGGCA | |
| | AGGGACTGGAATGGGTGTCCACCATTGCTTCCGATGGCGGTA | |
| | GTACAGCCTACGCCGCGTCAGTGGAGGGTCGGTTCACGATCA | |
| | GCCGGGACAACGCGAAGAGCACACTCTACCTCCAGCTGAACT | |
| | CTCTGAAGACCGAGGACACCGCCATGTACTATTGCACAAAGG | |
| | GCTACGGCGACGGCACCCCGGCACCCGGCCAGGGCACCCAG | |
| | GTGACAGTCTCTTCC | |
| hIL2Rg_ | CAGGTGCAGTTGCAGGAAAGTGGTGGAGGGAGTGTGCAGAC | 373 |
| VHH-3 | TGGGGGCTCTCTCCGCCTCAGCTGCACAGCCTCTGGATTTACC | |
| | TTCGATGATCGCGAGATGAACTGGTATCGCCAGGCTCCGGGA | |
| | AACGAGTGCGAACTGGTGTCTACAATCAGTTCTGACGGGTCC | |
| | ACCTATTACGCTGATAGTGTCAAGGGCCGCTTCACTATCTCTC | |
| | AGGACAACGCGAAGAACACCGTTTACTTGCAGATGGATAGCG | |
| | TGAAGCCTGAAGATACAGCGGTGTATTACTGCGCTGCCGACT | |
| | TTATGATTGCCATCCAGGCACCGGGGGCGGGGTGTTGGGGAC | |
| | AGGGAACTCAGGTGACTGTGTCCTCC | |
| hIL2Rg_ | CAGGTTCAACTCCAAGAGAGTGGTGGCGGAAGCGTGCAGGC | 374 |
| VHH-4 | GGGCGGTTCTCTGCGTCTGAGTTGCACTGCCAGCGGATTTACC | |
| | TTCGACGATTCCGACATGGGATGGTACAGACAGGCCCCTGGT | |
| | AACGAGTGCGAACTCGTGAGTACTATCAGCTCCGACGGCAAC | |
| | ACCTATTACACCGATTCTGTGAAGGGCAGGTTCACCATCTCCC | |
| | AGGACAACGCTAAGAACACTGTGTACCTGCAAATGAATAGCC | |
| | TGGGACCCGAGGACACAGCGGTCTATTACTGCGCGGCAGAGC | |
| | CGCGCGGCTATTACAGCAACTACGGCGGTAGACGCGAGTGCA | |
| | ACTACTGGGGGCAGGGGACGCAAGTGACTGTCTCCTCC | |
| hIL2Rg_ | CAAGTGCAGCTTCAGGAGTCCGGGGGTGGCAGCGTCCAGGCT | 375 |
| VHH-5 | GGGGGCAGCTTGCGCCTGTCTTGCGCTGCGTCTGGGTTCAGCT | |
| | TTAGCTCCTACCCTATGACCTGGGCTAGACAGGCCCCCGGCA | |
| | AGGGGCTGGAGTGGGTGAGTACAATCGCCTCCGACGGAGGT | |
| | AGTACGGCCTACGCAGCGTCCGTCGAGGGTCGCTTCACCATC | |
| | AGCCGGGATAACGCTAAGTCCACCCTGTACCTTCAGCTCAAT | |
| | TCTCTCAAAACGGAGGATACCGCCATGTACTATTGCACCAAG | |
| | GGATATGGCGACGGCACCCCAGCTCCTGGACAGGGCACACA | |
| | GGTCACCGTTAGCTCC | |
| hIL2Rg_ | CAGGTCCAGCTTCAGGAGTCTGGCGGGGGCGCAGTACAGGCA | 376 |
| VHH-6 | GGGGGTTCTCTGCGTCTGTCCTGCGCCGCGTCCGGCTTTACTT | |
| | TCAGCAACGCACACATGAGTTGGGTGCGCCAAGCGCCCGGCA | |
| | AGGGCCGGGAATGGATCAGTAGCATCTACAGTGGAGGCAGC | |
| | ACATGGTACGCCGACTCTGTTAAGGGTCGTTTTACGATCTCTC | |
| | GTGACAACTCCAAGAACACTTTGTACCTCCAGCTCAATTCTCT | |
| | CAAGACCGAGGACACCGCGATGTACTATTGTGCCGAGAACAG | |
| | GCTGCACTACTATTCCGACGATGACTCTCTCAGGGGCCAGGG | |
| | AACTCAAGTTACCGTGTCCAGC | |
| hIL2Rg_ | CAAGTGCAGCTCCAAGAGAGTGGTGGCGGGCTGGTTCAGCCA | 377 |
| VHH-7 | GGGGGCAGCTTGAGACTCTCCTGCGCAGCTTCAGGCTTTACC | |
| | TTCGATGACCGTGAGATGAACTGGTATCGTCAGGCCCCAGGC | |
| | AACGAGTGTGAGCTGGTTAGCACGATTTCTTCCGACGGTTCC | |
| | ACCTATTACGCCGACTCTGTGAAGGGACGTTTCACTATCTCCC | |
| | AGGACAATGCCAAGAACACCGTGTACCTCCAGATGGACAGC | |
| | GTGAAGCCGGAGGATACTGCTGTGTATTACTGCGCTGCCGAC | |
| | TTTATGATCGCCATCCAGGCCCCTGGCGCGGGTTGCTGGGGC | |
| | CAGGGCACTCAGGTGACCGTGTCTTCC | |
| hIL2Rg_ | CAAGTGCAACTGCAAGAGTCCGGCGGTGGATCTGTGCAGGCC | 378 |
| VHH-8 | GGAGGCAGCCTGCGGCTGAGCTGTGTAGCTTCCGGGTATACC | |
| | TTTAGCTCATACTGTATGGGCTGGTTTCGTCAGGCCCCCGGTA | |
| | AGGAGCGCGAGGGCGTGGCCGCTCTTGGTGGAGGCTCCACCT | |
| | ATTACGCCGATTCCGTGAAGGGCAGGTTTACTATCTCCCAGG | |
| | ACAACGCGAAGAATACGCTCTATCTCCAGATGAATAGCCTGA | |
| | AGCCCGAGGATACAGCTATGTATTACTGTGCTGCCGCTTGGG | |
| | TAGCCTGCCTGGAGTTCGGTGGCTCCTGGTACGATCTGGCAC | |
| | GGTACAAACATTGGGGGCAGGGCACCCAGGTCACCGTGTCTA | |
| | GC | |
| hIL2Rg_ | CAGGTCCAGTTGCAGGAATCTGGGGGCGGTTCCGTACAAGCA | 379 |
| VHH-9 | GGTGGCTCCCTTCGGTTGAGCTGTACCGCATCCGGCTTTACTT | |
| | TCGACGATAGCGATATGGGCTGGTATCGTCAGGCCCCAGGGG | |
| | GCGAGTGCGAGCTGGTTACAATCTCCTCTGACGGCAGTACCT | |
| | ATTACGCAGACTCCGTCAAGGGCAGGTTCACTATCAGTCAGG | |
| | ACAATGCAAAGAACACTGTGTATCTCCAGATGAACTCTCTGA | |
| | AGCCAGAAGATACTGCCGTGTATTACTGCGCTGCGGAACCGA | |
| | GAGGCTATTACTCTAATTATGGCGGGCGTCGGGAGTGTAATT | |
| | ATTGGGGACAGGGAACCCAGGTGACCGTGTCCTCC | |
| hIL2Rg_ | AGCCAGGACTCTGCCAAAAAGACTGTGTATCTCCAGATGAAC | 380 |
| VHH-10 | CAGGTGCAGCTCCAGGAGAGTGGCGGAGGCTCCGTGCAGGCT | |
| | GGGGGCTCTCTGCGTCTGAGCTGTGCCGCAAGCGGTAGCATT | |
| | TACAGCTCTGCCTACATCGGGTGGTTTCGTCAAGCGCCGGGC | |
| | AAAAAGCGCGAAGGCGTGGCCGGAATCTACACGCGCGATGG | |
| | CTCCACCGCTTATGCTGACAGCGTTAAGGGACGTTTTACGATC | |
| | TCCCTGAAACCTGAGGACACAGCCATGTATTACTGCGCCGCT | |
| | GGCCGCCGTACAAAGAGCTATGTTTACATCTTTCGCCCCGAA | |
| | GAGTACAACTACTGGGGCCAGGGAACCCAAGTGACTGTGTCC | |
| | AGT | |
| hIL2Rg_ | CAGGTTCAGTTGCAGGAGTCCGGCGGAGGCAGCGTGCAGGCC | 381 |
| VHH-11 | GGAGGCTCCTTGCGCTTGTCCTGTGCGGCTTCTGGCTTCACCT | |
| | TCTCATCTGCTCACATGAGTTGGGTGCGTCAGGCCCCAGGGA | |
| | AAGGTCGCGAGTGGATTGCCTCCATCTACAGCGGTGGGGGCA | |
| | CTTTTTATGCGGACAGCGTGAAGGGCCGCTTTACCATCAGCC | |
| | GTGACAACGCTAAGAACACCCTGTATCTCCAACTCAATTCCC | |
| | TCAAGACCGAGGATACAGCGATGTACTATTGTGCAACCAACC | |
| | GCCTTCACTATTACTCCGACGATGACAGCCTGCGCGGACAGG | |
| | GGACCCAGGTGACGGTGTCCAGC | |
| hIL2Rg_ | CAGGTGCAACTCCAGGAAAGTGGCGGAGGCTCAGTGCAGGC | 382 |
| VHH-12 | AGGTGGCTCTCTCCGCCTTTCCTGCGCTGCCAGCGGATTCACC | |
| | TTCTCTAACGCTCACATGAGCTGGGTTCGTCAGGCTCCCGGCA | |
| | AAGGCCGTGAATGGATTAGCTCCATCTATAGTGGCGGAAGTA | |
| | CTTGGTACGCAGATAGCGTCAAGGGCCGCTTCACTATTAGTC | |
| | GGGATAACTCCAAGAACACTCTGTACCTCCAGCTGAACTCAT | |
| | TGAAAACCGAGGACACGGCTATGTACTATTGTGCTGAGAACA | |
| | GGCTGCACTATTACTCCGACGATGACTCTCTGAGGGGTCAGG | |
| | GCACCCAGGTGACCGTCAGCTCC | |
| hIL2Rg_ | CAGGTCCAACTCCAGGAGTCCGGCGGAGGCAGCGTGCAGGCT | 383 |
| VHH-13 | GGAGGCTCTCTCCGCCTGAGCTGCACAGCTTCCAGATTCATCT | |
| | TCGATGACTCCGACATGGGCTGGTATCGCCAGGCTCCAGGGA | |
| | ACGAGTGCGAACTGGTGAGCACCATCTCTTCAGACGGTAGCA | |
| | CCTATTACGCCGACAGTGTGAAGGGGCGCTTCACCATCTCCC | |
| | GCGACAATGCTAAAAATACGGTGTATCTCCAGATGAACTCCC | |
| | TCAAACCGGAGGACACAGCTGTATATTACTGTGCTGCGGAAC | |
| | CACGGGGCTACTATAGCAACTATGGTGGAAGGCGCGAGTGCA | |
| | ACTACTGGGGTCAGGGCACACAGGTGACGGTTTCCTCC | |
| hIL2Rg_ | CAGGTGCAGCTCCAGGAGAGCGGCGGTGGCTCCGTGCAGGCT | 384 |
| VHH-14 | GGTGGCAGCCTGAAGCTGTCCTGCACCGTGAGTGGCTTCACA | |
| | GCCGACGATTCTGATATGGGCTGGTATCGCCAAGGCCCCGGC | |
| | AATGAGTGCGAGCTGGTAACCATTAGCTCAGACGGCTCTACA | |
| | TACTATGCCGATTCTGTTAAGGGCCGCTTTACTATCTCACAGG | |
| | ATAATGCCAAGAACACAGTGTACTTGCAGATGAACTCTCTGA | |
| | AACCGGAAGACACAGCTGTGTATTACTGTGCTGCGGAGCCTA | |
| | GAGGGTATTACAGCAATTACGGGGGCCGGAGAGAGTGTAAC | |
| | TATTGGGGGCAGGGCACCCAAGTGACCGTTTCCTCC | |
| hIL2Rg_ | CAGGTCCAGCTTCAGGAATCTGGGGGCGGTCTCGTGCAGCCC | 385 |
| VHH-15 | GGCGGGTCCCTGCGTCTGTCTTGTGCTGCGAGCGGCTTCACGT | |
| | TCTCAAGTGCCCACATGAGCTGGGTAAGGCAGGCACCGGGCA | |
| | AGGGGCGCGAGTGGATTGCAAGCATCTATTCAGGCGGGGGC | |
| | ACATTCTACGCCGACAGCGTGAAGGGACGTTTTACAATCTCC | |
| | AGAGATAACGCAAAGAACACTCTCTACCTCCAACTCAACTCC | |
| | TTGAAGGCGGAAGATACTGCAATGTATTACTGTGCTACTAAC | |
| | CGTCTTCATTATTACTCTGACGATGACTCCCTGCGGGGGCAGG | |
| | GTACACAGGTGACAGTGAGTTCC | |
| hIL2Rg_ | CAGGTGCAGCTGCAAGAATCTGGTGGAGGGCTGGTCCAGCCT | 386 |
| VHH-16 | GGGGGCTCCCTGCGCCTCTCATGTGTCGCATCTGGCTTCACCT | |
| | TCAGCAACGCCCACATGAGCTGGGTTCGCCAAGCCCCTGGGA | |
| | AGGGCCGGGAGTGGATCTCCAGTATCTATTCCGGCGGAAGCA | |
| | CTTGGTATGCAGACAGCGTCAAAGGACGGTTCACTATTTCTC | |
| | GTGATAATTCTAAGAACACCCTGTACCTTCAGCTGAACAGCC | |
| | TGAAGACCGAGGACACTGCTATGTACTATTGTGCTGAGAATC | |
| | GCCTGCATTACTATAGCGACGATGACAGTCTGCGCGGACAGG | |
| | GGACCCAGGTCACCGTGTCCTCT | |
| hIL2Rg_ | CAGGTTCAGTTGCAGGAATCAGGAGGCGGTCTGGTGCAGCCT | 387 |
| VHH-17 | GGGGGCTCTCTGCGTCTCTCCTGCGCCGCTTCCGGCTTCACAT | |
| | TCTCCAACGCCCACATGAGCTGGGTCCGCCAGGCCCCTGGGA | |
| | AGGGCCGCGAGTGGATCTCCAGTATCTACAGCGGGGGCTCCA | |
| | CTTGGTACGCAGACAGCGTCAAAGGGAGGTTTACCATTAGCC | |
| | GTGACAATTCTAAGAACACATTGTATTTGCAGCTGAACTCTCT | |
| | TAAAACCGAGGACACCGCCATGTACTATTGTGCTGAGAACAG | |
| | GCTCCACTATTACTCAGACGATGACTCACTTCGCGGGCAGGG | |
| | AACCCAGGTCACCGTCTCCTCT | |
| hIL2Rg_ | CAAGTCCAGCTCCAGGAAAGCGGCGGTGGCCTGGTGCAACCT | 388 |
| VHH-18 | GGCGGGTCTCTGCGCTTGTCATGCGCTGCCTCCGGCTTCACCT | |
| | TCTCATCTTACCCTATGACCTGGGCGCGTCAGGCTCCCGGCAA | |
| | GGGATTGGAGTGGGTGTCTACTATTGCCTCCGACGGTGGCAG | |
| | CACGGCCTACGCAGCGTCTGTAGAAGGACGCTTCACAATTAG | |
| | CAGAGACAACGCAAAATCTACTTTGTACCTTCAGCTCAACAG | |
| | CCTGAAGACCGAAGACACAGCTATGTATTACTGCACAAAAGG | |
| | CTACGGGGACGGCACGCCAGCGCCTGGACAGGGGACACAGG | |
| | TGACCGTATCTTCT | |
| hIL2Rg_ | CAGGTGCAGTTGCAGGAATCAGGGGGTGGCTCTGTGCAGGCC | 389 |
| VHH-19 | GGGGGCTCCCTGCGTCTGTCCTGTACTGCGAGCGGCTTCACCT | |
| | TTGATGACCGCGAGATGAACTGGTATCGCCAGGCTCCGGGGA | |
| | ACGAGTGCGAACTCGTGTCTACAATTAGCTCCGATGGTTCAA | |
| | CATACTATGCTGATTCTGTCAAAGGTCGCTTTACCATCTCACA | |
| | GGACAACGCCAAGAACACCGTCTACCTCCAGATGGACTCTGT | |
| | GAAGCCTGAAGATACCGCCGTATACTATTGCGCCGCTGACTT | |
| | TATGATTGCCATTCAGGCTCCGGGTGCTGGATGCTGGGGTCA | |
| | GGGGACTCAGGTGACCGTGTCTTCA | |
| hIL2Rg_ | CAAGTGCAGTTGCAGGAAAGCGGCGGTGGGTCCGTGCAAGC | 390 |
| VHH-20 | CGGAGGTTCTCTCCGCCTGTCTTGCACTGCCTCAGGTTTTACC | |
| | TTCGACGATTCCGATATGGGCTGGTACAGGCAGGCTCCCGGC | |
| | AATGAGTGCGAGCTGGTGTCTACGATCTCAAGTGATGGCTCC | |
| | ACCTACTATGCCGATAGCGTAAAAGGAAGGTTTACTATTAGC | |
| | CAGGATAACGCGAAGAACACGGTGTACCTCCAGATGAACAG | |
| | TCTCAAGCCGGAGGATACTGCCGTGTATTACTGTGCTGCCGA | |
| | GCCGCGTGGCTATTACTCCAACTACGGTGGCAGACGTGAATG | |
| | CAATTACTGGGGACAGGGTACTCAGGTTACCGTGTCCTCT | |
| hIL2Rg_ | CAGGTTCAACTTCAGGAATCCGGGGGCGGTTCCGTGCAAGCC | 391 |
| VHH-21 | GGGGGTAGCCTGCGTCTGTCTTGCGTGGCCAGCGGCTATACC | |
| | TCCTGTATGGGTTGGTTTCGGCAGGCTCCTGGGAAGGAGCGC | |
| | GAAGCCGTGGCGACCATCTACACACGGGGCCGCAGCATCTAT | |
| | TACGCTGACAGTGTGAAGGGCCGCTTCACCATCTCCCAGGAT | |
| | AACGCCAAGAATACCCTGTATCTGCAAATGAACTCCCTGAAG | |
| | CCTGAGGACATCGCCATGTATTCCTGCGCAGCTGGAGGGTAC | |
| | TCATGGTCCGCTGGGTGCGAGTTTAATTATTGGGGCCAAGGA | |
| | ACCCAGGTGACCGTCTCCTCA | |
| hIL2Rg_ | CAAGTGCAGCTCCAGGAGTCTGGCGGGGGCCTGGTTCAGCCT | 392 |
| VHH-22 | GGTGGGTCCCTGCGCCTGTCTTGCACGGCTTCCGGCTTTAGCT | |
| | TCTCCTCATATCCAATGACCTGGGCACGCCAGGCTCCTGGTA | |
| | AGGGCCTGGAGTGGGTCTCCACCATCGCCTCTGATGGTGGGT | |
| | CAACTGCCTATGCTGCCTCCGTCGAGGGTAGATTCACAATCA | |
| | GCAGAGACAACGCCAAATCCACGCTGTACCTGCAACTCAACT | |
| | CCTTGAAGACCGAGGACACAGCTATGTATTACTGTACCAAAG | |
| | GCTACGGCGACGGCACTCCTGCTCCCGGACAGGGGACCCAGG | |
| | TGACTGTGTCTAGC | |
| hIL2Rg_ | CAGGTCCAACTTCAGGAAAGCGGGGGTGGACTGGTACAGCC | 393 |
| VHH-23 | AGGGGGCAGTCTGCGCCTGTCCTGTGCCGCAAGCGGGTTTTC | |
| | TTTCTCCAGTTACCCCATGACCTGGGCTCGCCAAGCACCTGGA | |
| | AAGGGACTGGAGTGGGTGTCTACTATTGCGTCAGATGGTGGG | |
| | AGTACGGCTTACGCCGCGAGCGTGGAGGGTCGTTTTACGATC | |
| | AGTAGGGACAACGCCAAAAGCACTCTGTACCTCCAGCTTAAC | |
| | AGCCTGAAGACCGAGGACACCGCCATGTATTACTGTACCAAG | |
| | GGCTACGGAGACGGCACCCCTGCGCCGGGGCAAGGCACCCA | |
| | GGTGACCGTAAGTTCA | |

TABLE 13
murine anti-IL2Rg VHH DNA sequences

| Name | DNA Sequence | SEQ ID NO: |
|---|---|---|
| mIL2Rg_VHH1 | CAGGTGCAACTCCAGGAGTCCGGCGGGGGCTCC | 394 |
| | GTGCTGGCTGGCGGATCTTTGAGGCTGTCTTGCG | |
| | TGGCTTCTGGCTATGGCTATAATTACATCGGCTG | |
| | GTTCCGTCAGACACCCGGCAAGGAGCGCGAAGG | |
| | GGTGGCGGTCATTTACACAGGGGGTGGGGACAC | |
| | TTATTACGCCGACTCCGTCAAGGGTAGGTTTACC | |
| | GCTAGTCGCGATAATGCCAAAAGTACGCTGTAC | |
| | CTGCAAATGAACAGCTTGGAGCCAGAGGACACC | |
| | GCCATGTATTACGGAGTGGCTCGCTACTGTGTGG | |
| | GCAGTGTGTACGCTTGCCTGCGCGGAGGCCACG | |
| | ACGAGTACGCACACTGGGGCCAGGGAACCCAGG | |
| | TGACAGTGTCTAGC | |
| mIL2Rg_VHH2 | CAGGTGCAGCTCCAGGAGTCTGGGGGTGGCAGC | 395 |
| | GTCCAGCCAGGTGGCTCATTGAGACTGTCTTGTG | |
| | CTGCATCTGGCTCCACCTACGCTAATTACCTGAT | |
| | GGGATGGTTCAGGCAGGCCCCTGGTAAGGAGCG | |
| | TGAGGGCGTGGCCGCTATCTATTCTGGCGGTGG | |
| | GTCCACCTACTATGCTGACTCCGTCAAGGGACG | |
| | CTTCACTATTTCTCAAGACAATGCCAAGAACACT | |
| | TTGTACTTGCAAATGAACTCACTCAAACCTGAG | |
| | GACACCGCGATGTACTATTGCGCAGCGGCATCC | |
| | GCAGTGAAGGGAGACAAAGGGGATATCGTGGT | |
| | AGTTGTGACCGGCACCCAGCGTATGGAGTACGA | |
| | CTACTGGGGACATGGCACCCAGGTGACAGTTAG | |
| | CTCC | |
| mIL2Rg_VHH3 | CAGGTACAGTTGCAGGAGAGTGGTGGGGGTTCC | 396 |
| | GTCCAGGCCGGTGCCTCTCTTCGCCTCAGTTGTA | |
| | GCGTGAGCGGTTTCACGTTCGACGAGTCAGTGA | |
| | TGTCCTGGTTGCGCCAGGGTCCCGGCAATGAGT | |
| | GCGACGCGGTCGCTATTATCAGCTCCGATGACA | |
| | ACACCTATTACGACGATAGCGTGAAAGGCCGCT | |
| | TTACCATCTCCGAGGACAACGCCAAAAACATGG | |
| | TGTATCTGCAAATGAACTCACTGAAGCCGGAAG | |
| | ACACCGCAGTGTACTATTGCGCCGCGCGTCGGC | |
| | GCAGACCTGTGTACGATTCCGATTATGAACTCCG | |
| | GCCACGTCCGCTGTGTGGCGATTTCGGCGTGTGG | |
| | GGCCAGGGGACCCAGGTGACGGTCTCCTCC | |
| mIL2Rg_VHH4 | CAGGTGCAGCTCCAGGAATCTGGCGGGGGCTCT | 397 |
| | GTGCAGGCTGGTGGCTCCCTTCGCCTGTCCTGTA | |
| | TTGGCTCCGGTCTTCCTTTCGACGAGGATGACAT | |
| | GGGCTGGTATCGCCAGGCCCCTGGGAATGAGTG | |
| | TGAATTGGTCAGCTCAATCTCCAGTGACGGCAC | |
| | CGCCTATTACGCCGATTCCGTCAAGGGACGCTTC | |
| | ACTATCTCCAGAGACAACGCCAAGAACACTGTG | |
| | CTGTTGCAGATGAACTCCCTGAAGCCCGAGGAT | |
| | ACCGCTGTCTATTACTGCGCAGCCGGGGTCCAC | |
| | AGACAGTTCGGCGGTTCCAGTTCCTGCGGCGAC | |
| | GCCTTCTACGGCATGGATTACTGGGGCAAGGGA | |
| | ACTCAGGTCACAGTGTCTTCC | |
| mIL2Rg_VHH5 | CAGGTTCAGCTTCAGGAGTCCGGCGGGGGCTCC | 398 |
| | GTACAGGCAGGGGGCTCACTGCGTCTTTCCTGTG | |
| | TGGCGAGTGGCGACGTGTATGGCCGTAACAGCA | |
| | TGGCTTGGTTCCGGCAGGCACCTGGAAAGGAAC | |
| | GCGAGGGCGTTGCAGTTGGGTATTCCGTAGTGA | |
| | CAACCACTTACTATGCCGACAGTGTGAAGGGCC | |
| | GGTTTACGATCTCAGAGGACAACGATAAAAACA | |
| | CAGTGTACCTGGAGATGAACTCCCTGAAGCCGG | |
| | AAGACACTGCTATGTATTACTGCGCTGCCGATG | |
| | GCAACCTGTGGCGCGGACTCAGGCCCTCCGAGT | |
| | ACACTTATTGGGGTCAGGGCACCCAGGTGACCG | |
| | TTTCAAGT | |
| mIL2Rg_VHH6 | CAGGTCCAGCTTCAGGAGTCAGGTGGCGGTAGT | 399 |
| | GTCCAGGCAGGCGGTAGCCTGCGCCTTAGCTGT | |
| | GCTACATCCGGCTTCCCTTACTCACGCTATTGTA | |
| | TGGGCTGGTTCAGGCAAGCTCCCGGTAAAGAGC | |
| | GCGAGGGAGTGGCAGCCATCGAGCCTGACGGGA | |
| | GCACATCTTATGCTGACTCTGTAAAGGGGCGTTT | |
| | CACCATCTCTCAGGACAACGCCGTTAATACACT | |
| | GTACTTGCAAATGAATAACCTGAAGCCCGAGGA | |
| | CACAGCTATGTATTACTGCGCAGCCGACGAGCG | |
| | TTGCTTCTATTTGAAGGACTATGACCTCAGAAGG | |
| | CCAGCCCAGTACCGCTACTGGGGGCAGGGCACC | |
| | CAGGTTACCGTGTCATCT | |
| mIL2Rg_VHH7 | CAGGTGCAGTTGCAGGAGAGTGGCGGTGGCCTC | 400 |
| | GTGCAGCCTGGCGGAAGCCTCCGTCTGAGCTGC | |
| | ACTGTGTCCGGCTTCACTTTCGACGAGAGCGAC | |
| | ATGGGCTGGCTGAGGCAGAACCCTGGTAACGAG | |
| | TGCGGCGTTGTGAGTGTCATCACGTCTGATGACA | |
| | ACCCATACTATGATGACAGCGTCAAGGGCCGCT | |
| | TTACTATCTCCGAGGATAACGCCAAGAACATGG | |
| | TGTACCTCCAGATGAACTCACTGAAGCCCGAGG | |
| | ATACCGGCGTTTATTACTGTGCAACCAGGAGCC | |
| | GTCAGCCTGTGTACTCACGCGATTACGAGCTGC | |
| | GGCCCCGCCCCCTCTGTGGAGACTTTGGTGTGTG | |
| | GGGCCAGGGCACCCAGGTGACTGTTTCCAGC | |
| mIL2Rg_VHH8 | CAGGTGCAGTTGCAGGAGAGTGGAGGGGGCTCA | 401 |
| | GTGCAGGCTGGCGGGTCCTTGCGTCTGTCTTGCA | |
| | CCGCCTCTGGCTTCACCTTCGATGACTTCGATAT | |
| | GGGTTGGTATCGCCAGGCTCCAGGGAACGAGTG | |
| | CGAATTGGTCAGCACTATCAGCGACGATGGCTC | |
| | AACATATTACGCCGACTCTGTGAAGGGACGGTC | |
| | TAGCATTAGCCGGGACAACGCAAAGAACACCGT | |
| | CTATCTCCAGATGAACCGCTTGAAGCCTGAGGA | |
| | TACCGGAGTCTATTACTGCGCCGCTGAGGGCGC | |
| | GTTGGGCTCCAAGACTAATTGTGGCTGGGTGGG | |
| | CAACTTCGGATATTGGGGCCAGGGAACACAGGT | |
| | TACCGTTTCCAGC | |
| mIL2Rg_VHH9 | CAGGTGCAGTTGCAGGAGTCTGGAGGCGGTTCC | 402 |
| | GTTCAGGCCGGGGGCTCTCTGCGCCTGTCCTGCG | |
| | CTGCCTCCGGGTTTACATTTGACGATTTCGATAT | |
| | GGGCTGGTATCGCCAGGCCCCTGGCAACGAGTG | |
| | CGAACTGGTGTCTACTATCTCCGATGACGGCTCA | |
| | ACCTACTATGCAGACTCCGTAAAGGGCAGATCC | |
| | AGCATCTCCCGCGACAATGCCAAAAACACTGTG | |
| | TACCTCCAGATGAACTCCCTCAAGCCTGAGGAT | |
| | ACGGCGGTGTACTATTGTGCTGCCGAGGGTGCG | |
| | CTCGGTAGCAAGACTAATTGCGGCTGGGTGGGC | |
| | AACTTCGGGTACTGGGGTCAGGGGACCCAGGTA | |
| | ACCGTGTCTTCT | |
| mIL2Rg_VHH10 | CAGGTGCAGTTGCAGGAAAGCGGTGGGGGCCTG | 403 |
| | GTGCAGCCCGGAGGCAGCCTGCGCTTGAGCTGC | |
| | GCTGCCTCTGGCTTCACATTCGATGACTTCGATA | |
| | TGGGCTGGTATCGTCAAGCACCCGGAAACGAGT | |
| | GCGAGCTGGTGAGTACAATCAGTGATGACGGAT | |
| | CTACCTACTATGCCGACAGCGTCAAGGGAAGAT | |
| | CCAGCATCAGTCGCGACAACGCCAAGAGCACCG | |
| | TTTACCTCCAGATGAACCGCCTCAAGCCTGAGG | |
| | ACACAGGAGTCTATTACTGTGCTGCGGAGGGGG | |
| | CCTTGGGCAGCAAGACTAACTGTGGATGGGTGG | |
| | GAAACTTCGGGTATTGGGGTCAGGGTACACAGG | |
| | TCACAGTGTCTTCA | |
| mIL2Rg_VHH11 | CAAGTTCAGCTTCAGGAAAGTGGGGGCGGGCTG | 404 |
| | GTGCAGCCAGGGGGTTCCCTGAAGCTGAGCTGC | |
| | GCTGCCTCTGGGTTTACATTCTCTGATCGCGACA | |
| | TGGGCTGGTATCGCCAAGCGCCGGGCAATGAAT | |
| | GCGAAAGAGTGAGTACTATTTCTGACGATGGTT | |
| | CTACTTACTATGCTGACTCCGTGAAGGGCCGTAG | |
| | CTCCATTTCCAGGGACAACGCGAAGAACACCGT | |
| | ATACCTCCAGATGAACTCTCTGAAGCCCGAGGA | |
| | CACCGCTGTGTATTACTGCGCTGCCGAGGGGGC | |
| | TCTCGGCTCAAAGACCAACTGCGGATGGGTCGG | |
| | TAACTTCGGCTACTGGGGCCAGGGCACCCAAGT | |
| | GACAGTCTCCTCC | |
| mIL2Rg_VHH12 | CAGGTCCAGTTGCAGGAGAGCGGGGGTGGAAGC | 405 |
| | GTCCTCGCCGGAGGGAGCCTCCGTTTGAGCTGC | |
| | GTCGCCTCAGGCTACGGCTACAATTACATCGGA | |
| | TGGTTCAGACAGACGCCTGGTAAAGAGCGGGAA | |
| | GGCGTCGCCGTGATTTATATCGGTGGCGGAGAC | |
| | ACCTATTACGCTGACTCAGTGAAGGGGCGTTTC | |
| | ACCGCAAGCCGGGACAACGCTAAGAGCACCCTG | |
| | TACCTCCAGATGAACTCTCTCGAACCTGAGGAC | |
| | ACTGCAATGTATTACTGCGTGGCTCGTTACTGCG | |
| | TCGGGAGTGTCTACGCCTGCCTGAGGGGCGGGC | |
| | ATGATGAGTATGCCCACTGGGGACAAGGAACAC | |
| | AGGTGACTGTCTCCAGT | |
| mIL2Rg_VHH13 | CAGGTTCAGCTCCAGGAGTCTGGTGGCGGTTCC | 406 |
| | GTGCTGGCCGGGGGCTCTCTGCGCCTGTCTTGTG | |
| | TCGCCTCAGGGTACGGCTATAACTACATTGGCTG | |
| | GTTCAGACAGACCCCTGGGAAAGAGCGGGAGG | |
| | GTGTGGCTGTCATTTACACCGGCGGAGGCGACA | |
| | CCTACTATGCCGATTCAGTTAAGGGCAGGTTTAC | |
| | CGCGAGCCGTGACAACGCGAAGTCTACTCTGTA | |
| | CCTGCAAATGAACAGCCTGGAACCTGAGGATAC | |
| | TGCGATGTACTATTGTGTGGCCCGGTACTGCGTA | |
| | GGCTCAGTGTATGCCTGCCTGCGCGGGGGTCAC | |
| | GACGAGTACGCACACTGGGGACAGGGAACTCAG | |
| | GTCACCGTGTCTAGC | |
| mIL2Rg_VHH14 | CAGGTGCAACTCCAGGAGTCCGGCGGGGGCTCC | 407 |
| | GTCCAAGCTGGTGGCTCACTGAGGCTTAGCTGT | |
| | GCTGCCTCCGGCTTTACTTTCGACGATTTCGACA | |
| | TGGGTTGGTATCGCCAGGCTCCGGGCAATGAGT | |
| | GCGAGCTGGTCTCTACCATTTCCGATGACGGCTC | |
| | TACCTACTATGCCAACAGTGTTAAGGGTAGGTCT | |
| | TCCATCTCCCGCGACAACGCTAAGAATATGGTG | |
| | TACTTGCAGATGAACTCTCTGAAGCCTGAGGAC | |
| | ACTGCTGTCTACTATTGCGCTGCCGAAGGTGCCC | |
| | TGGGCTCAAAGACTAATTGCGGCTGGGTCGGTA | |
| | ACTTTGGCTACTGGGGTCAGGGGACTCAGGTGA | |
| | CCGTCAGCTCC | |
| mIL2Rg_VHH15 | CAGGTCCAGTTGCAGGAAAGCGGCGGGGGCTCT | 408 |
| | GTTCAGGCAGGCGGAAGCCTTCGTCTGTCCTGTA | |
| | CTGCCAGTGGTTTCACCTTTGATGACTTTGACAT | |
| | GGGCTGGTATCGGCAAGCCCCCGGAAACGAGTG | |
| | CGAGCTGGTATCCACCATTTCCGATGACGGGTCC | |
| | ACGTACTATGCTGATAGCGTGAAGGGCAGGTCT | |
| | TCCATCAGCCGGGACAACGCCAAGAACACAGTG | |
| | TATTTGCAGATGAACCGCCTCAAGCCAGAAGAC | |
| | ACCGGGGTATATTACTGTGCAGCGGAAGGTGCC | |
| | CTGGGTAGCAAGATGAACTGCGGATGGGTGGGT | |
| | AATTTTGGATACTGGGGCCAGGGCACGCAGGTT | |
| | ACAGTGTCCAGC | |

TABLE 14
FDA Antineoplastic Disease Antibodies and Indications

| Name | Tradename(s) | Target; format | Indication |
|---|---|---|---|
| [fam]- | Enhertu | HER2; Humanized IgG1 | HER2⁺ breast |
| trastuzumab | | ADC | cancer |
| deruxtecan | | | |
| Enfortumab | Padcev | Nectin-4; | Urothelial |
| vedotin | | Human IgG1 ADC | cancer |
| Polatuzumab | Polivy | CD79b; Humanized IgG1 | Diffuse large |
| vedotin | | ADC | B-cell |
| | | | lymphoma |
| Cemiplimab | Libtayo | PD-1; Human mAb | Cutaneous |
| | | | squamous cell |
| | | | carcinoma |
| Moxetumomab | Lumoxiti | CD22; Murine IgG1 dsFv | Hairy cell |
| pasudotox | | immunotoxin | leukemia |
| Mogamuizumab | Poteligeo | CCR4; Humanized IgG1 | Cutaneous T |
| | | | cell lymphoma |
| Tildrakizumab | Ilumya | IL23p19; | Plaque psoriasis |
| | | Humanized IgG1 | |
| Ibalizumab | Trogarzo | CD4; Humanized IgG4 | HIV infection |
| Durvalumab | IMFINZI | PD-L1; Human IgG1 | Bladder cancer |
| Inotuzumab | BESPONSA | CD22; Humanized IgG4, | Hematological |
| ozogamicin | | ADC | malignancy |
| Avelumab | Bavencio | PD-L1; Human IgG1 | Merkel cell |
| | | | carcinoma |
| Atezolizumab | Tecentriq | PD-L1; Humanized IgG1 | Bladder cancer |
| Olaratumab | Lartruvo | PDGRFα; Human IgG1 | Soft tissue |
| | | | sarcoma |
| Ixekizumab | Taltz | IL 17a; Humanized IgG4 | Psoriasis |
| Daratumumab | Darzalex | CD38; Human IgG1 | Multiple |
| | | | myeloma |
| Elotuzumab | Empliciti | SLAMF7; | Multiple |
| | | Humanized IgG1 | myeloma |
| Necitumumab | Portrazza | EGFR; Human IgG1 | Non-small cell |
| | | | lung cancer |
| Dinutuximab | Unituxin | GD2; Chimeric IgG1 | Neuroblastoma |
| Nivolumab | Opdivo | PD1; Human IgG4 | Melanoma, non- |
| | | | small cell lung |
| | | | cancer |
| Blinatumomab | Blincyto | CD19, CD3; Murine | Acute |
| | | bispecific tandem scFv | lymphoblastic |
| | | | leukemia |
| Pembrolizumab | Keytruda | PD1; Humanized IgG4 | Melanoma |
| Ramucirumab | Cyramza | VEGFR2; Human IgG1 | Gastric cancer |
| Siltuximab | Sylvant | IL6; Chimeric IgG1 | Castleman |
| | | | disease |
| Obinutuzumab | Gazyva | CD20; Humanized IgG1; | Chronic |
| | | Glycoengineered | lymphocytic |
| | | | leukemia |
| Ado- | Kadcyla | HER2; Humanized IgG1, | Breast |
| trastuzumab | | ADC | cancer |
| emtansine | | | |
| Pertuzumab | Perjeta | HER2; Humanized IgG1 | Breast Cancer |
| Brentuximab | Adcetris | CD30; Chimeric IgG1, | Hodgkin |
| vedotin | | ADC | lymphoma, |
| | | | systemic |
| | | | anaplastic |
| | | | large cell |
| | | | lymphoma |
| Ipilimumab | Yervoy | CTLA-4; Human IgG1 | Metastatic |
| | | | melanoma |
| Ofatumumab | Arzerra | CD20; Human IgG1 | Chronic |
| | | | lymphocytic |
| | | | leukemia |
| Certolizumab | Cimzia | TNF; Humanized Fab, | Crohn disease |
| pegol | | pegylated | |
| Catumaxomab | Removab | EPCAM/CD3;Rat/mouse | Malignant |
| | | bispecific mAb | ascites |
| Panitumumab | Vectibix | EGFR; Human IgG2 | Colorectal |
| | | | cancer |
| Bevacizumab | Avastin | VEGF; Humanized IgG1 | Colorectal |
| | | | cancer |
| Cetuximab | Erbitux | EGFR; Chimeric IgG1 | Colorectal |
| | | | cancer |
| Tositumomab- | Bexxar | CD20; Murine IgG2a | Non-Hodgkin |
| I131 | | | lymphoma |
| Ibritumomab | Zevalin | CD20; Murine IgG1 | Non-Hodgkin |
| tiuxetan | | | lymphoma |
| Gemtuzumab | Mylotarg | CD33; Humanized IgG4, | Acute myeloid |
| ozogamicin | | ADC | leukemia |
| Trastuzumab | Herceptin | HER2; Humanized IgG1 | Breast cancer |
| Infliximab | Remicade | TNF; Chimeric IgG1 | Crohn disease |
| Rituximab | MabThera, | CD20; Chimeric IgG1 | Non-Hodgkin |
| | Rituxan | | lymphoma |
| Edrecolomab | Panorex | EpCAM; Murine IgG2a | Colorectal |
| | | | cancer |

TABLE 15
FDA Immune Disease Antibodies and Indications

| Name | Target | Indication |
|---|---|---|
| belimumab | BLyS | Systemic lupus erythematosus |
| efalizumab | CD11a | Psoriasis |
| ocrelizumab | CD20 | Multiple sclerosis |
| rituximab | CD20 | Multiple sclerosis |
| basiliximab | CD25 | Transplantation rejection |
| daclizumab | CD25 | Transplantation rejection |
| muromonab | CD3 | Transplantation rejection |
| alemtuzumab | CD52 | Multiple sclerosis |
| omalizumab | IgE | Asthma |
| ustekinumab | IL12/IL23 | Plaque psoriasis |
| brodalumab | IL17a | Psoriasis, psoriatic arthritis, ankylosing |
| | | spondylitis |
| secukinumab | IL17a | Psoriasis, psoriatic arthritis, ankylosing |
| | | spondylitis |
| ixekizumab | IL17a | Psoriasis, psoriatic arthritis, ankylosing |
| | | spondylitis |
| canakinumab | IL1β | Cryopyrin-associated periodic syndrome, |
| | | tumor necrosis factor receptor associated |
| | | periodic syndrome, hyperimmunoglobulin D |
| | | syndrome, mevalonate kinase deficiency, |
| | | familial Mediterranean fever, rheumatoid |
| | | arthritis |
| dupilumab | IL4Rα | Asthma, dermatitis |
| mepolizumab | IL5 | Asthma |
| reslizumab | IL5 | Asthma |
| tocilizumab | IL6R | Rheumatoid arthritis |
| vedolizumab | Integrin- | Ulcerative colitis, Crohn's disease |
| | α4β7 | |
| denosumab | RANKL | Osteoporosis |
| certolizumab | TNFa | Chron's disease, rheumatoid arthritis |
| golimumab | TNFa | Rheumatoid arthritis, psoriatic arthritis, |
| | | ankylosing spondylitis |
| adalimumab | TNFα | Rheumatoid arthritis, juvenile idiopathic |
| | | arthritis, psoriatic arthritis, ankylosing |
| | | spondylitis, Crohn's disease, plaque psoriasis |
| infliximab | TNFα | Crohn's disease, ulcerative colitis, rheumatoid |
| | | arthritis, ankylosing spondylitis, psoriatic |
| | | arthritis, plaque psoriasis |
| ranibizumab | VEGF-A | Neovascular age-related macular degeneration, |
| | | macular edema |
| natalizumab | VLA-4 | Multiple sclerosis, relapsing rultiple sclerosis, |
| | | Crohn's disease |

1. An IL2Rb/IL2Rγ binding molecule that specifically binds to IL2Rb subunit (IL2Rb) and IL2Rγ subunit (IL2Rγ),
wherein the binding molecule causes the multimerization of IL2Rb and IL2Rγ when bound to IL2Rb and IL2Rγ, and
wherein the binding molecule comprises a single-domain antibody (sdAb) that specifically binds to IL2Rb (an IL2Rb sdAb) and a sdAb that specifically binds to IL2Rγ (an anti-IL2Rγ sdAb).

2. The IL2Rb/IL2Rγ binding molecule of claim 1, wherein the IL2Rb sdAb is a VHH antibody (an anti-IL2Rγ sdAb) and/or the anti-IL2Rγ sdAb is a VHH antibody (an anti IL2Rγ VHH antibody).

3. The IL2Rb/IL2Rγ binding molecule of claim 1, wherein the IL2Rb sdAb and the anti-IL2Rγ sdAb are joined by a peptide linker.

4. The IL2Rb/IL2Rγ binding molecule of claim 3, wherein the peptide linker comprises between 1 and 50 amino acids.

5. (canceled)

6. (canceled)

7. (canceled)

8. (canceled)

9. The IL2Rb/IL2Rγ binding molecule of claim 1, wherein the binding molecule comprises an anti-IL2Rb sdAb linked to the N-terminus of a linker and an anti-IL2Rγ sdAb linked to the C-terminus of the linker.

10. The IL2Rb/IL2Rγ binding molecule of claim 1, wherein the binding molecule comprises an anti-IL2Rγ sdAb linked to the N-terminus of a linker and an IL2Rb sdAb linked to the C-terminus of the linker.

11. (canceled)

12. (canceled)

13. (canceled)

14. (canceled)

15. (canceled)

16. (canceled)

17. An isolated nucleic acid encoding the IL2Rb/IL2Rγ binding molecule of claim 1.

18. (canceled)

19. An expression vector comprising the nucleic acid of claim 17.

20. An isolated host cell comprising the vector of claim 19.

21. A pharmaceutical composition comprising the IL2Rb/IL2Rγ binding molecule of claim 1 and a pharmaceutically acceptable carrier.

22. A method of treating an autoimmune or inflammatory disease, disorder, or condition or a viral infection in a subject in need thereof, comprising administering to the subject a therapeutically effective amount of an IL2Rb/IL2Rγ binding molecule of claim 1.

23. The method of claim 22, further comprising administering one or more supplementary agents selected from the group consisting of a corticosteroid, a Janus kinase inhibitor, a calcineurin inhibitor, a mTor inhibitor, an IMDH inhibitor, a biologic, a vaccine, and a therapeutic antibody.

24. The method of claim 23, wherein the therapeutic antibody is an antibody that binds a protein selected from the group consisting of BLyS, CD11a, CD20, CD25, CD3, CD52, IgEIL12/IL23, IL17a, IL1β, IL4Rα, IL5, IL6R, integrin-α4β7, RANKL, TNFα, VEGF-A, and VLA-4.

25. The method of claim 22, wherein the disease, disorder, or condition is selected from viral infections, Helicobacter pylori infection, HTLV, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome, juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome, psoriasis, psoriatic arthritis, dermatitis (eczema), exfoliative dermatitis or atopic dermatitis, Pityriasis rubra pilaris, pityriasis rosacea, parapsoriasis, Pityriasis lichenoides, Lichen planus, Lichen nitidus, ichthyosiform dermatosis, keratodermas, dermatosis, alopecia areata, pyoderma gangrenosum, vitiligo, pemphigoid, urticaria, prokeratosis, rheumatoid arthritis; seborrheic dermatitis, solar dermatitis; seborrheic keratosis, senile keratosis, actinic keratosis, photo-induced keratosis, keratosis follicularis; acne vulgaris; keloids; nevi; warts including verruca, condyloma or condyloma acuminatum, and human papilloma viral (HPV) infections.

26. A method of treating a neoplastic disease, disorder or condition in a subject in need thereof, comprising administering to the subject a therapeutically effective amount of an IL2Rb/IL2Rγ binding molecule of claim 1.

27. The method of claim 26, further comprising administering one or more supplementary agents selected from the group consisting of a chemotherapeutic, an immune checkpoint inhibitor, cell therapy, cytokine therapy, and a therapeutic antibody.

28. The method of claim 27, wherein the therapeutic antibody is an antibody that binds to at least one tumor antigen selected from the group consisting of HER2, nectin-4, CD79, CTLA4, CD22, CCR4, IL23p19, PDL1, IL17a, CD38, SLAMF7, CD20, CD30, CD33, CD52, EpCam, CEA, fpA33, TAG-72, CAIX, PSMA, PSA, folate binding protein, GD2, GD3, IL6, GM2, Le^{y}, VEGF, VEGFR, VEGFR2, PDGFRα, EGFR, ERBB2, ERBB3, MET, IGF1R, EPHA3, TRAIL R1, TRAIL R2, RANKL RAP, tenascin, integrin αVβ3, and integrin α4β1.

29. The method of claim 26, wherein the neoplastic disease, disorder or condition is selected from the group consisting of breast cancers, sarcomas, leukemias, lymphomas, genitourinary cancers, gastrointestinal cancers, lung cancers, myelomas, pancreatic cancers, liver cancers, kidney cancers, endocrine cancers, skin cancers, and brain or central and peripheral nervous (CNS) system tumors, malignant tumors, benign tumors, gliomas, neuroblastomas, astrocytomas, myelodysplastic disorders, cervical carcinoma-in-situ, intestinal polyposes, oral leukoplakias, histiocytoses, hyperprofroliferative scars, hemangiomas, hyperproliferative arterial stenosis, psoriasis, inflammatory arthritis, hyperkeratoses, and papulosquamous eruptions.


SEQUENCE LISTING (473 sequences)

SEQ ID NO: 1 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 2 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 3 - protein, 539 aa, Mus musculus
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSHYIEPYLEFEARRRLLGHSWEDASVLSLKQRQQWLFLEMLIPSTSYEVQVRVKAQRNNTGTWSPWSQPLTFRTRPADPMKEILPMSWLRYLLLVLGCFSGFFSCVYILVKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 4 - protein, 214 aa, Mus musculus
AVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSHYIEPYLEFEARRRLLGHSWEDASVLSLKQRQQWLFLEMLIPSTSYEVQVRVKAQRNNTGTWSPWSQPLTFRTRPADPMKE

SEQ ID NO: 5 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 6 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 7 - protein, 369 aa, Mus musculus
MLKLLLSPRSFLVLQLLLLRAGWSSKVLMSSANEDIKADLILTSTAPEHLSAPTLPLPEVQCFVFNIEYMNCTWNSSSEPQATNLTLHYRYKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEENPSLFALEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 8 - protein, 241 aa, Mus musculus
WSSKVLMSSANEDIKADLILTSTAPEHLSAPTLPLPEVQCFVFNIEYMNCTWNSSSEPQATNLTLHYRYKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEENPSLFALEA

SEQ ID NO: 9 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 169 aa, Mus musculus
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 11 - protein, 4 aa, synthetic construct
  REGION 1..4: note = Description of Artificial Sequence: Synthetic peptide
GGGS

SEQ ID NO: 12 - protein, 5 aa, synthetic construct
  REGION 1..5: note = Description of Artificial Sequence: Synthetic peptide
GGGGS

SEQ ID NO: 13 - protein, 4 aa, synthetic construct
  REGION 1..4: note = Description of Artificial Sequence: Synthetic peptide
GGSG

SEQ ID NO: 14 - protein, 5 aa, synthetic construct
  REGION 1..5: note = Description of Artificial Sequence: Synthetic peptide
GGSGG

SEQ ID NO: 15 - protein, 5 aa, synthetic construct
  REGION 1..5: note = Description of Artificial Sequence: Synthetic peptide
GSGSG

SEQ ID NO: 16 - protein, 5 aa, synthetic construct
  REGION 1..5: note = Description of Artificial Sequence: Synthetic peptide
GSGGG

SEQ ID NO: 17 - protein, 5 aa, synthetic construct
  REGION 1..5: note = Description of Artificial Sequence: Synthetic peptide
GGGSG

SEQ ID NO: 18 - protein, 5 aa, synthetic construct
  REGION 1..5: note = Description of Artificial Sequence: Synthetic peptide
GSSSG

SEQ ID NO: 19 - protein, 8 aa, synthetic construct
  REGION 1..8: note = Description of Artificial Sequence: Synthetic peptide
GGSGGGSG

SEQ ID NO: 20 - protein, 12 aa, synthetic construct
  REGION 1..12: note = Description of Artificial Sequence: Synthetic peptide
GGSGGGSGGGSG

SEQ ID NO: 21 - protein, 16 aa, synthetic construct
  REGION 1..16: note = Description of Artificial Sequence: Synthetic peptide
GGSGGGSGGGSGGGSG

SEQ ID NO: 22 - protein, 20 aa, synthetic construct
  REGION 1..20: note = Description of Artificial Sequence: Synthetic peptide
GGSGGGSGGGSGGGSGGGSG

SEQ ID NO: 23 - protein, 8 aa, synthetic construct
  REGION 1..8: note = Description of Artificial Sequence: Synthetic peptide
ASHHHHHH

SEQ ID NO: 24 - protein, 10 aa, synthetic construct
  REGION 1..10: note = Description of Artificial Sequence: Synthetic peptide
GSHHHHHHHH

SEQ ID NO: 25 - protein, 9 aa, synthetic construct
  REGION 1..9: note = Description of Artificial Sequence: Synthetic peptide
YTYDTSDMS

SEQ ID NO: 26 - protein, 9 aa, synthetic construct
  REGION 1..9: note = Description of Artificial Sequence: Synthetic peptide
FTFSNYWIF

SEQ ID NO: 27 - protein, 9 aa, synthetic construct
  REGION 1..9: note = Description of Artificial Sequence: Synthetic peptide
FRFSNYGMS

SEQ ID NO: 28 - protein, 10 aa, synthetic construct
  REGION 1..10: note = Description of Artificial Sequence: Synthetic peptide
YTTYSFNYMG

SEQ ID NO: 29 - protein, 9 aa, synthetic construct
  REGION 1..9: note = Description of Artificial Sequence: Synthetic peptide
DTKSIRCMG

SEQ ID NO: 30 - protein, 9 aa, synthetic construct
  REGION 1..9: note = Description of Artificial Sequence: Synthetic peptide
YTASRYCMA

SEQ ID NO: 31 - protein, 9 aa, synthetic construct
  REGION 1..9: note = Description of Artificial Sequence: Synthetic peptide
YEYCRIHMT

SEQ ID NO: 32 - protein, 10 aa, synthetic construct
  REGION 1..10: note = Description of Artificial Sequence: Synthetic peptide
YTYSSYYCMG

SEQ ID NO: 33 - protein, 16 aa, synthetic construct
  REGION 1..16: note = Description of Artificial Sequence: Synthetic peptide
DIDSGDWAAYADAVKG

SEQ ID NO: 34 - protein, 17 aa, synthetic construct
  REGION 1..17: note = Description of Artificial Sequence: Synthetic peptide
TSNTGGDTTKYADSVKG

SEQ ID NO: 35 - protein, 17 aa, synthetic construct
  REGION 1..17: note = Description of Artificial Sequence: Synthetic peptide
YINGDGSRTHYADSVKG

SEQ ID NO: 36 - protein, 17 aa, synthetic construct
  REGION 1..17: note = Description of Artificial Sequence: Synthetic peptide
VIYTGGGSTLYADSVKG

SEQ ID NO: 37 - protein, 16 aa, synthetic construct
  REGION 1..16: note = Description of Artificial Sequence: Synthetic peptide
AIDREGFATYADSVYD

SEQ ID NO: 38 - protein, 17 aa, synthetic construct
  REGION 1..17: note = Description of Artificial Sequence: Synthetic peptide
AIHPGGGTTYYADSVKG

SEQ ID NO: 39 - protein, 16 aa, synthetic construct
  REGION 1..16: note = Description of Artificial Sequence: Synthetic peptide
SIGSDGRKTYANSVTG

SEQ ID NO: 40 - protein, 16 aa, synthetic construct
  REGION 1..16: note = Description of Artificial Sequence: Synthetic peptide
AIDSDGSTSYADSVKG

SEQ ID NO: 41 - protein, 11 aa, synthetic construct
  REGION 1..11: note = Description of Artificial Sequence: Synthetic peptide
SYWKWGKLNNF

SEQ ID NO: 42 - protein, 7 aa, synthetic construct
  REGION 1..7: note = Description of Artificial Sequence: Synthetic peptide
GRCARSG

SEQ ID NO: 43 - protein, 13 aa, synthetic construct
  REGION 1..13: note = Description of Artificial Sequence: Synthetic peptide
GLSRDGWSLSAAS

SEQ ID NO: 44 - protein, 15 aa, synthetic construct
  REGION 1..15: note = Description of Artificial Sequence: Synthetic peptide
DDQRFASPLYAYFGY

SEQ ID NO: 45 - protein, 16 aa, synthetic construct
  REGION 1..16: note = Description of Artificial Sequence: Synthetic peptide
QNMCRVVRGAMTGVDY

SEQ ID NO: 46 - protein, 15 aa, synthetic construct
  REGION 1..15: note = Description of Artificial Sequence: Synthetic peptide
GSLWVPFGDRCAANY

SEQ ID NO: 47 - protein, 14 aa, synthetic construct
  REGION 1..14: note = Description of Artificial Sequence: Synthetic peptide
EYLYGLGCPDGSAY

SEQ ID NO: 48 - protein, 16 aa, synthetic construct
  REGION 1..16: note = Description of Artificial Sequence: Synthetic peptide
SYEVVDCYPSGYGQDY

SEQ ID NO: 49 - protein, 9 aa, synthetic construct
  REGION 1..9: note = Description of Artificial Sequence: Synthetic peptide
FTFSLYDMS

SEQ ID NO: 50 - protein, 9 aa, synthetic construct
  REGION 1..9: note = Description of Artificial Sequence: Synthetic peptide
KSFSDYPLG

SEQ ID NO: 51 - protein, 9 aa, synthetic construct
  REGION 1..9: note = Description of Artificial Sequence: Synthetic peptide
RSFSGYAIG

SEQ ID NO: 52 - protein, 9 aa, synthetic construct
  REGION 1..9: note = Description of Artificial Sequence: Synthetic peptide
RSINYYRMG

SEQ ID NO: 53 - protein, 10 aa, synthetic construct
  REGION 1..10: note = Description of Artificial Sequence: Synthetic peptide
RFSWGNYAMY

SEQ ID NO: 54 - protein, 8 aa, synthetic construct
  REGION 1..8: note = Description of Artificial Sequence: Synthetic peptide
RTFRRFMG

SEQ ID NO: 55 - protein, 9 aa, synthetic construct
  REGION 1..9: note = Description of Artificial Sequence: Synthetic peptide
RIFNTYSMG

SEQ ID NO: 56 - protein, 9 aa, synthetic construct
  REGION 1..9: note = Description of Artificial Sequence: Synthetic peptide
RTFGDYPIG

SEQ ID NO: 57 - protein, 9 aa, synthetic construct
  REGION 1..9: note = Description of Artificial Sequence: Synthetic peptide
RTFNSYAMG

SEQ ID NO: 58 - protein, 9 aa, synthetic construct
  REGION 1..9: note = Description of Artificial Sequence: Synthetic peptide
LTFRTYYMS

SEQ ID NO: 59 - protein, 9 aa, synthetic construct
  REGION 1..9: note = Description of Artificial Sequence: Synthetic peptide
RIFNTYSMG

SEQ ID NO: 60 - protein, 9 aa, synthetic construct
  REGION 1..9: note = Description of Artificial Sequence: Synthetic peptide
RTLSTYAMG

SEQ ID NO: 61 - protein, 9 aa, synthetic construct
  REGION 1..9: note = Description of Artificial Sequence: Synthetic peptide
RTISTYAMV

SEQ ID NO: 62 - protein, 9 aa, synthetic construct
  REGION 1..9: note = Description of Artificial Sequence: Synthetic peptide
SIFNNNAVY

SEQ ID NO: 63 - protein, 9 aa, synthetic construct
  REGION 1..9: note = Description of Artificial Sequence: Synthetic peptide
RIFNTYSMG

SEQ ID NO: 64 - protein, 9 aa, synthetic construct
  REGION 1..9: note = Description of Artificial Sequence: Synthetic peptide
RTFGYVAMG

SEQ ID NO: 65 - protein, 9 aa, synthetic construct
  REGION 1..9: note = Description of Artificial Sequence: Synthetic peptide
RSFRSYAIG

SEQ ID NO: 66 - protein, 9 aa, synthetic construct
  REGION 1..9: note = Description of Artificial Sequence: Synthetic peptide
RTFSSYAMG

SEQ ID NO: 67 - protein, 9 aa, synthetic construct
  REGION 1..9: note = Description of Artificial Sequence: Synthetic peptide
PTFTSYTMG

SEQ ID NO: 68 - protein, 9 aa, synthetic construct
  REGION 1..9: note = Description of Artificial Sequence: Synthetic peptide
FTFSTDWMY

SEQ ID NO: 69 - protein, 8 aa, synthetic construct
  REGION 1..8: note = Description of Artificial Sequence: Synthetic peptide
RISNYAMG

SEQ ID NO: 70 - protein, 9 aa, synthetic construct
  REGION 1..9: note = Description of Artificial Sequence: Synthetic peptide
RTGTHYAMG

SEQ ID NO: 71 - protein, 9 aa, synthetic construct
  REGION 1..9: note = Description of Artificial Sequence: Synthetic peptide
FTFSNYWMY

SEQ ID NO: 72 - protein, 9 aa, synthetic construct
  REGION 1..9: note = Description of Artificial Sequence: Synthetic peptide
SIFNNNAVY

SEQ ID NO: 73 - protein, 9 aa, synthetic construct
  REGION 1..9: note = Description of Artificial Sequence: Synthetic peptide
RAPASYAMA

SEQ ID NO: 74 - protein, 9 aa, synthetic construct
  REGION 1..9: note = Description of Artificial Sequence: Synthetic peptide
LASSSFFMT

SEQ ID NO: 75-101 - skipped (27 entries)

SEQ ID NO: 102 - protein, 17 aa, synthetic construct
  REGION 1..17: note = Description of Artificial Sequence: Synthetic peptide
GINSGGYSTYYAASAKG

SEQ ID NO: 103 - protein, 17 aa, synthetic construct
  REGION 1..17: note = Description of Artificial Sequence: Synthetic peptide
HISWSGKLTYYRSTVKG

SEQ ID NO: 104 - protein, 17 aa, synthetic construct
  REGION 1..17: note = Description of Artificial Sequence: Synthetic peptide
VVSWRGSSTYYADSVKG

SEQ ID NO: 105 - protein, 16 aa, synthetic construct
  REGION 1..16: note = Description of Artificial Sequence: Synthetic peptide
AIKWGGDGVYADSVKG

SEQ ID NO: 106 - protein, 17 aa, synthetic construct
  REGION 1..17: note = Description of Artificial Sequence: Synthetic peptide
AIGRNSMATYYRDSAKG

SEQ ID NO: 107 - protein, 17 aa, synthetic construct
  REGION 1..17: note = Description of Artificial Sequence: Synthetic peptide
AINWPGGGTYYGDSVKG

SEQ ID NO: 108 - protein, 17 aa, synthetic construct
  REGION 1..17: note = Description of Artificial Sequence: Synthetic peptide
AIRWSGGTTYYTDSVKG

SEQ ID NO: 109 - protein, 17 aa, synthetic construct
  REGION 1..17: note = Description of Artificial Sequence: Synthetic peptide
SISWGGSRQYYTDSVKG

SEQ ID NO: 110 - protein, 17 aa, synthetic construct
  REGION 1..17: note = Description of Artificial Sequence: Synthetic peptide
VITWNSGRTYYADSVKG

SEQ ID NO: 111 - protein, 17 aa, synthetic construct
  REGION 1..17: note = Description of Artificial Sequence: Synthetic peptide
VISWIGSTTLYADSVKG

SEQ ID NO: 112 - protein, 17 aa, synthetic construct
  REGION 1..17: note = Description of Artificial Sequence: Synthetic peptide
AIRWSGGTTYYTDSVKG

SEQ ID NO: 113 - protein, 17 aa, synthetic construct
  REGION 1..17: note = Description of Artificial Sequence: Synthetic peptide
AIRWASGRTYYGDSVKG

SEQ ID NO: 114 - protein, 17 aa, synthetic construct
  REGION 1..17: note = Description of Artificial Sequence: Synthetic peptide
VISRSGDRTYYADSVKG

SEQ ID NO: 115 - protein, 16 aa, synthetic construct
  REGION 1..16: note = Description of Artificial Sequence: Synthetic peptide
LITIGGRTGYADSVKG

SEQ ID NO: 116 - protein, 17 aa, synthetic construct
  REGION 1..17: note = Description of Artificial Sequence: Synthetic peptide
AIRWSGGTTYYTDSVKG

SEQ ID NO: 117 - protein, 17 aa, synthetic construct
  REGION 1..17: note = Description of Artificial Sequence: Synthetic peptide
SINWSGGSTAYADSVKG

SEQ ID NO: 118 - protein, 17 aa, synthetic construct
  REGION 1..17: note = Description of Artificial Sequence: Synthetic peptide
AISYDGRRTYYGRSLKD

SEQ ID NO: 119 - protein, 17 aa, synthetic construct
  REGION 1..17: note = Description of Artificial Sequence: Synthetic peptide
AISRSGGYTSYADSVKG

SEQ ID NO: 120 - protein, 16 aa, synthetic construct
  REGION 1..16: note = Description of Artificial Sequence: Synthetic peptide
VISKGGRTYYADSVKG

SEQ ID NO: 121 - protein, 17 aa, synthetic construct
  REGION 1..17: note = Description of Artificial Sequence: Synthetic peptide
LINTDGTSTSYTKSVKG

SEQ ID NO: 122 - protein, 17 aa, synthetic construct
  REGION 1..17: note = Description of Artificial Sequence: Synthetic peptide
VITRSGGSTYYADSVKG

SEQ ID NO: 123 - protein, 17 aa, synthetic construct
  REGION 1..17: note = Description of Artificial Sequence: Synthetic peptide
LILWNGEFTTYKDSVKG

SEQ ID NO: 124 - protein, 17 aa, synthetic construct
  REGION 1..17: note = Description of Artificial Sequence: Synthetic peptide
HINTNGGNTYYRHSVKG

SEQ ID NO: 125 - protein, 12 aa, synthetic construct
  REGION 1..12: note = Description of Artificial Sequence: Synthetic peptide
RGLTSPYVIPNI

SEQ ID NO: 126 - protein, 21 aa, synthetic construct
  REGION 1..21: note = Description of Artificial Sequence: Synthetic peptide
MKLFNYGGRYCVLKPLTMYQQ

SEQ ID NO: 127 - protein, 13 aa, synthetic construct
  REGION 1..13: note = Description of Artificial Sequence: Synthetic peptide
VPSGRSWYGRNRY

SEQ ID NO: 128 - protein, 14 aa, synthetic construct
  REGION 1..14: note = Description of Artificial Sequence: Synthetic peptide
MPLSSWSRGGYLEV

SEQ ID NO: 129 - protein, 14 aa, synthetic construct
  REGION 1..14: note = Description of Artificial Sequence: Synthetic peptide
KFMVADGWSRQYDY

SEQ ID NO: 130 - protein, 11 aa, synthetic construct
  REGION 1..11: note = Description of Artificial Sequence: Synthetic peptide
TRKYNLYKFAD

SEQ ID NO: 131 - protein, 13 aa, synthetic construct
  REGION 1..13: note = Description of Artificial Sequence: Synthetic peptide
RVRLSNTALLQRY

SEQ ID NO: 132 - protein, 13 aa, synthetic construct
  REGION 1..13: note = Description of Artificial Sequence: Synthetic peptide
RVRLSNTALLQRY

SEQ ID NO: 133 - protein, 8 aa, synthetic construct
  REGION 1..8: note = Description of Artificial Sequence: Synthetic peptide
APWAHNRE

SEQ ID NO: 134 - protein, 12 aa, synthetic construct
  REGION 1..12: note = Description of Artificial Sequence: Synthetic peptide
NFLREGKREPRY

SEQ ID NO: 135 - protein, 7 aa, synthetic construct
  REGION 1..7: note = Description of Artificial Sequence: Synthetic peptide
RVFARRY

SEQ ID NO: 136 - protein, 13 aa, synthetic construct
  REGION 1..13: note = Description of Artificial Sequence: Synthetic peptide
RSRPYLNYGDFGY

SEQ ID NO: 137 - protein, 11 aa, synthetic construct
  REGION 1..11: note = Description of Artificial Sequence: Synthetic peptide
GGYTGIETITA

SEQ ID NO: 138 - protein, 16 aa, synthetic construct
  REGION 1..16: note = Description of Artificial Sequence: Synthetic peptide
GLKFGFNFYSKTAYDY

SEQ ID NO: 139 - protein, 13 aa, synthetic construct
  REGION 1..13: note = Description of Artificial Sequence: Synthetic peptide
VPSGRSWYGRNRY

SEQ ID NO: 140 - protein, 16 aa, synthetic construct
  REGION 1..16: note = Description of Artificial Sequence: Synthetic peptide
STRFYIATMEQGSYDY

SEQ ID NO: 141 - protein, 14 aa, synthetic construct
  REGION 1..14: note = Description of Artificial Sequence: Synthetic peptide
HRSGTMFARYGMDY

SEQ ID NO: 142 - protein, 11 aa, synthetic construct
  REGION 1..11: note = Description of Artificial Sequence: Synthetic peptide
LIAPFYYGMDY

SEQ ID NO: 143 - protein, 13 aa, synthetic construct
  REGION 1..13: note = Description of Artificial Sequence: Synthetic peptide
QRVGATSKYEYDY

SEQ ID NO: 144 - protein, 11 aa, synthetic construct
  REGION 1..11: note = Description of Artificial Sequence: Synthetic peptide
GRTYWFYAMDY

SEQ ID NO: 145 - protein, 15 aa, synthetic construct
  REGION 1..15: note = Description of Artificial Sequence: Synthetic peptide
RRSQKLVTFGAEYPW

SEQ ID NO: 146 - protein, 7 aa, synthetic construct
  REGION 1..7: note = Description of Artificial Sequence: Synthetic peptide
RVFARRY

SEQ ID NO: 147 - protein, 14 aa, synthetic construct
  REGION 1..14: note = Description of Artificial Sequence: Synthetic peptide
ANSDVGLGYYGMDY

SEQ ID NO: 148 - protein, 11 aa, synthetic construct
  REGION 1..11: note = Description of Artificial Sequence: Synthetic peptide
RPGYWSSSYDY

SEQ ID NO: 149 - protein, 7 aa, synthetic construct
  REGION 1..7: note = Description of Artificial Sequence: Synthetic peptide
YLSGTYY

SEQ ID NO: 150 - protein, 13 aa, synthetic construct
  REGION 1..13: note = Description of Artificial Sequence: Synthetic peptide
YPRTLVRNREPIH

SEQ ID NO: 151 - protein, 9 aa, synthetic construct
  REGION 1..9: note = Description of Artificial Sequence: Synthetic peptide
FTFDDSDMG

SEQ ID NO: 152 - protein, 9 aa, synthetic construct
  REGION 1..9: note = Description of Artificial Sequence: Synthetic peptide
FSFSSYPMT

SEQ ID NO: 153 - protein, 9 aa, synthetic construct
  REGION 1..9: note = Description of Artificial Sequence: Synthetic peptide
FTFDDREMN

SEQ ID NO: 154 - protein, 9 aa, synthetic construct
  REGION 1..9: note = Description of Artificial Sequence: Synthetic peptide
FTFDDSDMG

SEQ ID NO: 155 - protein, 9 aa, synthetic construct
  REGION 1..9: note = Description of Artificial Sequence: Synthetic peptide
FSFSSYPMT

SEQ ID NO: 156 - protein, 9 aa, synthetic construct
  REGION 1..9: note = Description of Artificial Sequence: Synthetic peptide
FTFSNAHMS

SEQ ID NO: 157 - protein, 9 aa, synthetic construct
  REGION 1..9: note = Description of Artificial Sequence: Synthetic peptide
FTFDDREMN

SEQ ID NO: 158 - protein, 9 aa, synthetic construct
  REGION 1..9: note = Description of Artificial Sequence: Synthetic peptide
YTFSSYCMG

SEQ ID NO: 159 - protein, 9 aa, synthetic construct
  REGION 1..9: note = Description of Artificial Sequence: Synthetic peptide
FTFDDSDMG

SEQ ID NO: 160 - protein, 9 aa, synthetic construct
  REGION 1..9: note = Description of Artificial Sequence: Synthetic peptide
SIYSSAYIG

SEQ ID NO: 161 - protein, 9 aa, synthetic construct
  REGION 1..9: note = Description of Artificial Sequence: Synthetic peptide
FTFSSAHMS

SEQ ID NO: 162 - protein, 9 aa, synthetic construct
  REGION 1..9: note = Description of Artificial Sequence: Synthetic peptide
FTFSNAHMS

SEQ ID NO: 163 - protein, 9 aa, synthetic construct
  REGION 1..9: note = Description of Artificial Sequence: Synthetic peptide
FIFDDSDMG

SEQ ID NO: 164 - protein, 9 aa, synthetic construct
  REGION 1..9: note = Description of Artificial Sequence: Synthetic peptide
FTADDSDMG

SEQ ID NO: 165 - protein, 9 aa, synthetic construct
  REGION 1..9: note = Description of Artificial Sequence: Synthetic peptide
FTFSSAHMS

SEQ ID NO: 166 - protein, 9 aa, synthetic construct
  REGION 1..9: note = Description of Artificial Sequence: Synthetic peptide
FTFSNAHMS

SEQ ID NO: 167 - protein, 9 aa, synthetic construct
  REGION 1..9: note = Description of Artificial Sequence: Synthetic peptide
FTFSNAHMS

SEQ ID NO: 168 - protein, 9 aa, synthetic construct
  REGION 1..9: note = Description of Artificial Sequence: Synthetic peptide
FTFSSYPMT

SEQ ID NO: 169 - protein, 9 aa, synthetic construct
  REGION 1..9: note = Description of Artificial Sequence: Synthetic peptide
FTFDDREMN

SEQ ID NO: 170 - protein, 9 aa, synthetic construct
  REGION 1..9: note = Description of Artificial Sequence: Synthetic peptide
FTFDDSDMG

SEQ ID NO: 171 - protein, 6 aa, synthetic construct
  REGION 1..6: note = Description of Artificial Sequence: Synthetic peptide
YTSCMG

SEQ ID NO: 172 - protein, 9 aa, synthetic construct
  REGION 1..9: note = Description of Artificial Sequence: Synthetic peptide
FSFSSYPMT

SEQ ID NO: 173 - protein, 9 aa, synthetic construct
  REGION 1..9: note = Description of Artificial Sequence: Synthetic peptide
FSFSSYPMT

SEQ ID NO: 174 - protein, 16 aa, synthetic construct
  REGION 1..16: note = Description of Artificial Sequence: Synthetic peptide
TISSDGSTYYADSVKG

SEQ ID NO: 175 - protein, 17 aa, synthetic construct
  REGION 1..17: note = Description of Artificial Sequence: Synthetic peptide
TIASDGGSTAYAASVEG

SEQ ID NO: 176 - protein, 16 aa, synthetic construct
  REGION 1..16: note = Description of Artificial Sequence: Synthetic peptide
TISSDGSTYYADSVKG

SEQ ID NO: 177 - protein, 16 aa, synthetic construct
  REGION 1..16: note = Description of Artificial Sequence: Synthetic peptide
TISSDGNTYYTDSVKG

SEQ ID NO: 178 - protein, 17 aa, synthetic construct
  REGION 1..17: note = Description of Artificial Sequence: Synthetic peptide
TIASDGGSTAYAASVEG

SEQ ID NO: 179 - protein, 16 aa, synthetic construct
  REGION 1..16: note = Description of Artificial Sequence: Synthetic peptide
SIYSGGSTWYADSVKG

SEQ ID NO: 180 - protein, 16 aa, synthetic construct
  REGION 1..16: note = Description of Artificial Sequence: Synthetic peptide
TISSDGSTYYADSVKG

SEQ ID NO: 181 - protein, 15 aa, synthetic construct
  REGION 1..15: note = Description of Artificial Sequence: Synthetic peptide
ALGGGSTYYADSVKG

SEQ ID NO: 182 - protein, 16 aa, synthetic construct
  REGION 1..16: note = Description of Artificial Sequence: Synthetic peptide
TISSDGSTYYADSVKG

SEQ ID NO: 183 - protein, 17 aa, synthetic construct
  REGION 1..17: note = Description of Artificial Sequence: Synthetic peptide
GIYTRDGSTAYADSVKG

SEQ ID NO: 184 - protein, 16 aa, synthetic construct
  REGION 1..16: note = Description of Artificial Sequence: Synthetic peptide
SIYSGGGTFYADSVKG

SEQ ID NO: 185 - protein, 16 aa, synthetic construct
  REGION 1..16: note = Description of Artificial Sequence: Synthetic peptide
SIYSGGSTWYADSVKG

SEQ ID NO: 186 - protein, 16 aa, synthetic construct
  REGION 1..16: note = Description of Artificial Sequence: Synthetic peptide
TISSDGSTYYADSVKG

SEQ ID NO: 187 - protein, 16 aa, synthetic construct
  REGION 1..16: note = Description of Artificial Sequence: Synthetic peptide
TISSDGSTYYADSVKG

SEQ ID NO: 188 - protein, 16 aa, synthetic construct
  REGION 1..16: note = Description of Artificial Sequence: Synthetic peptide
SIYSGGGTFYADSVKG

SEQ ID NO: 189 - protein, 16 aa, synthetic construct
  REGION 1..16: note = Description of Artificial Sequence: Synthetic peptide
SIYSGGSTWYADSVKG

SEQ ID NO: 190 - protein, 16 aa, synthetic construct
  REGION 1..16: note = Description of Artificial Sequence: Synthetic peptide
SIYSGGSTWYADSVKG

SEQ ID NO: 191 - protein, 17 aa, synthetic construct
  REGION 1..17: note = Description of Artificial Sequence: Synthetic peptide
TIASDGGSTAYAASVEG

SEQ ID NO: 192 - protein, 16 aa, synthetic construct
  REGION 1..16: note = Description of Artificial Sequence: Synthetic peptide
TISSDGSTYYADSVKG

SEQ ID NO: 193 - protein, 16 aa, synthetic construct
  REGION 1..16: note = Description of Artificial Sequence: Synthetic peptide
TISSDGSTYYADSVKG

SEQ ID NO: 194 - protein, 17 aa, synthetic construct
  REGION 1..17: note = Description of Artificial Sequence: Synthetic peptide
TIYTRGRSIYYADSVKG

SEQ ID NO: 195 - protein, 17 aa, synthetic construct
  REGION 1..17: note = Description of Artificial Sequence: Synthetic peptide
TIASDGGSTAYAASVEG

SEQ ID NO: 196 - protein, 17 aa, synthetic construct
  REGION 1..17: note = Description of Artificial Sequence: Synthetic peptide
TIASDGGSTAYAASVEG

SEQ ID NO: 197 - protein, 13 aa, synthetic construct
  REGION 1..13: note = Description of Artificial Sequence: Synthetic peptide
DFMIAIQAPGAGC

SEQ ID NO: 198 - protein, 8 aa, synthetic construct
  REGION 1..8: note = Description of Artificial Sequence: Synthetic peptide
GYGDGTPA

SEQ ID NO: 199 - protein, 13 aa, synthetic construct
  REGION 1..13: note = Description of Artificial Sequence: Synthetic peptide
DFMIAIQAPGAGC

SEQ ID NO: 200 - protein, 17 aa, synthetic construct
  REGION 1..17: note = Description of Artificial Sequence: Synthetic peptide
EPRGYYSNYGGRRECNY

SEQ ID NO: 201 - protein, 8 aa, synthetic construct
  REGION 1..8: note = Description of Artificial Sequence: Synthetic peptide
GYGDGTPA

SEQ ID NO: 202 - protein, 12 aa, synthetic construct
  REGION 1..12: note = Description of Artificial Sequence: Synthetic peptide
NRLHYYSDDDSL

SEQ ID NO: 203 - protein, 13 aa, synthetic construct
  REGION 1..13: note = Description of Artificial Sequence: Synthetic peptide
DFMIAIQAPGAGC

SEQ ID NO: 204 - protein, 20 aa, synthetic construct
  REGION 1..20: note = Description of Artificial Sequence: Synthetic peptide
AWVACLEFGGSWYDLARYKH

SEQ ID NO: 205 - protein, 17 aa, synthetic construct
  REGION 1..17: note = Description of Artificial Sequence: Synthetic peptide
EPRGYYSNYGGRRECNY

SEQ ID NO: 206 - protein, 18 aa, synthetic construct
  REGION 1..18: note = Description of Artificial Sequence: Synthetic peptide
GRRTKSYVYIFRPEEYNY

SEQ ID NO: 207 - protein, 12 aa, synthetic construct
  REGION 1..12: note = Description of Artificial Sequence: Synthetic peptide
NRLHYYSDDDSL

SEQ ID NO: 208 - protein, 12 aa, synthetic construct
  REGION 1..12: note = Description of Artificial Sequence: Synthetic peptide
NRLHYYSDDDSL

SEQ ID NO: 209 - protein, 17 aa, synthetic construct
  REGION 1..17: note = Description of Artificial Sequence: Synthetic peptide
EPRGYYSNYGGRRECNY

SEQ ID NO: 210 - protein, 17 aa, synthetic construct
  REGION 1..17: note = Description of Artificial Sequence: Synthetic peptide
EPRGYYSNYGGRRECNY

SEQ ID NO: 211 - protein, 12 aa, synthetic construct
  REGION 1..12: note = Description of Artificial Sequence: Synthetic peptide
NRLHYYSDDDSL

SEQ ID NO: 212 - protein, 12 aa, synthetic construct
  REGION 1..12: note = Description of Artificial Sequence: Synthetic peptide
NRLHYYSDDDSL

SEQ ID NO: 213 - protein, 12 aa, synthetic construct
  REGION 1..12: note = Description of Artificial Sequence: Synthetic peptide
NRLHYYSDDDSL

SEQ ID NO: 214 - protein, 8 aa, synthetic construct
  REGION 1..8: note = Description of Artificial Sequence: Synthetic peptide
GYGDGTPA

SEQ ID NO: 215 - protein, 13 aa, synthetic construct
  REGION 1..13: note = Description of Artificial Sequence: Synthetic peptide
DFMIAIQAPGAGC

SEQ ID NO: 216 - protein, 17 aa, synthetic construct
  REGION 1..17: note = Description of Artificial Sequence: Synthetic peptide
EPRGYYSNYGGRRECNY

SEQ ID NO: 217 - protein, 13 aa, synthetic construct
  REGION 1..13: note = Description of Artificial Sequence: Synthetic peptide
GGYSWSAGCEFNY

SEQ ID NO: 218 - protein, 8 aa, synthetic construct
  REGION 1..8: note = Description of Artificial Sequence: Synthetic peptide
GYGDGTPA

SEQ ID NO: 219 - protein, 8 aa, synthetic construct
  REGION 1..8: note = Description of Artificial Sequence: Synthetic peptide
GYGDGTPA

SEQ ID NO: 220 - protein, 7 aa, synthetic construct
  REGION 1..7: note = Description of Artificial Sequence: Synthetic peptide
YGYNYIG

SEQ ID NO: 221 - protein, 9 aa, synthetic construct
  REGION 1..9: note = Description of Artificial Sequence: Synthetic peptide
STYANYLMG

SEQ ID NO: 222 - protein, 9 aa, synthetic construct
  REGION 1..9: note = Description of Artificial Sequence: Synthetic peptide
FTFDESVMS

SEQ ID NO: 223 - protein, 9 aa, synthetic construct
  REGION 1..9: note = Description of Artificial Sequence: Synthetic peptide
LPFDEDDMG

SEQ ID NO: 224 - protein, 9 aa, synthetic construct
  REGION 1..9: note = Description of Artificial Sequence: Synthetic peptide
DVYGRNSMA

SEQ ID NO: 225 - protein, 9 aa, synthetic construct
  REGION 1..9: note = Description of Artificial Sequence: Synthetic peptide
FPYSRYCMG

SEQ ID NO: 226 - protein, 9 aa, synthetic construct
  REGION 1..9: note = Description of Artificial Sequence: Synthetic peptide
FTFDESDMG

SEQ ID NO: 227 - protein, 9 aa, synthetic construct
  REGION 1..9: note = Description of Artificial Sequence: Synthetic peptide
FTFDDFDMG

SEQ ID NO: 228 - protein, 9 aa, synthetic construct
  REGION 1..9: note = Description of Artificial Sequence: Synthetic peptide
FTFDDFDMG

SEQ ID NO: 229 - protein, 9 aa, synthetic construct
  REGION 1..9: note = Description of Artificial Sequence: Synthetic peptide
FTFDDFDMG

SEQ ID NO: 230 - protein, 9 aa, synthetic construct
  REGION 1..9: note = Description of Artificial Sequence: Synthetic peptide
FTFSDRDMG

SEQ ID NO: 231 - protein, 7 aa, synthetic construct
  REGION 1..7: note = Description of Artificial Sequence: Synthetic peptide
YGYNYIG

SEQ ID NO: 232 - protein, 7 aa, synthetic construct
  REGION 1..7: note = Description of Artificial Sequence: Synthetic peptide
YGYNYIG

SEQ ID NO: 233 - protein, 9 aa, synthetic construct
  REGION 1..9: note = Description of Artificial Sequence: Synthetic peptide
FTFDDFDMG

SEQ ID NO: 234 - protein, 9 aa, synthetic construct
  REGION 1..9: note = Description of Artificial Sequence: Synthetic peptide
FTFDDFDMG

SEQ ID NO: 235 - protein, 17 aa, synthetic construct
  REGION 1..17: note = Description of Artificial Sequence: Synthetic peptide
VIYTGGGDTYYADSVKG

SEQ ID NO: 236 - protein, 17 aa, synthetic construct
  REGION 1..17: note = Description of Artificial Sequence: Synthetic peptide
AIYSGGGSTYYADSVKG

SEQ ID NO: 237 - protein, 16 aa, synthetic construct
  REGION 1..16: note = Description of Artificial Sequence: Synthetic peptide
IISSDDNTYYDDSVKG

SEQ ID NO: 238 - protein, 16 aa, synthetic construct
  REGION 1..16: note = Description of Artificial Sequence: Synthetic peptide
SISSDGTAYYADSVKG

SEQ ID NO: 239 - protein, 17 aa, synthetic construct
  REGION 1..17: note = Description of Artificial Sequence: Synthetic peptide
VGYSVVTTTYYADSVKG

SEQ ID NO: 240 - protein, 16 aa, synthetic construct
  REGION 1..16: note = Description of Artificial Sequence: Synthetic peptide
AIEPDGSTSYADSVKG

SEQ ID NO: 241 - protein, 16 aa, synthetic construct
  REGION 1..16: note = Description of Artificial Sequence: Synthetic peptide
VITSDDNPYYDDSVKG

SEQ ID NO: 242 - protein, 16 aa, synthetic construct
  REGION 1..16: note = Description of Artificial Sequence: Synthetic peptide
TISDDGSTYYADSVKG

SEQ ID NO: 243 - protein, 16 aa, synthetic construct
  REGION 1..16: note = Description of Artificial Sequence: Synthetic peptide
TISDDGSTYYADSVKG

SEQ ID NO: 244 - protein, 16 aa, synthetic construct
  REGION 1..16: note = Description of Artificial Sequence: Synthetic peptide
TISDDGSTYYADSVKG

SEQ ID NO: 245 - protein, 16 aa, synthetic construct
  REGION 1..16: note = Description of Artificial Sequence: Synthetic peptide
TISDDGSTYYADSVKG

SEQ ID NO: 246 - protein, 17 aa, synthetic construct
  REGION 1..17: note = Description of Artificial Sequence: Synthetic peptide
VIYIGGGDTYYADSVKG

SEQ ID NO: 247 - protein, 17 aa, synthetic construct
  REGION 1..17: note = Description of Artificial Sequence: Synthetic peptide
VIYTGGGDTYYADSVKG

SEQ ID NO: 248 - protein, 16 aa, synthetic construct
  REGION 1..16: note = Description of Artificial Sequence: Synthetic peptide
TISDDGSTYYANSVKG

SEQ ID NO: 249 - protein, 16 aa, synthetic construct
  REGION 1..16: note = Description of Artificial Sequence: Synthetic peptide
TISDDGSTYYADSVKG

SEQ ID NO: 250 - protein, 15 aa, synthetic construct
  REGION 1..15: note = Description of Artificial Sequence: Synthetic peptide
SVYACLRGGHDEYAH

SEQ ID NO: 251 - protein, 25 aa, synthetic construct
  REGION 1..25: note = Description of Artificial Sequence: Synthetic peptide
ASAVKGDKGDIVVVVTGTQRMEYDY

SEQ ID NO: 252 - protein, 24 aa, synthetic construct
  REGION 1..24: note = Description of Artificial Sequence: Synthetic peptide
RRRRPVYDSDYELRPRPLCGDFGV

SEQ ID NO: 253 - protein, 21 aa, synthetic construct
  REGION 1..21: note = Description of Artificial Sequence: Synthetic peptide
GVHRQFGGSSSCGDAFYGMDY

SEQ ID NO: 254 - protein, 15 aa, synthetic construct
  REGION 1..15: note = Description of Artificial Sequence: Synthetic peptide
DGNLWRGLRPSEYTY

SEQ ID NO: 255 - protein, 20 aa, synthetic construct
  REGION 1..20: note = Description of Artificial Sequence: Synthetic peptide
DERCFYLKDYDLRRPAQYRY

SEQ ID NO: 256 - protein, 24 aa, synthetic construct
  REGION 1..24: note = Description of Artificial Sequence: Synthetic peptide
RSRQPVYSRDYELRPRPLCGDFGV

SEQ ID NO: 257 - protein, 18 aa, synthetic construct
  REGION 1..18: note = Description of Artificial Sequence: Synthetic peptide
EGALGSKTNCGWVGNFGY

SEQ ID NO: 258 - protein, 18 aa, synthetic construct
  REGION 1..18: note = Description of Artificial Sequence: Synthetic peptide
EGALGSKTNCGWVGNFGY

SEQ ID NO: 259 - protein, 18 aa, synthetic construct
  REGION 1..18: note = Description of Artificial Sequence: Synthetic peptide
EGALGSKTNCGWVGNFGY

SEQ ID NO: 260 - protein, 18 aa, synthetic construct
  REGION 1..18: note = Description of Artificial Sequence: Synthetic peptide
EGALGSKTNCGWVGNFGY

SEQ ID NO: 261 - protein, 20 aa, synthetic construct
  REGION 1..20: note = Description of Artificial Sequence: Synthetic peptide
RYCVGSVYACLRGGHDEYAH

SEQ ID NO: 262 - protein, 20 aa, synthetic construct
  REGION 1..20: note = Description of Artificial Sequence: Synthetic peptide
RYCVGSVYACLRGGHDEYAH

SEQ ID NO: 263 - protein, 18 aa, synthetic construct
  REGION 1..18: note = Description of Artificial Sequence: Synthetic peptide
EGALGSKTNCGWVGNFGY

SEQ ID NO: 264 - protein, 18 aa, synthetic construct
  REGION 1..18: note = Description of Artificial Sequence: Synthetic peptide
EGALGSKMNCGWVGNFGY

SEQ ID NO: 265 - protein, 119 aa, synthetic construct
  REGION 1..119: note = Description of Artificial Sequence: Synthetic polypeptide
QVQLQESGGGSVQAGGSLRLSCVGSGYTYDTSDMSWYRQAPGKEREFVSDIDSGDWAAYADAVKGRFTISRDNAKKTVYLQMNSLEPEDTAMYYCKASYWKWGKLNNFWGPGTQVTVSS

SEQ ID NO: 266 - protein, 116 aa, synthetic construct
  REGION 1..116: note = Description of Artificial Sequence: Synthetic polypeptide
QVQLQESGGGLVQPGGSLRLSCVASGFTFSNYWIFWVRQAAGKGLEWLSTSNTGGDTTKYADSVKGRFTISRDSAKNTEYLQMNSLKPEDTAVYYCETGRCARSGGYQGTQVTVSS

SEQ ID NO: 267 - protein, 122 aa, synthetic construct
  REGION 1..122: note = Description of Artificial Sequence: Synthetic polypeptide
QVQLQESGGGLVQPGGSLKLSCAASGFRFSNYGMSWVRQAPGEGLEWVSYINGDGSRTHYADSVKGRFTISRDNAKNTLYLQLNSLKTEDTAMYYCEKGLSRDGWSLSAASRGQGTQVTVSS

SEQ ID NO: 268 - protein, 125 aa, synthetic construct
  REGION 1..125: note = Description of Artificial Sequence: Synthetic polypeptide
QVQLQESGGGSVQTGGSLRLSCAVSGYTTYSFNYMGWFRQAPGKEREGVAVIYTGGGSTLYADSVKGRFTISQDNAKNTVYLQMNSLKPEDTAMYYCAADDQRFASPLYAYFGYWGQGTQVTVSS

SEQ ID NO: 269 - protein, 124 aa, synthetic construct
  REGION 1..124: note = Description of Artificial Sequence: Synthetic polypeptide
QVQLQESGGGSVQVGGSLRLSCATSGDTKSIRCMGWFRQTPGKEREGIAAIDREGFATYADSVYDRFTIAQDNAQNTLYLEMNALKPEDTAMYYCAAQNMCRVVRGAMTGVDYWGKGTQVTVSS

SEQ ID NO: 270 - protein, 124 aa, synthetic construct
  REGION 1..124: note = Description of Artificial Sequence: Synthetic polypeptide
QVQLQESGGGSVQAGGSLRLSCAASEYTASRYCMAWFRQAPGKEREGVAAIHPGGGTTYYADSVKGRFSISQDSADNTLYLQMNSLKPEDTAMYYCAAGSLWVPFGDRCAANYWGQGTQVTVSS

SEQ ID NO: 271 - protein, 122 aa, synthetic construct
  REGION 1..122: note = Description of Artificial Sequence: Synthetic polypeptide
QVQLQESGGGSVQAGGSLRLSCAASGYEYCRIHMTWYRQGPGKEREFVSSIGSDGRKTYANSVTGRFTISRDNANHTVYLQMNSLSPEDTAMYYCKTEYLYGLGCPDGSAYWGQGTQVTVSS

SEQ ID NO: 272 - protein, 125 aa, synthetic construct
  REGION 1..125: note = Description of Artificial Sequence: Synthetic polypeptide
QVQLQESGGGSVQVGGSLKLSCAASGYTYSSYYCMGWFRQAPGKEREGVAAIDSDGSTSYADSVKGRFTISQDDAKNTLYLQMNSLKPEDTAMYYCAASYEVVDCYPSGYGQDYWGKGTQVTVSS

SEQ ID NO: 273 - protein, 121 aa, synthetic construct
  REGION 1..121: note = Description of Artificial Sequence: Synthetic polypeptide
QVQLQESGGGLVQPGGSLRLSCAASGFTFSLYDMSWVRQAPGKGLEWVSGINSGGYSTYYAASAKGRFTISRDNAKNTLYLQLSSVKTEDTAMYYCAQRGLTSPYVIPNIRLQGTQVTVSS

SEQ ID NO: 274 - protein, 130 aa, synthetic construct
  REGION 1..130: note = Description of Artificial Sequence: Synthetic polypeptide
EVQLVESGGRLVQAGDSLRLSCVASGKSFSDYPLGWFRQAPGKAREYVAHISWSGKLTYYRSTVKGRFTISRDNAENKLYLQMNALKPEDTAVYYCAAMKLFNYGGRYCVLKPLTMYQQWSQGTQVTVSS

SEQ ID NO: 275 - protein, 122 aa, synthetic construct
  REGION 1..122: note = Description of Artificial Sequence: Synthetic polypeptide
EVQLVESGGGLVQAGGSLRLSCAASGRSFSGYAIGWFRQAPGKEREFVAVVSWRGSSTYYADSVKGRFTISRDNAKGTVYLQMNSLKPEDTAAYYCAAVPSGRSWYGRNRYWGQGTQVTVSS

SEQ ID NO: 276 - protein, 122 aa, synthetic construct
  REGION 1..122: note = Description of Artificial Sequence: Synthetic polypeptide
EVQLVESGGGLVQAGGSLRLSCVISGRSINYYRMGWFRQAPGNRRQFVAAIKWGGDGVYADSVKGRFTISRDNTKNTVYLQMDSLKPEDTGTYYCAKMPLSSWSRGGYLEVWGQGTLVTVSS

SEQ ID NO: 277 - protein, 124 aa, synthetic construct
  REGION 1..124: note = Description of Artificial Sequence: Synthetic polypeptide
EVQLVESGGGLVQAGDSLRLSCAASERFSWGNYAMYWFRQAPGKEREFVAAIGRNSMATYYRDSAKGRFVISRDNAKNTLYLEMNALKPEDTARYYCAAKFMVADGWSRQYDYWGQGTLVTVSS

SEQ ID NO: 278 - protein, 119 aa, synthetic construct
  REGION 1..119: note = Description of Artificial Sequence: Synthetic polypeptide
EVQLVESGGGLVQAGGALRLSCAASGRTFRRFMGWFRQAPGKEREFVAAINWPGGGTYYGDSVKGRFTISRDNAKNTVYLQMNSLKPEDTANYYCAATRKYNLYKFADWGQGTQVTVSS

SEQ ID NO: 279 - protein, 122 aa, synthetic construct
  REGION 1..122: note = Description of Artificial Sequence: Synthetic polypeptide
EVQLVESGGRLVQAGDSLRLSCVASGRIFNTYSMGWFRQVPGKERDFVAAIRWSGGTTYYTDSVKGRFTISRDNAKNTVYLQMNSLKPEDTAVYYCWVRVRLSNTALLQRYWGQGTLVTVSS

SEQ ID NO: 280 - protein, 122 aa, synthetic construct
  REGION 1..122: note = Description of Artificial Sequence: Synthetic polypeptide
EVQLVESGGGLVQAGGSLRLFCASSERTFGDYPIGWFRQAPGKEREFVASISWGGSRQYYTDSVKGRFTITRDNDKNTVYLQMNSLKPEDTAVYYCWVRVRLSNTALLQRYWGQGTLVTVSS

SEQ ID NO: 281 - protein, 117 aa, synthetic construct
  REGION 1..117: note = Description of Artificial Sequence: Synthetic polypeptide
EVQLVESGGGLVQTGGSLRLSCAASGRTFNSYAMGWFRQSPGKEREFVAVITWNSGRTYYADSVKGRFTISRDNAKNTVYLQMNSLKPEDTAVYYCNSAPWAHNREWGQGTLVTVSS

SEQ ID NO: 282 - protein, 121 aa, synthetic construct
  REGION 1..121: note = Description of Artificial Sequence: Synthetic polypeptide
EVQLVESGGGLVQAGGSLRLSCAASGLTFRTYYMSWFRQAPGKEREFVGVISWIGSTTLYADSVKGRFSISRDNAKNTVYLQMNNLKPEDTAVYYCAANFLREGKREPRYWGQGTQVTVSS

SEQ ID NO: 283 - protein, 116 aa, synthetic construct
  REGION 1..116: note = Description of Artificial Sequence: Synthetic polypeptide
EVQLVESGGRLVQAGDSLRLSCVASGRIFNTYSMGWFRQVPGKERDFVAAIRWSGGTTYYTDSVKGRFTISRDNAKNTVYLQMNSLKPEDTAVYYCYLRVFARRYWGQGTQVTVSS

SEQ ID NO: 284 - protein, 122 aa, synthetic construct
  REGION 1..122: note = Description of Artificial Sequence: Synthetic polypeptide
EVQLVESGGGLVQAGGSLRLSCAASGRTLSTYAMGWFRQAPGKEREFVAAIRWASGRTYYGDSVKGRFTISRDSAKNTVYLQMNSLKPEDTAVYYCAARSRPYLNYGDFGYWGQGTQVTVSS

SEQ ID NO: 285 - protein, 120 aa, synthetic construct
  REGION 1..120: note = Description of Artificial Sequence: Synthetic polypeptide
EVQLVESGGGLVQAGGSLRLSCAASGRTISTYAMVWFRQASGKEREFVGVISRSGDRTYYADSVKGRFTISRDNLGNIVRLQLNSLKPEDTAVYYCARGGYTGIETITARGRGTLVTVSS

SEQ ID NO: 286 - protein, 124 aa, synthetic construct
  REGION 1..124: note = Description of Artificial Sequence: Synthetic polypeptide
EVQLVESGGGLVQTGDSLRLSCAAPESIFNNNAVYWYRQFPGKEREYVGLITIGGRTGYADSVKGRFTISRDNANNVAFLQMDSLKPEDTAVYYCATGLKFGFNFYSKTAYDYWGQGTQVTVSS

SEQ ID NO: 287 - protein, 122 aa, synthetic construct
  REGION 1..122: note = Description of Artificial Sequence: Synthetic polypeptide
EVQLVESGGRLVQAGDSLRLSCVASGRIFNTYSMGWFRQVPGKERDFVAAIRWSGGTTYYTDSVKGRFTISRDNAKNTVYLQMKDLKPQDTAVYYCAAVPSGRSWYGRNRYWGQGTLVTVSS

SEQ ID NO: 288 - protein, 125 aa, synthetic construct
  REGION 1..125: note = Description of Artificial Sequence: Synthetic polypeptide
EVQLVESGGGLVQAGGSLRLSCVSSGRTFGYVAMGWFRQAPGKEREFVASINWSGGSTAYADSVKGRFTISRDNAKNTVYLQMNSLKPEDTAVYYCAGSTRFYIATMEQGSYDYWGQGTQVTVSS

SEQ ID NO: 289 - protein, 123 aa, synthetic construct
  REGION 1..123: note = Description of Artificial Sequence: Synthetic polypeptide
EVQLVESGGSVVQPGDSLRLACTASGRSFRSYAIGWFRQASGKERVFVAAISYDGRRTYYGRSLKDRFTISRDNAKNTVYLQMNSLKPEDTAVYYCATHRSGTMFARYGMDYWGKGTLVTVSS

SEQ ID NO: 290 - protein, 120 aa, synthetic construct
  REGION 1..120: note = Description of Artificial Sequence: Synthetic polypeptide
EVQLVESGGGLVQAGGSLRLSCAASGRTFSSYAMGWFRQAPGKEREFVTAISRSGGYTSYADSVKGRFTISRDNAKNTVYLQMNSLKPEDTAVYYCAKLIAPFYYGMDYWTKGTQVTVSS

SEQ ID NO: 291 - protein, 121 aa, synthetic construct
  REGION 1..121: note = Description of Artificial Sequence: Synthetic polypeptide
EVQLVESGGGLMQAGGALRLSCTASGPTFTSYTMGWFRQSPGKRREFVAVISKGGRTYYADSVKGRFTISRDNAKNTFYLQMSSLKPEDTAVYYCAGQRVGATSKYEYDYWGQGTQVTVSS

SEQ ID NO: 292 - protein, 120 aa, synthetic construct
  REGION 1..120: note = Description of Artificial Sequence: Synthetic polypeptide
EVQLVESGGGLVRAGGSLRLSCAASGFTFSTDWMYWVRRAPGKGLEWVSLINTDGTSTSYTKSVKGRFTVSRDNAKNTLYLQMNSLKPEDTALYYCARGRTYWFYAMDYWGKGTQVTVSS

SEQ ID NO: 293 - protein, 123 aa, synthetic construct
  REGION 1..123: note = Description of Artificial Sequence: Synthetic polypeptide
EVQLVESGGGLVQAGDSLRLSCAASGRISNYAMGWFRQAPGKEREFVAVITRSGGSTYYADSVKGRFTISRDNGKNTIDLQMNRLKPEDTAVYYCAVRRSQKLVTFGAEYPWWGQGTLVTVSS

SEQ ID NO: 294 - protein, 116 aa, synthetic construct
  REGION 1..116: note = Description of Artificial Sequence: Synthetic polypeptide
EVQLVESGGGLVQAGGSLRLSCTTSGRTGTHYAMGWFRQAPGKEREFVSLILWNGEFTTYKDSVKGRFTISREKGENTVYLQMNSLKPEDTAVYYCYLRVFARRYWGQGTQVTVSS

SEQ ID NO: 295 - protein, 123 aa, synthetic construct
  REGION 1..123: note = Description of Artificial Sequence: Synthetic polypeptide
EVQLVESGGGLVQPGGSLRLSCEVSGFTFSNYWMYWIRQAPGKGLEWVSHINTNGGNTYYRHSVKGRFTISRDNAKNTLYLQMNGLKSEDTAVYYCAKANSDVGLGYYGMDYWGKGTQVTVSS

SEQ ID NO: 296 - protein, 119 aa, synthetic construct
  REGION 1..119: note = Description of Artificial Sequence: Synthetic polypeptide
EVQLVESGGGSVQPGGSLRLSCAAPESIFNNNAVYWYRQFPGKEREYVGLITIGGRTGYADSVKGRFTISRDNANNVAFLQMDNLKPEDTAVYYCAARPGYWSSSYDYWGQGTQVTVSS

SEQ ID NO: 297 - protein, 116 aa, synthetic construct
  REGION 1..116: note = Description of Artificial Sequence: Synthetic polypeptide
EVQLVESGGGLVQAGGSLRLSCVFSGRAPASYAMAWFRQAVGNEREFVAAINWSGRRTYYADSVKGRFTISKDNAQNTAYLQMTNLEPEDTATYYCNAYLSGTYYWGQGTQVTVSS

SEQ ID NO: 298 - protein, 122 aa, synthetic construct
  REGION 1..122: note = Description of Artificial Sequence: Synthetic polypeptide
EVQLVESGGGLVRAGDSLRLSCAVSGLASSSFFMTWFRQGQGKEREFVATISWTGRTSYYAASVKGRFTVSRDNAKNTVYLQMNSLNSEDTAVYFCAAYPRTLVRNREPIHWGQGTQVTVSS

SEQ ID NO: 299 - protein, 121 aa, synthetic construct
  REGION 1..121: note = Description of Artificial Sequence: Synthetic polypeptide
QVQLQESGGGSVQAGGSLRLSCAASGFTFDDSDMGWYRQAPGNECDLVSTISSDGSTYYADSVKGRFTISQDNAKNTVYLQMDSVKPEDTAVYYCAADFMIAIQAPGAGCWGQGTQVTVSS

SEQ ID NO: 300 - protein, 117 aa, synthetic construct
  REGION 1..117: note = Description of Artificial Sequence: Synthetic polypeptide
QVQLQESGGGSVPAGGSLKLSCAASGFSFSSYPMTWARQAPGKGLEWVSTIASDGGSTAYAASVEGRFTISRDNAKSTLYLQLNSLKTEDTAMYYCTKGYGDGTPAPGQGTQVTVSS

SEQ ID NO: 301 - protein, 121 aa, synthetic construct
  REGION 1..121: note = Description of Artificial Sequence: Synthetic polypeptide
QVQLQESGGGSVQTGGSLRLSCTASGFTFDDREMNWYRQAPGNECELVSTISSDGSTYYADSVKGRFTISQDNAKNTVYLQMDSVKPEDTAVYYCAADFMIAIQAPGAGCWGQGTQVTVSS

SEQ ID NO: 302 - protein, 125 aa, synthetic construct
  REGION 1..125: note = Description of Artificial Sequence: Synthetic polypeptide
QVQLQESGGGSVQAGGSLRLSCTASGFTFDDSDMGWYRQAPGNECELVSTISSDGNTYYTDSVKGRFTISQDNAKNTVYLQMNSLGPEDTAVYYCAAEPRGYYSNYGGRRECNYWGQGTQVTVSS

SEQ ID NO: 303 - protein, 117 aa, synthetic construct
  REGION 1..117: note = Description of Artificial Sequence: Synthetic polypeptide
QVQLQESGGGSVQAGGSLRLSCAASGFSFSSYPMTWARQAPGKGLEWVSTIASDGGSTAYAASVEGRFTISRDNAKSTLYLQLNSLKTEDTAMYYCTKGYGDGTPAPGQGTQVTVSS

SEQ ID NO: 304 - protein, 120 aa, synthetic construct
  REGION 1..120: note = Description of Artificial Sequence: Synthetic polypeptide
QVQLQESGGGAVQAGGSLRLSCAASGFTFSNAHMSWVRQAPGKGREWISSIYSGGSTWYADSVKGRFTISRDNSKNTLYLQLNSLKTEDTAMYYCAENRLHYYSDDDSLRGQGTQVTVSS

SEQ ID NO: 305 - protein, 121 aa, synthetic construct
  REGION 1..121: note = Description of Artificial Sequence: Synthetic polypeptide
QVQLQESGGGLVQPGGSLRLSCAASGFTFDDREMNWYRQAPGNECELVSTISSDGSTYYADSVKGRFTISQDNAKNTVYLQMDSVKPEDTAVYYCAADFMIAIQAPGAGCWGQGTQVTVSS

SEQ ID NO: 306 - protein, 127 aa, synthetic construct
  REGION 1..127: note = Description of Artificial Sequence: Synthetic polypeptide
QVQLQESGGGSVQAGGSLRLSCVASGYTFSSYCMGWFRQAPGKEREGVAALGGGSTYYADSVKGRFTISQDNAKNTLYLQMNSLKPEDTAMYYCAAAWVACLEFGGSWYDLARYKHWGQGTQVTVSS

SEQ ID NO: 307 - protein, 124 aa, synthetic construct
  REGION 1..124: note = Description of Artificial Sequence: Synthetic polypeptide
QVQLQESGGGSVQAGGSLRLSCTASGFTFDDSDMGWYRQAPGGECELVTISSDGSTYYADSVKGRFTISQDNAKNTVYLQMNSLKPEDTAVYYCAAEPRGYYSNYGGRRECNYWGQGTQVTVSS

SEQ ID NO: 308 - protein, 127 aa, synthetic construct
  REGION 1..127: note = Description of Artificial Sequence: Synthetic polypeptide
QVQLQESGGGSVQAGGSLRLSCAASGSIYSSAYIGWFRQAPGKKREGVAGIYTRDGSTAYADSVKGRFTISQDSAKKTVYLQMNSLKPEDTAMYYCAAGRRTKSYVYIFRPEEYNYWGQGTQVTVSS

SEQ ID NO: 309 - protein, 120 aa, synthetic construct
  REGION 1..120: note = Description of Artificial Sequence: Synthetic polypeptide
QVQLQESGGGSVQAGGSLRLSCAASGFTFSSAHMSWVRQAPGKGREWIASIYSGGGTFYADSVKGRFTISRDNAKNTLYLQLNSLKTEDTAMYYCATNRLHYYSDDDSLRGQGTQVTVSS

SEQ ID NO: 310 - protein, 120 aa, synthetic construct
  REGION 1..120: note = Description of Artificial Sequence: Synthetic polypeptide
QVQLQESGGGSVQAGGSLRLSCAASGFTFSNAHMSWVRQAPGKGREWISSIYSGGSTWYADSVKGRFTISRDNSKNTLYLQLNSLKTEDTAMYYCAENRLHYYSDDDSLRGQGTQVTVSS

SEQ ID NO: 311 - protein, 125 aa, synthetic construct
  REGION 1..125: note = Description of Artificial Sequence: Synthetic polypeptide
QVQLQESGGGSVQAGGSLRLSCTASRFIFDDSDMGWYRQAPGNECELVSTISSDGSTYYADSVKGRFTISRDNAKNTVYLQMNSLKPEDTAVYYCAAEPRGYYSNYGGRRECNYWGQGTQVTVSS

SEQ ID NO: 312 - protein, 124 aa, synthetic construct
  REGION 1..124: note = Description of Artificial Sequence: Synthetic polypeptide
QVQLQESGGGSVQAGGSLKLSCTVSGFTADDSDMGWYRQGPGNECELVTISSDGSTYYADSVKGRFTISQDNAKNTVYLQMNSLKPEDTAVYYCAAEPRGYYSNYGGRRECNYWGQGTQVTVSS

SEQ ID NO: 313 - protein, 120 aa, synthetic construct
  REGION 1..120: note = Description of Artificial Sequence: Synthetic polypeptide
QVQLQESGGGLVQPGGSLRLSCAASGFTFSSAHMSWVRQAPGKGREWIASIYSGGGTFYADSVKGRFTISRDNAKNTLYLQLNSLKAEDTAMYYCATNRLHYYSDDDSLRGQGTQVTVSS

SEQ ID NO: 314 - protein, 120 aa, synthetic construct
  REGION 1..120: note = Description of Artificial Sequence: Synthetic polypeptide
QVQLQESGGGLVQPGGSLRLSCVASGFTFSNAHMSWVRQAPGKGREWISSIYSGGSTWYADSVKGRFTISRDNSKNTLYLQLNSLKTEDTAMYYCAENRLHYYSDDDSLRGQGTQVTVSS

SEQ ID NO: 315 - protein, 120 aa, synthetic construct
  REGION 1..120: note = Description of Artificial Sequence: Synthetic polypeptide
QVQLQESGGGLVQPGGSLRLSCAASGFTFSNAHMSWVRQAPGKGREWISSIYSGGSTWYADSVKGRFTISRDNSKNTLYLQLNSLKTEDTAMYYCAENRLHYYSDDDSLRGQGTQVTVSS

SEQ ID NO: 316 - protein, 117 aa, synthetic construct
  REGION 1..117: note = Description of Artificial Sequence: Synthetic polypeptide
QVQLQESGGGLVQPGGSLRLSCAASGFTFSSYPMTWARQAPGKGLEWVSTIASDGGSTAYAASVEGRFTISRDNAKSTLYLQLNSLKTEDTAMYYCTKGYGDGTPAPGQGTQVTVSS

SEQ ID NO: 317 - protein, 121 aa, synthetic construct
  REGION 1..121: note = Description of Artificial Sequence: Synthetic polypeptide
QVQLQESGGGSVQAGGSLRLSCTASGFTFDDREMNWYRQAPGNECELVSTISSDGSTYYADSVKGRFTISQDNAKNTVYLQMDSVKPEDTAVYYCAADFMIAIQAPGAGCWGQGTQVTVSS

SEQ ID NO: 318 - protein, 125 aa, synthetic construct
  REGION 1..125: note = Description of Artificial Sequence: Synthetic polypeptide
QVQLQESGGGSVQAGGSLRLSCTASGFTFDDSDMGWYRQAPGNECELVSTISSDGSTYYADSVKGRFTISQDNAKNTVYLQMNSLKPEDTAVYYCAAEPRGYYSNYGGRRECNYWGQGTQVTVSS

SEQ ID NO: 319 - protein, 119 aa, synthetic construct
  REGION 1..119: note = Description of Artificial Sequence: Synthetic polypeptide
QVQLQESGGGSVQAGGSLRLSCVASGYTSCMGWFRQAPGKEREAVATIYTRGRSIYYADSVKGRFTISQDNAKNTLYLQMNSLKPEDIAMYSCAAGGYSWSAGCEFNYWGQGTQVTVSS

SEQ ID NO: 320 - protein, 117 aa, synthetic construct
  REGION 1..117: note = Description of Artificial Sequence: Synthetic polypeptide
QVQLQESGGGLVQPGGSLRLSCTASGFSFSSYPMTWARQAPGKGLEWVSTIASDGGSTAYAASVEGRFTISRDNAKSTLYLQLNSLKTEDTAMYYCTKGYGDGTPAPGQGTQVTVSS

SEQ ID NO: 321 - protein, 117 aa, synthetic construct
  REGION 1..117: note = Description of Artificial Sequence: Synthetic polypeptide
QVQLQESGGGLVQPGGSLRLSCAASGFSFSSYPMTWARQAPGKGLEWVSTIASDGGSTAYAASVEGRFTISRDNAKSTLYLQLNSLKTEDTAMYYCTKGYGDGTPAPGQGTQVTVSS

SEQ ID NO: 322 - protein, 127 aa, synthetic construct
  REGION 1..127: note = Description of Artificial Sequence: Synthetic polypeptide
QVQLQESGGGSVLAGGSLRLSCVASGYGYNYIGWFRQTPGKEREGVAVIYTGGGDTYYADSVKGRFTASRDNAKSTLYLQMNSLEPEDTAMYYGVARYCVGSVYACLRGGHDEYAHWGQGTQVTVSS

SEQ ID NO: 323 - protein, 134 aa, synthetic construct
  REGION 1..134: note = Description of Artificial Sequence: Synthetic polypeptide
QVQLQESGGGSVQPGGSLRLSCAASGSTYANYLMGWFRQAPGKEREGVAAIYSGGGSTYYADSVKGRFTISQDNAKNTLYLQMNSLKPEDTAMYYCAAASAVKGDKGDIVVVVTGTQRMEYDYWGHGTQVTVSS

SEQ ID NO: 324 - protein, 132 aa, synthetic construct
  REGION 1..132: note = Description of Artificial Sequence: Synthetic polypeptide
QVQLQESGGGSVQAGASLRLSCSVSGFTFDESVMSWLRQGPGNECDAVAIISSDDNTYYDDSVKGRFTISEDNAKNMVYLQMNSLKPEDTAVYYCAARRRRPVYDSDYELRPRPLCGDFGVWGQGTQVTVSS

SEQ ID NO: 325 - protein, 129 aa, synthetic construct
  REGION 1..129: note = Description of Artificial Sequence: Synthetic polypeptide
QVQLQESGGGSVQAGGSLRLSCIGSGLPFDEDDMGWYRQAPGNECELVSSISSDGTAYYADSVKGRFTISRDNAKNTVLLQMNSLKPEDTAVYYCAAGVHRQFGGSSSCGDAFYGMDYWGKGTQVTVSS

SEQ ID NO: 326 - protein, 124 aa, synthetic construct
  REGION 1..124: note = Description of Artificial Sequence: Synthetic polypeptide
QVQLQESGGGSVQAGGSLRLSCVASGDVYGRNSMAWFRQAPGKEREGVAVGYSVVTTTYYADSVKGRFTISEDNDKNTVYLEMNSLKPEDTAMYYCAADGNLWRGLRPSEYTYWGQGTQVTVSS

SEQ ID NO: 327 - protein, 128 aa, synthetic construct
  REGION 1..128: note = Description of Artificial Sequence: Synthetic polypeptide
QVQLQESGGGSVQAGGSLRLSCATSGFPYSRYCMGWFRQAPGKEREGVAAIEPDGSTSYADSVKGRFTISQDNAVNTLYLQMNNLKPEDTAMYYCAADERCFYLKDYDLRRPAQYRYWGQGTQVTVSS

SEQ ID NO: 328 - protein, 132 aa, synthetic construct
  REGION 1..132: note = Description of Artificial Sequence: Synthetic polypeptide
QVQLQESGGGLVQPGGSLRLSCTVSGFTFDESDMGWLRQNPGNECGVVSVITSDDNPYYDDSVKGRFTISEDNAKNMVYLQMNSLKPEDTGVYYCATRSRQPVYSRDYELRPRPLCGDFGVWGQGTQVTVSS

SEQ ID NO: 329 - protein, 126 aa, synthetic construct
  REGION 1..126: note = Description of Artificial Sequence: Synthetic polypeptide
QVQLQESGGGSVQAGGSLRLSCTASGFTFDDFDMGWYRQAPGNECELVSTISDDGSTYYADSVKGRSSISRDNAKNTVYLQMNRLKPEDTGVYYCAAEGALGSKTNCGWVGNFGYWGQGTQVTVSS

SEQ ID NO: 330 - protein, 126 aa, synthetic construct
  REGION 1..126: note = Description of Artificial Sequence: Synthetic polypeptide
QVQLQESGGGSVQAGGSLRLSCAASGFTFDDFDMGWYRQAPGNECELVSTISDDGSTYYADSVKGRSSISRDNAKNTVYLQMNSLKPEDTAVYYCAAEGALGSKTNCGWVGNFGYWGQGTQVTVSS

SEQ ID NO: 331 - protein, 126 aa, synthetic construct
  REGION 1..126: note = Description of Artificial Sequence: Synthetic polypeptide
QVQLQESGGGLVQPGGSLRLSCAASGFTFDDFDMGWYRQAPGNECELVSTISDDGSTYYADSVKGRSSISRDNAKSTVYLQMNRLKPEDTGVYYCAAEGALGSKTNCGWVGNFGYWGQGTQVTVSS

SEQ ID NO: 332 - protein, 126 aa, synthetic construct
  REGION 1..126: note = Description of Artificial Sequence: Synthetic polypeptide
QVQLQESGGGLVQPGGSLKLSCAASGFTFSDRDMGWYRQAPGNECERVSTISDDGSTYYADSVKGRSSISRDNAKNTVYLQMNSLKPEDTAVYYCAAEGALGSKTNCGWVGNFGYWGQGTQVTVSS

SEQ ID NO: 333 - protein, 127 aa, synthetic construct
  REGION 1..127: note = Description of Artificial Sequence: Synthetic polypeptide
QVQLQESGGGSVLAGGSLRLSCVASGYGYNYIGWFRQTPGKEREGVAVIYIGGGDTYYADSVKGRFTASRDNAKSTLYLQMNSLEPEDTAMYYCVARYCVGSVYACLRGGHDEYAHWGQGTQVTVSS

SEQ ID NO: 334 - protein, 127 aa, synthetic construct
  REGION 1..127: note = Description of Artificial Sequence: Synthetic polypeptide
QVQLQESGGGSVLAGGSLRLSCVASGYGYNYIGWFRQTPGKEREGVAVIYTGGGDTYYADSVKGRFTASRDNAKSTLYLQMNSLEPEDTAMYYCVARYCVGSVYACLRGGHDEYAHWGQGTQVTVSS

SEQ ID NO: 335 - protein, 126 aa, synthetic construct
  REGION 1..126: note = Description of Artificial Sequence: Synthetic polypeptide
QVQLQESGGGSVQAGGSLRLSCAASGFTFDDFDMGWYRQAPGNECELVSTISDDGSTYYANSVKGRSSISRDNAKNMVYLQMNSLKPEDTAVYYCAAEGALGSKTNCGWVGNFGYWGQGTQVTVSS

SEQ ID NO: 336 - protein, 126 aa, synthetic construct
  REGION 1..126: note = Description of Artificial Sequence: Synthetic polypeptide
QVQLQESGGGSVQAGGSLRLSCTASGFTFDDFDMGWYRQAPGNECELVSTISDDGSTYYADSVKGRSSISRDNAKNTVYLQMNRLKPEDTGVYYCAAEGALGSKMNCGWVGNFGYWGQGTQVTVSS

SEQ ID NO: 337 - DNA, 375 bp, synthetic construct
  misc_feature 1..375: note = Description of Artificial Sequence: Synthetic polynucleotide
  source 1..375: mol_type = other DNA
caggttcagcttcaggagtccggtggaggctccatccaggccgggggctctctccgcctgtcttgcgccgcttccagatacctctacagtatcgactacatggcttggtttcgtcagagcccaggaaaagagcgggaacccgtggcagtaatctacactgcctcaggtgccacattttaccccgactctgtcaagggcaggttcaccatctctcaggataatgccaagatgacagtgtacttgcagatgaactccctgaaatctgaggataccgctatgtattactgtgccgcagtgcgcaagaccgattcttacctgttcgacgctcagagttttacctactggggccagggcactcaggtcaccgtcagcagc

SEQ ID NO: 338 - DNA, 375 bp, synthetic construct
  misc_feature 1..375: note = Description of Artificial Sequence: Synthetic polynucleotide
  source 1..375: mol_type = other DNA
caggtgcagttgcaggagtccggcgggggttccgtgcaagcaggcggatctctgcgcctgtcctgcgtggcctctcgttatttgtatagcaccaactacatggcttggttccgtcagtccccaggcaaagagcgcgaagccgtagccgtaatctatacggcctctggggcaacactctataccgactcagtgaagggacgcttcacgatttcccaagacaatgcaaagatgaccgtgtacttgcagatgaaccgcctgaagagcgaggacacggctatgtattactgcgcagccgtgcgcaagaccgactcctacttgtttgacgctcagtccttcacttattggggccagggtacacaggtcaccgtgagcagt

SEQ ID NO: 339 - DNA, 375 bp, synthetic construct
  misc_feature 1..375: note = Description of Artificial Sequence: Synthetic polynucleotide
  source 1..375: mol_type = other DNA
caagtacagctccaggagagcggcggtggatctatccaagcagggggtagccttaggttgtcctgtgtggcgtccagatacctgtatagcacgaactacatggcatggttcagacagtccccaggcaaggaacgcgaggcagtcgccgttatttacactgcatctggggccaccctctatacggacagcgtgaagggaaggtttacaatctcccaggacaacgcgaagatgaccgtgtaccttcagatgaaccgcctgaagtccgaggacaccgccatgtattactgtgcagcggtgcgcaagaccgacagctatctgttcgacgcgcagtcattcacttattggggccaaggaacccaagtgaccgtcagctca

SEQ ID NO: 340 - DNA, 375 bp, synthetic construct
  misc_feature 1..375: note = Description of Artificial Sequence: Synthetic polynucleotide
  source 1..375: mol_type = other DNA
caggtgcagctccaagagtccgggggaggctctatccaggcgggaggcagtctgcgcttgtcctgcgccgcaagtcgttatctgtactccattgattacatggcatggttccgccagtccccaggtaaggaacgtgaacctgccgctgtgatctacaccgcttctggagcaaccttttatcctgatagcgttaagggtcgcttcaccatctctcaggataacgccaaaatgacagtgtacctccagatgaacagcctgaagtctgaggacactgccatgtactattgtgcggctgtgcgcaagaccgactcctatctgtttgatgcacagagctttacctattggggtcagggcacccaggtgactgtgtctagc

SEQ ID NO: 341 - DNA, 375 bp, synthetic construct
  misc_feature 1..375: note = Description of Artificial Sequence: Synthetic polynucleotide
  source 1..375: mol_type = other DNA
caggtccagttgcaggagtccggtggaggttccatccaggcgggtgggtcccttcgtctctcctgcgtggcctctaagtacctgtattcaaccaactacatggcatggttcagacagtctcccggcaaagagcgtgaggcagtggccgcgatctatacagcttctggggccaccctgtactctgattccaataagggaaggttcactatctcacaggataacgccaaaatgaccgtctaccttcagatgaacagcctcaagtctgaagacacggcaatgtattactgtgcagccgtgcgcaaaactgggagctacctgtttgacgctcagtctttcacttattggggccagggtacgcaggtgacagtctcttct

SEQ ID NO: 342 - DNA, 387 bp, synthetic construct
  misc_feature 1..387: note = Description of Artificial Sequence: Synthetic polynucleotide
  source 1..387: mol_type = other DNA
caggtgcaactccaggagagcggaggcggttctgttcaggcaggaggttccctgagactgtcctgtgccgcgtctcgctttacgtattcatcctactgcatgggatggttcagacaagcgccggggaaagaaagggaaggcgtggcctccattgactccgacggctcaacttcatacactgatagcgtgaaaggccggttcaccatctctaaggacaacgcgaagaacaccctgtatctccagatgaacagcctcaagcctgaggatactgccatgtactattgcgcactcgacctgatgtctactgtggtcccaggcttctgcgggttcctgctctctgctggcatggactactgggggaagggcactcaggtaacggttagctcc

SEQ ID NO: 343 - DNA, 381 bp, synthetic construct
  misc_feature 1..381: note = Description of Artificial Sequence: Synthetic polynucleotide
  source 1..381: mol_type = other DNA
caggtgcagcttcaggaatctggcgggggctccgtgcaggccgggggctccctcagactttcctgtgccgtctccggttacacatttaacagtaactgtatgggctggttccgccaggcaccaggcaaggagagggaaggtgtggccacaatctatactggtgttgggagtacgtactatgctgattccgtgaaaggtcgcttcacaatttcccaggacaacgcgaagaacactgtgtacttgcagatgaatagcctgaagcctgaagataccgcaatgtattactgcgctgccgagccactctcccgcgtatatggtggaagttgccccacccccactttcggttactggggccagggcactcaagtgaccgtgtcctct

SEQ ID NO: 344 - DNA, 393 bp, synthetic construct
  misc_feature 1..393: note = Description of Artificial Sequence: Synthetic polynucleotide
  source 1..393: mol_type = other DNA
caggttcagcttcaggagtctgggggcggttcagtgcaggctggcggttctctccgcctgtcctgcgctgccagcggctatacttacagcatgtactgcatgggctggttccggcaagcccccggcaaagagcgtgagggcgtcgctcaaatcaacagcgacgggtcaaccagctacgccgattctgtcaagggcagatttactatcagcaaggacaacgccaaaaacacactgtacctccagatgaactctttgaagcctgaggacaccgcgatgtattactgcgccgctgacagccgcgtgtacggtggcagctggtatgagaggctgtgcggcccgtacacctacgagtacaactattggggacagggcacgcaggtgacagttagctcc

SEQ ID NO: 345 - DNA, 363 bp, synthetic construct
  misc_feature 1..363: note = Description of Artificial Sequence: Synthetic polynucleotide
  source 1..363: mol_type = other DNA
caggtgcagttgcaggagagcgggggcggtctggtccagccgggcgggtcactgcgcctgtcttgtgccgcttcaggatttacctttagtttgtacgacatgagttgggttaggcaagcgcctggcaagggtctggagtgggtgtctggcatcaactcaggaggctatagcacctattacgcggcctccgccaagggccgcttcaccatctctagggataacgcaaagaacactctttacctccagctcagctctgttaagactgaggatactgccatgtattactgtgcccagcgcggcctcaccagcccgtatgtgattccgaacattcgcttgcagggcacacaggtgactgtgtccagc

SEQ ID NO: 346 - DNA, 390 bp, synthetic construct
  misc_feature 1..390: note = Description of Artificial Sequence: Synthetic polynucleotide
  source 1..390: mol_type = other DNA
gaggtccaactggtggagagcggcggaaggctggtgcaggctggcgactccctgcgcttgagctgtgtggcaagcggaaagtccttttccgattaccctctcggttggttccgtcaggctcctggaaaagctagggagtatgtggcccacatctcttggagcggcaaactgacttactatcgctcaacagtgaagggccggtttactatcagccgcgataacgctgaaaataaactgtacctccagatgaacgccctgaagcccgaggatactgccgtgtattactgtgctgccatgaagttgttcaactatggcgggcgttactgtgttctcaagcccctgacaatgtaccaacagtggagccagggtactcaggtcacagttagctcc

SEQ ID NO: 347 - DNA, 366 bp, synthetic construct
  misc_feature 1..366: note = Description of Artificial Sequence: Synthetic polynucleotide
  source 1..366: mol_type = other DNA
gaggtccagctcgttgagagcggcgggggcctggtgcaggccggtggcagcctccgtctctcctgtgccgcttctggccgcagtttctccgggtatgctatcgggtggttcagacaggcaccaggcaaggagcgcgagtttgttgctgtcgtgagctggcggggttctagcacctactatgccgactcagtcaagggccgcttcacaattagcagggacaacgccaagggcactgtatacctccagatgaactccctgaagccagaggataccgccgcgtattactgcgctgccgtgccatctggccgctcctggtacggtaggaaccgttactggggtcagggaactcaggtcaccgtgtcctca

SEQ ID NO: 348 - DNA, 366 bp, synthetic construct
  misc_feature 1..366: note = Description of Artificial Sequence: Synthetic polynucleotide
  source 1..366: mol_type = other DNA
gaagtgcagctcgttgaaagcggcgggggcctcgtgcaagctggaggctcacttcgcctttcttgtgtcatcagtggccgctctatcaattattaccggatgggctggttccgccaggcccctggcaaccgcaggcaattcgtggcggctatcaagtggggtggcgacggtgtgtacgccgactccgtgaaggggcgctttaccattagtcgggacaacaccaagaacaccgtatacttgcagatggacagtctgaagcccgaagacaccggaacatattactgcgccaaaatgcctctttctagctggtccagaggtggctaccttgaggtgtggggtcaaggcacgctggtgaccgtgtcttct

SEQ ID NO: 349 - DNA, 372 bp, synthetic construct
  misc_feature 1..372: note = Description of Artificial Sequence: Synthetic polynucleotide
  source 1..372: mol_type = other DNA
gaagtgcaactcgtggaaagtggaggcggtctcgtccaggcgggggacagcctgcgtctgtcttgcgccgcatccgagcgtttttcttggggcaactatgctatgtattggttcaggcaggcccctggcaaggaacgcgagttcgtggctgccattggccgcaacagcatggccacgtattacagagatagcgccaagggccgcttcgtcatcagccgtgacaacgctaagaacaccctgtacctggaaatgaacgccttgaagcctgaagatactgctaggtactattgcgccgcgaagttcatggtggccgacggctggagcagacagtatgactactggggccagggcactctggtaacggtctcctcc

SEQ ID NO: 350 - DNA, 357 bp, synthetic construct
  misc_feature 1..357: note = Description of Artificial Sequence: Synthetic polynucleotide
  source 1..357: mol_type = other DNA
gaagttcagcttgtggaaagcggcggtgggcttgtccaggctggtggagcgctgcgcctctcctgcgcagcgagtggcaggaccttccgccgtttcatgggttggtttcgccaggccccagggaaggagcgcgagtttgttgctgccatcaactggcctggaggtggcacctactatggcgatagcgtgaagggccgtttcacaatctccagggacaacgccaagaataccgtctacctgcaaatgaactccctgaagccggaggacaccgcgaactattactgcgccgcaacccgcaagtacaacctgtataaattcgcggactggggccagggcacccaggtgacagtgtcatct

SEQ ID NO: 351 - DNA, 366 bp, synthetic construct
  misc_feature 1..366: note = Description of Artificial Sequence: Synthetic polynucleotide
  source 1..366: mol_type = other DNA
gaggtccagctcgtcgagtccggcgggcggctggtgcaggctggcgacagccttcgcctgtcctgtgtggcatccggcagaatctttaacacctactcaatgggttggtttaggcaggttcccggaaaggagagggatttcgtggctgccatcagatggtccggtggcaccacatattacactgattctgtcaaggggcgcttcaccattagtcgcgataacgcaaaaaacaccgtgtacctgcaaatgaatagcctgaagcctgaggacaccgccgtatattactgttgggtgcgcgttcgcctgagcaacacagccctgcttcagcgctactggggtcagggaaccttggttaccgtgtcaagc

SEQ ID NO: 352 - DNA, 366 bp, synthetic construct
  misc_feature 1..366: note = Description of Artificial Sequence: Synthetic polynucleotide
  source 1..366: mol_type = other DNA
gaagtccagctcgtggagtccgggggaggtctggttcaagctgggggttctttgcgcctcttttgcgcgtccagcgagcgtactttcggagattacccaatcggatggttccgtcaagccccaggcaaggagcgcgagtttgtcgcgtccatcagctggggtggctcacgtcagtactatactgactccgttaagggccgcttcacgattacaagagataatgataagaacaccgtgtatctccagatgaactccctcaagcccgaggacactgctgtttactattgctgggtgcgggtgcgtctgtcaaacacggcactgcttcagcgctattggggacagggcaccctggtcaccgtctcctca

SEQ ID NO: 353 - DNA, 351 bp, synthetic construct
  misc_feature 1..351: note = Description of Artificial Sequence: Synthetic polynucleotide
  source 1..351: mol_type = other DNA
gaagtccagctggtcgagtcaggcgggggactggtgcagactgggggtagtctgcgcctgagctgcgcagcttcaggaagaaccttcaactcctacgctatgggctggttcagacagagcccaggcaaagagcgggagttcgtggcggtgattacgtggaactctggccgcacgtactatgctgacagtgtcaaaggcagatttaccatcagtagggataacgccaagaacacagtgtatctccagatgaactctctgaagcccgaggatactgctgtgtattactgtaacagcgccccctgggctcacaatcgtgagtgggggcaggggaccctcgttaccgtcagcagc

SEQ ID NO: 354 - DNA, 363 bp, synthetic construct
  misc_feature 1..363: note = Description of Artificial Sequence: Synthetic polynucleotide
  source 1..363: mol_type = other DNA
gaggtgcagctggtggaatctggtggagggctggtgcaggctggcggttccctccgtctgtcttgtgcggcctcagggctgaccttcaggacctactatatgtcatggttccgccaagcgcccggcaaggaacgcgagttcgtcggagtgatctcttggatcggctccactaccctctacgccgattctgtgaaaggtaggttttccatctcacgcgataatgctaagaacaccgtctacctccagatgaataacttgaaacccgaggacaccgccgtctactattgcgcggccaacttcctcagagagggaaagcgcgaacctcggtattggggacaagggacccaggtgaccgtttcctcc

SEQ ID NO: 355 - DNA, 348 bp, synthetic construct
  misc_feature 1..348: note = Description of Artificial Sequence: Synthetic polynucleotide
  source 1..348: mol_type = other DNA
gaggtgcagttggttgagtctggcggaaggctcgttcaagctggtgacagcctgcggctgtcttgcgtcgcttctggacgcatcttcaacacatattcaatgggctggttcagacaggtgcctggcaaggagcgcgacttcgtggcagctatccgttggagcgggggcactacgtattacaccgattctgtgaaggggcgcttcacaatctccagggataatgcaaagaacaccgtgtaccttcagatgaacagcttgaagcctgaagataccgcagtgtactattgttatctgagggtgttcgctcggcgctattggggccagggcacacaggtgacagtgtcctcc

SEQ ID NO: 356 - DNA, 366 bp, synthetic construct
  misc_feature 1..366: note = Description of Artificial Sequence: Synthetic polynucleotide
  source 1..366: mol_type = other DNA
gaagtgcagctggtcgagagcgggggtggacttgtgcaggctggtggctcccttaggctgagctgcgccgcttccggcagaactctctctacctatgctatgggttggttccgtcaggcccccggcaaggagcgcgagttcgtcgcggccatccgctgggcttctggccgtacttattacggtgacagcgtgaagggtcggttcaccatctctcgtgacagtgcgaaaaataccgtgtacctccagatgaactccctgaagccggaggacacggcggtttattactgcgcggccaggagcaggccttacctgaactacggagactttgggtactggggccaggggacccaggtcaccgtgtcatcc

SEQ ID NO: 357 - DNA, 360 bp, synthetic construct
  misc_feature 1..360: note = Description of Artificial Sequence: Synthetic polynucleotide
  source 1..360: mol_type = other DNA
gaagtccagctcgtggagtctgggggtggactcgtacaagccgggggatcacttcgcttgtcctgcgcggcttctggcaggaccatctcaacttacgcaatggtttggttcaggcaagcctctggtaaggagcgtgagtttgtgggcgttatctcccgcagtggagaccgcacttactatgctgattctgtgaagggcagattcactatcagtcgcgataatctgggcaacattgtgcgtttgcagctcaattcacttaaacctgaagacacagccgtttattactgcgcacgcggcggatataccgggattgagacaattacggctcggggtcgcggcacattggtcaccgtgtccagc

SEQ ID NO: 358 - DNA, 372 bp, synthetic construct
  misc_feature 1..372: note = Description of Artificial Sequence: Synthetic polynucleotide
  source 1..372: mol_type = other DNA
gaggttcagctcgttgagagtggtggaggcctcgtgcagaccggggattcccttcgcctttcctgtgcagctccagagtccatcttcaacaataacgccgtttactggtacaggcagttccccggcaaggagagggagtatgttggtctcatcaccatcggtggcaggaccgggtacgcggactctgtgaaaggccgctttaccatctccagagacaacgccaataacgtggcctttttgcagatggattccctcaagcccgaggatactgctgtctactattgtgccacgggcttgaagttcggcttcaacttctacagtaagactgcctacgactactggggacaagggacccaggtgaccgtcagctct

SEQ ID NO: 359 - DNA, 366 bp, synthetic construct
  misc_feature 1..366: note = Description of Artificial Sequence: Synthetic polynucleotide
  source 1..366: mol_type = other DNA
gaggtgcagctcgtggagtctggaggtcgcctggtgcaggctggcgattccctgcgcctgtcctgtgtggcctctggtcgcattttcaacacttattctatgggttggttcagacaggttcctggaaaggaaagagacttcgtggcagccattcggtggagtggtggcaccacttattacacagactccgtgaagggtcgctttactatctctcgggataacgccaaaaacactgtctacctccagatgaaagacctgaagccccaggacaccgccgtctattactgtgctgccgtcccctctggccgcagctggtacggtcgcaaccgttactggggccagggcactctggtgaccgtcagctct

SEQ ID NO: 360 - DNA, 375 bp, synthetic construct
  misc_feature 1..375: note = Description of Artificial Sequence: Synthetic polynucleotide
  source 1..375: mol_type = other DNA
gaggtgcagttggtggagagcggcggtggcctggtccaggcgggcgggtccctccgcctgagttgtgtgtcttcaggccggacctttggatatgtcgctatgggttggttccgtcaagccccaggtaaggaacgcgagttcgtggcgagcattaactggagcggcgggtccacggcctatgcggactccgtaaagggccggttcactatcagccgcgacaacgctaagaataccgtgtacttgcagatgaacagcctgaagcctgaggatacagccgtgtattactgcgctggatcaacccgcttctatatcgcgacgatggaacagggctcctacgattactggggccaaggtactcaggtgaccgtaagcagc

SEQ ID NO: 361 - DNA, 369 bp, synthetic construct
  misc_feature 1..369: note = Description of Artificial Sequence: Synthetic polynucleotide
  source 1..369: mol_type = other DNA
gaggtgcaactggtggaatcaggaggctccgtggtccagccaggggacagccttcgtcttgcctgcaccgcctctggtcgcagtttcaggtcttacgcgattggctggtttaggcaggcatccggcaaggaaagggtgtttgtggctgccatctcttatgacggtaggcgcacctactatgggcgttcattgaaggaccgtttcactatctctcgggacaacgctaagaacacagtgtacttgcagatgaactccctcaagcccgaggacactgccgtgtactattgcgctacccatcgctccggtacaatgttcgctcggtatggtatggattactggggtaagggtactttggttaccgtgtccagc

SEQ ID NO: 362 - DNA, 360 bp, synthetic construct
  misc_feature 1..360: note = Description of Artificial Sequence: Synthetic polynucleotide
  source 1..360: mol_type = other DNA
gaggtgcagctggtggagagcggcggtggcctggtgcaagcaggcggatctctgcgtctgtcttgtgctgcgtcaggccgcaccttctcctcttatgctatggggtggtttagacaagctcctggaaaggagagggagtttgtgactgccatctccagatccggtggatacactagctacgccgatagtgttaagggccggttcactatctctcgcgacaatgccaagaacaccgtgtatcttcagatgaactccctgaaacccgaggacaccgccgtctactattgtgcgaaactgatcgctccattctattacggcatggattactggaccaaggggacccaggtgacagtgtctagc

SEQ ID NO: 363 - DNA, 363 bp, synthetic construct
  misc_feature 1..363: note = Description of Artificial Sequence: Synthetic polynucleotide
  source 1..363: mol_type = other DNA
gaagtgcagctggtggaaagcggcggaggtctgatgcaggcaggtggagcccttaggctctcttgtaccgcctctgggcctacttttacctcttatacgatgggctggttccgccaatctcctggcaagcgtcgcgagtttgtggccgtcatctccaaaggcgggcggacctattacgccgactccgtgaagggacgcttcactatttcccgcgacaacgctaagaataccttctatctccagatgtcctctctgaagcctgaggacacagcagtgtattactgcgccgggcagcgtgtgggcgcgactagcaagtatgagtatgattactgggggcagggcacccaagtgaccgtgtcatcc

SEQ ID NO: 364 - DNA, 360 bp, synthetic construct
  misc_feature 1..360: note = Description of Artificial Sequence: Synthetic polynucleotide
  source 1..360: mol_type = other DNA
gaagtgcaactggtggagagcggagggggtctggtacgcgcaggtggctccctgaggctctcctgcgctgcgtccggcttcacttttagtaccgactggatgtactgggtaagacgcgctccaggaaaggggctggagtgggtgtcccttatcaacactgacgggacttctacctcctatactaagtctgtgaaggggcgcttcacagtctcccgcgataatgccaagaacaccttgtaccttcagatgaactccctcaagccggaggacacagctctgtattactgtgcacgcggaagaacctactggttttacgcgatggattactggggcaagggcacccaggtgaccgtctcatct

SEQ ID NO: 365 - DNA, 369 bp, synthetic construct
  misc_feature 1..369: note = Description of Artificial Sequence: Synthetic polynucleotide
  source 1..369: mol_type = other DNA
gaggtccagttggtggaatctggaggcggactggtgcaggctggagacagtctgagattgtcttgtgccgcttctggccggatcagcaactacgcaatgggctggttccggcaggcacccggtaaagaaagggagttcgtcgctgtcatcaccaggagcggcggaagcacatactatgctgatagtgttaagggccgcttcaccatttccagagataacggcaaaaacacgattgatcttcagatgaacagactgaagcctgaagacacagcagtgtactattgtgccgtgaggcgcagtcaaaaactggtaacctttggcgctgagtatccttggtggggccagggaacattggtgactgtcagctcc

SEQ ID NO: 366 - DNA, 348 bp, synthetic construct
  misc_feature 1..348: note = Description of Artificial Sequence: Synthetic polynucleotide
  source 1..348: mol_type = other DNA
gaggtgcagttggtggagagcggcggaggcttggttcaagctgggggctcactcaggctgtcttgcactacctctgggcgtacaggcacccattatgcgatgggttggtttaggcaagcgcccggcaaggaacgcgagttcgttagtctcatcctgtggaacggcgagtttacgacctataaagattctgttaagggccgcttcaccatctcccgtgagaaaggcgaaaacacggtctacttgcaaatgaactctctgaaacccgaggatactgcggtgtattactgctacctgagggtgtttgctaggcgctactggggccagggaacccaggtgaccgtgtccagt

SEQ ID NO: 367 - DNA, 369 bp, synthetic construct
  misc_feature 1..369: note = Description of Artificial Sequence: Synthetic polynucleotide
  source 1..369: mol_type = other DNA
gaagtgcagctggtggaaagtggaggcggactggtgcagccagggggcagcctccgcctttcttgtgaggtgtccggctttaccttcagcaactactggatgtactggattcgccaagcccctgggaagggactggagtgggtgtcccacattaacaccaacggtggcaacacttattaccgccatagtgttaaaggtagattcactatcagcagggataacgctaagaataccctgtacttgcagatgaacggcctgaagtccgaggacaccgctgtgtattactgtgccaaggctaactccgatgtcgggttgggttattacggcatggattactggggtaagggaactcaggtcacagtgagttct

SEQ ID NO: 368 - DNA, 357 bp, synthetic construct
  misc_feature 1..357: note = Description of Artificial Sequence: Synthetic polynucleotide
  source 1..357: mol_type = other DNA
gaggtgcagctggtggaaagtggcgggggctctgttcagcccggcggatctctgcgcctgagctgtgctgcaccagagtccatcttcaacaataacgctgtttactggtatcggcaatttccgggcaaagaaagggagtacgtgggcctcatcacgattggtgggcgcactggatacgccgactctgtcaagggccgctttactatcagtcgtgataacgccaacaatgttgcttttctccagatggataacctgaagccggaagatactgcggtatattactgtgccgctaggcctggatattggtccagttcctacgattattggggtcagggaacccaagtaacagtgtcctct

SEQ ID NO: 369 - DNA, 348 bp, synthetic construct
  misc_feature 1..348: note = Description of Artificial Sequence: Synthetic polynucleotide
  source 1..348: mol_type = other DNA
gaagtgcagctggtggaaagcggcggtggcctcgtgcaggcgggcgggtccctgagactgtcatgcgtcttctctggccgcgccccggctagttatgcaatggcttggtttcgccaggccgtgggcaacgagagggagtttgtcgctgcgatcaactggtccggcaggcgcacttactatgccgactcagtgaagggccgcttcactatttccaaggacaatgcacagaacaccgcctatctccagatgaccaacttggaaccagaggatactgccacgtattactgtaatgcttacttgagcggaacatattactggggccagggcacccaggtgaccgtctctagc

SEQ ID NO: 370 - DNA, 366 bp, synthetic construct
  misc_feature 1..366: note = Description of Artificial Sequence: Synthetic polynucleotide
  source 1..366: mol_type = other DNA
gaggtccagctggtcgagtctggcggtggcttggtccgcgctggggactcactgcgcctgagttgtgctgtgtccggcctggccagctcctctttctttatgacttggttccgccaagggcagggcaaggagcgggaatttgtggccactatcagttggactggccgtacatcctattacgctgccagcgtgaaaggccgctttaccgttagtcgggacaatgccaagaataccgtgtaccttcagatgaactctctgaactctgaggatacagcagtctacttctgtgcagcctacccgcgtacactggtgcgtaatcgcgagccgatccattggggtcagggaacccaggtgactgtgtcctcc

SEQ ID NO: 371 - DNA, 363 bp, synthetic construct
  misc_feature 1..363: note = Description of Artificial Sequence: Synthetic polynucleotide
  source 1..363: mol_type = other DNA
caggtccagctccaggagagcgggggcggttctgtgcaagccggaggctcattgagactctcatgcgctgcaagtggttttaccttcgatgacagcgatatgggatggtatcgtcaggctccgggcaatgagtgtgatctggtctccactatctcctctgatggttccacatactatgctgactctgtcaaggggcgctttaccatctcccaagataatgccaagaacaccgtgtaccttcagatggattcagttaagcccgaggacacagccgtctattactgcgctgcggattttatgattgccatccaagctcccggagcgggatgctggggccagggaacccaggtcactgtgagcagt

SEQ ID NO: 372 - DNA, 351 bp, synthetic construct
  misc_feature 1..351: note = Description of Artificial Sequence: Synthetic polynucleotide
  source 1..351: mol_type = other DNA
caggtgcagttgcaggagtccggcgggggttctgtgccagcgggtgggagcctcaagctctcctgtgccgcttccggcttctcattctcctcttaccctatgacctgggcacgccaagcgcccggcaagggactggaatgggtgtccaccattgcttccgatggcggtagtacagcctacgccgcgtcagtggagggtcggttcacgatcagccgggacaacgcgaagagcacactctacctccagctgaactctctgaagaccgaggacaccgccatgtactattgcacaaagggctacggcgacggcaccccggcacccggccagggcacccaggtgacagtctcttcc

SEQ ID NO: 373 - DNA, 363 bp, synthetic construct
  misc_feature 1..363: note = Description of Artificial Sequence: Synthetic polynucleotide
  source 1..363: mol_type = other DNA
caggtgcagttgcaggaaagtggtggagggagtgtgcagactgggggctctctccgcctcagctgcacagcctctggatttaccttcgatgatcgcgagatgaactggtatcgccaggctccgggaaacgagtgcgaactggtgtctacaatcagttctgacgggtccacctattacgctgatagtgtcaagggccgcttcactatctctcaggacaacgcgaagaacaccgtttacttgcagatggatagcgtgaagcctgaagatacagcggtgtattactgcgctgccgactttatgattgccatccaggcaccgggggcggggtgttggggacagggaactcaggtgactgtgtcctcc

SEQ ID NO: 374 - DNA, 375 bp, synthetic construct
  misc_feature 1..375: note = Description of Artificial Sequence: Synthetic polynucleotide
  source 1..375: mol_type = other DNA
caggttcaactccaagagagtggtggcggaagcgtgcaggcgggcggttctctgcgtctgagttgcactgccagcggatttaccttcgacgattccgacatgggatggtacagacaggcccctggtaacgagtgcgaactcgtgagtactatcagctccgacggcaacacctattacaccgattctgtgaagggcaggttcaccatctcccaggacaacgctaagaacactgtgtacctgcaaatgaatagcctgggacccgaggacacagcggtctattactgcgcggcagagccgcgcggctattacagcaactacggcggtagacgcgagtgcaactactgggggcaggggacgcaagtgactgtctcctcc

SEQ ID NO: 375 - DNA, 351 bp, synthetic construct
  misc_feature 1..351: note = Description of Artificial Sequence: Synthetic polynucleotide
  source 1..351: mol_type = other DNA
caagtgcagcttcaggagtccgggggtggcagcgtccaggctgggggcagcttgcgcctgtcttgcgctgcgtctgggttcagctttagctcctaccctatgacctgggctagacaggcccccggcaaggggctggagtgggtgagtacaatcgcctccgacggaggtagtacggcctacgcagcgtccgtcgagggtcgcttcaccatcagccgggataacgctaagtccaccctgtaccttcagctcaattctctcaaaacggaggataccgccatgtactattgcaccaagggatatggcgacggcaccccagctcctggacagggcacacaggtcaccgttagctcc

SEQ ID NO: 376 - DNA, 360 bp, synthetic construct
  misc_feature 1..360: note = Description of Artificial Sequence: Synthetic polynucleotide
  source 1..360: mol_type = other DNA
caggtccagcttcaggagtctggcgggggcgcagtacaggcagggggttctctgcgtctgtcctgcgccgcgtccggctttactttcagcaacgcacacatgagttgggtgcgccaagcgcccggcaagggccgggaatggatcagtagcatctacagtggaggcagcacatggtacgccgactctgttaagggtcgttttacgatctctcgtgacaactccaagaacactttgtacctccagctcaattctctcaagaccgaggacaccgcgatgtactattgtgccgagaacaggctgcactactattccgacgatgactctctcaggggccagggaactcaagttaccgtgtccagc

SEQ ID NO: 377 - DNA, 363 bp, synthetic construct
  misc_feature 1..363: note = Description of Artificial Sequence: Synthetic polynucleotide
  source 1..363: mol_type = other DNA
caagtgcagctccaagagagtggtggcgggctggttcagccagggggcagcttgagactctcctgcgcagcttcaggctttaccttcgatgaccgtgagatgaactggtatcgtcaggccccaggcaacgagtgtgagctggttagcacgatttcttccgacggttccacctattacgccgactctgtgaagggacgtttcactatctcccaggacaatgccaagaacaccgtgtacctccagatggacagcgtgaagccggaggatactgctgtgtattactgcgctgccgactttatgatcgccatccaggcccctggcgcgggttgctggggccagggcactcaggtgaccgtgtcttcc

SEQ ID NO: 378 - DNA, 381 bp, synthetic construct
  misc_feature 1..381: note = Description of Artificial Sequence: Synthetic polynucleotide
  source 1..381: mol_type = other DNA
caagtgcaactgcaagagtccggcggtggatctgtgcaggccggaggcagcctgcggctgagctgtgtagcttccgggtatacctttagctcatactgtatgggctggtttcgtcaggcccccggtaaggagcgcgagggcgtggccgctcttggtggaggctccacctattacgccgattccgtgaagggcaggtttactatctcccaggacaacgcgaagaatacgctctatctccagatgaatagcctgaagcccgaggatacagctatgtattactgtgctgccgcttgggtagcctgcctggagttcggtggctcctggtacgatctggcacggtacaaacattgggggcagggcacccaggtcaccgtgtctagc

SEQ ID NO: 379 - DNA, 372 bp, synthetic construct
  misc_feature 1..372: note = Description of Artificial Sequence: Synthetic polynucleotide
  source 1..372: mol_type = other DNA
caggtccagttgcaggaatctgggggcggttccgtacaagcaggtggctcccttcggttgagctgtaccgcatccggctttactttcgacgatagcgatatgggctggtatcgtcaggccccagggggcgagtgcgagctggttacaatctcctctgacggcagtacctattacgcagactccgtcaagggcaggttcactatcagtcaggacaatgcaaagaacactgtgtatctccagatgaactctctgaagccagaagatactgccgtgtattactgcgctgcggaaccgagaggctattactctaattatggcgggcgtcgggagtgtaattattggggacagggaacccaggtgaccgtgtcctcc

SEQ ID NO: 380 - DNA, 381 bp, synthetic construct
  misc_feature 1..381: note = Description of Artificial Sequence: Synthetic polynucleotide
  source 1..381: mol_type = other DNA
caggtgcagctccaggagagtggcggaggctccgtgcaggctgggggctctctgcgtctgagctgtgccgcaagcggtagcatttacagctctgcctacatcgggtggtttcgtcaagcgccgggcaaaaagcgcgaaggcgtggccggaatctacacgcgcgatggctccaccgcttatgctgacagcgttaagggacgttttacgatcagccaggactctgccaaaaagactgtgtatctccagatgaactccctgaaacctgaggacacagccatgtattactgcgccgctggccgccgtacaaagagctatgtttacatctttcgccccgaagagtacaactactggggccagggaacccaagtgactgtgtccagt

SEQ ID NO: 381 - DNA, 360 bp, synthetic construct
  misc_feature 1..360: note = Description of Artificial Sequence: Synthetic polynucleotide
  source 1..360: mol_type = other DNA
caggttcagttgcaggagtccggcggaggcagcgtgcaggccggaggctccttgcgcttgtcctgtgcggcttctggcttcaccttctcatctgctcacatgagttgggtgcgtcaggccccagggaaaggtcgcgagtggattgcctccatctacagcggtgggggcactttttatgcggacagcgtgaagggccgctttaccatcagccgtgacaacgctaagaacaccctgtatctccaactcaattccctcaagaccgaggatacagcgatgtactattgtgcaaccaaccgccttcactattactccgacgatgacagcctgcgcggacaggggacccaggtgacggtgtccagc

SEQ ID NO: 382 - DNA, 360 bp, synthetic construct
  misc_feature 1..360: note = Description of Artificial Sequence: Synthetic polynucleotide
  source 1..360: mol_type = other DNA
caggtgcaactccaggaaagtggcggaggctcagtgcaggcaggtggctctctccgcctttcctgcgctgccagcggattcaccttctctaacgctcacatgagctgggttcgtcaggctcccggcaaaggccgtgaatggattagctccatctatagtggcggaagtacttggtacgcagatagcgtcaagggccgcttcactattagtcgggataactccaagaacactctgtacctccagctgaactcattgaaaaccgaggacacggctatgtactattgtgctgagaacaggctgcactattactccgacgatgactctctgaggggtcagggcacccaggtgaccgtcagctcc

SEQ ID NO: 383 - DNA, 375 bp, synthetic construct
  misc_feature 1..375: note = Description of Artificial Sequence: Synthetic polynucleotide
  source 1..375: mol_type = other DNA
caggtccaactccaggagtccggcggaggcagcgtgcaggctggaggctctctccgcctgagctgcacagcttccagattcatcttcgatgactccgacatgggctggtatcgccaggctccagggaacgagtgcgaactggtgagcaccatctcttcagacggtagcacctattacgccgacagtgtgaaggggcgcttcaccatctcccgcgacaatgctaaaaatacggtgtatctccagatgaactccctcaaaccggaggacacagctgtatattactgtgctgcggaaccacggggctactatagcaactatggtggaaggcgcgagtgcaactactggggtcagggcacacaggtgacggtttcctcc

SEQ ID NO: 384 - DNA, 372 bp, synthetic construct
  misc_feature 1..372: note = Description of Artificial Sequence: Synthetic polynucleotide
  source 1..372: mol_type = other DNA
caggtgcagctccaggagagcggcggtggctccgtgcaggctggtggcagcctgaagctgtcctgcaccgtgagtggcttcacagccgacgattctgatatgggctggtatcgccaaggccccggcaatgagtgcgagctggtaaccattagctcagacggctctacatactatgccgattctgttaagggccgctttactatctcacaggataatgccaagaacacagtgtacttgcagatgaactctctgaaaccggaagacacagctgtgtattactgtgctgcggagcctagagggtattacagcaattacgggggccggagagagtgtaactattgggggcagggcacccaagtgaccgtttcctcc

SEQ ID NO: 385 - DNA, 360 bp, synthetic construct
  misc_feature 1..360: note = Description of Artificial Sequence: Synthetic polynucleotide
  source 1..360: mol_type = other DNA
caggtccagcttcaggaatctgggggcggtctcgtgcagcccggcgggtccctgcgtctgtcttgtgctgcgagcggcttcacgttctcaagtgcccacatgagctgggtaaggcaggcaccgggcaaggggcgcgagtggattgcaagcatctattcaggcgggggcacattctacgccgacagcgtgaagggacgttttacaatctccagagataacgcaaagaacactctctacctccaactcaactccttgaaggcggaagatactgcaatgtattactgtgctactaaccgtcttcattattactctgacgatgactccctgcgggggcagggtacacaggtgacagtgagttcc

SEQ ID NO: 386 - DNA, 360 bp, synthetic construct
  misc_feature 1..360: note = Description of Artificial Sequence: Synthetic polynucleotide
  source 1..360: mol_type = other DNA
caggtgcagctgcaagaatctggtggagggctggtccagcctgggggctccctgcgcctctcatgtgtcgcatctggcttcaccttcagcaacgcccacatgagctgggttcgccaagcccctgggaagggccgggagtggatctccagtatctattccggcggaagcacttggtatgcagacagcgtcaaaggacggttcactatttctcgtgataattctaagaacaccctgtaccttcagctgaacagcctgaagaccgaggacactgctatgtactattgtgctgagaatcgcctgcattactatagcgacgatgacagtctgcgcggacaggggacccaggtcaccgtgtcctct

SEQ ID NO: 387 - DNA, 360 bp, synthetic construct
  misc_feature 1..360: note = Description of Artificial Sequence: Synthetic polynucleotide
  source 1..360: mol_type = other DNA
caggttcagttgcaggaatcaggaggcggtctggtgcagcctgggggctctctgcgtctctcctgcgccgcttccggcttcacattctccaacgcccacatgagctgggtccgccaggcccctgggaagggccgcgagtggatctccagtatctacagcgggggctccacttggtacgcagacagcgtcaaagggaggtttaccattagccgtgacaattctaagaacacattgtatttgcagctgaactctcttaaaaccgaggacaccgccatgtactattgtgctgagaacaggctccactattactcagacgatgactcacttcgcgggcagggaacccaggtcaccgtctcctct

SEQ ID NO: 388 - DNA, 351 bp, synthetic construct
  misc_feature 1..351: note = Description of Artificial Sequence: Synthetic polynucleotide
  source 1..351: mol_type = other DNA
caagtccagctccaggaaagcggcggtggcctggtgcaacctggcgggtctctgcgcttgtcatgcgctgcctccggcttcaccttctcatcttaccctatgacctgggcgcgtcaggctcccggcaagggattggagtgggtgtctactattgcctccgacggtggcagcacggcctacgcagcgtctgtagaaggacgcttcacaattagcagagacaacgcaaaatctactttgtaccttcagctcaacagcctgaagaccgaagacacagctatgtattactgcacaaaaggctacggggacggcacgccagcgcctggacaggggacacaggtgaccgtatcttct

SEQ ID NO: 389 - DNA, 363 bp, synthetic construct
  misc_feature 1..363: note = Description of Artificial Sequence: Synthetic polynucleotide
  source 1..363: mol_type = other DNA
caggtgcagttgcaggaatcagggggtggctctgtgcaggccgggggctccctgcgtctgtcctgtactgcgagcggcttcacctttgatgaccgcgagatgaactggtatcgccaggctccggggaacgagtgcgaactcgtgtctacaattagctccgatggttcaacatactatgctgattctgtcaaaggtcgctttaccatctcacaggacaacgccaagaacaccgtctacctccagatggactctgtgaagcctgaagataccgccgtatactattgcgccgctgactttatgattgccattcaggctccgggtgctggatgctggggtcaggggactcaggtgaccgtgtcttca

SEQ ID NO: 390 - DNA, 375 bp, synthetic construct
  misc_feature 1..375: note = Description of Artificial Sequence: Synthetic polynucleotide
  source 1..375: mol_type = other DNA
caagtgcagttgcaggaaagcggcggtgggtccgtgcaagccggaggttctctccgcctgtcttgcactgcctcaggttttaccttcgacgattccgatatgggctggtacaggcaggctcccggcaatgagtgcgagctggtgtctacgatctcaagtgatggctccacctactatgccgatagcgtaaaaggaaggtttactattagccaggataacgcgaagaacacggtgtacctccagatgaacagtctcaagccggaggatactgccgtgtattactgtgctgccgagccgcgtggctattactccaactacggtggcagacgtgaatgcaattactggggacagggtactcaggttaccgtgtcctct

SEQ ID NO: 391 - DNA, 357 bp, synthetic construct
  misc_feature 1..357: note = Description of Artificial Sequence: Synthetic polynucleotide
  source 1..357: mol_type = other DNA
caggttcaacttcaggaatccgggggcggttccgtgcaagccgggggtagcctgcgtctgtcttgcgtggccagcggctatacctcctgtatgggttggtttcggcaggctcctgggaaggagcgcgaagccgtggcgaccatctacacacggggccgcagcatctattacgctgacagtgtgaagggccgcttcaccatctcccaggataacgccaagaataccctgtatctgcaaatgaactccctgaagcctgaggacatcgccatgtattcctgcgcagctggagggtactcatggtccgctgggtgcgagtttaattattggggccaaggaacccaggtgaccgtctcctca

SEQ ID NO: 392 - DNA, 351 bp, synthetic construct
  misc_feature 1..351: note = Description of Artificial Sequence: Synthetic polynucleotide
  source 1..351: mol_type = other DNA
caagtgcagctccaggagtctggcgggggcctggttcagcctggtgggtccctgcgcctgtcttgcacggcttccggctttagcttctcctcatatccaatgacctgggcacgccaggctcctggtaagggcctggagtgggtctccaccatcgcctctgatggtgggtcaactgcctatgctgcctccgtcgagggtagattcacaatcagcagagacaacgccaaatccacgctgtacctgcaactcaactccttgaagaccgaggacacagctatgtattactgtaccaaaggctacggcgacggcactcctgctcccggacaggggacccaggtgactgtgtctagc

SEQ ID NO: 393 - DNA, 351 bp, synthetic construct
  misc_feature 1..351: note = Description of Artificial Sequence: Synthetic polynucleotide
  source 1..351: mol_type = other DNA
caggtccaacttcaggaaagcgggggtggactggtacagccagggggcagtctgcgcctgtcctgtgccgcaagcgggttttctttctccagttaccccatgacctgggctcgccaagcacctggaaagggactggagtgggtgtctactattgcgtcagatggtgggagtacggcttacgccgcgagcgtggagggtcgttttacgatcagtagggacaacgccaaaagcactctgtacctccagcttaacagcctgaagaccgaggacaccgccatgtattactgtaccaagggctacggagacggcacccctgcgccggggcaaggcacccaggtgaccgtaagttca

SEQ ID NO: 394 - DNA, 381 bp, synthetic construct
  misc_feature 1..381: note = Description of Artificial Sequence: Synthetic polynucleotide
  source 1..381: mol_type = other DNA
caggtgcaactccaggagtccggcgggggctccgtgctggctggcggatctttgaggctgtcttgcgtggcttctggctatggctataattacatcggctggttccgtcagacacccggcaaggagcgcgaaggggtggcggtcatttacacagggggtggggacacttattacgccgactccgtcaagggtaggtttaccgctagtcgcgataatgccaaaagtacgctgtacctgcaaatgaacagcttggagccagaggacaccgccatgtattacggagtggctcgctactgtgtgggcagtgtgtacgcttgcctgcgcggaggccacgacgagtacgcacactggggccagggaacccaggtgacagtgtctagc

SEQ ID NO: 395 - DNA, 402 bp, synthetic construct
  misc_feature 1..402: note = Description of Artificial Sequence: Synthetic polynucleotide
  source 1..402: mol_type = other DNA
caggtgcagctccaggagtctgggggtggcagcgtccagccaggtggctcattgagactgtcttgtgctgcatctggctccacctacgctaattacctgatgggatggttcaggcaggcccctggtaaggagcgtgagggcgtggccgctatctattctggcggtgggtccacctactatgctgactccgtcaagggacgcttcactatttctcaagacaatgccaagaacactttgtacttgcaaatgaactcactcaaacctgaggacaccgcgatgtactattgcgcagcggcatccgcagtgaagggagacaaaggggatatcgtggtagttgtgaccggcacccagcgtatggagtacgactactggggacatggcacccaggtgacagttagctcc

SEQ ID NO: 396 - DNA, 396 bp, synthetic construct
  misc_feature 1..396: note = Description of Artificial Sequence: Synthetic polynucleotide
  source 1..396: mol_type = other DNA
caggtacagttgcaggagagtggtgggggttccgtccaggccggtgcctctcttcgcctcagttgtagcgtgagcggtttcacgttcgacgagtcagtgatgtcctggttgcgccagggtcccggcaatgagtgcgacgcggtcgctattatcagctccgatgacaacacctattacgacgatagcgtgaaaggccgctttaccatctccgaggacaacgccaaaaacatggtgtatctgcaaatgaactcactgaagccggaagacaccgcagtgtactattgcgccgcgcgtcggcgcagacctgtgtacgattccgattatgaactccggccacgtccgctgtgtggcgatttcggcgtgtggggccaggggacccaggtgacggtctcctcc

SEQ ID NO: 397 - DNA, 387 bp, synthetic construct
  misc_feature 1..387: note = Description of Artificial Sequence: Synthetic polynucleotide
  source 1..387: mol_type = other DNA
caggtgcagctccaggaatctggcgggggctctgtgcaggctggtggctcccttcgcctgtcctgtattggctccggtcttcctttcgacgaggatgacatgggctggtatcgccaggcccctgggaatgagtgtgaattggtcagctcaatctccagtgacggcaccgcctattacgccgattccgtcaagggacgcttcactatctccagagacaacgccaagaacactgtgctgttgcagatgaactccctgaagcccgaggataccgctgtctattactgcgcagccggggtccacagacagttcggcggttccagttcctgcggcgacgccttctacggcatggattactggggcaagggaactcaggtcacagtgtcttcc

SEQ ID NO: 398 - DNA, 372 bp, synthetic construct
  misc_feature 1..372: note = Description of Artificial Sequence: Synthetic polynucleotide
  source 1..372: mol_type = other DNA
caggttcagcttcaggagtccggcgggggctccgtacaggcagggggctcactgcgtctttcctgtgtggcgagtggcgacgtgtatggccgtaacagcatggcttggttccggcaggcacctggaaaggaacgcgagggcgttgcagttgggtattccgtagtgacaaccacttactatgccgacagtgtgaagggccggtttacgatctcagaggacaacgataaaaacacagtgtacctggagatgaactccctgaagccggaagacactgctatgtattactgcgctgccgatggcaacctgtggcgcggactcaggccctccgagtacacttattggggtcagggcacccaggtgaccgtttcaagt

SEQ ID NO: 399 - DNA, 384 bp, synthetic construct
  misc_feature 1..384: note = Description of Artificial Sequence: Synthetic polynucleotide
  source 1..384: mol_type = other DNA
caggtccagcttcaggagtcaggtggcggtagtgtccaggcaggcggtagcctgcgccttagctgtgctacatccggcttcccttactcacgctattgtatgggctggttcaggcaagctcccggtaaagagcgcgagggagtggcagccatcgagcctgacgggagcacatcttatgctgactctgtaaaggggcgtttcaccatctctcaggacaacgccgttaatacactgtacttgcaaatgaataacctgaagcccgaggacacagctatgtattactgcgcagccgacgagcgttgcttctatttgaaggactatgacctcagaaggccagcccagtaccgctactgggggcagggcacccaggttaccgtgtcatct

SEQ ID NO: 400 - DNA, 396 bp, synthetic construct
  misc_feature 1..396: note = Description of Artificial Sequence: Synthetic polynucleotide
  source 1..396: mol_type = other DNA
caggtgcagttgcaggagagtggcggtggcctcgtgcagcctggcggaagcctccgtctgagctgcactgtgtccggcttcactttcgacgagagcgacatgggctggctgaggcagaaccctggtaacgagtgcggcgttgtgagtgtcatcacgtctgatgacaacccatactatgatgacagcgtcaagggccgctttactatctccgaggataacgccaagaacatggtgtacctccagatgaactcactgaagcccgaggataccggcgtttattactgtgcaaccaggagccgtcagcctgtgtactcacgcgattacgagctgcggccccgccccctctgtggagactttggtgtgtggggccagggcacccaggtgactgtttccagc

SEQ ID NO: 401 - DNA, 378 bp, synthetic construct
  misc_feature 1..378: note = Description of Artificial Sequence: Synthetic polynucleotide
  source 1..378: mol_type = other DNA
caggtgcagttgcaggagagtggagggggctcagtgcaggctggcgggtccttgcgtctgtcttgcaccgcctctggcttcaccttcgatgacttcgatatgggttggtatcgccaggctccagggaacgagtgcgaattggtcagcactatcagcgacgatggctcaacatattacgccgactctgtgaagggacggtctagcattagccgggacaacgcaaagaacaccgtctatctccagatgaaccgcttgaagcctgaggataccggagtctattactgcgccgctgagggcgcgttgggctccaagactaattgtggctgggtgggcaacttcggatattggggccagggaacacaggttaccgtttccagc

SEQ ID NO: 402 - DNA, 378 bp, synthetic construct
  misc_feature 1..378: note = Description of Artificial Sequence: Synthetic polynucleotide
  source 1..378: mol_type = other DNA
caggtgcagttgcaggagtctggaggcggttccgttcaggccgggggctctctgcgcctgtcctgcgctgcctccgggtttacatttgacgatttcgatatgggctggtatcgccaggcccctggcaacgagtgcgaactggtgtctactatctccgatgacggctcaacctactatgcagactccgtaaagggcagatccagcatctcccgcgacaatgccaaaaacactgtgtacctccagatgaactccctcaagcctgaggatacggcggtgtactattgtgctgccgagggtgcgctcggtagcaagactaattgcggctgggtgggcaacttcgggtactggggtcaggggacccaggtaaccgtgtcttct

SEQ ID NO: 403 - DNA, 378 bp, synthetic construct
  misc_feature 1..378: note = Description of Artificial Sequence: Synthetic polynucleotide
  source 1..378: mol_type = other DNA
caggtgcagttgcaggaaagcggtgggggcctggtgcagcccggaggcagcctgcgcttgagctgcgctgcctctggcttcacattcgatgacttcgatatgggctggtatcgtcaagcacccggaaacgagtgcgagctggtgagtacaatcagtgatgacggatctacctactatgccgacagcgtcaagggaagatccagcatcagtcgcgacaacgccaagagcaccgtttacctccagatgaaccgcctcaagcctgaggacacaggagtctattactgtgctgcggagggggccttgggcagcaagactaactgtggatgggtgggaaacttcgggtattggggtcagggtacacaggtcacagtgtcttca

SEQ ID NO: 404 - DNA, 378 bp, synthetic construct
  misc_feature 1..378: note = Description of Artificial Sequence: Synthetic polynucleotide
  source 1..378: mol_type = other DNA
caagttcagcttcaggaaagtgggggcgggctggtgcagccagggggttccctgaagctgagctgcgctgcctctgggtttacattctctgatcgcgacatgggctggtatcgccaagcgccgggcaatgaatgcgaaagagtgagtactatttctgacgatggttctacttactatgctgactccgtgaagggccgtagctccatttccagggacaacgcgaagaacaccgtatacctccagatgaactctctgaagcccgaggacaccgctgtgtattactgcgctgccgagggggctctcggctcaaagaccaactgcggatgggtcggtaacttcggctactggggccagggcacccaagtgacagtctcctcc

SEQ ID NO: 405 - DNA, 381 bp, synthetic construct
  misc_feature 1..381: note = Description of Artificial Sequence: Synthetic polynucleotide
  source 1..381: mol_type = other DNA
caggtccagttgcaggagagcgggggtggaagcgtcctcgccggagggagcctccgtttgagctgcgtcgcctcaggctacggctacaattacatcggatggttcagacagacgcctggtaaagagcgggaaggcgtcgccgtgatttatatcggtggcggagacacctattacgctgactcagtgaaggggcgtttcaccgcaagccgggacaacgctaagagcaccctgtacctccagatgaactctctcgaacctgaggacactgcaatgtattactgcgtggctcgttactgcgtcgggagtgtctacgcctgcctgaggggcgggcatgatgagtatgcccactggggacaaggaacacaggtgactgtctccagt

SEQ ID NO: 406 - DNA, 381 bp, synthetic construct
  misc_feature 1..381: note = Description of Artificial Sequence: Synthetic polynucleotide
  source 1..381: mol_type = other DNA
caggttcagctccaggagtctggtggcggttccgtgctggccgggggctctctgcgcctgtcttgtgtcgcctcagggtacggctataactacattggctggttcagacagacccctgggaaagagcgggagggtgtggctgtcatttacaccggcggaggcgacacctactatgccgattcagttaagggcaggtttaccgcgagccgtgacaacgcgaagtctactctgtacctgcaaatgaacagcctggaacctgaggatactgcgatgtactattgtgtggcccggtactgcgtaggctcagtgtatgcctgcctgcgcgggggtcacgacgagtacgcacactggggacagggaactcaggtcaccgtgtctagc

SEQ ID NO: 407 - DNA, 378 bp, synthetic construct
  misc_feature 1..378: note = Description of Artificial Sequence: Synthetic polynucleotide
  source 1..378: mol_type = other DNA
caggtgcaactccaggagtccggcgggggctccgtccaagctggtggctcactgaggcttagctgtgctgcctccggctttactttcgacgatttcgacatgggttggtatcgccaggctccgggcaatgagtgcgagctggtctctaccatttccgatgacggctctacctactatgccaacagtgttaagggtaggtcttccatctcccgcgacaacgctaagaatatggtgtacttgcagatgaactctctgaagcctgaggacactgctgtctactattgcgctgccgaaggtgccctgggctcaaagactaattgcggctgggtcggtaactttggctactggggtcaggggactcaggtgaccgtcagctcc

SEQ ID NO: 408 - DNA, 378 bp, synthetic construct
  misc_feature 1..378: note = Description of Artificial Sequence: Synthetic polynucleotide
  source 1..378: mol_type = other DNA
caggtccagttgcaggaaagcggcgggggctctgttcaggcaggcggaagccttcgtctgtcctgtactgccagtggtttcacctttgatgactttgacatgggctggtatcggcaagcccccggaaacgagtgcgagctggtatccaccatttccgatgacgggtccacgtactatgctgatagcgtgaagggcaggtcttccatcagccgggacaacgccaagaacacagtgtatttgcagatgaaccgcctcaagccagaagacaccggggtatattactgtgcagcggaaggtgccctgggtagcaagatgaactgcggatgggtgggtaattttggatactggggccagggcacgcaggttacagtgtccagc

SEQ ID NO: 409-452 - skipped (44 entries)

SEQ ID NO: 453 - protein, 214 aa, Homo sapiens
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 454 - skipped

SEQ ID NO: 455 - protein, 225 aa, Mus musculus
LEFIAYGTELPSPSYVWFEARFFQHILHWKPIPNQSESTYYEVALKQYGNSTWNDIHICRKAQALSCDLTTFTLDLYHRSYGYRARVRAVDNSQYSNWTTTETRFTVDEVILTVDSVTLKAMDGIIYGTIHPPRPTITPAGDEYEQVFKDLRVYKISIRKFSELKNATKRVKQETFTLTVPIGVRKFCVKVLPRLESRINKAEWSEEQCLLITTEQYFTVTNLSI

SEQ ID NO: 456 - protein, 4 aa, synthetic construct
  REGION 1..4: note = Description of Artificial Sequence: Synthetic peptide
GGSG

SEQ ID NO: 457 - protein, 8 aa, synthetic construct
  REGION 1..8: note = Description of Artificial Sequence: Synthetic peptide
GGSGGGSG

SEQ ID NO: 458 - protein, 12 aa, synthetic construct
  REGION 1..12: note = Description of Artificial Sequence: Synthetic peptide
GGSGGGSGGGSG

SEQ ID NO: 459 - protein, 16 aa, synthetic construct
  REGION 1..16: note = Description of Artificial Sequence: Synthetic peptide
GGSGGGSGGGSGGGSG

SEQ ID NO: 460 - protein, 20 aa, synthetic construct
  REGION 1..20: note = Description of Artificial Sequence: Synthetic peptide
GGSGGGSGGGSGGGSGGGSG

SEQ ID NO: 461 - protein, 16 aa, synthetic construct
  REGION 1..16: note = Description of Artificial Sequence: Synthetic peptide
LITIGGRTGYADSVKG

SEQ ID NO: 462 - protein, 17 aa, synthetic construct
  REGION 1..17: note = Description of Artificial Sequence: Synthetic peptide
AINWSGRRTYYADSVKG

SEQ ID NO: 463 - protein, 17 aa, synthetic construct
  REGION 1..17: note = Description of Artificial Sequence: Synthetic peptide
TISWTGRTSYYAASVKG

SEQ ID NO: 464 - protein, 800 aa, synthetic construct
  REGION 1..800: note = Description of Artificial Sequence: Synthetic polypeptide
  VARIANT 1..20: note = This region may encompass 1-20 residues
  VARIANT 21..40: note = This region may encompass 1-20 residues
  VARIANT 41..60: note = This region may encompass 1-20 residues
  VARIANT 61..80: note = This region may encompass 1-20 residues
  VARIANT 81..100: note = This region may encompass 1-20 residues
  VARIANT 101..120: note = This region may encompass 1-20 residues
  VARIANT 121..140: note = This region may encompass 1-20 residues
  VARIANT 141..160: note = This region may encompass 1-20 residues
  VARIANT 161..180: note = This region may encompass 1-20 residues
  VARIANT 181..200: note = This region may encompass 1-20 residues
  VARIANT 201..220: note = This region may encompass 1-20 residues
  VARIANT 221..240: note = This region may encompass 1-20 residues
  VARIANT 241..260: note = This region may encompass 1-20 residues
  VARIANT 261..280: note = This region may encompass 1-20 residues
  VARIANT 281..300: note = This region may encompass 1-20 residues
  VARIANT 301..320: note = This region may encompass 1-20 residues
  VARIANT 321..340: note = This region may encompass 1-20 residues
  VARIANT 341..360: note = This region may encompass 1-20 residues
  VARIANT 361..380: note = This region may encompass 1-20 residues
  VARIANT 381..400: note = This region may encompass 1-20 residues
  VARIANT 401..420: note = This region may encompass 1-20 residues
  VARIANT 421..440: note = This region may encompass 1-20 residues
  VARIANT 441..460: note = This region may encompass 1-20 residues
  VARIANT 461..480: note = This region may encompass 1-20 residues
  VARIANT 481..500: note = This region may encompass 1-20 residues
  VARIANT 501..520: note = This region may encompass 1-20 residues
  VARIANT 521..540: note = This region may encompass 1-20 residues
  VARIANT 541..560: note = This region may encompass 1-20 residues
  VARIANT 561..580: note = This region may encompass 1-20 residues
  VARIANT 581..600: note = This region may encompass 1-20 residues
  VARIANT 601..620: note = This region may encompass 1-20 residues
  VARIANT 621..640: note = This region may encompass 1-20 residues
  VARIANT 641..660: note = This region may encompass 1-20 residues
  VARIANT 661..680: note = This region may encompass 1-20 residues
  VARIANT 681..700: note = This region may encompass 1-20 residues
  VARIANT 701..720: note = This region may encompass 1-20 residues
  VARIANT 721..740: note = This region may encompass 1-20 residues
  VARIANT 741..760: note = This region may encompass 1-20 residues
  VARIANT 761..780: note = This region may encompass 1-20 residues
  VARIANT 781..800: note = This region may encompass 1-20 residues
  VARIANT 1..800: note = This sequence may encompass 1-20 "(Gly)m(Ser)o" repeating units, wherein m = 1 to 20, o = 1 to 20
  REGION 1..800: note = See specification as filed for detailed description of substitutions and preferred embodiments
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSS

SEQ ID NO: 465 - protein, 100 aa, synthetic construct
  REGION 1..100: note = Description of Artificial Sequence: Synthetic polypeptide
  VARIANT 1..100: note = This sequence may encompass 1-20 "Gly Ser Gly Gly Ser" repeating units
  REGION 1..100: note = See specification as filed for detailed description of substitutions and preferred embodiments
GSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGS

SEQ ID NO: 466 - protein, 1200 aa, synthetic construct
  REGION 1..1200: note = Description of Artificial Sequence: Synthetic polypeptide
  VARIANT 1..20: note = This region may encompass 1-20 residues
  VARIANT 21..40: note = This region may encompass 1-20 residues
  VARIANT 41..60: note = This region may encompass 1-20 residues
  VARIANT 61..80: note = This region may encompass 1-20 residues
  VARIANT 81..100: note = This region may encompass 1-20 residues
  VARIANT 101..120: note = This region may encompass 1-20 residues
  VARIANT 121..140: note = This region may encompass 1-20 residues
  VARIANT 141..160: note = This region may encompass 1-20 residues
  VARIANT 161..180: note = This region may encompass 1-20 residues
  VARIANT 181..200: note = This region may encompass 1-20 residues
  VARIANT 201..220: note = This region may encompass 1-20 residues
  VARIANT 221..240: note = This region may encompass 1-20 residues
  VARIANT 241..260: note = This region may encompass 1-20 residues
  VARIANT 261..280: note = This region may encompass 1-20 residues
  VARIANT 281..300: note = This region may encompass 1-20 residues
  VARIANT 301..320: note = This region may encompass 1-20 residues
  VARIANT 321..340: note = This region may encompass 1-20 residues
  VARIANT 341..360: note = This region may encompass 1-20 residues
  VARIANT 361..380: note = This region may encompass 1-20 residues
  VARIANT 381..400: note = This region may encompass 1-20 residues
  VARIANT 401..420: note = This region may encompass 1-20 residues
  VARIANT 421..440: note = This region may encompass 1-20 residues
  VARIANT 441..460: note = This region may encompass 1-20 residues
  VARIANT 461..480: note = This region may encompass 1-20 residues
  VARIANT 481..500: note = This region may encompass 1-20 residues
  VARIANT 501..520: note = This region may encompass 1-20 residues
  VARIANT 521..540: note = This region may encompass 1-20 residues
  VARIANT 541..560: note = This region may encompass 1-20 residues
  VARIANT 561..580: note = This region may encompass 1-20 residues
  VARIANT 581..600: note = This region may encompass 1-20 residues
  VARIANT 601..620: note = This region may encompass 1-20 residues
  VARIANT 621..640: note = This region may encompass 1-20 residues
  VARIANT 641..660: note = This region may encompass 1-20 residues
  VARIANT 661..680: note = This region may encompass 1-20 residues
  VARIANT 681..700: note = This region may encompass 1-20 residues
  VARIANT 701..720: note = This region may encompass 1-20 residues
  VARIANT 721..740: note = This region may encompass 1-20 residues
  VARIANT 741..760: note = This region may encompass 1-20 residues
  VARIANT 761..780: note = This region may encompass 1-20 residues
  VARIANT 781..800: note = This region may encompass 1-20 residues
  VARIANT 801..820: note = This region may encompass 1-20 residues
  VARIANT 821..840: note = This region may encompass 1-20 residues
  VARIANT 841..860: note = This region may encompass 1-20 residues
  VARIANT 861..880: note = This region may encompass 1-20 residues
  VARIANT 881..900: note = This region may encompass 1-20 residues
  VARIANT 901..920: note = This region may encompass 1-20 residues
  VARIANT 921..940: note = This region may encompass 1-20 residues
  VARIANT 941..960: note = This region may encompass 1-20 residues
  VARIANT 961..980: note = This region may encompass 1-20 residues
  VARIANT 981..1000: note = This region may encompass 1-20 residues
  VARIANT 1001..1020: note = This region may encompass 1-20 residues
  VARIANT 1021..1040: note = This region may encompass 1-20 residues
  VARIANT 1041..1060: note = This region may encompass 1-20 residues
  VARIANT 1061..1080: note = This region may encompass 1-20 residues
  VARIANT 1081..1100: note = This region may encompass 1-20 residues
  VARIANT 1101..1120: note = This region may encompass 1-20 residues
  VARIANT 1121..1140: note = This region may encompass 1-20 residues
  VARIANT 1141..1160: note = This region may encompass 1-20 residues
  VARIANT 1161..1180: note = This region may encompass 1-20 residues
  VARIANT 1181..1200: note = This region may encompass 1-20 residues
  VARIANT 1..1200: note = This sequence may encompass 1-20 "(Gly)m(Ser)o(Gly)m" repeating units, wherein m = 1 to 20,; o = 1 to 20
  REGION 1..1200: note = See specification as filed for detailed description of substitutions and preferred embodiments
GGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGG

SEQ ID NO: 467 - protein, 2000 aa, synthetic construct
  REGION 1..2000: note = Description of Artificial Sequence: Synthetic polypeptide
  VARIANT 1..20: note = This region may encompass 1-20 residues
  VARIANT 21..40: note = This region may encompass 1-20 residues
  VARIANT 41..60: note = This region may encompass 1-20 residues
  VARIANT 61..80: note = This region may encompass 1-20 residues
  VARIANT 81..100: note = This region may encompass 1-20 residues
  VARIANT 101..120: note = This region may encompass 1-20 residues
  VARIANT 121..140: note = This region may encompass 1-20 residues
  VARIANT 141..160: note = This region may encompass 1-20 residues
  VARIANT 161..180: note = This region may encompass 1-20 residues
  VARIANT 181..200: note = This region may encompass 1-20 residues
  VARIANT 201..220: note = This region may encompass 1-20 residues
  VARIANT 221..240: note = This region may encompass 1-20 residues
  VARIANT 241..260: note = This region may encompass 1-20 residues
  VARIANT 261..280: note = This region may encompass 1-20 residues
  VARIANT 281..300: note = This region may encompass 1-20 residues
  VARIANT 301..320: note = This region may encompass 1-20 residues
  VARIANT 321..340: note = This region may encompass 1-20 residues
  VARIANT 341..360: note = This region may encompass 1-20 residues
  VARIANT 361..380: note = This region may encompass 1-20 residues
  VARIANT 381..400: note = This region may encompass 1-20 residues
  VARIANT 401..420: note = This region may encompass 1-20 residues
  VARIANT 421..440: note = This region may encompass 1-20 residues
  VARIANT 441..460: note = This region may encompass 1-20 residues
  VARIANT 461..480: note = This region may encompass 1-20 residues
  VARIANT 481..500: note = This region may encompass 1-20 residues
  VARIANT 501..520: note = This region may encompass 1-20 residues
  VARIANT 521..540: note = This region may encompass 1-20 residues
  VARIANT 541..560: note = This region may encompass 1-20 residues
  VARIANT 561..580: note = This region may encompass 1-20 residues
  VARIANT 581..600: note = This region may encompass 1-20 residues
  VARIANT 601..620: note = This region may encompass 1-20 residues
  VARIANT 621..640: note = This region may encompass 1-20 residues
  VARIANT 641..660: note = This region may encompass 1-20 residues
  VARIANT 661..680: note = This region may encompass 1-20 residues
  VARIANT 681..700: note = This region may encompass 1-20 residues
  VARIANT 701..720: note = This region may encompass 1-20 residues
  VARIANT 721..740: note = This region may encompass 1-20 residues
  VARIANT 741..760: note = This region may encompass 1-20 residues
  VARIANT 761..780: note = This region may encompass 1-20 residues
  VARIANT 781..800: note = This region may encompass 1-20 residues
  VARIANT 801..820: note = This region may encompass 1-20 residues
  VARIANT 821..840: note = This region may encompass 1-20 residues
  VARIANT 841..860: note = This region may encompass 1-20 residues
  VARIANT 861..880: note = This region may encompass 1-20 residues
  VARIANT 881..900: note = This region may encompass 1-20 residues
  VARIANT 901..920: note = This region may encompass 1-20 residues
  VARIANT 921..940: note = This region may encompass 1-20 residues
  VARIANT 941..960: note = This region may encompass 1-20 residues
  VARIANT 961..980: note = This region may encompass 1-20 residues
  VARIANT 981..1000: note = This region may encompass 1-20 residues
  VARIANT 1001..1020: note = This region may encompass 1-20 residues
  VARIANT 1021..1040: note = This region may encompass 1-20 residues
  VARIANT 1041..1060: note = This region may encompass 1-20 residues
  VARIANT 1061..1080: note = This region may encompass 1-20 residues
  VARIANT 1081..1100: note = This region may encompass 1-20 residues
  VARIANT 1101..1120: note = This region may encompass 1-20 residues
  VARIANT 1121..1140: note = This region may encompass 1-20 residues
  VARIANT 1141..1160: note = This region may encompass 1-20 residues
  VARIANT 1161..1180: note = This region may encompass 1-20 residues
  VARIANT 1181..1200: note = This region may encompass 1-20 residues
  VARIANT 1201..1220: note = This region may encompass 1-20 residues
  VARIANT 1221..1240: note = This region may encompass 1-20 residues
  VARIANT 1241..1260: note = This region may encompass 1-20 residues
  VARIANT 1261..1280: note = This region may encompass 1-20 residues
  VARIANT 1281..1300: note = This region may encompass 1-20 residues
  VARIANT 1301..1320: note = This region may encompass 1-20 residues
  VARIANT 1321..1340: note = This region may encompass 1-20 residues
  VARIANT 1341..1360: note = This region may encompass 1-20 residues
  VARIANT 1361..1380: note = This region may encompass 1-20 residues
  VARIANT 1381..1400: note = This region may encompass 1-20 residues
  VARIANT 1401..1420: note = This region may encompass 1-20 residues
  VARIANT 1421..1440: note = This region may encompass 1-20 residues
  VARIANT 1441..1460: note = This region may encompass 1-20 residues
  VARIANT 1461..1480: note = This region may encompass 1-20 residues
  VARIANT 1481..1500: note = This region may encompass 1-20 residues
  VARIANT 1501..1520: note = This region may encompass 1-20 residues
  VARIANT 1521..1540: note = This region may encompass 1-20 residues
  VARIANT 1541..1560: note = This region may encompass 1-20 residues
  VARIANT 1561..1580: note = This region may encompass 1-20 residues
  VARIANT 1581..1600: note = This region may encompass 1-20 residues
  VARIANT 1601..1620: note = This region may encompass 1-20 residues
  VARIANT 1621..1640: note = This region may encompass 1-20 residues
  VARIANT 1641..1660: note = This region may encompass 1-20 residues
  VARIANT 1661..1680: note = This region may encompass 1-20 residues
  VARIANT 1681..1700: note = This region may encompass 1-20 residues
  VARIANT 1701..1720: note = This region may encompass 1-20 residues
  VARIANT 1721..1740: note = This region may encompass 1-20 residues
  VARIANT 1741..1760: note = This region may encompass 1-20 residues
  VARIANT 1761..1780: note = This region may encompass 1-20 residues
  VARIANT 1781..1800: note = This region may encompass 1-20 residues
  VARIANT 1801..1820: note = This region may encompass 1-20 residues
  VARIANT 1821..1840: note = This region may encompass 1-20 residues
  VARIANT 1841..1860: note = This region may encompass 1-20 residues
  VARIANT 1861..1880: note = This region may encompass 1-20 residues
  VARIANT 1881..1900: note = This region may encompass 1-20 residues
  VARIANT 1901..1920: note = This region may encompass 1-20 residues
  VARIANT 1921..1940: note = This region may encompass 1-20 residues
  VARIANT 1941..1960: note = This region may encompass 1-20 residues
  VARIANT 1961..1980: note = This region may encompass 1-20 residues
  VARIANT 1981..2000: note = This region may encompass 1-20 residues
  VARIANT 1..2000: note = This sequence may encompass 1-20 "(Gly)m(Ser)o(Gly)m(Ser)o(Gly)m" repeating units, wherein; m = 1 to 20, o = 1 to 20
  REGION 1..2000: note = See specification as filed for detailed description of substitutions and preferred embodiments
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGG

SEQ ID NO: 468 - protein, 480 aa, synthetic construct
  REGION 1..480: note = Description of Artificial Sequence: Synthetic polypeptide
  VARIANT 5..24: note = This region may encompass 1-20 residues
  VARIANT 29..48: note = This region may encompass 1-20 residues
  VARIANT 53..72: note = This region may encompass 1-20 residues
  VARIANT 77..96: note = This region may encompass 1-20 residues
  VARIANT 101..120: note = This region may encompass 1-20 residues
  VARIANT 125..144: note = This region may encompass 1-20 residues
  VARIANT 149..168: note = This region may encompass 1-20 residues
  VARIANT 173..192: note = This region may encompass 1-20 residues
  VARIANT 197..216: note = This region may encompass 1-20 residues
  VARIANT 221..240: note = This region may encompass 1-20 residues
  VARIANT 245..264: note = This region may encompass 1-20 residues
  VARIANT 269..288: note = This region may encompass 1-20 residues
  VARIANT 293..312: note = This region may encompass 1-20 residues
  VARIANT 317..336: note = This region may encompass 1-20 residues
  VARIANT 341..360: note = This region may encompass 1-20 residues
  VARIANT 365..384: note = This region may encompass 1-20 residues
  VARIANT 389..408: note = This region may encompass 1-20 residues
  VARIANT 413..432: note = This region may encompass 1-20 residues
  VARIANT 437..456: note = This region may encompass 1-20 residues
  VARIANT 461..480: note = This region may encompass 1-20 residues
  VARIANT 1..480: note = This sequence may encompass 1-20 "(Gly)(Ser)(Gly)(Gly)(Ser)m" repeating units, wherein m = 1 to 20
  REGION 1..480: note = See specification as filed for detailed description of substitutions and preferred embodiments
GSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSS

SEQ ID NO: 469 - protein, 480 aa, synthetic construct
  REGION 1..480: note = Description of Artificial Sequence: Synthetic polypeptide
  VARIANT 4..23: note = This region may encompass 1-20 residues
  VARIANT 28..47: note = This region may encompass 1-20 residues
  VARIANT 52..71: note = This region may encompass 1-20 residues
  VARIANT 76..95: note = This region may encompass 1-20 residues
  VARIANT 100..119: note = This region may encompass 1-20 residues
  VARIANT 124..143: note = This region may encompass 1-20 residues
  VARIANT 148..167: note = This region may encompass 1-20 residues
  VARIANT 172..191: note = This region may encompass 1-20 residues
  VARIANT 196..215: note = This region may encompass 1-20 residues
  VARIANT 220..239: note = This region may encompass 1-20 residues
  VARIANT 244..263: note = This region may encompass 1-20 residues
  VARIANT 268..287: note = This region may encompass 1-20 residues
  VARIANT 292..311: note = This region may encompass 1-20 residues
  VARIANT 316..335: note = This region may encompass 1-20 residues
  VARIANT 340..359: note = This region may encompass 1-20 residues
  VARIANT 364..383: note = This region may encompass 1-20 residues
  VARIANT 388..407: note = This region may encompass 1-20 residues
  VARIANT 412..431: note = This region may encompass 1-20 residues
  VARIANT 436..455: note = This region may encompass 1-20 residues
  VARIANT 460..479: note = This region may encompass 1-20 residues
  VARIANT 1..480: note = This sequence may encompass 1-20 "(Gly)(Ser)(Gly)(Ser)m(Gly)" repeating units, wherein m = 1 to 20
  REGION 1..480: note = See specification as filed for detailed description of substitutions and preferred embodiments
GSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSG

SEQ ID NO: 470 - protein, 460 aa, synthetic construct
  REGION 1..460: note = Description of Artificial Sequence: Synthetic polypeptide
  VARIANT 4..23: note = This region may encompass 1-20 residues
  VARIANT 27..46: note = This region may encompass 1-20 residues
  VARIANT 50..69: note = This region may encompass 1-20 residues
  VARIANT 73..92: note = This region may encompass 1-20 residues
  VARIANT 96..115: note = This region may encompass 1-20 residues
  VARIANT 119..138: note = This region may encompass 1-20 residues
  VARIANT 142..161: note = This region may encompass 1-20 residues
  VARIANT 165..184: note = This region may encompass 1-20 residues
  VARIANT 188..207: note = This region may encompass 1-20 residues
  VARIANT 211..230: note = This region may encompass 1-20 residues
  VARIANT 234..253: note = This region may encompass 1-20 residues
  VARIANT 257..276: note = This region may encompass 1-20 residues
  VARIANT 280..299: note = This region may encompass 1-20 residues
  VARIANT 303..322: note = This region may encompass 1-20 residues
  VARIANT 326..345: note = This region may encompass 1-20 residues
  VARIANT 349..368: note = This region may encompass 1-20 residues
  VARIANT 372..391: note = This region may encompass 1-20 residues
  VARIANT 395..414: note = This region may encompass 1-20 residues
  VARIANT 418..437: note = This region may encompass 1-20 residues
  VARIANT 441..460: note = This region may encompass 1-20 residues
  VARIANT 1..460: note = This sequence may encompass 1-20 "(Gly)(Gly)(Gly)(Ser)m" repeating units, wherein m = 1 to 20
  REGION 1..460: note = See specification as filed for detailed description of substitutions and preferred embodiments
GGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSS

SEQ ID NO: 471 - protein, 6 aa, synthetic construct
  REGION 1..6: note = Description of Artificial Sequence: Synthetic His tag
  VARIANT 1..6: note = This sequence may encompass 3-6 residues
HHHHHH

SEQ ID NO: 472 - protein, 6 aa, synthetic construct
  REGION 1..6: note = Description of Artificial Sequence: Synthetic 6xHis tag
HHHHHH

SEQ ID NO: 473 - protein, 8 aa, synthetic construct
  REGION 1..8: note = Description of Artificial Sequence: Synthetic 8xHis tag
HHHHHHHH